(19) United States
(12) Patent Application Publication
(10) Pub. No.: US 2026/0152742 A1
(43) Pub. Date: Jun. 4, 2026

(54) METHODS OF FORMING CIRCULARIZED RNA

(71) Applicants: Peking University, Beijing (CN); Beijing Changping Laboratory, Beijing (CN); Therorna Inc., Beijing (CN)

(72) Inventors: Wensheng WEI, Beijing (CN); Yong SHEN, Beijing (CN); Lu GAO, Beijing (CN)

(21) Appl. No.: 19/399,542

(22) Filed: Nov. 24, 2025

Related U.S. Application Data
(63) Continuation of application No. PCT/CN2024/094971, filed on May 23, 2024.

(30) Foreign Application Priority Data
May 23, 2023 (WO) PCT/CN2023/095856
Mar. 5, 2024 (WO) PCT/CN2024/080065

(51) Int. Cl. C12N 15/113; C12N 15/85

(52) U.S. Cl. CPC C12N 15/113; C12N 15/85; C12N 2310/124; C12N 2840/203

(57) ABSTRACT

The present application relates to methods of forming circularized RNA (circRNA) using intact Group I introns provided in cis or fragments of Group I introns provided in trans. The present application also relates to methods of forming circRNA using intact Group II introns provided in cis or fragments of Group II introns provided in trans.

### CROSS-REFERENCE TO RELATED APPLICATIONS

This application is a continuation of PCT International Application No. PCT/CN2024/094971, filed May 23, 2024, which claims the benefit of PCT International Application No. PCT/CN2023/095856, filed May 23, 2023, and of PCT International Application No. PCT/CN2024/080065, filed Mar. 5, 2024, the entire contents of each of which are hereby incorporated by reference herein.

### FIELD

The present application relates to methods of forming circularized RNA (circRNA) using intact Group I introns provided in cis or fragments of Group I introns provided in trans. The present application also relates to methods of forming circRNA using intact Group II introns provided in cis or fragments of Group II introns provided in trans.

### REFERENCE TO AN ELECTRONIC SEQUENCE LISTING

The contents of the electronic sequence listing (165392001701SEQLIST.xml; Size: 328,083 bytes; and Date of Creation: Nov. 21, 2025) is herein incorporated by reference in its entirety.

### BACKGROUND

Circular RNAs, also known as circularized RNAs (circRNAs), are covalently closed single-stranded RNA transcripts comprising a large class of non-coding RNAs. They are classically generated by a non-canonical RNA splicing event called backsplicing in eukaryotic cells (Zhang, X. O. et al. Complementary sequence-mediated exon circularization. Cell 159, 134-147, doi: 10.1016/j.cell.2014.09.001 (2014); Chen, L. L. The biogenesis and emerging roles of circular RNAs. Nat Rev Mol Cell Biol 17, 205-211, doi: 10.1038/nrm.2015.32 (2016); Kristensen, L. S. et al. The biogenesis, biology and characterization of circular RNAs. Nat Rev Genet 20, 675-691, doi: 10.1038/s41576-019-0158-7 (2019)). Some viral genomes happen to be circular RNAs, such as hepatitis D virus and plant viroids (Chen, Y. G. et al. N6-Methyladenosine Modification Controls Circular RNA Immunity. Mol Cell 76, 96-109 e109, doi: 10.1016/j.molcel.2019.07.016 (2019). In recent years, thousands of circRNAs have been identified in eukaryotes, including fungi, plants, insects, fish, and mammals via high-throughput RNA sequencing and circRNA-specific bioinformatics (Kristensen, L. S. et al. The biogenesis, biology and characterization of circular RNAs. Nat Rev Genet 20, 675-691, doi: 10.1038/s41576-019-0158-7 (2019). Unlike linear mRNA, circRNA is highly stable as its covalently closed ring structure protects it from exonuclease-mediated degradation (Micura, R. Cyclic oligoribonucleotides (RNA) by solid-phase synthesis. Chem-Eur J 5, 2077-2082 (1999); Muller, S. & Appel, B. In vitro circularization of RNA. RNA Biol 14, 1018-1027 (2017); Schindewolf, C., Braun, S. & Domdey, H. In vitro generation of a circular exon from a linear pre-mRNA transcript. Nucleic Acids Res 24, 1260-1266 (1996)).

RNA is increasingly being used as a therapeutic compound or as part of a therapeutic method. This includes use of different types of RNAs for gene silencing, including, for example, siRNA, miRNA, and gRNA. More recently, the development of RNA-based vaccines against SARS-COV2 has shown the potential for broad application of RNA-based vaccines. Using circRNAs in these RNA-based therapeutics likely presents several advantages over the conventional use of linear RNAs. For example, circRNA is more stable than linear RNA because it is more resistant to enzymatic catalysis. Furthermore, circRNAs do not require nucleotide modifications, while canonical linear RNA agents incorporate nucleotide modifications for improved stability.

Despite the absence of a cap structure for translation initiation, circRNA can be engineered to initiate translation efficiently using internal ribosomal entry site (IRES) or IRES-like elements (see, e.g., Wesselhoeft, R. A., Kowalski, P. S. & Anderson, D. G. Engineering circular RNA for potent and stable translation in eukaryotic cells. Nat Commun 9, 2629 (2018)). The use of IRES in circRNA has achieved effective protein expression in cells and animals, even in non-human primates (see, e.g., Qu, L. et al. Circular RNA vaccines against SARS-COV-2 and emerging variants. Cell 185, 1728-1744 e1716 (2022)). Besides, through strategically engineering of IRES elements and other regulatory components within circRNAs, enhanced protein expression has been achieved (Chen, R. et al. Engineering circular RNA for enhanced protein production. Nat Biotechnol 41, 262-272 (2023)). As the relatively large size of IRES and protein coding sequence, efficiently and conveniently achieving in vitro circularization remains a challenge. Existing approaches to RNA circularization encompass chemical synthesis and ligation (see, e.g., Micura, R. Cyclic oligoribonucleotides (RNA) by solid-phase synthesis. Chem-Eur J 5, 2077-2082 (1999)), ligases-mediated circularization (see, e.g., Qu, L. et al. Circular RNA vaccines against SARS-COV-2 and emerging variants. Cell 185, 1728-1744 e1716 (2022)), and self-splicing ribozymes-mediated circularization (see, e.g., Wesselhoeft, R. A., Kowalski, P. S. & Anderson, D. G. Engineering circular RNA for potent and stable translation in eukaryotic cells. Nat Commun 9, 2629 (2018)), each with its inherent limitations (see, e.g. Petkovic, S. & Muller, S. RNA circularization strategies in vivo and in vitro. Nucleic Acids Res 43, 2454-2465 (2015)). The chemical synthesis and ligation method is constrained by the size of RNA, allowing efficient generation only in small circRNAs. Circularization facilitated by ligases faces a common hindrance. While it exhibits remarkable efficacy in producing circular RNAs, efficiently generating circRNAs spanning kilobases presents a persistent challenge. Additionally, this process introduces extra proteins or splints, posing further complications for circRNA purification. The similarity in size between unligated RNA and circRNA also adds to the intricacies of this purification process.

Currently, ribozymes-mediated RNA circularization has been developed for self-splicing group I and group II introns. For group I introns, the Anabaena sp. strain PCC 7120 (Ana) intron has been engineered to produce circRNAs ranging in length to kilobases by PIE (Wesselhoeft, R. A., Kowalski, P. S. & Anderson, D. G. Engineering circular RNA for potent and stable translation in eukaryotic cells. Nat Commun 9, 2629 (2018)), and has been widely adopted for circRNA applications (see, e.g., Qu, L. et al. Circular RNA vaccines against SARS-COV-2 and emerging variants. Cell 185, 1728-1744 e1716 (2022); Li, H. et al. Circular RNA cancer vaccines drive immunity in hard-to-treat malignancies. Theranostics 12, 6422-6436 (2022)). Additionally, the trans-splicing activity of Tetrahymena thermophila intron has also been employed for RNA circularization (see, e.g., Cui, J. et al. A precise and efficient circular RNA synthesis system based on a ribozyme derived from Tetrahymena thermophila. Nucleic Acids Res 51, e78 (2023)), which is limited in specific intron with trans-splicing activity. Besides, the efficiency is relatively low compared to PIE. Group II introns can also function in RNA circularization through PIE (Chen, C. et al. A flexible, efficient, and scalable platform to produce circular RNAs as new therapeutics. BioRxiv (2022)). Despite the moderate efficiency of circularization using the group II intron approach, there is a potential for generating circRNA without the inclusion of foreign sequences. This absence of foreign sequences is regarded as a critical factor for the immunogenicity of circRNA (Liu, C. X. et al. RNA circles with minimized immunogenicity as potent PKR inhibitors. Mol Cell 82, 420-434 e426 (2022)).

Among these methods, PIE applied in Ana group I intron is the most commonly used, achieving highly efficient RNA circularization and extending the limit of RNA circularization to approximately 5,000 nucleotides (Wesselhoeft, R. A., Kowalski, P. S. & Anderson, D. G. Engineering circular RNA for potent and stable translation in eukaryotic cells. Nat Commun 9, 2629 (2018)). However, PIE faces challenges, including the necessity for intron splitting, which restricts its applicability to certain specific introns (see, e.g., Wesselhoeft, R. A., Kowalski, P. S. & Anderson, D. G. Engineering circular RNA for potent and stable translation in eukaryotic cells. Nat Commun 9, 2629 (2018)). Furthermore, the byproducts generated in PIE are challenging to remove conveniently, and the generation of large circRNAs is not achieved effectively by PIE.

The most common current methods of forming circRNAs require a linear RNA precursor that has Group I intron fragments on either end, which limits the extent to which the sequence and structure of the linear RNA and resulting circRNA can be varied. There is thus a need for methods of producing circRNAs from linear RNA that can accommodate a wider variety of sequences and structures.

### BRIEF SUMMARY

The present application provides methods of forming circRNA.

One aspect of the present application provides a linear RNA precursor, comprising from the 5′ end to the 3′ end: (a) a catalytic intron; (b) a 3′ exon sequence; (c) an effector RNA sequence, and (d) a 5′ exon sequence, wherein the catalytic intron is capable of splicing the 3′ exon sequence and the 5′ exon sequence from the linear RNA precursor, thereby forming a circular RNA (“circRNA”) comprising the effector RNA.

One aspect of the present application provides a RNA circularization system, comprising: (i) a linear RNA precursor from the 5′ end to the 3′ end, comprising (a) a 3′ catalytic intron fragment; (b) a 3′ exon sequence; (c) an effector RNA sequence, and (d) a 5′ exon sequence, and (ii) a free 5′ catalytic intron fragment, wherein the 3′ catalytic fragment and the 5′ catalytic intron fragment are capable of splicing the 3′ exon sequence and the 5′ exon sequence from the linear RNA precursor, thereby forming a circular RNA (“circRNA”) comprising the effector RNA. In some embodiments, the 5′ intron fragment and the linear RNA precursor are present in a molar ratio of at least about 2:1, respectively.

In some embodiments, the catalytic intron is a catalytic Group I intron. In some embodiments, the catalytic Group I intron is derived from a naturally occurring intron selected from the group consisting of: a member of the IC3 family of Group I introns and a member of the IE2 family of Group I introns.

In some embodiments, the catalytic intron is a catalytic Group II intron. In some embodiments, the catalytic Group II intron is derived from a naturally occurring intron of a species selected from the group consisting of: Bacillus thuringiensis, Clostridium perfringens, Anoxybacillus pushchinoensis, Desulforamulus ferrireducens, Bacillus smithii, and Oceanobacillus iheyensis.

In some embodiments, the 3′ exon sequence or 5′ exon sequence is no more than about 60 nucleotides long. In some embodiments, the 3′ exon sequence or the 5′ exon sequence is no more than about 10 nucleotides long. In some embodiments, the catalytic intron comprises a heterologous sequence. In some embodiments, the catalytic intron, the 3′ catalytic intron fragment, and/or the 5′ catalytic intron fragment comprise a heterologous sequence. In some embodiments, the heterologous sequence is inserted in a loop region of the catalytic intron. In some embodiments, the heterologous sequence is inserted in a stem region of the catalytic intron. In some embodiments, the heterologous sequence is inserted in a loop region of the catalytic intron, the 3′ catalytic intron fragment, or the 5′ catalytic intron fragment. In some embodiments, the heterologous sequence is inserted in a stem region of the catalytic intron, the 3′ catalytic intron fragment, or the 5′ catalytic intron fragment.

In some embodiments, the effector RNA sequence comprises a coding RNA sequence. In some embodiments, the coding RNA sequence encodes a therapeutic polypeptide. In some embodiments, the therapeutic polypeptide is selected from the group consisting of an antigenic polypeptide, a functional protein, a receptor protein, and a targeting protein. In some embodiments, the RNA precursor further comprises a Kozak sequence, an internal ribosomal entry site (IRES) sequence, or a portion thereof operably linked to the coding RNA sequence. In some embodiments, the effector RNA sequence is a sequence of a non-coding RNA selected from the group consisting of a guide RNA (gRNA), a deaminase-recruiting RNA (dRNA), a siRNA, a miRNA, a shRNA, and a long intervening non-coding (line) RNA. In some embodiments, the effector RNA sequence is about 50 to about 5000 nucleotides (nt) long.

In some embodiments, the catalytic intron comprises a heterologous sequence that tunes the catalytic activity of the catalytic. In some embodiments, the 3′ catalytic intron fragment comprises a heterologous sequence that tunes the catalytic activity of the 3′ catalytic intron fragment.

Another aspect of the present application provides DNA construct comprising a coding DNA sequence encoding the linear RNA precursor of any one of the preceding embodiments. In some embodiments, the DNA construct further comprises a promoter operably linked to the coding DNA sequence. In some embodiments, the promoter is a T7 promoter. In some embodiments, the construct is a viral vector or a plasmid.

Another aspect of the present application provides a method of preparing a circRNA, comprising a) providing the linear RNA precursor of any one of the preceding embodiments; and b) activating the catalytic intron in the linear RNA precursor, wherein the activation of the catalytic intron results in splicing of the 3′ exon sequence and the 5′ exon sequence from the linear RNA precursor, thereby forming the circRNA comprising the effector RNA.

Another aspect of the present application provides a method of preparing a circRNA, comprising a) providing the RNA circularization system of any one of the preceding embodiments; and b) activating the 3′ catalytic intron fragment in the linear RNA precursor and the free 5′ catalytic intron fragment, wherein the activation of the catalytic intron results in splicing of the 3′ exon sequence and the 5′ exon sequence from the linear RNA precursor, thereby forming the circRNA comprising the effector RNA. In some embodiments, the linear RNA precursor and the free 5′ intron fragment are provided simultaneously.

In some embodiments, the linear RNA precursor is provided by in vitro transcription from a DNA construct encoding the linear RNA precursor. In some embodiments, the activation of the catalytic intron comprises incubating the linear RNA precursor in a reaction medium. In some embodiments, the activation of the 3′ catalytic intron fragment and the 5′ intron fragment comprises incubating the linear RNA precursor and the free 5′ catalytic intron fragment in a reaction medium. In some embodiments, the reaction medium comprises a divalent metal ion. In some embodiments, the divalent metal ion is Mg²⁺. In some embodiments, the reaction medium comprises spermidine. In some embodiments, the incubation is carried out at a temperature of about 30-60° C. In some embodiments, the method further comprises isolating the circRNA. In some embodiments, the linear RNA precursor is provided by introducing a linear RNA precursor of any one of the preceding embodiments or a DNA construct encoding the linear RNA precursor to an individual. In some embodiments, the RNA circularization system is provided by introducing a RNA circularization system of any one of the preceding embodiments or a DNA construct encoding the linear RNA precursor and/or the free 5′ intron fragment to an individual.

In some embodiments, the catalytic intron comprises a heterologous sequence that tunes the catalytic activity of the catalytic intron. In some embodiments, the 3′ catalytic intron fragment comprises a heterologous sequence that tunes the catalytic activity of the 3′ catalytic intron fragment.

Another aspect of the present application provides a circular RNA prepared using the method of any one of the preceding embodiments.

In some embodiments, the circular RNA comprises a short scar sequence and/or a hidden scar sequence. In some embodiments, the circular RNA exhibits reduced immunogenicity compared to a corresponding circular RNA comprising a longer scar sequence and/or wherein the circular RNA exhibits reduced immunogenicity compared to a corresponding circular RNA comprising a scar sequence that is not hidden. In some embodiments, the short scar sequence or the hidden scar sequence is in an IRES sequence or in a UTR sequence. In some embodiments, the scar sequence comprises an NNUA motif. In some embodiments, the 3′ exon sequence comprises a 3′ A base, and wherein the 5′ exon sequence comprises a 5′ NNU motif. In some embodiments, a dephosphorylation step is performed after the formation of the circRNA. In some embodiments, the circular RNA exhibits reduced immunogenicity compared to a corresponding circular RNA comprising a scar sequence.

In some embodiments, the activating occurs in a buffer that lacks Na⁺, that lacks K⁺, and/or that lacks other monovalent cations. In some embodiments, the splicing occurs in a buffer comprising less than 1 mM Mg²⁺. In some embodiments, the splicing occurs in a buffer comprising less than 0.5 mM Mg²⁺.

### BRIEF DESCRIPTION OF THE DRAWINGS

FIGS. 1A-1I show schematics and data demonstrating Group I intron-mediated RNA circularization with trans splicing. FIG. 1A shows schematics of Group I intron autocatalysis in the context of (left) a natural genomic context and (right) the traditional PIE circularization method mediated by a Group I intron. Each linear segment represents a piece of linear RNA. A color key is shown to the far left in grayscale shading, and applies to all Figures unless otherwise noted. Exon sequences are shaded in black. Intron sequences are shaded in white. The vertical dotted line on the left panel shows roughly where the 5′ end of the intron meets the 3′ end of the intron. These 5′ and 3′ ends then correspond to the 5′ and 3′ intron fragments described in the right panel and in FIG. 1B. A Gene of Interest (GOI) is shown in dark gray. The two-step reaction proceeds from top to bottom of each panel, with the first step indicated on the top of each panel, and the second step indicated on the bottom. G-indicates the presence of a single G nucleotide at the indicated 5′ end. G-OH indicates the 3′ hydroxyl group of a guanosine nucleotide engaging in a transesterification reaction at the 5′ splice site, resulting in excision of the 5′ intron half. The piece of RNA labeled “Intermediate” represents the intermediate formed during the PIE process. A circRNA and two free intron fragments (which are neither covalently attached to each other nor to the circRNA) are generated from the PIE method, shown at the bottom right. FIG. 1B shows a schematic of circularization by trans splicing of a Group I intron for the data presented in FIGS. 1C-1H. The RNA segments are displayed as in FIG. 1A. The label “intermediate” indicates the linear RNA precursor starting point of the trans splicing method. At top are a linear 1,820-nt linear RNA precursor and a 155-nt free 5′ catalytic Group I intron fragment. The linear RNA precursor for trans splicing had, from its 5′ to 3′ end, a homology arm sequence (light gray), a 3′ catalytic Group I intron fragment (white), a 3′ exon sequence (black), a homology arm sequence (light gray), an effector RNA sequence comprising a GOI (dark gray), a homology arm sequence (light gray), and a 5′ exon sequence (black). The free 5′ catalytic Group I intron fragment had, from 5′ to 3′, a 5′-terminal G nucleotide, a 5′ intron fragment, and a homology arm. The products of trans splicing were a 165-nt 3′ intron fragment with a homology arm sequence on its 5′ end, a 1,655-nt circRNA, and the 155-nt free 5′ catalytic Group I intron fragment. FIG. 1C shows an agarose gel RNA electrophoresis image showing circRNA generation by trans splicing under different conditions: with GTP and 1× buffer (first four lanes on the left), with 1/10× buffer and GTP (middle four lanes), with ½× buffer and GTP (right-most four lanes), each of which were tested with varying ratios (0, 1, 5, and 25) of the free 5′ intron fragment to the “intermediate” linear RNA precursor. Reference nucleotide lengths are depicted along the right side. The reactions were allowed to run for 15 minutes at 50 degrees C. FIG. 1D shows an agarose gel RNA electrophoresis image showing the effect of reaction time on circularization efficiency. The lanes from left to right represent reactions that were allowed to run for 0, 0.25, 1, 4, 8, or 16 hours, respectively, at 50 degrees C., using a 5:1 molar ratio of free 5′ intron fragments to “intermediate” linear RNA precursors. Reference nucleotide lengths are depicted along the right side. FIG. 1E shows the same reaction products from the right-most 5 lanes of FIG. 1D (representing 0.25, 1, 4, 8, or 16 hour reaction times, respectively), but in which the RNA products were treated with RNase R (right-most 5 columns) to verify the generation of circRNA. Negative controls in which samples from the same 5 reactions were treated with RNase R buffer alone are shown in the left-most 5 columns. Reference nucleotide lengths are depicted along the right side. FIG. 1F shows an agarose gel RNA electrophoresis image showing the effect of increasing the molar ratio of free 5′ intron fragments to “intermediate” linear RNA precursors. The left-most five lanes show reactions with ratios of 0, 1.6, 8, 40, and 200, respectively, in 1/10× buffer with GTP, with the reactions allowed to run for 16 hours at 50 degrees C. Results of a control reaction that was run with buffer and GTP at 37 degrees C. for 16 hours are shown in the right-most lane. Reference nucleotide lengths are depicted along the right side. FIG. 1G shows plots depicting EGFP expression from the RNA products produced in the reactions described in FIG. 1F transfected into HEK293T cells, which partially represents RNA circularization. The y-axis on the left plot depicts the % of EGFP positive cells from each sample; the y-axis on the right plot depicts mean fluorescence intensity (MFI) of EGFP from each sample. The ratios used in each sample are displayed along the x-axis, corresponding to the ratios described in FIG. 1F. Data are shown as the mean±S. D (n=2). FIG. 1H shows an agarose gel RNA electrophoresis image and schematics showing the effect of an additional G nucleotide at the 5′ end of the 5′ intron fragment on the effectiveness of the trans splicing method. A schematic of the 5′ intron fragment used in each reaction is displayed above each gel, with the shading corresponding to that described in FIG. 1A. Length ladders were run in the far left, middle, and far right lanes, with reference nucleotide lengths depicted along the right side. The reactions were allowed to run for 0.5, 1, 2, 4, or 8 hours as indicated above each reaction lane, with 1/10× buffer at 50 degrees C. FIG. 1I shows a schematic diagram of the interactions believed to mediate trans splicing.

FIGS. 2A-2M show schematics and data demonstrating Group I intron-mediated RNA circularization with cis splicing. The schematics are shaded as described in FIG. 1A unless otherwise noted. “Exon1” is also referred to herein as the “5′ exon” and “Exon2” is also referred to herein as the “3′ exon”. FIG. 2A shows a schematic diagram of Group I intron mediated circularization with the cis splicing method. FIG. 2B shows an agarose gel RNA electrophoresis image (left) showing the effect of an additional G at the 5′ of the RNA in the cis splicing method. Schematic diagrams of the constructs tested are shown on the right. A marker lane with molecular reference weights is shown along the left in the “M” column. “Ana” indicates the Anabaena Group I intron sequence provided in SEQ ID NO: 9 was used. 1G indicates the presence of a single G nucleotide on the 5′ end of the Ana intron (SEQ ID NO: 7). 2G indicates the presence of two G nucleotides on the 5′ end of the Ana intron (SEQ ID NO: 8). FIG. 2C shows an agarose gel RNA electrophoresis image and schematics showing circRNA generation by cis splicing of linear RNA precursors comprising different group I introns, each with at least two Gs on the 5′ end: Ana (SEQ ID NO: 8; intron), Ctu (SEQ ID NO: 13), Par (SEQ ID NO: 14), and Gvi (SEQ ID NO: 15). Schematics depicting the linear RNA constructs tested are shown above the gel. The lanes labeled “0 nt” had no extension on the 3′ end. The lanes labeled “7 nt” had a 7-nucleotide extension (SEQ ID NO: 12; shown in white on the far right end of the schematic at the top right) added to the 3′ end of the intron sequence to test the sequence restriction in the exon-intron boundary. The length of the spliced intron was 320, 477, 411, and 350 nucleotides for Ana, Ctu, Par, and Gvi, respectively. Marker lanes are indicated as described in FIG. 2B. FIG. 2D shows plots depicting green fluorescent protein (EGFP) expression data in HEK293T cells that were transfected with the RNA from FIG. 2C. White shading depicts data from constructs comprising the Ana intron; black shading depicts data from constructs comprising the Ctu intron; dark gray shading depicts data from constructs comprising the Par intron; and light gray shading depicts data from constructs comprising the Gvi intron. The y-axes indicate the % of EGFP positive cells (left) or the mean fluorescence intensity (MFI, right). The x-axis indicates the length of the 3′ extension described in FIG. 2C. Data are shown as the mean±S.D (n=3). FIG. 2E shows an agarose gel RNA electrophoresis image and schematics showing circRNA generation by cis splicing using RNA that were generated from the RNA shown in FIG. 2C and incubated at 50 degrees C. at 16 hours with Mg²⁺. The gel and schematics are labeled as described in FIG. 2C unless otherwise noted. “−” indicates a 0-nt 3′ extension. “+” indicates a 7-nt 3′ extension. The Ana constructs had a 16-nt Exon1 sequence and a 51-nt Exon 2 sequence; the other constructs had 15-nt Exon1 and Exon2 sequences. Exon 1 is also referred to herein as the “5′ exon”, and Exon 2 is also referred to herein as the “3′ exon”. FIG. 2F shows plots depicting EGFP expression data in HEK293T cells that were transfected with the RNA from FIG. 2E. The plots are labeled and shaded as described for FIG. 2D unless otherwise noted. The plots on the left are from samples that were untreated after the in vitro transcription (IVT) reactions. The plots on the right are from samples that were incubated at 50 degrees C. at 16 hours with Mg²⁺. Data are shown as the mean±S.D (n=2). FIG. 2G shows an agarose gel RNA electrophoresis image and schematics showing the effect of internal homology arm sequences (light gray) on the efficiency of circularization. Hetero: Irrelevant arm. Homo: Homology arm. Short: 9-nt. Long: 19-nt. IRES 3 and 8 indicate different split sites in the IRES, wherein split site 3 splits the IRES into a first part comprising the sequence set forth in SEQ ID NO: 28 and a second part comprising the sequence set forth in SEQ ID NO: 29, and split site 8 splits the IRES into a first part comprising the sequence set forth in SEQ ID NO: 26 and a second part comprising the sequence set forth in SEQ ID NO: 27. The gel and schematics are labeled as described in FIG. 2C unless otherwise noted. FIG. 2H shows an agarose gel RNA electrophoresis image and schematics showing the effect of exon length on the efficiency of circularization. The gel is labeled as in FIG. 2E unless otherwise noted. Co indicates a construct comprising the Co intron (SEQ ID NO: 32). The lengths in nucleotides of the Exon1 and Exon2 sequences used in each construct are indicated above each lane. FIG. 2I shows an agarose gel RNA electrophoresis image and schematics showing the effect of 3′ end single nucleotide variation on splicing efficiency. The gel and schematics are labeled as described in FIG. 2C. The base at the 3′ end (U, A, C, or G) of each construct is indicated above each lane. FIG. 2J shows an agarose gel RNA electrophoresis image and schematics showing the effect on splicing efficiency of A insertions of different lengths (15, 25, or 35 As) in specific sites (A insertion site 1 or 2) in the Group I intron according to the schematic depicted at the top, in which light gray indicates the position of the A insertion. FIG. 2K shows an agarose gel RNA electrophoresis image and schematics showing results of RNA purification from the A insertion site 2 constructs described in FIG. 2J using Oligo dT beads. In: Input; SN: supernatant; B: beads. The gels and the schematics of the products after the reaction are labeled according to FIG. 2J. FIG. 2L shows an agarose gel RNA electrophoresis image and schematics showing that at least several additional Group I introns (Pob, Tpa, Pte, and Cpro) in the IE2 family, in addition to Co, Gvi, and Par, have the ability to mediate RNA circularization. Self-splicing was allowed to occur in constructs according to the schematic shown at top, with white indicating the Group I intron sequence, black indicating a 15 nucleotide exon sequence, and gray indicating a spacer sequence. FIG. 2M shows an agarose gel RNA electrophoresis image showing results of a comparison between the cis splicing method (left 5 lanes) and the canonical PIE method (right 5 lanes), each using the Ana intron, in which the time of the IVT was varied between 1, 2, 4, 8, and 16 hours, with the results of each shown in a different lane as indicated above the gel. A marker lane with molecular reference weights is shown along the right in the “M” column.

FIGS. 3A-3L show RNA circularization through trans splicing, including additional exemplary schematics of RNA circularization. FIG. 3A shows a schematic of PIE method for RNA circularization. “RNA Payload” is also referred to herein as “gene of interest” or “GOI”. FIG. 3B shows a schematic of trans splicing for RNA circularization. FIG. 3C shows the evaluation of the 5′ intron (absent on the left, present on the right), GTP, and Mg²⁺ requirements for trans splicing. Each lane is labeled with “+” or without “−” the inclusion of GTP and/or Mg²⁺. “M” represents a molecular ladder. FIG. 3D shows a schematic of reverse transcription and PCR analysis of circRNA, followed by Sanger sequencing of the PCR product. FIG. 3E shows cell images taken 24 hours post-transfection with linear precursor or circular RNA. Scale bars are in the bottom right corner of each image. FIG. 3F shows fluorescence-activated cell sorting (FACS) results of measuring EGFP expression levels. Data represented as mean #S.D. (n=3), with each dot denoting a biological replicate. Unpaired two-sided Student's t test was used for comparisons. These results are reported as mean EGFP expression (in %, vertical axis of left panel) and mean fluorescence intensity (MFI) of the EGFP (vertical axis of right panel). FIG. 3G shows a schematic for a RNase R assay for circRNA validation. FIG. 3H shows agarose gel electrophoresis results for a linear precursor and circRNA treated with RNase R. “M” represents a molecular ladder. FIG. 3I shows a schematic of a Poly(A) assay for circRNA validation. FIG. 3J shows agarose gel electrophoresis results for a linear precursor and circRNA treated with poly(A) polymerase. FIG. 3K shows a schematic of the trans splicing method for RNA circularization using a high ratio of 5′ intron to intermediate. FIG. 3L shows agarose gel electrophoresis result of intermediate reacting with different ratios of 5′ introns.

FIGS. 4A-4I show RNA circularization through cis splicing, including additional exemplary schematics of RNA circularization. FIG. 4A shows the RNA structure in cis splicing-mediated RNA circularization. “Payload” is also referred to herein as “gene of interest” or “GOI”. FIG. 4B shows a bar plot depicting results of agarose gel electrophoresis, demonstrating the impact of deleting the homology arm within the intron on circularization efficiency. FIG. 4C shows a schematic representation of cis splicing for RNA circularization. “Payload” is also referred to herein as “gene of interest” or “GOI”. FIG. 4D shows agarose gel results depicting the GTP and Mg²⁺ requirements of cis splicing. An inactive group I intron was generated by mutating the first 20 bases of the wild-type group I intron. “M” represents a molecular ladder. FIG. 4E shows Sanger sequencing of the PCR product resulting from circRNA reverse transcription using the indicated primer from FIG. 3D. FIG. 4F shows cell images taken at 24 hours post-transfection with linear precursor or circRNA. Scale bars are in the bottom right corner of each photo. FIG. 4G shows results of measuring EGFP expression levels (in %, vertical axis of left panel) and mean fluorescence intensity (MFI) of the EGFP (vertical axis of right panel). For FIGS. 4B and 4G, data are shown as mean±S.D. (n=3), with each dot representing a biological replicate. Unpaired two-sided Student's t-test used for comparison. FIG. 4H shows agarose gel electrophoresis results of linear precursor and circRNA treated with RNase R. FIG. 4I shows agarose gel electrophoresis results of linear precursor and circRNA treated with poly(A) polymerase. Schematic diagram of the DNA templates used in cis splicing. For FIGS. 4F-4I, “Linear” represents linear RNA generated using an inactive group I intron.

FIGS. 5A-5F show the impact of the IVT template on cis splicing. FIG. 5A shows a schematic of a cis splicing precursor and its processing during in vitro transcription (symbolized by the black arrow) into IVT products containing either one (bottom left) or two (bottom right) G's at the 5′ end. In the precursor, G's are flanked on the 5′ end by a T7 promoter, which is shown as a boxed corresponding nucleotide sequence. FIG. 5B shows the impact of the quantity of G's on RNA yield. Data presented as mean #S.D. (n=6). FIG. 5C shows agarose gel electrophoresis results of 1G (left) and 2G (right) samples' circularization efficiency. FIG. 5D shows a bar plot comparing circularization efficiency between samples with 1G and samples with 2G. Data shown as mean±S.D. (n=3). FIG. 5E shows a schematic of the three types of DNA templates used in cis splicing. FIG. 5F shows agarose gel electrophoresis results of testing different IVT templates on circularization efficiency. The contents of lanes labeled “1”, “2”, and “3” correspond to the templates visualized in FIG. 5E. “M” represents a molecular ladder. In FIGS. 5B and 5D, each dot symbol represents a biological replicate. Unpaired two-sided Student's t-test conducted for comparisons as indicated. In FIGS. 5C and 5F, circularization efficiency is indicated below each lane.

FIGS. 6A-6L show the workflow and results of optimization of cis splicing. FIG. 6A shows a schematic of the cis splicing construct. “3′ Exon” and “5′ Exon” are also referred to herein as “Exon 2” and “Exon 1”. FIG. 6B shows a workflow for preparing samples for evaluating circularization rate and efficiency. FIGS. 6C-6H show the results on circularization efficiency of modifying various DNA template elements. FIGS. 6C-6E show agarose gel electrophoresis results of testing various exon lengths. FIG. 6C shows tests of several 5′ exon lengths, the lengths of which label their respective lanes. FIG. 6D shows tests of several 3′ exon lengths, the lengths of which label their respective lanes. FIG. 6E shows tests of 5′ and 3′ exon lengths in tandem, the lengths of which label their respective lanes. FIG. 6F shows agarose gel electrophoresis results of varying GC content levels in the homology arm, the tested GC nucleotide numbers of which are used to label their respective lanes. FIG. 6G shows agarose gel electrophoresis results of testing several homology arm lengths, the nucleotide numbers of which are used to label their respective lanes. FIG. 6H shows agarose gel electrophoresis results of testing several flexible region locations, the nucleotide positioning of which along the homology arm are used to label their respective lanes for Location 1 (left) and Location 2 (right). FIG. 6I shows a schematic of the final version of the cis splicing construct. FIG. 6J shows agarose gel electrophoresis results of circularization efficiency for the original (“C”) and final versions (“V1” and “V2”) of the cis splicing construct under conditions listed beneath each panel. “Gluc”=Gaussia Luciferase. FIG. 6K shows, for transfected cells and an untransfected control, FACS results of EGFP expression level (in %, vertical axis of left panel) and mean fluorescence intensity (MFI) of the EGFP (vertical axis of right panel). FIG. 6L shows microplate reader results of Gaussia Luciferase (GLuc) activity (plotted along the vertical axis) measured in transfected cells and an untransfected control. For FIGS. 6K-6L, the horizontal axis plots the untransfected control (“Untransfected”), transfected initial cis splicing construct (“Ctrl”), and final versions of the cis splicing constructs (“V1” and “V2”); data presented as mean±S.D. (n=3); “ns” represents no significant difference between means; each dot represents a biological replicate; and unpaired two-sided Student's t-tests were conducted for comparisons as indicated. For FIGS. 6C-6G and 6J, the DNA template used for IVT was PCR product with 2′OMe modification; for FIG. 6H, the DNA template was linearized plasmid. For FIGS. 6C-6H, “M” represents a molecular ladder, and the heat treatment used is indicated under each gel display.

FIGS. 7A-7G show advantages of cis splicing over PIE. FIG. 7A shows agarose gel electrophoresis results of RNA circularization efficiency of cis splicing (left) and PIE (right) within IVT reactions. FIG. 7B shows agarose gel electrophoresis results of RNA circularization efficiency at 55° C. for multiple reaction times, the duration of which in minutes label each respective lane. FIGS. 7C and 7D show RNA circularization efficiency of cis splicing (FIG. 7C) and PIE (FIG. 7D) when treated with concentrations of MgCl₂, with concentrations labeling each respective lane. FIG. 7E shows a schematic of RNase R sensitivity of RNAs in cis splicing and PIE, with 5′ and 3′ ends of RNA labeled. FIG. 7F shows agarose gel electrophoresis results of RNA precursors of cis splicing (left) and PIE (right), treated with varying RNase R durations (in minutes, labeling each lane). FIG. 7G shows agarose gel electrophoresis results of IVT 2-hour RNA product of cis splicing, treated with varying RNase R durations (in minutes, labeling each lane). For FIGS. 7A-7D and 7F-7G, “M” represents a molecular ladder. For FIGS. 7C-7D, the reaction was conducted at 55° C. in 50 mM HEPES (pH=6.8) and 150 mM NaCl with varying Mg²⁺ concentrations. In FIGS. 7A-7G, internal ribosome entry site-based EGFP (IRES-EGFP) was employed as the payload of circRNA.

FIGS. 8A-8E show oligo(dT) beads-mediated circRNA purification of cis splicing. FIG. 8A shows a schematic of poly(A) insertion (“An”) into an intron in cis splicing. “Payload” is also referred to herein as “gene of interest” or “GOI”. FIG. 8B shows agarose gel electrophoresis results of RNA circularization efficiency under insertion of varying numbers of inserted adenines, numbers of which label respective lanes. FIG. 8C shows a schematic of a workflow for oligo(dT) beads-mediated circRNA purification. FIG. 8D shows agarose gel electrophoresis results of supernatant RNA circularization efficiency under insertion of varying numbers of inserted adenines (numbered in the topmost row above the lanes), either with (“+”) oligo(dT) beads for one round of incubation or without (“−”) oligo(dT) beads or incubation. FIG. 8E shows agarose gel electrophoresis results of supernatant RNA circularization efficiency under insertion of no adenines (“A₀”), 34 adenines (“A₃₄”), or 75 adenines (“A₇₅”) and varying rounds of bead purification, the number of which label each respective lane.

FIGS. 9A-9G show other group I introns for RNA circularization using cis splicing. FIG. 9A shows a schematic of the reporter used to evaluate group I intron splicing activity in vitro. FIG. 9B shows agarose gel electrophoresis results of intron splicing and exon ligation mediated by functional group I introns (each labeling a lane) in vitro. FIG. 9C shows a schematic of group I introns in cis splicing, within which Group I introns and corresponded exons could be changed. FIG. 9D shows agarose gel electrophoresis results of putative circRNA generated via mediated-intron splicing of different group I introns, either after IVT (top panel) or with an additional treatment of 55° C. (bottom panel). FIG. 9E shows agarose gel electrophoresis results of putative circRNA generated via different group I introns (each labeling its respective lane) treated with poly(A) polymerase (“+”), or without poly(A) polymerase treatment (“−”). FIG. 9F shows Sanger sequencing results of PCR products (using the primer of FIG. 3D) of varying Group I introns' resultant circRNA reverse transcribed. The sequences to the left of the precise ligation point represent the 5′ exon, and the sequences to the right of the precise ligation represent the 3′ exon. FIG. 9G shows, for transfected cells and an untransfected control (“Ctrl”), FACS results of EGFP expression level (in %, vertical axis of left panel, “EGFP positive (%)”) and mean fluorescence intensity (MFI) of the EGFP (vertical axis of right panel). Data are presented as the mean #S.D. (n=3). Each dot represents a biological replicate. Unpaired two-sided Student's t-test was performed for comparison as indicated. For FIGS. 9B and 9D-9G, “Ctu”=Closterium tumidum; “Rar”=Rasamsonia argillacea; “Pob”=Penicillium oblatum; “Tpa”=Trichocoma paradoxa; “Co”=Cordyceps sp. 97009; “Pte”=Paecilomyces tenuipes; “Cpro”=Polycephalomyces prolificus; “Gvi”=Talaromyces viridulus; and “Ana”=Anabaena sp. strain PC (7120.

FIGS. 10A-10E show the impact of sequence optimization on protein expression in cis splicing. FIG. 10A shows the configuration of sample lengths used in FIG. 10B. Each row from “5′ Exon” to “GC content of homology arm (n)” corresponds to a component tested in one lane of FIGS. 6C-6H, with each sample in FIG. 10A possessing one of the component lengths, location, or GC contents previously tested in FIG. 6. Each sample (1-7) possesses all the conditions listed in the same column's rows beneath it. FIG. 10B shows FACS results demonstrating the expression level of EGFP (in %, vertical axis of left panel) and mean fluorescence intensity (MFI) of the EGFP (vertical axis of right panel) of various constructs (horizontal axis). Sample IDs characterized in FIG. 10A are plotted along the horizontal axis (1-7). FIG. 10C shows the configuration of sample lengths used in FIGS. 10D and 10E. Each row from “5′ Exon” to “GC content of homology arm (n)” corresponds to a component tested in one lane of FIGS. 6C-6H, with each sample in FIG. 10C possessing one of the component lengths, location, or GC contents previously tested in FIG. 6. Each sample (8-16) possesses all the conditions listed in the same column's rows beneath it. FIG. 10D shows agarose gel electrophoresis results of circularization efficiency for various samples. FIG. 10E shows FACS results demonstrating the expression level of EGFP (in %, vertical axis of left panel) and mean fluorescence intensity (MFI) of the EGFP (vertical axis of right panel) of various constructs (horizontal axis). Sample IDs characterized in FIG. 10C are plotted along the horizontal axis (8-16). In FIGS. 10B and 10E, “Unt”=an untransfected control; “Lin”=a linear precursor; “Ctrl”=an original version before sequence optimization; the MFI of EGFP was normalized to Ctrl; data are presented as the mean±S.D. (n=3); each dot represents a biological replicate. In FIG. 10C, “*”=use of a 20-nt linker with a random sequence, while others are AC linker; “**”=use of a 19-nt arm without homology. In FIG. 10D, “M”=molecular ladder, “Ctrl”=an original version before sequence optimization.

FIGS. 11A-11D show agarose gel electrophoresis results of optimizing conditions for a balance between circularization and RNA integrity in cis splicing. FIG. 11A shows results of testing various HEPES pH (labeled above each respective lane) on circularization and integrity. FIG. 11B shows results of testing various MgCl₂ (mM) concentrations (labeled above each respective lane) on circularization and integrity. FIG. 11C shows results of testing various temperatures (labeled above each respective lane) on circularization and integrity, for a 1-hour duration (top panel) and a 4-hour duration (bottom panel). FIG. 11D shows results of various reaction times (labeled above each respective lane) on circularization and integrity, at both 37 degrees C. (top panel) and 55 degrees C. (bottom panel). All buffers contained Mg²⁺, 50 mM HEPES, and 150 mM NaCl. M=molecular ladder.

FIGS. 12A-12D show agarose gel electrophoresis results comparing cis splicing with PIE under varying reaction conditions. FIG. 12A shows results of circularization efficiency for both methods at 55 degrees C. for 1 hour at 10 mM MgCl₂ after IVT reaction, wherein the RNAs contained IRES-EGFP. FIG. 12B shows results of circularization efficiency for both methods at 55 degrees C. for 1 hour at 10 mM MgCl₂ after IVT reaction, wherein the RNAs contained IRES-Gluc. FIG. 12C shows results of circularization efficiency for both methods with RNA containing IRES-EGFP at 55 degrees C. for different durations at 20 mM MgCl₂ FIG. 12D shows results of circularization efficiency for both methods with RNA containing IRES-EGFP at 55 degrees C. for different durations at 40 mM MgCl₂. M=molecular ladder.

FIGS. 13A-13C show agarose gel electrophoresis results of circularization efficiency compared between cis splicing and PIE using an alternative payload, IRES-Gluc. FIG. 13A shows these results across reaction time (within IVT, labeling each respective lane). The bound intron is indicated by a black arrow. FIG. 13B shows these results across reaction time with RNase R (labeling each respective lane). FIG. 13C shows these results across reaction time with RNase R treatments (labeling each respective lane) for IVT 2-hour RNA product in cis splicing. M=molecular ladder.

FIGS. 14A-14B show agarose gel electrophoresis results of circularization efficiency compared between cis splicing and PIE in RNase R treatment. FIG. 14A shows this with IRES-EGFP used as payload. FIG. 14B shows this with IRES-Gluc used as payload. M=molecular ladder.

FIGS. 15A-15F show gel electrophoresis and circularization efficiency measurements from experiments testing Mg²⁺ requirements in buffers containing different concentrations of monovalent cations. FIG. 15A shows agarose gel electrophoresis results of circularization efficiency compared between cis splicing (left) and PIE (right) in varying concentrations of MgCl₂ (listed in 0.1x mM across the top). Bands with lengths corresponding to the precursor and circularized RNA (circRNA) are indicated with arrowheads. FIGS. 15B-15F show measurements of the circularization efficiency of the ABE8e-Fluc-EGFP reporter encoding a large RNA sequence of over 8,000 nucleotides, SEQ ID NO: 199, in varying cis splicing and PIE splicing conditions. FIG. 15B shows agarose gel electrophoresis results of the cis splicing RNA circularization of the ABE8e-Fluc-EGFP reporter in lanes “V1”, “V2”, and “V3”, which correspond to three reporter versions tested. Each version has a different homology arm length; “V1”: 19-nt, “V2”: 34-nt, and “V3”: 151-nt. A control of equal molarity intron (that is, spliced intron produced in vitro and loaded in equal molarity to the precursors from lanes V1-V3 in order to help resolve the bands of precursor and circular RNA) was included in the lane labeled “Ctrl” to determine relative splicing efficiencies of the circularization reactions from the other lanes. These reactions occurred in buffer containing under 0.625 mM MgCl₂. FIG. 15C and FIG. 15D show the agarose gel electrophoresis results of circularization efficiency of large RNA sequences in low concentrations of MgCl₂ (mM) as described in Example 4. RNA circularization by cis splicing and PIE are shown in separate lanes. FIG. 15C shows the precursor (left lanes) and the product after circularization (right lanes), including before (− lanes) and after (+ lanes) poly (A) treatment. No change in size was expected in circular products after poly (A) treatment. The spliced intron band is labeled below the 500-nt marker. FIG. 15D shows the products after circularization, including before (− lanes) and after (+ lanes) RNase R treatment after poly (A) treatment. All products were treated with poly (A) treatment. No change in size is expected in circular products after RNase R treatment. A marker lane with molecular reference weights is shown on the left in the “M” lane. FIG. 15E and FIG. 15F show the protein expression detection results after transfection of the final products to HEK293T cells. The final product was treated with RNase R prior to transfection. FIG. 15E shows a plot of the luciferase activity detected in cells after transfection of products derived from cis splicing or PIE splicing as described in Example 4. Lanes labeled “V1”, “V2”, “V3”, and “PIE” represent the circular product encoded by the ABE8e-Fluc-EGFP reporter. Luciferase activity from cells transfected with either the precursor or the post-circularization products are shown. FIG. 15F shows the Western blots detecting protein expression of ABE8e from the cis splicing described in Example 4 with beta-tubulin as a loading control. Measured protein levels from cells transfected with either the precursor (left) or the post-circularization products (right) are shown.

FIGS. 16A-16F show schematics and data demonstrating Group I intron-mediated RNA circularization with cis splicing that produces scarless circRNAs with decreased immunogenicity as described in Example 5. FIG. 16A shows a schematic of a vector containing a Group I intron, a 3′ exon, a split IRES represented by “IRES Part 2” and “IRES Part 1”, a payload, and a 5′ exon. Each linear segment represents a piece of linear RNA. Exon sequences are shaded in black or varying shades of gray. Intron sequences are in white. After splicing, the 3′ exon and the 5′ exon ligate to produce a circular product. The payload comprises a gene of interest (GOI) sequence.

FIG. 16B shows the schematic of the ligated CVB3 IRES, which includes IRES Part 1 with nucleotides 1-381 and IRES Part 2 with nucleotides 382-741. The split site is between the 381^{st} and 382^{nd} nucleotide of the CMVB3 IRES. FIG. 16C shows the process used to test the immunogenicity of the circular RNAs in Example 5, produced from the aforementioned split site vector. circRNA was purified prior to transfection in A549 cells. After a 6-hour incubation, cells were harvested, and RNA was extracted to measure gene expression levels. FIG. 16D shows the fold change of gene expression levels for innate immune genes: RIG-I, TNF-alpha, and IFN-beta. The x-axis represents the samples, including controls or circRNA with varying scar lengths (6 and 30) with or without scars hidden in the IRES (with “hi” denoting a hidden scar). Controls included “T4RNL2” (circRNA generated via T4 RNA ligase 2) as a low immunogenicity control and “poly(I:C) and “linear 5′-3P” as high immunogenic controls. Samples were derived by the process described in FIG. 16C. Asterisked brackets represent samples that underwent a dephosphorylation treatment. FIG. 16E shows a schematic containing a Group I intron and the split motif required for cis splicing to produce scarless circRNA. The motif “NNUA” was split “NNU|A”, and the sequence “GNN” is included in the Group I intron to base pair with the “NNU” motif, with G and U as a wobble pair, and the “N” nucleotides pairing as Watson-Crick pairs. FIG. 16F shows the agarose gel electrophoresis results of circularization using split IRES to produce scarless circRNA by cis splicing. RNA circularization by cis splicing of IRES-EGFP and POLR2A is shown. The precursor, scarless circRNA, and intron are denoted on the gel by the arrows. Several motif “NNU|A” split sites were tested, including split sites 1, 2, and 3 for IRES-EGFP and split sites 1 and 2 for POLR2A.

FIGS. 17A-17E show schematics and data demonstrating Group II intron-mediated RNA circularization with cis splicing. FIG. 17A shows a schematic of a vector containing a Group II intron, a 3′ exon, a payload, and a 5′ exon, in which the EBS1 and IBS1 binding sites have non-perfect base-pairing. After splicing, the 3′ exon and the 5′ exon ligate to produce a circular product. FIG. 17B shows a schematic of a vector containing a Group II intron, a 3′ exon, a payload, and a 5′ exon, in which the EBS1 and IBS1 binding sites have perfect base-pairing, which limits the occurrence of splicing and produces a linear RNA. For both FIG. 17A and FIG. 17B, each linear segment represents a piece of linear RNA, exon sequences are shaded in black or gray, and intron sequences are in white. The payload comprises a gene of interest (GOI) sequence. FIG. 17C shows agarose gel electrophoresis results of circularization efficiency of samples with Group II introns by cis splicing as described in Example 6. The products after circularization and after poly (A) treatment are included. The spliced introns (white asterisk) and circularized RNAs (circRNAs, black asterisk) are shown. Lane labels 1-6 represent Group II intron-1, Group II intron-2, Group II intron-3, Group II intron-4, Group II intron-5, and Group II intron-6, respectively. FIG. 17D shows agarose gel electrophoresis results of circularization efficiency of samples with Group II introns by cis splicing before and after RNase R treatment. The circularized RNAs (circRNAs, black asterisk) are shown. No change in size was expected in circular products after RNase R treatment. A marker lane with molecular reference weights is shown along the left in the “M” column. FIG. 17E shows Sanger sequencing results of the reverse transcribed (RT)-PCR products from circRNAs using the indicated primer “F” and “R”, which cover the ligation site, as shown in the graphic at left. The gray double arrows represents the 5′ exon portion, and the black double arrows represents the 3′ exon portion. Precise ligation is indicated by an arrow pointing up. Sequencing data are shown at right for Group II intron-2, Group II intron-3, Group II intron-4, and Group II intron-5.

FIG. 18 shows agarose gel electrophoresis results of circularization efficiency of samples with six different Group I introns by cis splicing. The spliced introns (“Spliced intron”, white asterisks) and circularized RNAs (“CircRNA”, black asterisks) are indicated. Lane label “M” indicates the molecular ladder. Lane labels indicate the following origin species for the Group I intron used in each linear precursor RNA construct, from left to right: “Ana”=Group I intron sequence from Anabaena (provided in SEQ ID NO: 9); “Cmu”=Group I intron sequence from Coelastrella multistriata (provided in SEQ ID NO: 154); “Tar”=Group I intron sequence from Trebouxia arboricola (provided in SEQ ID NO: 155); “Tsp”=Group I intron sequence from Trebouxia sp. (provided in SEQ ID NO: 156); “Hpa”=Group I intron sequence from Hypocrea pallida (provided in SEQ ID NO: 157); “Azo”=Group I intron sequence from Azoarcus olearius (provided in SEQ ID NO: 159); and “Tet”=Group I intron sequence from Tetrahymena thermophila (provided in SEQ ID NO: 158).

### DETAILED DESCRIPTION

The present application provides methods of producing a circRNA from a linear RNA that contains a Group I intron or a fragment thereof but lacks any Group I intron sequence on its 3′ end. In some embodiments, the present application provides a method of producing a circRNA using self-splicing without the need to provide fragments of a Group I intron on each of the 5′ and 3′ ends of a linear RNA. The method allows production of circRNA from a linear RNA that has a Group I intron sequence only at the 5′ end without the need for a Group I intron or fragment thereof at the 3′ end of the linear RNA.

The present application also provides methods of producing a circRNA from a linear RNA that contains a Group II intron or a fragment thereof but lacks any Group II intron sequence on its 3′ end. In some embodiments, the present application provides a method of producing a circRNA using self-splicing without the need to provide fragments of a Group II intron on each of the 5′ and 3′ ends of a linear RNA. The method allows production of circRNA from a linear RNA that has a Group II intron sequence only at the 5′ end without the need for a Group II intron or fragment thereof at the 3′ end of the linear RNA.

CircRNAs are typically prepared using one of three general techniques: chemical methods (using, e.g., cyanogen bromide), enzymatic methods (using, e.g., a ligase), or ribozymatic methods, which use self-splicing introns (see generally, Wesselhoeft, R. A., Kowalski, P. S. & Anderson, D. G. Engineering circular RNA for potent and stable translation in eukaryotic cells. Nat Commun 9, 2629 (2018)). The current state-of-the art technique for preparing circRNAs using the ribozymatic method is generally known as permuted intron-exon (PIE) splicing, which requires a linear RNA precursor that has a Group I intron sequence (generally a 3′ fragment of a Group I intron) on the 5′ end and a fragment of a Group I intron (generally a 5′ fragment of a Group I intron) on the 3′ end. Self-splicing then occurs to generate a circRNA in which the intron fragments have been spliced out. This occurs by the 3′ hydroxyl group of a guanosine nucleotide engaging in a transesterification reaction at the 5′ splice site. The 5′ intron half is excised, and the freed hydroxyl group at the end of the intermediate engages in a second transesterification at the 3′ splice site, resulting in circularization of the intervening region and excision of the 3′ intron.

The present application is based at least in some parts on the surprising discovery that ribozymatic circularization can take place using a linear RNA precursor that lacks, on its 3′ end, a Group I intron or fragment thereof. The present application is based at least in other parts on the surprising discovery that ribozymatic circularization can take place using a linear RNA precursor that lacks, on its 3′ end, a Group II intron or fragment thereof. The methods described herein provide the ability to produce a wider variety of circRNA structures containing a wider variety of sequences via self-splicing than are possible with the previously-known self-splicing methods.

### I. Definitions

Terms are used herein as generally used in the art, unless otherwise defined as follows.

The term “linear RNA” refers to a RNA molecule having a 5′ end and a 3′ end. A linear RNA may have secondary structures, including helices and loop regions.

The term “linear RNA precursor” refers to a linear RNA that is used as a starting material for producing circRNA, and comprises at least a portion or at least a fragment of a catalytic Group I intron sequence, as well as a 3′ exon sequence, an effector RNA sequence, and a 5′ exon sequence. The term is used synonymously herein with the term “intermediate” in the context of the starting material provided for splicing in cis or in trans.

The terms “polynucleotide,” “nucleic acid,” “nucleotide sequence,” and “nucleic acid sequence” are used interchangeably. They refer to a polymeric form of nucleotides of any length, either deoxyribonucleotides or ribonucleotides, or analogs thereof.

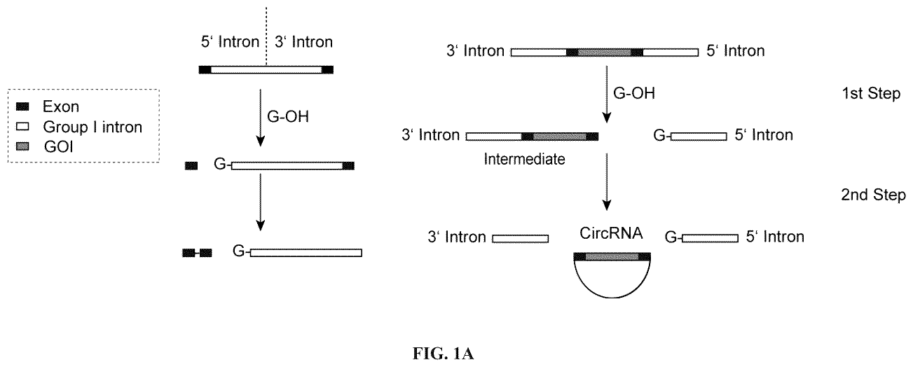

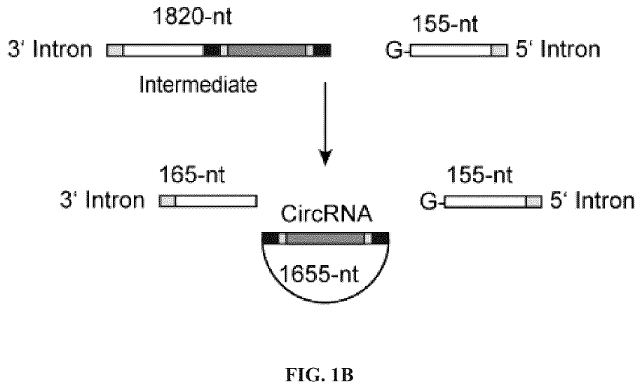

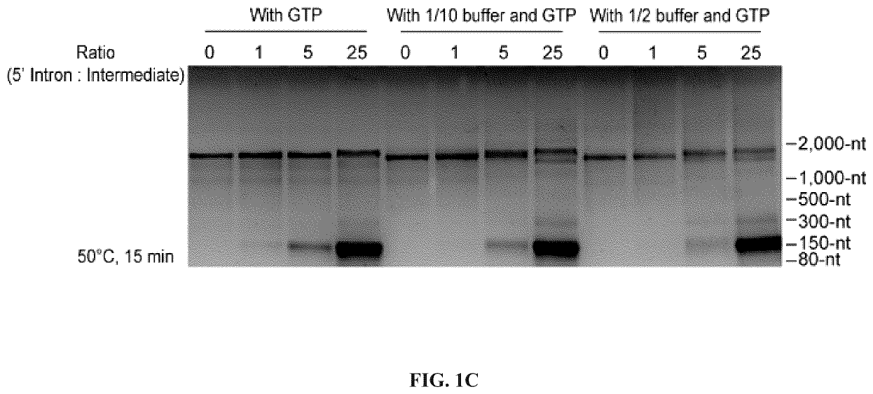

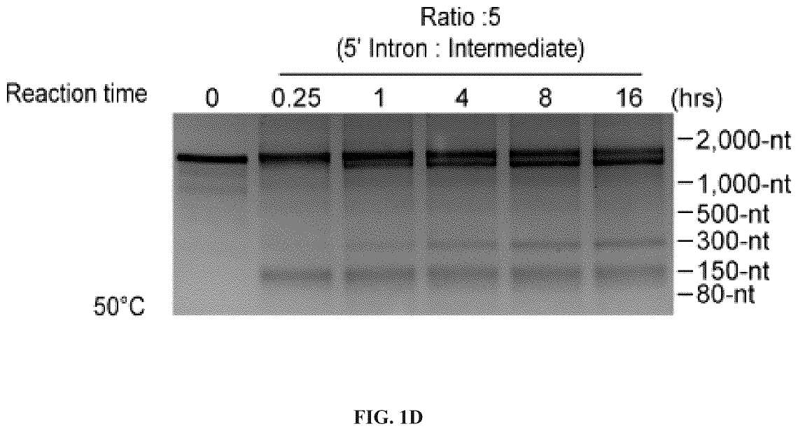

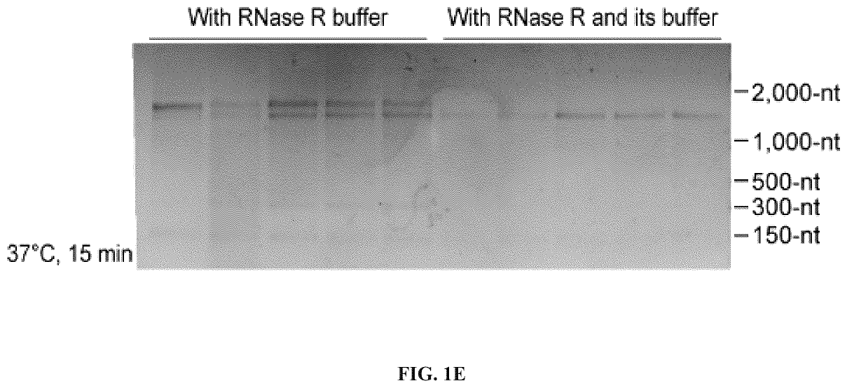

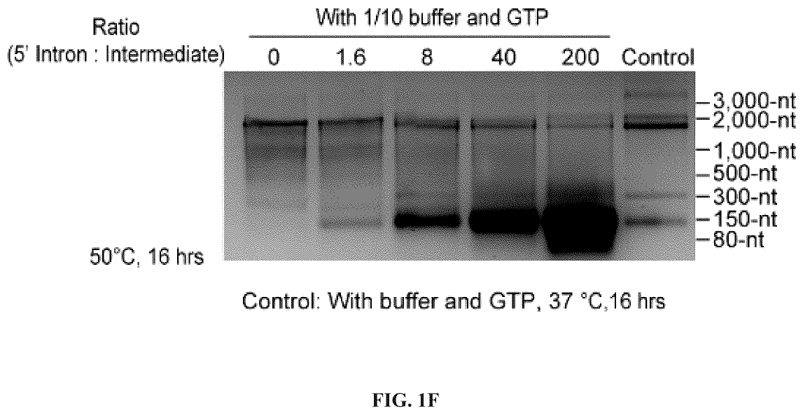

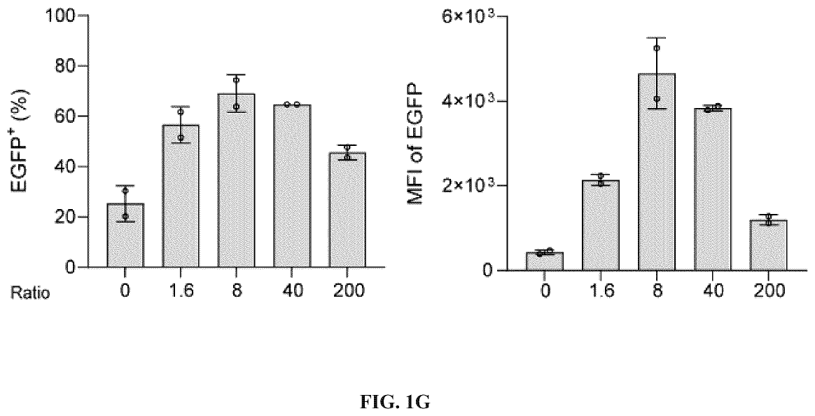

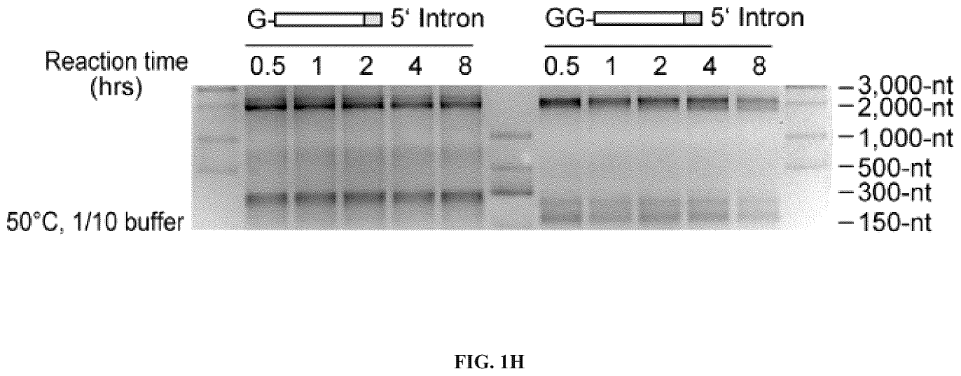

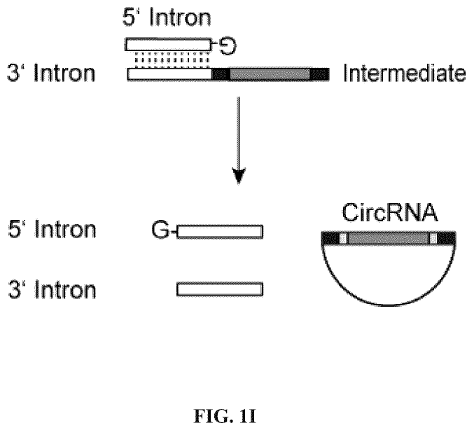

The term “catalytic Group I intron” refers to a self-splicing ribozyme that 1) can catalyze its own excision from an RNA precursor; 2) has a G nucleotide at the 5′ end; and 3) allows heterologous sequence inserted therein, so long as the activity of the catalytic Group I intron is not disrupted. In its native environment, the 5′ end of a catalytic Group I intron is flanked by a 5′ exon (referred to interchangeably herein as “Exon1”), which comprises a 5′ exon sequence that is recognized by the 5′ end of the catalytic Group I intron; and the 3′ end of a catalytic Group I intron is flanked by a 3′ exon (referred to interchangeably herein as “Exon2”), which comprises a 3′ exon sequence that is recognized by the 3′ end of the catalytic Group I intron. The terms “5′ exon sequence” and “3′ exon sequence” used herein in the context of a Group I intron or fragment thereof are labeled according to the order of the exons with respect to the Group I intron in its natural environment, e.g., as shown in FIG. 1A, left panel.

The term “catalytic Group I intron fragment” is used interchangeably herein with the terms “Group I intron fragment” and “intron fragment” in the context of a Group I intron and refers to a portion of a catalytic Group I intron that may or may not comprise a G nucleotide at the 5′ end and may or may not retain catalytic activity when a heterologous sequence is inserted therein. A catalytic Group I intron fragment may be a 5′ catalytic Group I intron fragment or a 3′ catalytic Group I intron fragment, both of which retain their folding and catalytic function (i.e., self-splicing activity). The 5′ catalytic Group I intron fragment recognizes the 5′ exon sequence, and the 3′ catalytic Group I intron fragment recognizes the 3′ exon sequence.

The term “free 5′ catalytic Group I intron fragment” is used interchangeably herein with “free 5′ intron fragment”, “free Group I intron fragment”, and “free 5′ intron” in a Group I context, and refers to a catalytic Group I intron fragment that comprises the 5′ portion of a catalytic Group I intron, has at least one G nucleotide on its 5′ end, and is not covalently attached to a sequence that comprises a 3′ portion of a catalytic Group I intron. The free Group I intron fragment can be embedded in a longer RNA sequence.

The term “catalytic Group II intron” refers to a self-splicing ribozyme that 1) can catalyze its own excision from an RNA precursor; and 2) allows heterologous sequence inserted therein, so long as the activity of the catalytic Group II intron is not disrupted. In its native environment, the 5′ end of a catalytic Group II intron is flanked by a 5′ exon (referred to interchangeably herein as “Exon1”), which comprises a 5′ exon sequence that is recognized by the 5′ end of the catalytic Group II intron; and the 3′ end of a catalytic Group II intron is flanked by a 3′ exon (referred to interchangeably herein as “Exon2”), which comprises a 3′ exon sequence that is recognized by the 3′ end of the catalytic Group II intron. The terms “5′ exon sequence” and “3′ exon sequence” used herein in the context of a Group II intron or fragment thereof are labeled according to the order of the exons with respect to the Group II intron in its natural environment.

The term “catalytic Group II intron fragment” is used interchangeably herein with the terms “Group II intron fragment” and “intron fragment” in the context of a Group II intron and refers to a portion of a catalytic Group II intron that may or may not comprise a G nucleotide at the 5′ end and may or may not retain catalytic activity when a heterologous sequence is inserted therein. A catalytic Group II intron fragment may be a 5′ catalytic Group II intron fragment or a 3′ catalytic Group II intron fragment, both of which retain their folding and catalytic function (i.e., self-splicing activity). The 5′ catalytic Group II intron fragment recognizes the 5′ exon sequence, and the 3′ catalytic Group II intron fragment recognizes the 3′ exon sequence.

The term “free 5′ catalytic Group II intron fragment” is used interchangeably herein with “free 5′ intron fragment”, “free Group II intron fragment”, and “free 5′ intron” in a Group II context, and refers to a catalytic Group II intron fragment that comprises the 5′ portion of a catalytic Group II intron, has at least one G nucleotide on its 5′ end, and is not covalently attached to a sequence that comprises a 3′ portion of a catalytic Group II intron. The free Group II intron fragment can be embedded in a longer RNA sequence.

The term “tune” as used herein refers to modulating the catalytic activity of a Group I intron or Group II intron. The modulation may have the effect of attenuating the catalytic activity to any measurable degree or enhancing the catalytic activity to any measurable degree.

As used herein, “complementarity” refers to the ability of a nucleic acid to form hydrogen bond(s) with another nucleic acid by traditional Watson-Crick base-pairing. A percent complementarity indicates the percentage of residues in a nucleic acid molecule which can form hydrogen bonds (i.e., Watson-Crick base pairing) with a second nucleic acid (e.g., about 5, 6, 7, 8, 9, 10 out of 10, being about 50%, 60%, 70%, 80%, 90%, and 100% complementary respectively). “Perfectly complementary” means that all the contiguous residues of a nucleic acid sequence form hydrogen bonds with the same number of contiguous residues in a second nucleic acid sequence. “Substantially complementary” as used herein refers to a degree of complementarity that is at least about any one of 70%, 75%, 80%, 85%, 90%, 95%, 97%, 98%, 99%, or 100% over a region of about 40, 50, 60, 70, 80, 100, 150, 200, 250 or more nucleotides, or refers to two nucleic acids that hybridize under stringent conditions.

As used herein the term “wild type” is a term of the art understood by skilled persons and means the typical form of an organism, strain, gene or characteristic as it occurs in nature as distinguished from mutant or variant forms.

The present disclosure provides several types of compositions that are polynucleotide or polypeptide based, including variants and derivatives. These include, for example, substitutional, insertional, deletion and covalent variants and derivatives. The term “derivative” is synonymous with the term “variant” and generally refers to a molecule that has been modified and/or changed in any way relative to a reference molecule or a starting molecule.

As such, polynucleotides encoding peptides or polypeptides containing substitutions, insertions and/or additions, deletions and covalent modifications with respect to reference sequences, in particular, the polypeptide sequences disclosed herein, are included within the scope of this disclosure. For example, sequence tags or amino acids, such as one or more lysines, can be added to peptide sequences (e.g., at the N-terminal or C-terminal ends). Sequence tags can be used for peptide detection, purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal residues or N-terminal residues) alternatively may be deleted depending on the use of the sequence, as for example, expression of the sequence as part of a larger sequence that is soluble, or linked to a solid support.

The term “identity” refers to the overall relatedness between polymeric molecules, for example, between polynucleotide molecules (e.g. DNA molecules and/or RNA molecules) and/or between polypeptide molecules. Calculation of the percent identity of two polynucleic acid sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleic acid sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleic acid sequences can be determined using the algorithm of Meyers and Miller (CABIOS, 1989, 4:11-17), which has been incorporated into the ALIGN program (version 2.0) using a PAM 120 weight residue table, a gap length penalty of 12 and a gap penalty of 4. The percent identity between two nucleic acid sequences can, alternatively, be determined using the GAP program in the GCG software package using an NWSgapdna. CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12 (1), 387 (1984)), BLASTP, BLASTN, and FASTA Altschul, S. F. et al., J. Molec. Biol., 215, 403 (1990)).

“Percent (%) amino acid sequence identity” with respect to the polypeptide sequences identified herein is defined as the percentage of amino acid residues in a candidate sequence that are identical with the amino acid residues in the polypeptide being compared, after aligning the sequences considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, Megalign (DNASTAR), or MUSCLE software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program MUSCLE (Edgar, R. C., Nucleic Acids Research 32 (5): 1792-1797, 2004; Edgar, R. C., BMC Bioinformatics 5 (1): 113, 2004, each of which are incorporated herein by reference in their entirety for all purposes).

The terms “non-naturally occurring” or “engineered” are used interchangeably, and when referring to nucleic acid molecules or polypeptides mean that the nucleic acid molecule or the polypeptide is at least substantially free from at least one other component with which they are naturally associated in nature and as found in nature.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “introducing” or “introduction” used herein means delivering one or more polynucleotides, such as linear RNA precursors, Group I intron fragments, Group II intron fragments, circRNAs, and/or or one or more constructs including vectors as described herein, one or more transcripts thereof, to a host cell of any organism. The methods of the present application can employ many delivery systems, including but not limited to, viral, liposome, electroporation, microinjection and conjugation, to achieve the introduction of the circRNA or construct as described herein into a host cell. Conventional viral and non-viral based gene transfer methods can be used to introduce nucleic acids into, for example, mammalian cells or target tissues. Such methods can be used to administer nucleic acids encoding the linear RNA precursors, Group I intron fragments, Group II intron fragments, and/or circRNA of the present application to cells in culture, or in a host organism. Non-viral vector delivery systems include DNA plasmids, RNA (e.g. a transcript of a construct described herein), naked nucleic acid, and nucleic acid complexed with a delivery vehicle, such as a liposome. Viral vector delivery systems include DNA and RNA viruses, which have either episomal or integrated genomes for delivery to the host cell.

As used herein, “operably linked,” when referring to a first nucleic acid sequence that is operably linked with a second nucleic acid sequence, means a situation when the first nucleic acid sequence is placed in a functional relationship with the second nucleic acid sequence. For instance, a promoter is operably linked to a coding sequence if the promoter effects the transcription of the coding sequence. Likewise, the coding sequence of a signal peptide is operably linked to the coding sequence of a polypeptide if the signal peptide effects the extracellular secretion of that polypeptide. Generally, operably linked nucleic acid sequences are contiguous and, where necessary to join two protein coding regions, the open reading frames are aligned.

The terms “polypeptide” or “peptide” are used herein to encompass all kinds of naturally occurring and synthetic proteins, including protein fragments of all lengths, fusion proteins and modified proteins, including without limitation, glycoproteins, as well as all other types of modified proteins (e.g., proteins resulting from phosphorylation, acetylation, myristoylation, palmitoylation, glycosylation, oxidation, formylation, amidation, polyglutamylation, ADP-ribosylation, pegylation, biotinylation, etc.).

It is understood that embodiments of the invention described herein include “consisting” and/or “consisting essentially of” embodiments.

Reference to “about” a value or parameter herein includes (and describes) variations that are directed to that value or parameter per se. For example, description referring to “about X” includes description of “X”.

As used herein, reference to “not” a value or parameter generally means and describes “other than” a value or parameter. For example, the method is not used to treat disease of type X means the method is used to treat disease of types other than X.

The term “about X-Y” used herein has the same meaning as “about X to about Y.”

As used herein and in the appended claims, the singular forms “a,” “an,” or “the” include plural referents unless the context clearly dictates otherwise.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Producing CircRNAs

The present application provides methods for producing circRNAs from linear RNA precursors comprising a catalytic Group I intron or fragment thereof that undergo Group I intron-mediated RNA circularization, and circRNAs prepared using the described methods. In known methods for producing circRNAs by Group I intron-mediated RNA circularization, the linear RNA precursor comprises from the 5′-end to the 3′ end: a 3′ catalytic Group I intron fragment, a 3′ exon sequence, an effector RNA sequence, a 5′ exon sequence, and a 5′ catalytic Group I intron fragment, and circularization of the linear RNA precursor comprises activation of the 3′ catalytic Group I intron fragment and the 5′ catalytic Group I intron fragment to splice the 3′ exon sequence and the 5′ exon sequence from the linear RNA precursor, thereby forming the circular RNA comprising the effector RNA. The present application provides methods for producing circRNAs by Group I intron-mediated RNA circularization in which the linear RNA precursor lacks a 5′ catalytic Group I intron fragment.

In some embodiments, the Group I intron-mediated RNA circularization involves splicing in cis. In some embodiments, the Group I intron-mediated RNA circularization involves splicing in trans.

The present application further provides methods for producing circRNAs from linear RNA precursors comprising a catalytic Group II intron or fragment thereof that undergo Group II intron-mediated RNA circularization, and circRNAs prepared using the described methods. In known methods for producing circRNAs by Group II intron-mediated RNA circularization, the linear RNA precursor comprises at least a 3′ catalytic Group II intron fragment on the 5′ end and a 5′ catalytic Group II intron fragment on the 3′ end, such as, for example, in known versions of the PIE method as described in Obi, P, and Chen, YG. The design and synthesis of circular RNAs. Methods. 2021 December; 196:85-103. Epub 2021 Mar. 2. PMID: 33662562; PMCID: PMC8670866 (herein incorporated by reference in its entirety). For example, in the PIE method, circularization can take place using a construct comprising, from the 5′-end to the 3′ end: a 3′ catalytic Group II intron fragment, a 3′ exon sequence, an effector RNA sequence, a 5′ exon sequence, and a 5′ catalytic Group II intron fragment, and circularization of the linear RNA precursor comprises activation of the 3′ catalytic Group II intron fragment and the 5′ catalytic Group II intron fragment to splice the 3′ exon sequence and the 5′ exon sequence from the linear RNA precursor, thereby forming the circular RNA comprising the effector RNA. The present application provides methods for producing circRNAs by Group II intron-mediated RNA circularization in which the linear RNA precursor lacks a 5′ catalytic Group II intron fragment.

In some embodiments, the Group II intron-mediated RNA circularization involves splicing in cis. In some embodiments, the Group II intron-mediated RNA circularization involves splicing in trans.

The present application further provides nucleic acid constructs (e.g., linear RNA and vectors, etc.) for preparation of the circRNAs described herein, and methods for preparing the circRNAs by ribozyme autocatalysis of linear RNAs. In some embodiments, the circRNA is produced by circularizing a linear RNA in vitro. In some embodiments, the circRNA is produced by circularizing a linear RNA in vivo.

### Linear RNA Precursor for Splicing in Cis Using a Catalytic Group I Intron or Fragment Thereof

In some embodiments, the present application provides a linear RNA precursor capable of forming the circRNA of any one of the embodiments described herein via cis splicing, in which the linear RNA precursor does not comprise a 5′ catalytic Group I intron fragment on its 3′ end. In some embodiments, the present application provides a linear RNA precursor capable of forming the circRNA of any one of the embodiments described herein, wherein the linear RNA precursor can be circularized by autocatalysis of a catalytic Group I intron. In some embodiments, the linear RNA precursor comprises, from the 5′ end to the 3′ end: a catalytic Group I intron; a 3′ exon sequence (referred to synonymously herein as Exon2); an effector RNA sequence (also described herein as a “payload”), and a 5′ exon sequence (referred to synonymously herein as Exon1). In some embodiments, the linear RNA precursor comprises a gene of interest (GOI) sequence in the effector sequence. In some embodiments, the GOI comprises an IRES-EGFP sequence. In some embodiments, the catalytic Group I intron is capable of splicing the 3′ exon sequence and the 5′ exon sequence from the linear RNA precursor, thereby forming a circRNA comprising the effector RNA.

The linear RNA precursor does not require antisense sequences on the 5′ and 3′ ends in order to splice in cis in any of the embodiments described herein. Thus, in some embodiments, linear RNA precursor for cis splicing does not comprise antisense sequences on the 5′ and 3′ ends. In some embodiments, linear RNA precursor for cis splicing comprises antisense sequences on the 5′ and 3′ ends. The methods of cis splicing described herein further do not require G/U wobble base pairing in order to form circRNA in any of the embodiments described herein. For example, in some embodiments, the linear RNA precursor for cis splicing does not comprise a sequence on the 5′ end of the catalytic Group I intron that comprises a guanine nucleotide that forms a G/U wobble base pair with a U nucleotide that is present in a sequence on or after the 3′ end of the catalytic Group I intron. In some embodiments, the linear RNA precursor for cis splicing comprises a G/U wobble base pair.

In some embodiments, the sequence of the linear RNA precursor used for splicing in cis comprises the sequence set forth in any of SEQ ID NOs: 7, 8, 13, 14, 15, 23, 32, or 49-54, or a variant, modification, or derivative thereof. In some embodiments, the catalytic Group I intron sequence used for splicing in cis comprises the sequence set forth in any of SEQ ID NOs: 9, 16-18, or 55-52, or a variant, modification, or derivative thereof. In some embodiments, the 3′ exon sequence used for splicing in cis comprises the sequence set forth in any of SEQ ID NOs: 10, 19-20, 24, 30-31, 37-40, or 45-48, or 67, or a variant, modification, or derivative thereof. In some embodiments, the IRES-EGFP sequence used for splicing in cis comprises the sequence set forth in SEQ ID NO: 5, or a variant, modification, or derivative thereof. In some embodiments, the 5′ exon sequence used for splicing in cis comprises the sequence set forth in any of SEQ ID NOs: 11, 21-22, 25, 33-36, 41-44, or 63-66, or a variant, modification, or derivative thereof.

In some embodiments, the linear RNA precursor does not comprise a homology arm. In some embodiments, the linear RNA precursor comprises a 5′ homology arm sequence flanking the 3′ end of Exon2, and/or a 3′ homology arm sequence flanking the 5′ of Exon1, wherein the 5′ homology arm sequence and the 3′ homology arm sequence hybridize with each other. In some embodiments, the 5′ homology arm sequence and the 3′ homology arm sequence are each about 5, 10, 15, 20, 25, 30, 35, 40, 45, 50, 55, 60, 65, 70, 75, 80, 85, 90, 95, or 100 nucleotides in length. In some embodiments, the 5′ homology arm sequence and the 3′ homology arm sequence are each about 5-10, 10-15, 15-20, 20-25, 25-30, 35-40, 45-50, 55-60, 65-70, or more than 70 nucleotides in length. In some embodiments, the 5′ homology arm sequence and the 3′ homology arm sequence are each about 0, 4, 9, 14, 19, 24, 29, 34 nucleotides in length. The GC content of the homology arm sequence may vary. In some embodiments, the GC content of the homology arm sequence is 0-5, 5-10, 10-15, 15-20, 20-25, 25-30, 35-40, 45-50, 55-60, 65-70, or more than 70%. In some embodiments, the GC content of the homology arm sequence is 0-5, 5-10, 10-15, or 15-20%.

In some embodiments, the linear RNA precursor comprises a flexible linker sequence between the 5′ exon and the 3′ homology arm. In some embodiments, the linear RNA precursor comprises a flexible linker sequence between the 3′ exon and the 5′ homology arm. In some embodiments, the linear RNA precursor comprises a flexible linker sequence between the the 3′ homology arm and the effector RNA sequence. In some embodiments, the linear RNA precursor comprises a flexible linker sequence between the 5′ homology arm and the effector RNA sequence.

In some embodiments, the linear RNA precursor comprises a 2′-OMe modification. In some embodiments, the 2′-OMe modification is on the 3′ end of the linear RNA precursor.

### Linear RNA Precursor and Free 5′ Catalytic Group I Intron Fragment for Splicing in Trans Using a Catalytic Group I Intron or Fragment Thereof

In some embodiments, the present application provides an RNA circularization system comprising a linear RNA and a free 5′ catalytic Group I intron fragment capable of forming the circRNA of any one of the embodiments described herein via trans splicing. In some embodiments, the present application provides a linear RNA precursor capable of forming the circRNA of any one of the embodiments described herein, wherein the linear RNA precursor can be circularized by autocatalysis of a Group I intron without a 5′ catalytic Group I intron fragment on the 3′ end of the linear RNA precursor, by splicing in trans. In some embodiments, the linear RNA precursor comprises, from the 5′ end to the 3′ end: a 3′ catalytic Group I intron fragment; a 3′ exon sequence; an effector RNA sequence, and a 5′ exon sequence. In some embodiments, a free 5′ catalytic Group I intron fragment is used in combination with a canonical PIE-style linear RNA precursor (e.g., in combination with a linear RNA precursor comprises from the 5′-end to the 3′ end: a 3′ catalytic Group I intron fragment, a 3′ exon sequence, an effector RNA sequence, a 5′ exon sequence, and a 5′ catalytic Group I intron fragment) to facilitate circularization of the canonical PIE-style linear RNA precursor. In some embodiments, the linear RNA precursor comprises, from the 5′ end to the 3′ end: a 3′ catalytic Group I intron fragment; a 3′ exon sequence; an effector RNA sequence, and a 5′ exon sequence, and does not comprise a 5′ catalytic Group I intron fragment. In some embodiments, the linear RNA precursor comprises a gene of interest (GOI) sequence in the effector sequence. In some embodiments, the GOI comprises an IRES-EGFP sequence. In some embodiments, the splicing in trans involves the linear RNA precursor contacting a free 5′ catalytic Group I intron fragment. In some embodiments, the 3′ catalytic Group I intron fragment of the linear RNA precursor and the free 5′ catalytic Group I intron fragment are capable of accomplishing splicing in trans by splicing the 3′ exon sequence and the 5′ exon sequence from the linear RNA precursor, thereby forming a circRNA comprising the effector RNA.

The linear RNA precursor does not require antisense sequences on the 5′ and 3′ ends in order to splice in trans in any of the embodiments described herein. Thus, in some embodiments, linear RNA precursor for trans splicing does not comprise antisense sequences on the 5′ and 3′ ends. In some embodiments, linear RNA precursor for trans splicing comprises antisense sequences on the 5′ and 3′ ends. The methods of trans splicing described herein further do not require G/U wobble base pairing in order to form circRNA in any of the embodiments described herein. For example, in some embodiments, the linear RNA precursor for trans splicing does not comprise a sequence on the 5′ end of the 3′ catalytic Group I intron fragment that comprises a guanine nucleotide that forms a G/U wobble base pair with a U nucleotide that is present in a sequence on or after the 3′ end of the 3′ catalytic Group I intron fragment. In some embodiments, the linear RNA precursor for trans splicing does not comprise a sequence on the 5′ end of the 3′ catalytic Group I intron fragment that comprises a guanine nucleotide that forms a G/U wobble base pair with a U nucleotide that is present on the same strand of RNA as the free 5′ catalytic Group I intron fragment. In some embodiments, the linear RNA precursor for trans splicing comprises a G/U wobble base pair.

In some embodiments, the sequence of the linear RNA precursor used for splicing in trans comprises the sequence set forth in SEQ ID NO: 2, or a variant, modification, or derivative thereof. In some embodiments, the 5′ intron sequence used for splicing in trans comprises the sequence set forth in SEQ ID NO: 1, or a variant, modification, or derivative thereof. In some embodiments, the 3′ intron sequence used for splicing in trans comprises the sequence set forth in SEQ ID NO: 3, or a variant, modification, or derivative thereof. In some embodiments, the 3′ exon sequence used for splicing in trans comprises the sequence set forth in SEQ ID NO: 4, or a variant, modification, or derivative thereof. In some embodiments, the IRES-EGFP sequence used for splicing in trans comprises the sequence set forth in SEQ ID NO: 5, or a variant, modification, or derivative thereof. In some embodiments, the 5′ exon sequence used for splicing in trans comprises the sequence set forth in SEQ ID NO: 6, or a variant, modification, or derivative thereof.

In some embodiments, the free 5′ catalytic Group I intron fragment does not have a G nucleotide on its 5′ end. In some embodiments, the free 5′ catalytic Group I intron fragment has 1 or more, 2 or more, 3 or more, 4 or more, 5 or more, 6 or more, or 7 or more G nucleotides on its 5′ end. In some embodiments, the free 5′ catalytic Group I intron fragment has a single G nucleotide on its 5′ end. In some embodiments, the free 5′ catalytic Group I intron fragment has two G nucleotides on its 5′ end.

In some embodiments, the free 5′ catalytic Group I intron fragment and the linear RNA precursor are present at a 1:1 molar ratio. In some embodiments, the free 5′ catalytic Group I intron fragment and the linear RNA precursor are present at more than a 1:1 molar ratio. In some embodiments, the free 5′ catalytic Group I intron fragment and the linear RNA precursor are present at less than a 1:1 molar ratio. In some embodiments, the free 5′ catalytic Group I intron fragment and the linear RNA precursor are present at a 5′ intron:linear RNA precursor molar ratio of 0-1:1, 1:1-2:1, 2:1-3:1, 3:1-4:1, 4:1-5:1, 5:1-6:1, 6:1-7:1, 7:1-8:1, 8:1-9:1, 9:1-10:1, 10:1-15:1, 15:1-20:1, 20:1-25:1, 25:1-30:1, 30:1-35:1, 35:1-40:1, 40:1-45:1, 45:1-50:1, 50:1-55:1, 55:1-60:1, 60:1-65:1, 65:1-70:1, 70:1-75:1, 75:1-80:1, 80:1-85:1, 85:1-90:1, 90:1-95:1, 95:1-100:1, 100:1-110:1, 110:1-120:1, 120:1-130:1, 130:1-140:1, 140:1-150:1, 150:1-160:1, 160:1-170:1, 170:1-180:1, 180:1-190:1, 190:1-200:1, or above 200:1. In some embodiments, the free 5′ catalytic Group I intron fragment and the linear RNA precursor are present at a 5′ intron:linear RNA precursor molar ratio of 1:1, 1.6:1, 5:1, 8:1, 25:1, 40:1, or 200:1. In some embodiments, the free 5′ catalytic Group I intron fragment and the linear RNA precursor are present at a 5′ intron:linear RNA precursor molar ratio of 5:1 to 10:1.

In some embodiments, the linear RNA precursor further comprises a 5′ homology arm sequence flanking the 5′ of the 3′ catalytic Group I intron fragment, and the free 5′ intron fragment comprises a 3′ homology arm sequence on its 3′ end, wherein the 5′ homology arm sequence and the 3′ homology arm sequence hybridize with each other. In some embodiments, the 5′ homology arm sequence and the 3′ homology arm sequence are each about 5, 10, 15, 20, 25, 30, 35, 40, 45, 50, 55, 60, 65, 70, 75, 80, 85, 90, 95, or 100 nucleotides in length. In some embodiments, the linear RNA precursor does not comprise a 5′ homology arm sequence. In some embodiments, the 5′ homology arm sequence and the 3′ homology arm sequence are each about 0-5, 5-10, 10-15, 15-20, 20-25, 25-30, 35-40, 45-50, 55-60, 65-70, or more than 70 nucleotides in length. In some embodiments, the 5′ homology arm sequence and the 3′ homology arm sequence are each about 0, 4, 9, 14, 19, 24, 29, 34 nucleotides in length. The GC content of the homology arm sequence may vary. In some embodiments, the GC content of the homology arm sequence is 0-5, 5-10, 10-15, 15-20, 20-25, 25-30, 35-40, 45-50, 55-60, 65-70, or more than 70%. In some embodiments, the GC content of the homology arm sequence is 0-5, 5-10, 10-15, or 15-20%.

In some embodiments, the linear RNA precursor comprises a 2′-OMe modification. In some embodiments, the 2′-OMe modification is on the 3′ end of the linear RNA precursor.

### Linear RNA Precursor for Splicing in Cis Using a Catalytic Group II Intron or Fragment Thereof

In some embodiments, the present application provides a linear RNA precursor capable of forming the circRNA of any one of the embodiments described herein via cis splicing, in which the linear RNA precursor does not comprise a 5′ catalytic Group II intron fragment on its 3′ end. In some embodiments, the present application provides a linear RNA precursor capable of forming the circRNA of any one of the embodiments described herein, wherein the linear RNA precursor can be circularized by autocatalysis of a catalytic Group II intron. In such embodiments, the 5′ end of the linear RNA precursor a G residue that is not adjacent to a second G residue, and the 5′ G residue has either no phosphate groups or only one phosphate group. If the first G residue from 5′ to 3′ of the linear RNA precursor has two or three phosphate groups, circularization is impaired. Finally, linear RNA precursors comprising a catalytic Group II intron or catalytic Group II intron fragment as used in the methods described herein should have imperfect base pairing between the EBS1 and IBS1 sites.

In some embodiments, the linear RNA precursor comprises, from the 5′ end to the 3′ end: a catalytic Group II intron; a 3′ exon sequence (referred to synonymously herein as Exon2); an effector RNA sequence (also described herein as a “payload”), and a 5′ exon sequence (referred to synonymously herein as Exon1). In some embodiments, the linear RNA precursor comprises a gene of interest (GOI) sequence in the effector sequence. In some embodiments, the GOI comprises an IRES-EGFP sequence. In some embodiments, the catalytic Group II intron is capable of splicing the 3′ exon sequence and the 5′ exon sequence from the linear RNA precursor, thereby forming a circRNA comprising the effector RNA.

The linear RNA precursor does not require antisense sequences on the 5′ and 3′ ends in order to splice in cis in any of the embodiments described herein. Thus, in some embodiments, linear RNA precursor for cis splicing does not comprise antisense sequences on the 5′ and 3′ ends. In some embodiments, linear RNA precursor for cis splicing comprises antisense sequences on the 5′ and 3′ ends. The methods of cis splicing described herein further do not require G/U wobble base pairing in order to form circRNA in any of the embodiments described herein. For example, in some embodiments, the linear RNA precursor for cis splicing does not comprise a sequence on the 5′ end of the catalytic Group II intron that comprises a guanine nucleotide that forms a G/U wobble base pair with a U nucleotide that is present in a sequence on or after the 3′ end of the catalytic Group II intron. In some embodiments, the linear RNA precursor for cis splicing comprises a G/U wobble base pair.

In some embodiments, the linear RNA precursor does not have a G nucleotide on its 5′ end. In some embodiments, the linear RNA precursor has a single G nucleotide on its 5′ end.

In some embodiments, the sequence of the linear RNA precursor used for splicing in cis comprises the sequence set forth in any of SEQ ID NOs: 175-198, or a variant, modification, or derivative thereof. In some embodiments, the catalytic Group II intron sequence used for splicing in cis comprises the sequence set forth in any of SEQ ID NO: 175 (also described herein as Group II intron 1), SEQ ID NO: 176 (also described herein as Group II intron 2), SEQ ID NO: 177 (also described herein as Group II intron 3), SEQ ID NO: 178 (also described herein as Group II intron 4), SEQ ID NO: 179 (also described herein as Group II intron 5), and SEQ ID NO: 180 (also described herein as Group II intron 6), or a variant, modification, or derivative thereof. In some embodiments, the 3′ exon sequence used for splicing in cis comprises the sequence set forth in any of SEQ ID NO: 187 (also described herein as Group II intron 1 Exon2), SEQ ID NO: 188 (also described herein as Group II intron 2 Exon2), SEQ ID NO: 189 (also described herein as Group II intron 3 Exon2), SEQ ID NO: 190 (also described herein as Group II intron 4 Exon2), SEQ ID NO: 191 (also described herein as Group II intron 5 Exon2), SEQ ID NO: 192 (also described herein as Group II intron 6 Exon2), or a variant, modification, or derivative thereof. In some embodiments, the IRES-EGFP sequence used for splicing in cis comprises the sequence set forth in SEQ ID NO: 5, or a variant, modification, or derivative thereof. In some embodiments, the 5′ exon sequence used for splicing in cis comprises the sequence set forth in any of SEQ ID NO: 181 (also described herein as Group II intron 1 Exon1), SEQ ID NO: 182 (also described herein as Group II intron 2 Exon1), SEQ ID NO: 183 (also described herein as Group II intron 3 Exon1), SEQ ID NO: 184 (also described herein as Group II intron 4 Exon1), SEQ ID NO: 185 (also described herein as Group II intron 5 Exon1), SEQ ID NO: 186 (also described herein as Group II intron 6 Exon1), or a variant, modification, or derivative thereof.

In some embodiments, the linear RNA precursor does not comprise a homology arm. In some embodiments, the linear RNA precursor comprises a 5′ homology arm sequence flanking the 3′ end of Exon2, and/or a 3′ homology arm sequence flanking the 5′ of Exon1, wherein the 5′ homology arm sequence and the 3′ homology arm sequence hybridize with each other. In some embodiments, the 5′ homology arm sequence and the 3′ homology arm sequence are each about 5, 10, 15, 20, 25, 30, 35, 40, 45, 50, 55, 60, 65, 70, 75, 80, 85, 90, 95, or 100 nucleotides in length. In some embodiments, the 5′ homology arm sequence and the 3′ homology arm sequence are each about 5-10, 10-15, 15-20, 20-25, 25-30, 35-40, 45-50, 55-60, 65-70, or more than 70 nucleotides in length. In some embodiments, the 5′ homology arm sequence and the 3′ homology arm sequence are each about 0, 4, 9, 14, 19, 24, 29, 34 nucleotides in length. The GC content of the homology arm sequence may vary. In some embodiments, the GC content of the homology arm sequence is 0-5, 5-10, 10-15, 15-20, 20-25, 25-30, 35-40, 45-50, 55-60, 65-70, or more than 70%. In some embodiments, the GC content of the homology arm sequence is 0-5, 5-10, 10-15, or 15-20%.

In some embodiments, the linear RNA precursor comprises a flexible linker sequence between the 5′ exon and the 3′ homology arm. In some embodiments, the linear RNA precursor comprises a flexible linker sequence between the 3′ exon and the 5′ homology arm. In some embodiments, the linear RNA precursor comprises a flexible linker sequence between the 3′ homology arm and the effector RNA sequence. In some embodiments, the linear RNA precursor comprises a flexible linker sequence between the 5′ homology arm and the effector RNA sequence.

In some embodiments, the linear RNA precursor comprises a 2′-OMe modification. In some embodiments, the 2′-OMe modification is on the 3′ end of the linear RNA precursor.

### Linear RNA Precursor and Free 5′ Catalytic Group II Intron Fragment for Splicing in Trans Using a Catalytic Group II Intron or Fragment Thereof

In some embodiments, the present application provides an RNA circularization system comprising a linear RNA and a free 5′ catalytic Group II intron fragment capable of forming the circRNA of any one of the embodiments described herein via trans splicing. In some embodiments, the present application provides a linear RNA precursor capable of forming the circRNA of any one of the embodiments described herein, wherein the linear RNA precursor can be circularized by autocatalysis of a Group II intron without a 5′ catalytic Group II intron fragment on the 3′ end of the linear RNA precursor, by splicing in trans. In some embodiments, the linear RNA precursor comprises, from the 5′ end to the 3′ end: a 3′ catalytic Group II intron fragment; a 3′ exon sequence; an effector RNA sequence, and a 5′ exon sequence. In some embodiments, a free 5′ catalytic Group I intron fragment is used in combination with a canonical PIE-style linear RNA precursor (e.g., in combination with a linear RNA precursor comprises from the 5′-end to the 3′ end: a 3′ catalytic Group II intron fragment, a 3′ exon sequence, an effector RNA sequence, a 5′ exon sequence, and a 5′ catalytic Group II intron fragment) to facilitate circularization of the canonical PIE-style linear RNA precursor. In some embodiments, the linear RNA precursor comprises, from the 5′ end to the 3′ end: a 3′ catalytic Group II intron fragment; a 3′ exon sequence; an effector RNA sequence, and a 5′ exon sequence, and does not comprise a 5′ catalytic Group II intron fragment. In some embodiments, the linear RNA precursor comprises a gene of interest (GOI) sequence in the effector sequence. In some embodiments, the GOI comprises an IRES-EGFP sequence. In some embodiments, the splicing in trans involves the linear RNA precursor contacting a free 5′ catalytic Group II intron fragment. In some embodiments, the 3′ catalytic Group II intron fragment of the linear RNA precursor and the free 5′ catalytic Group II intron fragment are capable of accomplishing splicing in trans by splicing the 3′ exon sequence and the 5′ exon sequence from the linear RNA precursor, thereby forming a circRNA comprising the effector RNA.

The linear RNA precursor does not require antisense sequences on the 5′ and 3′ ends in order to splice in trans in any of the embodiments described herein. Thus, in some embodiments, linear RNA precursor for trans splicing does not comprise antisense sequences on the 5′ and 3′ ends. In some embodiments, linear RNA precursor for trans splicing comprises antisense sequences on the 5′ and 3′ ends. The methods of trans splicing described herein further do not require G/U wobble base pairing in order to form circRNA in any of the embodiments described herein. For example, in some embodiments, the linear RNA precursor for trans splicing does not comprise a sequence on the 5′ end of the 3′ catalytic Group II intron fragment that comprises a guanine nucleotide that forms a G/U wobble base pair with a U nucleotide that is present in a sequence on or after the 3′ end of the 3′ catalytic Group II intron fragment. In some embodiments, the linear RNA precursor for trans splicing does not comprise a sequence on the 5′ end of the 3′ catalytic Group II intron fragment that comprises a guanine nucleotide that forms a G/U wobble base pair with a U nucleotide that is present on the same strand of RNA as the free 5′ catalytic Group II intron fragment. In some embodiments, the linear RNA precursor for trans splicing comprises a G/U wobble base pair.

In some embodiments, the sequence of the linear RNA precursor used for splicing in trans comprises one or more sequences set forth in any one of SEQ ID NOs: 175-192, and/or a variant, modification, or derivative thereof. In some embodiments, the 5′ intron sequence used for splicing in trans comprises a 5′ fragment from a sequence set forth in any of SEQ ID NO: 175 (also described herein as Group II intron 1), SEQ ID NO: 176 (also described herein as Group II intron 2), SEQ ID NO: 177 (also described herein as Group II intron 3), SEQ ID NO: 178 (also described herein as Group II intron 4), SEQ ID NO: 179 (also described herein as Group II intron 5), and SEQ ID NO: 180 (also described herein as Group II intron 6), or a variant, modification, or derivative thereof. In some embodiments, the 3′ intron sequence used for splicing in trans comprises a 3′ fragment from a sequence set forth in any of SEQ ID NO: 175 (also described herein as Group II intron 1), SEQ ID NO: 176 (also described herein as Group II intron 2), SEQ ID NO: 177 (also described herein as Group II intron 3), SEQ ID NO: 178 (also described herein as Group II intron 4), SEQ ID NO: 179 (also described herein as Group II intron 5), and SEQ ID NO: 180 (also described herein as Group II intron 6), or a variant, modification, or derivative thereof. In some embodiments, the 3′ exon sequence used for splicing in trans comprises the sequence set forth in any of SEQ ID NO: 187 (also described herein as Group II intron 1 Exon2), SEQ ID NO: 188 (also described herein as Group II intron 2 Exon2), SEQ ID NO: 189 (also described herein as Group II intron 3 Exon2), SEQ ID NO: 190 (also described herein as Group II intron 4 Exon2), SEQ ID NO: 191 (also described herein as Group II intron 5 Exon2), SEQ ID NO: 192 (also described herein as Group II intron 6 Exon2), or a variant, modification, or derivative thereof. In some embodiments, the IRES-EGFP sequence used for splicing in trans comprises the sequence set forth in SEQ ID NO: 5, or a variant, modification, or derivative thereof. In some embodiments, the 5′ exon sequence used for splicing in trans comprises the sequence set forth in any of SEQ ID NO: 181 (also described herein as Group II intron 1 Exon1), SEQ ID NO: 182 (also described herein as Group II intron 2 Exon1), SEQ ID NO: 183 (also described herein as Group II intron 3 Exon1), SEQ ID NO: 184 (also described herein as Group II intron 4 Exon1), SEQ ID NO: 185 (also described herein as Group II intron 5 Exon1), SEQ ID NO: 186 (also described herein as Group II intron 6 Exon1), or a variant, modification, or derivative thereof.

In some embodiments, the free 5′ catalytic Group II intron fragment does not have a G nucleotide on its 5′ end. In some embodiments, the free 5′ catalytic Group II intron fragment has a single G nucleotide on its 5′ end.

In some embodiments, the free 5′ catalytic Group II intron fragment and the linear RNA precursor are present at a 1:1 molar ratio. In some embodiments, the free 5′ catalytic Group II intron fragment and the linear RNA precursor are present at more than a 1:1 molar ratio. In some embodiments, the free 5′ catalytic Group II intron fragment and the linear RNA precursor are present at less than a 1:1 molar ratio. In some embodiments, the free 5′ catalytic Group II intron fragment and the linear RNA precursor are present at a 5′ intron:linear RNA precursor molar ratio of 0-1:1, 1:1-2:1, 2:1-3:1, 3:1-4:1, 4:1-5:1, 5:1-6:1, 6:1-7:1, 7:1-8:1, 8:1-9:1, 9:1-10:1, 10:1-15:1, 15:1-20:1, 20:1-25:1, 25:1-30:1, 30:1-35:1, 35:1-40:1, 40:1-45:1, 45:1-50:1, 50:1-55:1, 55:1-60:1, 60:1-65:1, 65:1-70:1, 70:1-75:1, 75:1-80:1, 80:1-85:1, 85:1-90:1, 90:1-95:1, 95:1-100:1, 100:1-110:1, 110:1-120:1, 120:1-130:1, 130:1-140:1, 140:1-150:1, 150:1-160:1, 160:1-170:1, 170:1-180:1, 180:1-190:1, 190:1-200:1, or above 200:1. In some embodiments, the free 5′ catalytic Group II intron fragment and the linear RNA precursor are present at a 5′ intron:linear RNA precursor molar ratio of 1:1, 1.6:1, 5:1, 8:1, 25:1, 40:1, or 200:1. In some embodiments, the free 5′ catalytic Group II intron fragment and the linear RNA precursor are present at a 5′ intron:linear RNA precursor molar ratio of 5:1 to 10:1.

In some embodiments, the linear RNA precursor further comprises a 5′ homology arm sequence flanking the 5′ of the 3′ catalytic Group II intron fragment, and the free 5′ intron fragment comprises a 3′ homology arm sequence on its 3′ end, wherein the 5′ homology arm sequence and the 3′ homology arm sequence hybridize with each other. In some embodiments, the 5′ homology arm sequence and the 3′ homology arm sequence are each about 5, 10, 15, 20, 25, 30, 35, 40, 45, 50, 55, 60, 65, 70, 75, 80, 85, 90, 95, or 100 nucleotides in length. In some embodiments, the linear RNA precursor does not comprise a 5′ homology arm sequence. In some embodiments, the 5′ homology arm sequence and the 3′ homology arm sequence are each about 0-5, 5-10, 10-15, 15-20, 20-25, 25-30, 35-40, 45-50, 55-60, 65-70, or more than 70 nucleotides in length. In some embodiments, the 5′ homology arm sequence and the 3′ homology arm sequence are each about 0, 4, 9, 14, 19, 24, 29, 34 nucleotides in length. The GC content of the homology arm sequence may vary. In some embodiments, the GC content of the homology arm sequence is 0-5, 5-10, 10-15, 15-20, 20-25, 25-30, 35-40, 45-50, 55-60, 65-70, or more than 70%. In some embodiments, the GC content of the homology arm sequence is 0-5, 5-10, 10-15, or 15-20%.

In some embodiments, the linear RNA precursor comprises a 2′-OMe modification. In some embodiments, the 2′-OMe modification is on the 3′ end of the linear RNA precursor.

### Group I Introns

Any Group I intron known in the art could be used to generate circRNA via self-splicing. Examples of Group I introns useful for the methods of this application are described in Puttaraju, M. & Been, M., Nucleic Acids Res. 20, 5357-5364 (1992); Ford, E. & Ares, M., Proc. Natl Acad. Sci. 91, 3117-3121 (1994); Vicens, Q., Paukstelis, P. J., Westhof, E., Lambowitz, A. M. & Cech, T. R., RNA 14, 2013-2029 (2008), which are incorporated herein by reference.

In some embodiments, the catalytic Group I intron is derived from a bacterial phage Group I intron, such as a Group I intron of a T4 phage. In some embodiments, the catalytic Group I intron is derived from a bacterial Group I intron. In some embodiments, the catalytic Group I intron is derived from a cyanobacteria Group I intron.

In some embodiments, the catalytic Group I intron or fragments thereof are derived from a naturally occurring intron. Examples of naturally occurring introns are known in the art and may be found on, for example, the Group I intron Sequence and Structure Database (Zhou Y, Lu C, Wu Q J, Wang Y, Sun Z T, Deng J C, Zhang Y. GISSD: Group I Intron Sequence and Structure Database. Nucleic Acids Res. 2008 January; 36 (Database issue): D31-7. doi: 10.1093/nar/gkm766. Epub 2007 Oct. 16. PMID: 17942415; PMCID: PMC2238919); each of which is incorporated herein by reference.

In some embodiments, the naturally occurring intron is a member of the IC3 family of Group I introns. In some embodiments, the member of the IC3 family of Group I introns is an Anabaena (Ana) Group I intron. In some embodiments, the naturally occurring intron is a member of the IE2 family of Group I introns. In some embodiments, the member of the IE2 family of Group I introns is a Rasamsonia argillacea (Par, also referred to herein as Rar) Group I intron, a Talaromyces viridulus (Gvi, also referred to herein as Tvi) Group I intron, a Polycephalomyces prolificus (Cpro, also referred to herein as Ppr) Group I intron, a Penicillium oblatum (Pob) Group I intron, a Cordyceps tenuipes (Paecilomyces tenuipes; Pte) Group I intron, a Cordyceps sp. 97009 (Co, also referred to herein as Cor) Group I intron, or a Trichocoma paradoxa (Tpa) Group I intron. In some embodiments, the member of the IE2 family of Group I introns is an intron from a gene annotated as IE2_Par.S516-1 (SEQ ID NO: 56), IE2_Cpro.L2066 (SEQ ID NO: 57), IE2_Pob.S516 (SEQ ID NO: 58), IE2_Pte.L2066 (SEQ ID NO: 59), IE2_Gvi.S516 (SEQ ID NO: 60), IE2_Co_sp-3.L2066 (SEQ ID NO: 61), or IE2_Tpa.S516-1 (SEQ ID NO: 62). In some embodiments, the naturally occurring Group I intron is from Cmu (Coelastrella multistriata) (e.g., SEQ ID NO: 154, the naturally occurring Exon1 sequence associated with which is provided in SEQ ID NO: 160, and the naturally occurring Exon2 sequence associated with which is GCCAGCA), Tar (e.g., SEQ ID NO: 155, the naturally occurring Exon1 sequence associated with which is provided in SEQ ID NO: 161, and the naturally occurring Exon 2 sequence associated with which is provided in SEQ ID NO: 166), Tsp (Trebouxia sp.) (e.g., SEQ ID NO: 156, the naturally occurring Exon1 sequence associated with which is provided in SEQ ID NO: 162, and the naturally occurring Exon 2 sequence associated with which is provided in SEQ ID NO: 167), Hpa (Hypocrea pallida) (e.g., SEQ ID NO: 157, the naturally occurring Exon 1 sequence associated with which is provided in SEQ ID NO: 163, and the naturally occurring Exon 2 sequence associated with which is provided in SEQ ID NO: 168), Tetrahymena (Tetrahymena thermophila) (e.g., SEQ ID NO: 158, the naturally occurring Exon1 sequence associated with which is provided in SEQ ID NO: 164, and the naturally occurring Exon 2 sequence associated with which is provided in SEQ ID NO: 169), and/or Azoarcus (Azoarcus olearius) (e.g., SEQ ID NO: 159, the naturally occurring Exon1 sequence associated with which is provided in SEQ ID NO: 165, and the naturally occurring Exon 2 sequence associated with which is provided in SEQ ID NO: 170). In some embodiments, the naturally occurring intron is a member of the IC1 family of Group I introns. In some embodiments, the member of the IC1 family of Group I introns is a Closterium tumidum (Ctu) Group I intron. In some embodiments, the member of the IC1 family of Group I introns is an intron from a gene annotated as IC1_Ctu.S1506 (SEQ ID NO: 55). In some embodiments, a naturally occurring intron is a homolog, analog, variant, chimeric, or otherwise modified version of any of the aforementioned embodiments. In some embodiments, the catalytic Group I intron or fragments thereof are derived from a sequence having at least 90%, at least 95%, at least 98%, or at least 99% sequence identity over the full length of any of the aforementioned Group I intron sequences. In some embodiments, the catalytic Group I intron or fragments thereof comprise a sequence having at least 90%, at least 95%, at least 98%, or at least 99% sequence identity over the full length of any of the aforementioned Group I intron sequences (such as, e.g., at least 90%, at least 95%, at least 98%, or at least 99% sequence identity over the full length of any intron sequences in any of SEQ ID NOs: 1, 2, 3, 7-9, 13-18, 23, 32, 49-62, 69, 72, 73, 116-138, 148-159, 171-174). In some embodiments, the catalytic Group I intron or fragments thereof comprise a sequence having 100% sequence identity over the full length of any intron sequences in any of the aforementioned Group I intron sequences (such as, e.g., any of SEQ ID NOs: 1, 2, 3, 7-9, 13-18, 23, 32, 49-62, 69, 72, 73, 116-138, 148-159, 171-174). In some embodiments, the catalytic Group I intron or fragments thereof consist of a sequence having 100% sequence identity over the full length of any intron sequences in any of the aforementioned Group I intron sequences (such as, e.g., any of SEQ ID Nos: 1, 2, 3, 7-9, 13-18, 23, 32, 49-62, 69, 72, 73, 116-138, 148-159, 171-174).

In some embodiments, a full length construct (e.g., comprising IRES-EGFP-HBA1) comprising a Group I Cmu (Coelastrella multistriata) intron and associated sequences as provided in SEQ ID NO: 171, a Group I Tar (Trebouxia arboricola) intron and associated sequences as provided in SEQ ID NO: 172, a Group I Tsp (Trebouxia sp.) intron and associated sequences as provided in SEQ ID NO: 173, or a Group I Hpa (Hypocrea pallida) intron and associated sequences as provided in SEQ ID NO: 174, is spliced circularized using the methods provided herein.

In some embodiments, the linear RNA precursor comprises a contiguous full-length sequence of a catalytic Group I intron. In some embodiments, the linear RNA precursor comprises a contiguous sequence of a catalytic 3′ Group I intron fragment. In some embodiments, the sequence of the catalytic Group I intron or catalytic 3′ Group I intron fragment is naturally occurring. In some embodiments, the sequence of the catalytic Group I intron or catalytic 3′ Group I intron fragment is non-naturally occurring.

In some embodiments, the catalytic Group I intron, 3′ catalytic Group I intron fragment, and/or 5′ catalytic Group I intron fragment comprises a heterologous sequence. In some embodiments, the catalytic Group I intron comprises a heterologous sequence. In some embodiments, the 3′ catalytic Group I intron fragment comprises a heterologous sequence. In some embodiments, the 5′ catalytic Group I intron fragment comprises a heterologous sequence. In some embodiments, the 3′ catalytic Group I intron fragment and the 5′ catalytic Group I intron fragment both comprise a heterologous sequence.

In some embodiments, the heterologous sequence comprises a polyA sequence. In some embodiments, the polyA sequence comprises up to 5 contiguous A nucleotides, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, or more than 100 contiguous A nucleotides. In some embodiments, the heterologous sequence comprises 10 contiguous A nucleotides. In some embodiments, the heterologous sequence comprises 15 contiguous A nucleotides. In some embodiments, the heterologous sequence comprises 25 contiguous A nucleotides. In some embodiments, the heterologous sequence comprises 34 contiguous A nucleotides. In some embodiments, the heterologous sequence comprises 35 contiguous A nucleotides. In some embodiments, the heterologous sequence comprises 45 contiguous A nucleotides. In some embodiments, the heterologous sequence comprises 55 contiguous A nucleotides. In some embodiments, the heterologous sequence comprises 65 contiguous A nucleotides. In some embodiments, the heterologous sequence comprises 75 contiguous A nucleotides.

In some embodiments, a heterologous sequence comprising a polyA sequence (such as, for example, a linear RNA precursor comprising a polyA sequence or a spliced intron and/or intron fragment comprising a poly A sequence) can be separated from a sequence not comprising a polyA sequence (such as, for example, a circularized RNA). Such separation can be accomplished using, for example, oligo d(T) beads. In some embodiments, separation using oligo d(T) beads comprises one or more rounds of incubation with the beads. In some embodiments, separation using oligo d(T) beads comprises 1, 2, 3, 4, 5, 6, 7, 8, 9, 10, or more than 10 rounds of incubation with the beads. In some embodiments, separation using oligo d(T) beads comprises 1, 2, 3, or 4 rounds of incubation with the beads. In some embodiments, separation of a sequence comprising about 20-80 contiguous A nucleotides using oligo d(T) beads comprises 1, 2, 3, or 4 rounds of incubation with the beads. In some embodiments, separation of a sequence comprising about 20-80 contiguous A nucleotides using oligo d(T) beads comprises 3 rounds of incubation with the beads. In some embodiments, separation of a sequence comprising about 34 contiguous A nucleotides using oligo d(T) beads comprises 1, 2, 3, or 4 rounds of incubation with the beads. In some embodiments, separation of a sequence comprising about 34 contiguous A nucleotides using oligo d(T) beads comprises 3 rounds of incubation with the beads. In some embodiments, separation of a sequence comprising about 75 contiguous A nucleotides using oligo d(T) beads comprises 1, 2, 3, or 4 rounds of incubation with the beads. In some embodiments, separation of a sequence comprising about 75 contiguous A nucleotides using oligo d(T) beads comprises 3 rounds of incubation with the beads.

In some embodiments, the heterologous sequence is inserted in a loop region of the catalytic Group I intron, 3′ catalytic Group I intron fragment, and/or 5′ catalytic Group I intron fragment. In some embodiments, the heterologous sequence is inserted in a stem region of the catalytic Group I intron, 3′ catalytic Group I intron fragment, and/or 5′ catalytic Group I intron fragment. In some embodiments, the heterologous sequence is inserted at the 3′ end of the catalytic Group I intron, 3′ catalytic Group I intron fragment, and/or 5′ catalytic Group I intron fragment.

In some embodiments, the heterologous sequence can be used to separate reaction products comprising the heterologous sequence, such as separating the catalytic Group I intron, 3′ catalytic Group I intron fragment, and/or 5′ catalytic Group I intron fragment from each other and/or from the circRNA after circularization. In some embodiments, the separation can be by biochemical characteristic of the heterologous sequence, such as by binding to a polyA sequence or other methods known in the art. In some embodiments, the separation can be by physical characteristic of the heterologous sequence, such as by size, charge, hydrophobicity, or other methods known in the art. In some embodiments, the presence of the heterologous sequence does not compromise the catalytic activity of the Group I intron, 3′ catalytic Group I intron fragment, and/or 5′ catalytic Group I intron fragment.

Any method known in the art can be used to assay whether the presence of a heterologous sequence compromises the catalytic activity of the Group I intron, 3′ catalytic Group I intron fragment, and/or 5′ catalytic Group I intron fragment. In some embodiments, assaying whether the presence of a heterologous sequence compromises the catalytic activity of the Group I intron, 3′ catalytic Group I intron fragment, and/or 5′ catalytic Group I intron fragment comprises comparing the products of a linearization reaction in which the Group I intron, 3′ catalytic Group I intron fragment, and/or 5′ catalytic Group I intron fragment includes the heterologous sequence to the products of a linearization reaction in which the Group I intron, 3′ catalytic Group I intron fragment, and/or 5′ catalytic Group I intron fragment does not include the heterologous sequence, in which a reduction of the yield of circRNA under the same conditions in the same amount of time indicates that the presence of a heterologous sequence compromises the catalytic activity of the Group I intron, 3′ catalytic Group I intron fragment, and/or 5′ catalytic Group I intron fragment. In some embodiments, the methods described in the Examples herein may be used to assess whether the catalytic activity of a Group I intron or fragment thereof is disrupted, such as evaluating the proportions of reaction products vs. starting materials by evaluating band strengths corresponding to their respective molecular weights by gel electrophoresis, wherein lighter band(s) corresponding to the reaction products and/or darker band(s) corresponding to the starting materials indicate the catalytic activity is disrupted; and/or by including a gene encoding a measurable reporter, such as a fluorescent protein, in the effector RNA sequence and then transfecting the products of the circularization reaction into HEK293T cells and measuring relative protein production, wherein lower protein production indicates the catalytic activity is disrupted.

In some embodiments, the presence of the heterologous sequence does compromise the catalytic activity of the Group I intron, 3′ catalytic Group I intron fragment, and/or 5′ catalytic Group I intron fragment. In some embodiments, the heterologous sequence can be selected to tune the catalytic activity of the Group I intron. 3′ catalytic Group I intron fragment, and/or 5′ catalytic Group I intron fragment. In some embodiments, the tuning has the effect of attenuating circularization efficiency (i.e., the ratio between the final circRNA and its linear precursor), reaction speed (i.e., how fast the circularization reaction occurs), and/or yield (i.e., the final yield of circRNA relative to the amount of starting linear precursor) relative to a circularization reaction that does not comprise the heterologous sequence. In some embodiments, the tuning has the effect of enhancing circularization efficiency, reaction speed, and/or yield relative to a circularization reaction that does not comprise the heterologous sequence. In some embodiments, the enhancing comprises an increase in circularization efficiency, reaction speed, and/or yield by at least 0.5%, 0.5%-1%, 1%-2%, 2%-5%, 5%-10%, 10%-20%, 20%-30%, 30%-40%, 40%-50%, 50%-60%, 60%-70%, 70%-80%, 80%-90%, 90%-100%, 100%-200%, or more than 200% relative to a circularization reaction that does not comprise the heterologous sequence. In some embodiments, the attenuating comprises an decrease in circularization efficiency, reaction speed, and/or yield by at least 0.5%, 0.5%-1%, 1%-2%, 2%-5%, 5%-10%, 10%-20%, 20%-30%, 30%-40%, 40%-50%, 50%-60%, 60%-70%, 70%-80%, 80%-90%, 90%-100%, 100%-200%, or more than 200% relative to a circularization reaction that does not comprise the heterologous sequence.

### Group II Introns

Any of the methods described herein may alternatively be accomplished using a catalytic Group II intron that undergoes the hydrolysis pathway for splicing (i.e., is self-splicing) in place of a catalytic Group I intron under certain conditions. For example, the same principles described herein for cis splicing in the context of a catalytic Group I intron are also applicable for a catalytic Group II intron, as long as at least the following conditions are met: 1) there are no additional G residues introduced to the 5′ end of the linear RNA precursor (that is, no additional G residue should be added on the 5′ end of the linear RNA precursor); 2) there is no more than one phosphate group (i.e., 0 phosphate groups or 1 phosphate group) on the first G residue in the linear RNA precursor (i.e., on the G residue on or closest to the 5′ end of the linear RNA precursor); and 3) there is not perfect base pairing between the first exon binding sequence (EBS1) and the first intron binding sequence (IBS1) in the linear RNA precursor, as it may lead to cleavage activity near the circRNA ligation site.

Any Group II intron known in the art and meeting the conditions laid out above could be used to generate circRNA via self-splicing. In some embodiments, the Group II intron or fragments thereof are derived from a naturally occurring intron. Examples of naturally occurring Group II introns are known in the art and include, for example, the Group II sequence and structure database (Dai, L., Toor, N., Olson, R., Keeping, A., and Zimmerly, S. (2003), Database for mobile group II introns. Nucleic Acids Res. 31:424-426., Simon M., Dawn, Clarke A. C., Nicholas, McNeil A., Bonnie, Johnson Ian, Pantuso Davin, Dai Lixin, Chai Dinggeng, and Zimmerly, S. (2008), Group II introns in Eubacteria and Archaea: ORF-less introns and new varieties. RNA. 14:1704-1713., and Manuel A. Candales, Adrian Duong, Keyar S. Hood, Tony Li, Ryan A. E. Neufeld, Runda Sun, Bonnie A. McNeil, Li Wu, Ashley M. Jarding, and Steven Zimmerly (2012). Database for bacterial group II introns. Nucleic Acids Research D 187-190). In some embodiments, the Group II intron is derived from a catalytic intron in the Group IIC family; each of which is incorporated herein by reference. In some embodiments, the Group II intron is derived from Bacillus thuringiensis (e.g., SEQ ID NO: 175). In some embodiments, the Group II intron is derived from Clostridium perfringens (e.g., SEQ ID NO:176). In some embodiments, the Group II intron is derived from Anoxybacillus pushchinoensis (e.g., SEQ ID NO: 177). In some embodiments, the Group II intron is derived from Desulforamulus ferrireducens (e.g., SEQ ID NO:178). In some embodiments, the Group II intron is derived from Bacillus smithii (e.g., SEQ ID NO:179). In some embodiments, the Group II intron is derived from Oceanobacillus iheyensis (e.g., SEQ ID NO: 180).

In some embodiments, the catalytic Group II intron is derived from a bacterial Group II intron. In some embodiments, the catalytic Group II intron is derived from an archaebacteria Group II intron. In some embodiments, the catalytic Group II intron is derived from a mitochondrial group II intron. In some embodiments, the catalytic Group II intron is derived from a chloroplast group II intron. In some embodiments, the catalytic Group II intron is derived from a Group IIA intron, Group IIB intron, or Group IIC intron.

In some embodiments, the catalytic activity of the Group II intron, 3′ catalytic Group II intron fragment, and/or 5′ catalytic Group II intron fragment occurs in the absence of GTP. In some embodiments, the catalytic activity of the Group II intron, 3′ catalytic Group II intron fragment, and/or 5′ catalytic Group II intron fragment occurs in the presence of GTP. In some embodiments, the catalytic activity of the Group II intron comprises Watson-Crick base-pairings between the intron “EBS” (Exon Binding Sites) and the exon “IBS” (Intron Binding Sites) sequences. In some embodiments, the catalytic activity of the Group II intron, 3′ catalytic Group II intron fragment, and/or 5′ catalytic Group II intron fragment involves a lariat formation. References for Group II mechanisms include Costa M. Group II Introns: Flexibility and Repurposing. Front Mol Biosci. 2022 Jul. 5; 9:916157. doi: 10.3389/fmolb.2022.916157, the contents of each of which are herein incorporated by reference in their entirety.

In some embodiments, the catalytic Group II intron or fragments thereof comprise domains. In some embodiments, the domains comprise domain I, domain II, domain III, domain IVa, domain IVb, domain V, and/or domain VI. In some embodiments, domain IV comprises the intron-encoded protein (IEP). In some embodiments, domain V comprises the catalytic triad ‘AGC’ sequence. In some embodiments, domain V comprises the catalytic triad ‘CGC’ sequence. In some embodiments, domain V binds catalytically important metal ions. In some embodiments, domain VI comprises the bulged A motif. In some embodiments, the bulged A motif is the branch site during the splicing reaction.

In some embodiments, Group II intron comprises an Oceanobacillus iheyensis Group II intron. In some embodiments, Group II intron comprises Sinorhizobium meliloti Group II intron. References known in the art provide examples of Group II introns, including Molina-Sánchez M D, García-Rodríguez F M, Andrés-León E, Toro N. Identification of Group II Intron RmInt1 Binding Sites in a Bacterial Genome. Front Mol Biosci. 2022 Feb. 25; 9:834020. doi: 10.3389/fmolb.2022.834020, and Marcia M, Somarowthu S, Pyle A M. Now on display: a gallery of group II intron structures at different stages of catalysis. Mob DNA. 2013 May 1; 4 (1): 14. doi: 10.1186/1759-8753-4-14, the contents of each of which are herein incorporated by reference in their entirety.

In some embodiments, a naturally occurring intron is a homolog, analog, variant, chimeric, or otherwise modified version of any of the aforementioned embodiments. In some embodiments, the catalytic Group II intron or fragments thereof are derived from a sequence having at least 90%, at least 95%, at least 98%, or at least 99% sequence identity over the full length of any of the aforementioned Group II intron sequences. In some embodiments, the catalytic Group II intron or fragments thereof comprise a sequence having at least 90%, at least 95%, at least 98%, or at least 99% sequence identity over the full length of any of the aforementioned Group II intron sequences (such as, e.g., at least 90%, at least 95%, at least 98%, or at least 99% sequence identity over the full length of any of SEQ ID NOs: 175-180). In some embodiments, the catalytic Group II intron or fragments thereof comprise a sequence having 100% sequence identity over the full length of any of the aforementioned Group II intron sequences (such as, e.g., any of SEQ ID NOs: 175-180). In some embodiments, the catalytic Group II intron or fragments thereof consist of a sequence having 100% sequence identity over the full length of any of the aforementioned Group II intron sequences (such as, e.g., any of SEQ ID NOs: 175-180).

In some embodiments, the linear RNA precursor comprises a contiguous full-length sequence of a catalytic Group II intron. In some embodiments, the linear RNA precursor comprises a contiguous sequence of a catalytic 3′ Group II intron fragment. In some embodiments, the sequence of the catalytic Group II intron or catalytic 3′ Group II intron fragment is naturally occurring. In some embodiments, the sequence of the catalytic Group II intron or catalytic 3′ Group II intron fragment is non-naturally occurring.

In some embodiments, the catalytic Group II intron, 3′ catalytic Group II intron fragment, and/or 5′ catalytic Group II intron fragment comprises a heterologous sequence. In some embodiments, the catalytic Group II intron comprises a heterologous sequence. In some embodiments, the 3′ catalytic Group II intron fragment comprises a heterologous sequence. In some embodiments, the 5′ catalytic Group II intron fragment comprises a heterologous sequence. In some embodiments, the 3′ catalytic Group II intron fragment and the 5′ catalytic Group II intron fragment both comprise a heterologous sequence.

In some embodiments, the heterologous sequence comprises a polyA sequence. In some embodiments, the polyA sequence comprises up to 5 contiguous A nucleotides, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, or more than 100 contiguous A nucleotides. In some embodiments, the heterologous sequence comprises 10 contiguous A nucleotides. In some embodiments, the heterologous sequence comprises 15 contiguous A nucleotides. In some embodiments, the heterologous sequence comprises 25 contiguous A nucleotides. In some embodiments, the heterologous sequence comprises 34 contiguous A nucleotides. In some embodiments, the heterologous sequence comprises 35 contiguous A nucleotides. In some embodiments, the heterologous sequence comprises 45 contiguous A nucleotides. In some embodiments, the heterologous sequence comprises 55 contiguous A nucleotides. In some embodiments, the heterologous sequence comprises 65 contiguous A nucleotides. In some embodiments, the heterologous sequence comprises 75 contiguous A nucleotides.

In some embodiments, a heterologous sequence comprising a polyA sequence (such as, for example, a linear RNA precursor comprising a polyA sequence or a spliced intron and/or intron fragment comprising a poly A sequence) can be separated from a sequence not comprising a polyA sequence (such as, for example, a circularized RNA). Such separation can be accomplished using, for example, oligo d(T) beads. In some embodiments, separation using oligo d(T) beads comprises one or more rounds of incubation with the beads. In some embodiments, separation using oligo d(T) beads comprises 1, 2, 3, 4, 5, 6, 7, 8, 9, 10, or more than 10 rounds of incubation with the beads. In some embodiments, separation using oligo d(T) beads comprises 1, 2, 3, or 4 rounds of incubation with the beads. In some embodiments, separation of a sequence comprising about 20-80 contiguous A nucleotides using oligo d(T) beads comprises 1, 2, 3, or 4 rounds of incubation with the beads. In some embodiments, separation of a sequence comprising about 20-80 contiguous A nucleotides using oligo d(T) beads comprises 3 rounds of incubation with the beads. In some embodiments, separation of a sequence comprising about 34 contiguous A nucleotides using oligo d(T) beads comprises 1, 2, 3, or 4 rounds of incubation with the beads. In some embodiments, separation of a sequence comprising about 34 contiguous A nucleotides using oligo d(T) beads comprises 3 rounds of incubation with the beads. In some embodiments, separation of a sequence comprising about 75 contiguous A nucleotides using oligo d(T) beads comprises 1, 2, 3, or 4 rounds of incubation with the beads. In some embodiments, separation of a sequence comprising about 75 contiguous A nucleotides using oligo d(T) beads comprises 3 rounds of incubation with the beads.

In some embodiments, the heterologous sequence is inserted in a loop region of the catalytic Group II intron, 3′ catalytic Group II intron fragment, and/or 5′ catalytic Group II intron fragment. In some embodiments, the heterologous sequence is inserted in a stem region of the catalytic Group II intron, 3′ catalytic Group II intron fragment, and/or 5′ catalytic Group II intron fragment. In some embodiments, the heterologous sequence is inserted at the 3′ end of the catalytic Group II intron, 3′ catalytic Group II intron fragment, and/or 5′ catalytic Group II intron fragment.

In some embodiments, the heterologous sequence can be used to separate reaction products comprising the heterologous sequence, such as separating the catalytic Group II intron, 3′ catalytic Group II intron fragment, and/or 5′ catalytic Group II intron fragment from each other and/or from the circRNA after circularization. In some embodiments, the separation can be by biochemical characteristic of the heterologous sequence, such as by binding to a polyA sequence or other methods known in the art. In some embodiments, the separation can be by physical characteristic of the heterologous sequence, such as by size, charge, hydrophobicity, or other methods known in the art. In some embodiments, the presence of the heterologous sequence does not compromise the catalytic activity of the Group II intron, 3′ catalytic Group II intron fragment, and/or 5′ catalytic Group II intron fragment.

Any method known in the art can be used to assay whether the presence of a heterologous sequence compromises the catalytic activity of the Group II intron, 3′ catalytic Group II intron fragment, and/or 5′ catalytic Group II intron fragment. In some embodiments, assaying whether the presence of a heterologous sequence compromises the catalytic activity of the Group II intron, 3′ catalytic Group II intron fragment, and/or 5′ catalytic Group II intron fragment comprises comparing the products of a linearization reaction in which the Group II intron, 3′ catalytic Group II intron fragment, and/or 5′ catalytic Group II intron fragment includes the heterologous sequence to the products of a linearization reaction in which the Group II intron, 3′ catalytic Group II intron fragment, and/or 5′ catalytic Group II intron fragment does not include the heterologous sequence, in which a reduction of the yield of circRNA under the same conditions in the same amount of time indicates that the presence of a heterologous sequence compromises the catalytic activity of the Group II intron, 3′ catalytic Group II intron fragment, and/or 5′ catalytic Group II intron fragment. In some embodiments, the methods described in the Examples herein may be used to assess whether the catalytic activity of a Group II intron or fragment thereof is disrupted, such as evaluating the proportions of reaction products vs. starting materials by evaluating band strengths corresponding to their respective molecular weights by gel electrophoresis, wherein lighter band(s) corresponding to the reaction products and/or darker band(s) corresponding to the starting materials indicate the catalytic activity is disrupted; and/or by including a gene encoding a measurable reporter, such as a fluorescent protein, in the effector RNA sequence and then transfecting the products of the circularization reaction into HEK293T cells and measuring relative protein production, wherein lower protein production indicates the catalytic activity is disrupted.

In some embodiments, the presence of the heterologous sequence does compromise the catalytic activity of the Group II intron, 3′ catalytic Group II intron fragment, and/or 5′ catalytic Group II intron fragment. In some embodiments, the heterologous sequence can be selected to tune the catalytic activity of the Group II intron. 3′ catalytic Group II intron fragment, and/or 5′ catalytic Group II intron fragment. In some embodiments, the tuning has the effect of attenuating circularization efficiency (i.e., the ratio between the final circRNA and its linear precursor), reaction speed (i.e., how fast the circularization reaction occurs), and/or yield (i.e., the final yield of circRNA relative to the amount of starting linear precursor) relative to a circularization reaction that does not comprise the heterologous sequence. In some embodiments, the tuning has the effect of enhancing circularization efficiency, reaction speed, and/or yield relative to a circularization reaction that does not comprise the heterologous sequence. In some embodiments, the enhancing comprises an increase in circularization efficiency, reaction speed, and/or yield by at least 0.5%, 0.5%-1%, 1%-2%, 2%-5%, 5%-10%, 10%-20%, 20%-30%, 30%-40%, 40%-50%, 50%-60%, 60%-70%, 70%-80%, 80%-90%, 90%-100%, 100%-200%, or more than 200% relative to a circularization reaction that does not comprise the heterologous sequence. In some embodiments, the attenuating comprises an decrease in circularization efficiency, reaction speed, and/or yield by at least 0.5%, 0.5%-1%, 1%-2%, 2%-5%, 5%-10%, 10%-20%, 20%-30%, 30%-40%, 40%-50%, 50%-60%, 60%-70%, 70%-80%, 80%-90%, 90%-100%, 100%-200%, or more than 200% relative to a circularization reaction that does not comprise the heterologous sequence.

### Exon Sequences

The linear RNA precursor comprises a 3′ exon sequence (referred to interchangeably herein as “Exon2”) and a 5′ exon sequence (referred to interchangeably herein as “Exon1”). In some embodiments, the exon sequences are derived from the same genomic sequence as the naturally occurring intron from which the catalytic Group I intron or fragments thereof or catalytic Group II intron or fragments thereof are derived, such that the exon sequences and Group I intron sequences correspond, or such that the exon sequences and Group II intron sequences correspond. In some embodiments, the 3′ exon sequence is no more than about 4, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides long. In some embodiments, the 3′ exon sequence is 1, 3, 6, 11, 16, 30, or 52 nucleotides long.

In some embodiments, the 5′ exon sequence is no more than 4, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, or about 200 nucleotides long. In some embodiments, the 5′ exon sequence is 1, 3, 6, 11, 16, 30, or 52 nucleotides long. In some embodiments, the 3′ exon sequence and the 5′ exon sequence are each 1, 3, 6, 11, 16, 30, or 52 nucleotides long.

In some embodiments, the 3′ exon sequence and/or the 5′ exon sequence are derived from a member of the IC3 family of Group I introns. In some embodiments, the member of the IC3 family of Group I introns is an Anabaena (Ana) Group I intron. In some embodiments, the 3′ exon sequence and/or the 5′ exon sequence are derived from a member of the IE2 family of Group I introns. In some embodiments, the member of the IE2 family of Group I introns is a Rasamsonia argillacea (Par) Group I intron, a Talaromyces viridulus (Gvi) Group I intron, a Polycephalomyces prolificus (Cpro) Group I intron, a Penicillium oblatum (Pob) Group I intron, a Cordyceps tenuipes (Paecilomyces tenuipes; Pte) Group I intron, a Cordyceps sp. 97009 (Co) Group I intron, or a Trichocoma paradoxa (Tpa) Group I intron. In some embodiments, the member of the IE2 family of Group I introns is an intron from a gene annotated as IE2_Par.S516-1, IE2_Cpro.L2066, IE2_Pob.S516, IE2_Pte.L2066, IE2_Gvi.S516, IE2_Co_sp-3.L2066, or IE2_Tpa. S516-1. In some embodiments, the 3′ exon sequence and/or the 5′ exon sequence are derived from a member of the IC1 family of Group I introns. In some embodiments, the member of the IC1 family of Group I introns is a Closterium tumidum (Ctu) Group I intron. In some embodiments, the member of the IC1 family of Group I introns is an intron from a gene annotated as IC1_Ctu.S1506. In some embodiments, the =3′ exon sequence and/or the 5′ exon sequence is from Coelastrella multistriata (Cmu) (e.g., SEQ ID NO: 154, the naturally occurring Exon1 sequence associated with which is provided in SEQ ID NO: 160, and the naturally occurring Exon2 sequence associated with which is GCCAGCA), Trebouxia arboricola (Tar) (e.g., SEQ ID NO: 155, the naturally occurring Exon 1 sequence associated with which is provided in SEQ ID NO: 161, and the naturally occurring Exon 2 sequence associated with which is provided in SEQ ID NO: 166), Trebouxia sp. (Tsp) (e.g., SEQ ID NO: 156, the naturally occurring Exon1 sequence associated with which is provided in SEQ ID NO: 162, and the naturally occurring Exon 2 sequence associated with which is provided in SEQ ID NO: 167), Hypocrea pallida (Hpa) (e.g., SEQ ID NO: 157, the naturally occurring Exon1 sequence associated with which is provided in SEQ ID NO: 163, and the naturally occurring Exon 2 sequence associated with which is provided in SEQ ID NO: 168), Tetrahymena (Tetrahymena thermophila) (e.g., SEQ ID NO: 158, the naturally occurring Exon1 sequence associated with which is provided in SEQ ID NO: 164, and the naturally occurring Exon 2 sequence associated with which is provided in SEQ ID NO: 169), and/or Azoarcus (Azoarcus olearius) (e.g., SEQ ID NO: 159, the naturally occurring Exon1 sequence associated with which is provided in SEQ ID NO: 165, and the naturally occurring Exon 2 sequence associated with which is provided in SEQ ID NO: 170). In some embodiments, the 3′ exon sequence and/or the 5′ exon sequence are derived from a homolog, analog, variant, chimeric, or otherwise modified version of any of the aforementioned embodiments. In some embodiments, the catalytic Group I exon or fragments thereof are derived from a sequence having at least 90%, at least 95%, at least 98%, or at least 99% sequence identity over the full length of any of the aforementioned Group I exon sequences. In some embodiments, the catalytic Group I exon or fragments thereof comprise a sequence having at least 90%, at least 95%, at least 98%, or at least 99% sequence identity over the full length of any of the aforementioned Group I exon sequences (such as, e.g., at least 90%, at least 95%, at least 98%, or at least 99% sequence identity over the full length of any exon sequences in any of SEQ ID NOs: 2, 4, 6, 10, 11, 19-22, 24, 25, 30, 31, 33-48, 63-67, 76-80, 160-170). In some embodiments, the catalytic Group I exon or fragments thereof comprise a sequence having 100% sequence identity over the full length of any exon sequences in any of the aforementioned Group I exon sequences (such as, e.g., any of SEQ ID NOs: 2, 4, 6, 10, 11, 19-22, 24, 25, 30, 31, 33-48, 63-67, 76-80, 160-170). In some embodiments, the catalytic Group I exon or fragments thereof consist of a sequence having 100% sequence identity over the full length of any exon sequences in any of the aforementioned Group I exon sequences (such as, e.g., any of SEQ ID Nos: 2, 4, 6, 10, 11, 19-22, 24, 25, 30, 31, 33-48, 63-67, 76-80, 160-170).

In some embodiments, the 3′ exon sequence and/or the 5′ exon sequence are derived from a Group II intron. In some embodiments, the 3′ exon sequence and/or the 5′ exon sequence are derived from a member of the IIC family of Group II introns. In some embodiments, the catalytic Group II exon or fragments thereof are derived from a sequence having at least 90%, at least 95%, at least 98%, or at least 99% sequence identity over the full length of any of the aforementioned Group II exon sequences. In some embodiments, the catalytic Group II exon or fragments thereof comprise a sequence having at least 90%, at least 95%, at least 98%, or at least 99% sequence identity over the full length of any of the aforementioned Group II exon sequences (such as, e.g., at least 90%, at least 95%, at least 98%, or at least 99% sequence identity over the full length of any exon sequences in any of SEQ ID NOs: 181-192). In some embodiments, the catalytic Group II exon or fragments thereof comprise a sequence having 100% sequence identity over the full length of any exon sequences in any of the aforementioned Group II exon sequences (such as, e.g., any of SEQ ID NOs: 181-192). In some embodiments, the catalytic Group II exon or fragments thereof consist of a sequence having 100% sequence identity over the full length of any exon sequences in any of the aforementioned Group II exon sequences (such as, e.g., any of SEQ ID Nos: 181-192).

In some embodiments, the exon sequences are modified or mutated from a naturally occurring exon sequence. In some embodiments, an additional nucleotide sequence is present on the 3′ end of Exon1. In some embodiments, an additional 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more nucleotides are present on the 3′ end of Exon1. In some embodiments, an additional 7 nucleotides are present on the 3′ end of Exon1. In some embodiments, the additional nucleotide sequence comprises SEQ ID NO: 12.

The minimal requirements for an exon to function may vary between different exons. In some embodiments, the minimal length of Exon2 in order for the Exon2 sequence to function varies from 0-nt to more nucleotides according to the RNA structure. In some embodiments, the minimal length of Exon 1 in order for the Exon1 sequence to function is at least about 6 nucleotides in order to bind with the IGS (internal guide sequence) in the intron.

### Scar Sequences

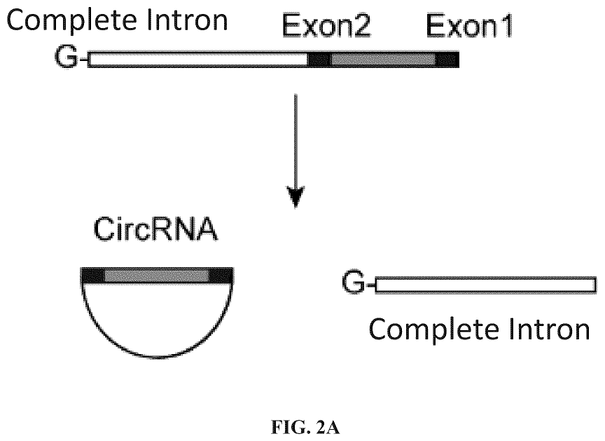

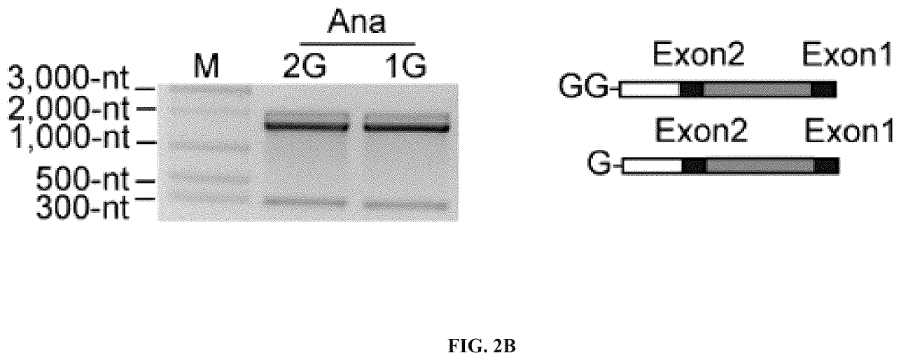

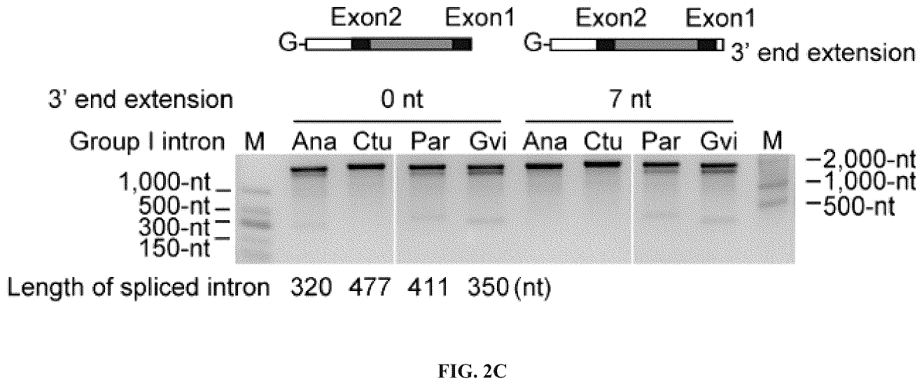

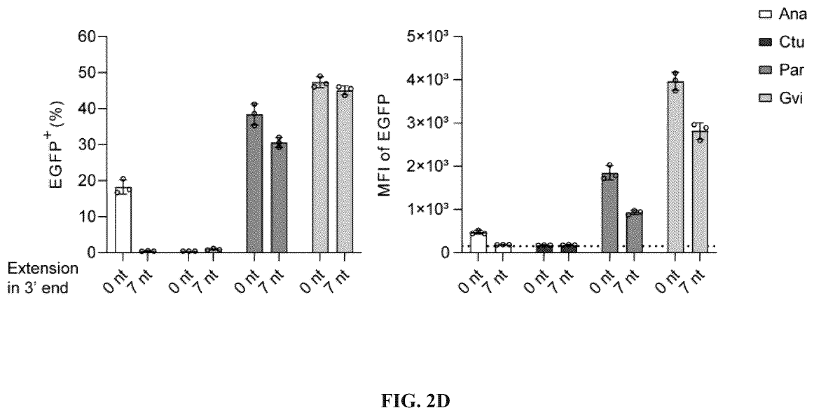

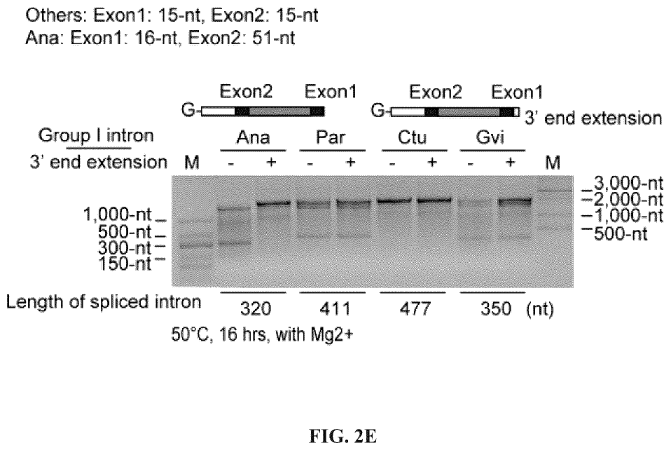

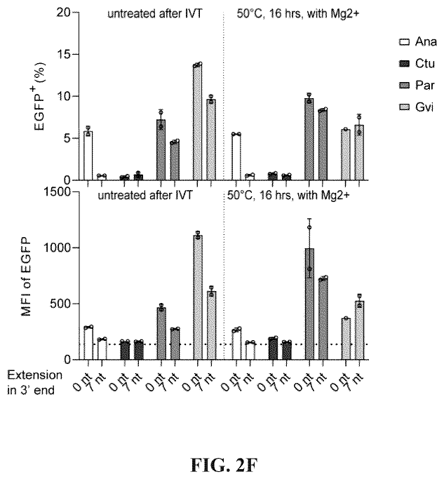

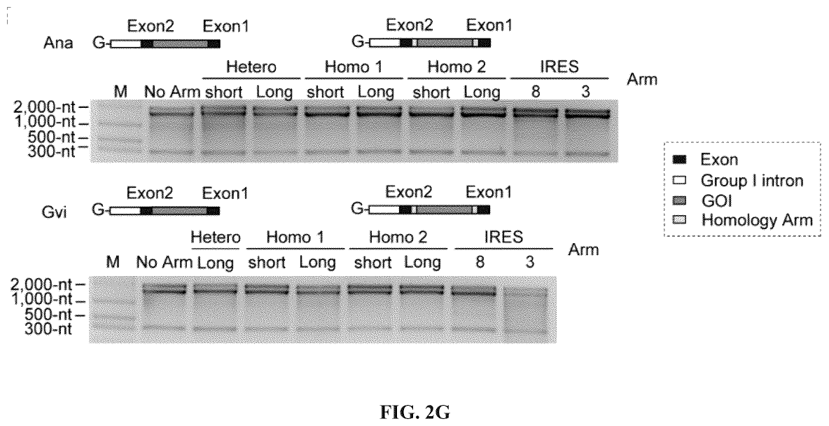

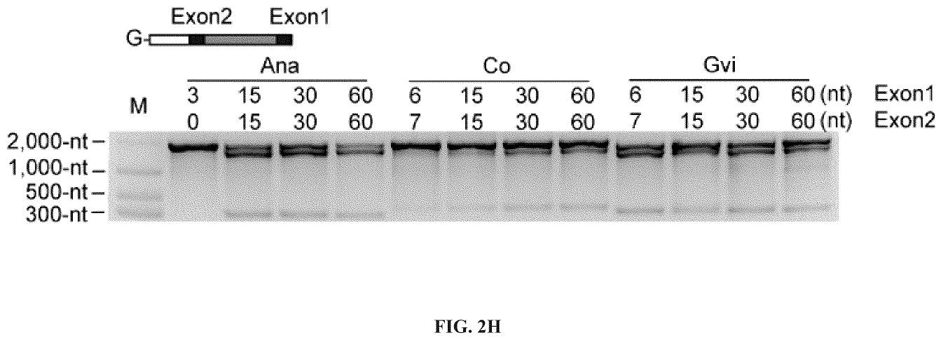

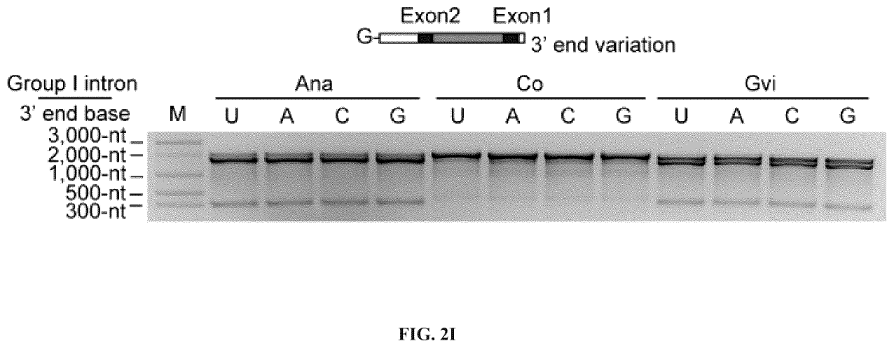

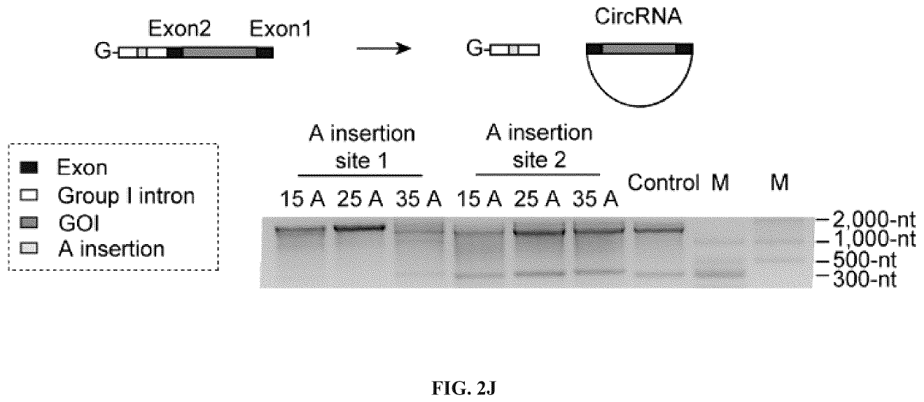

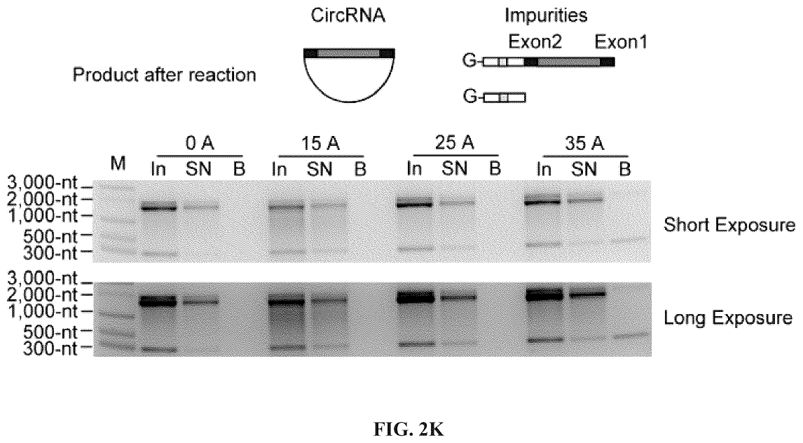

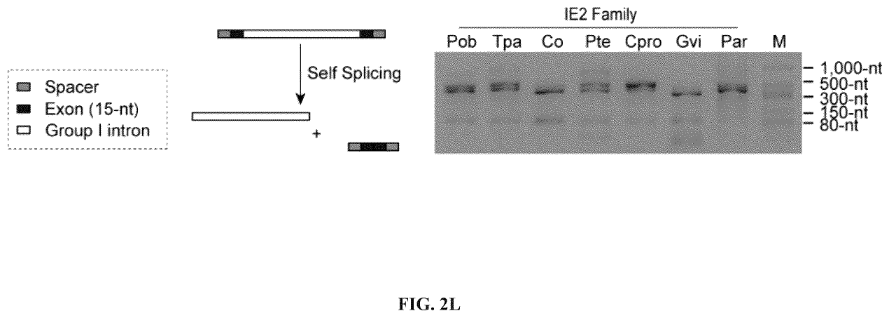

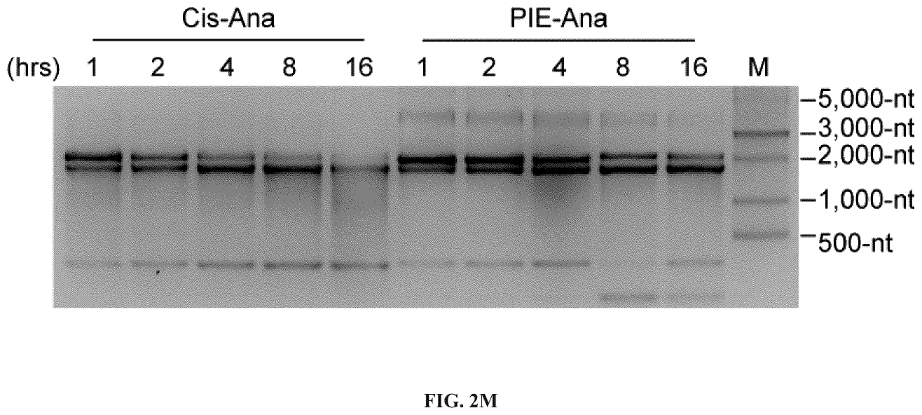

In some embodiments, one or more exon sequences and/or other sequences (e.g., other non-effector sequences, such as homology arms and/or linker sequences, if present in the linear RNA) from a linear RNA precursor are retained in a circularized RNA product. Such sequences in a circRNA (e.g., residual exon sequences, such as the Exon1 and Exon2 portions of the circRNA in the bottom left of FIG. 2A) may be described as “scar” sequences. In some embodiments, scar sequences are immunogenic. In some embodiments, a circRNA product has one or more scar sequences.

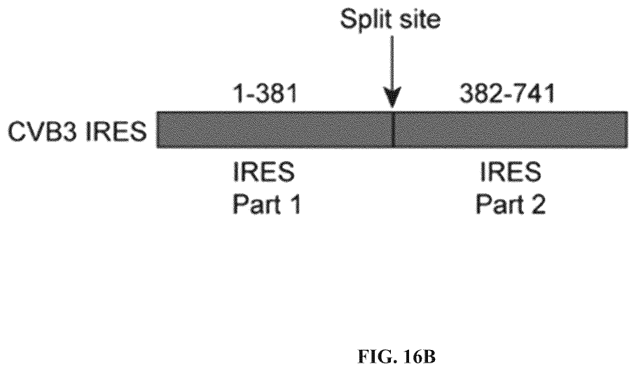

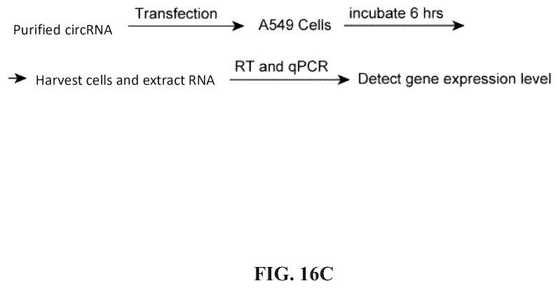

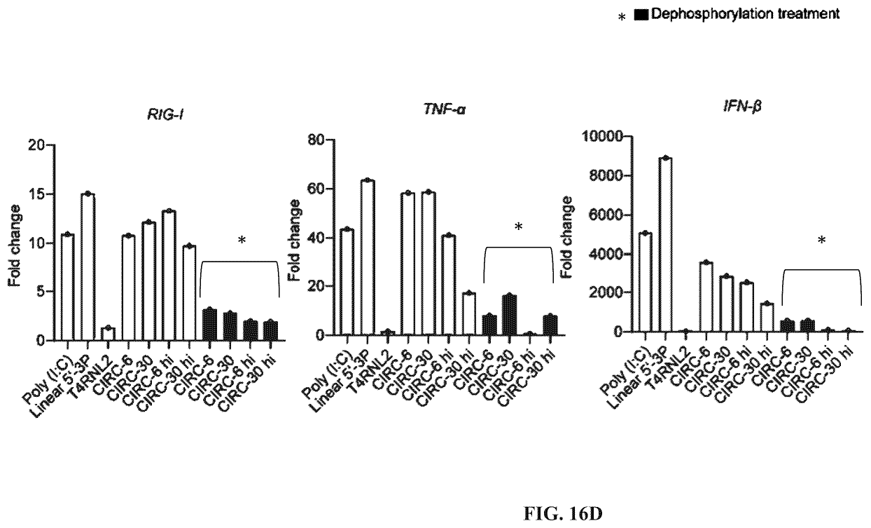

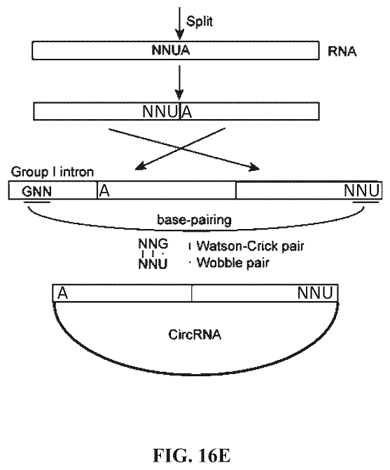

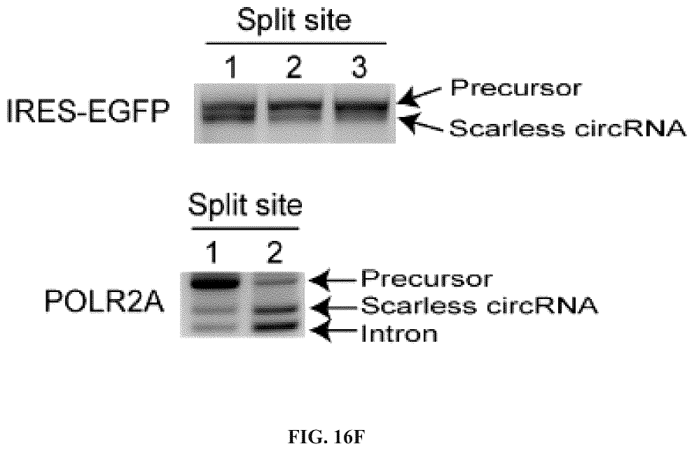

In some embodiments, a scar sequence is a short scar sequence (i.e., the scar sequence is truncated compared to a corresponding wild-type sequence). In some embodiments, a scar sequence is “hidden” or “camouflaged” in another sequence (such as, for example, being located within an IRES sequence or within an untranslated region (UTR)) in a circRNA product while maintaining the integrity of the open reading frame sequence and the integrity of the translation ability of the IRES and UTRs. Such “hidden” scars may also be described as “invisible”, and circRNAs comprising such an “invisible” or “hidden” scars may also be described as “scarless” or not comprising a scar (i.e., no scar). The total length of a circRNA circularized by cis splicing and comprising a scar, a short scar, a hidden scar, or no scar, is the same as the total length of the respective linear precursor. The hidden scar or scarless effect is generated by designing the linear RNA precursor sequence such that the sequences used as exons, when circularized, end up within another sequence outside the payload (e.g., within a linearized IRES sequence; see, e.g., FIG. 16A). For example, in the context of a linear RNA precursor having an Ana intron in cis splicing, a split site for the exon sequences (that is, to define the 3′ end of the 5′ exon sequence and the 5′ end of the 3′ exon sequence) may be designed within an NNUA motif to generate an “invisible” or hidden scar (i.e., a scarless effect; see, e.g., FIG. 16E), where “N” represents A, U, C, or G. For example, as shown in FIG. 16E, during circularization of a linear RNA precursor having an Ana Group I intron and a split site designed between the U and A residues of the NNUA motif, the U in “NNUA” base pairs with a G residue of the Group I intron as a wobble pair, while the “NN” residues of the motif pair in Watson-Crick pairs with corresponding Watson-Crick pairing residues in the Group I intron. For example, the NNUA motif may be CUUA in a linear RNA precursor having an Ana intron, such that the exon1 sequence comprises a CUU sequence that base pairs with a GAG motif in the Ana intron during splicing.

In some embodiments, the intron sequence naturally comprises a motif that base pairs with a chosen split motif sequence (e.g., the intron sequence comprises a GAG motif to base pair with a CUU motif in exon1 during splicing, or comprises another GNN motif that is able to base pair with an NNU motif in exon1 during splicing). In some embodiments, the intron sequence is mutated to base pair with a chosen split motif sequence (e.g., engineering an intron lacking a GAG motif to comprise a GAG motif to base pair with a CUU motif in exon1 during splicing, or otherwise engineering a GNN motif into an intron sequence to base pair with an NNU motif in exon1 during splicing). In some embodiments (such as, e.g., as shown in FIG. 16E), one or more base pairs between the intron sequence and the corresponding exon split sequence motif exhibit wobble pairing (that is, non-Watson-Crick pairing; e.g., G-U pairing). In some embodiments, two base pairs between the intron sequence and the corresponding exon split sequence motif exhibit Watson-Crick pairing (i.e., A-U or G-C) and one base pair exhibits wobble pairing (such as, e.g., as shown in FIG. 16E).

Thus, in some embodiments, an exon is split within an NNUA motif to produce the 5′ exon sequence and the 3′ exon sequence for use in any of the methods described herein (such as, for example, for design of a linear RNA precursor comprising a Group I intron, such as an Ana intron, for cis splicing). For example, in some embodiments, the 3′ end of the 5′ exon sequence in a linear RNA precursor comprises the residues NNU (e.g., AAU, UUU, GGU, CCU, CUU, etc.), NN, or N from the original NNUA motif, while the 5′ end of the 3′ exon sequence in the linear RNA precursor comprises an A base, an AU, or an AUN from the original NNUA motif, respectively. In such embodiments, the NNU (e.g., CUU) sequence in exon1 base pairs with a corresponding sequence (e.g., GAG) that is either naturally present or engineered into the intron sequence.

In some embodiments (such as, e.g., for linear RNA precursors comprising an intron other than the Ana intron), a motif other than NNUA is used to design the split site for the exon. Thus, the same principles laid out above and in FIG. 16E may be applied to other split motifs in order to generate scarless circRNAs using the methods provided herein.

Any split site (e.g., within an IRES or a UTR) may be chosen in which to hide the scar. The split site splits the split sequence (e.g., an IRES sequence) into two parts, which may be of equal or unequal lengths. The part comprising the 5′ portion of the sequence that is split may be referred to as part 1 (e.g., IRES part 1), while the part comprising the 3′ portion of the sequence that is split may be referred to as part 2 (e.g., IRES part 2). Thus, in some embodiments, the linear RNA precursor comprises, from 5′ to 3′, an intron sequence or fragment thereof, part 2 of the split sequence (e.g., IRES part 2) beginning with the base or bases that will function as the 3′ exon sequence (e.g., beginning with an A base), a payload, and part 1 of the split sequence, ending with the bases that will function as the 5′ exon (e.g., ending with an NNU sequence). For example, the 3′ exon sequence may comprise a 3′ A base and the corresponding 5′ exon sequence may comprise a 5′ NNU motif, wherein N represents an A, U, C, or G residue. In some embodiments, the 3′ exon sequence consists of a 3′ A base and the corresponding 5′ exon sequence consists of a 5′ NNU motif, wherein N represents an A, U, C, or G residue.

The split site may be at any location in which a scar is desired to be hidden, so long as the motif of the spit site can base pair with a corresponding motif in the intron sequence (e.g., can base pair with at least three bases in a corresponding motif in the intron sequence, such as, e.g., as shown in FIG. 16E). For example, a split site may be in any NNUA motif in an IRES sequence. In such embodiments, a linear RNA may comprise an intron comprising a motif that can base pair to the NNU portion of the motif, such as, e.g., a GAG motif in an Ana intron. In some embodiments, the split site is at any location between residues 1 and 741 of an IRES sequence. In some embodiments, the split site is at any location between residues 100 and 700, between residues 200 and 600, between residues 300 and 500, between residues 300 and 400, between residues 350 and 400, between residues 370 and 390, between residues 375 and 386 of an IRES sequence. In some embodiments, the split site is between residues 381 and 382 of an IRES sequence. In some embodiments, the split site is between any of the aforementioned residues in a CVB IRES sequence (such as, e.g., between residues 381 and 382 of a CVB3 IRES sequence).

In some embodiments, a hidden scar sequence exhibits reduced immunogenicity compared to a corresponding non-hidden scar. Immunogenicity may be measured in a variety of ways. For example, immunogenicity may be measured by quantifying expression of immunity-related or immune system-related genes (such as, for example, retinoic acid-inducible gene I (RIG-I), Tumour Necrosis Factor alpha (TNF-alpha), interferon beta (IFN-beta), and/or any other immunity-related or immune system-related genes known in the art) in the presence of a circRNA product. In some embodiments, immune genes comprise RIG-I, PKR, melanoma-differentiation-associated gene 5 (MI) A₅), 2′-5′-oligoadentylate synthase 1 (OASL), OAS-like protein (OASL), IFNβ, TNFα, and/or interleukin-6 (IL-6). Immunogenicity in circRNAs is further described in references, Tai J, Chen Y G. Differences in the immunogenicity of engineered circular RNAs. J Mol Cell Biol. 2023 Jun. 1; 15 (1): mjad002 and Liu, C. X. et al. RNA circles with minimized immunogenicity as potent PKR inhibitors. Mol Cell 82, 420-434 e426 (2022)), the contents of which are herein incorporated by reference in their entirety.

Relative immunogenicity may then be quantified by comparing a measured immunogenic measurement (for instance, as described above) with a corresponding measurement in the absence of the circRNA product or in the presence of a corresponding control construct of known immunogenicity (for example, that is known to trigger an immune response). Accordingly, reduced immunogenicity may be measured by, for example, a reduction in measured transcription and/or reduction in measured translation of one or more immunity-related or immune system-related genes in the presence of a circRNA as compared to in the presence of a corresponding control construct (such as, for example, a control construct known to induce an immunogenic response). Similarly, increased immunogenicity may be measured by, for example, an increase in measured transcription and/or increase in measured translation of one or more immunity-related or immune system-related genes in the presence of a circRNA as compared to in the presence of a corresponding control construct (such as, for example, a control construct known to not induce an immunogenic response or to induce only a minimal immunogenic response).

In some embodiments, a circRNA has one scar. In some embodiments, a circRNA has one scar derived from two sequences from the linear RNA precursor (e.g., a first portion of the scar sequence and a second portion of the scar sequence, wherein the first portion of the scar sequence includes a 3′ exon sequence, and the second portion of the scar sequence includes a 5′ exon sequence) In some embodiments, a scar sequence in a circRNA is hidden in an IRES sequence. In some embodiments, a scar sequence in a circRNA is hidden in a UTR sequence. In some embodiments, a scar sequence in a circRNA is hidden in a 5′ UTR sequence. In some embodiments, a scar sequence in a circRNA is hidden in a 3′ UTR sequence.

In some embodiments, a short scar is shorter than its corresponding wild-type sequence (e.g., a scar may be a fragment of a wild-type exon sequence) in a linear RNA precursor by 1, 2, 3, 4, 5, 6, 7, 8, 9, 10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-125, 125-150, 150-175, or 175-200 nucleotides. In some embodiments, a short scar is no more than 40-50, 30-40, 20-30, 20, 19, 18, 17, 16, 15, 14, 13, 12, 11, 10, 9, 8, 7, 6, 5, 4, 3, 2, or 1 nucleotides long. In some embodiments, a short scar is 40-50, 30-40, 20-30, 20, 19, 18, 17, 16, 15, 14, 13, 12, 11, 10, 9, 8, 7, 6, 5, 4, 3, 2, or 1 nucleotides long. In some embodiments, a short scar is 6 nucleotides long. In some embodiments, a short scar is 3 nucleotides long. In some embodiments, a short scar is 2 nucleotides long. In some embodiments, a short scar is 1 nucleotide long. In any of the above embodiments, the circRNA may have reduced immunogenicity compared to a corresponding circRNA having one or more longer scar sequences.

In some embodiments, no 5′ exon sequences or fragments thereof from a linear RNA precursor are present outside of an IRES sequence in a circRNA, which may result in reduced immunogenicity compared to a circRNA comprising 5′ exon sequences outside of an IRES sequence. In some embodiments, no 3′ exon sequences or fragments thereof from a linear RNA precursor are present outside of an IRES sequence in a circRNA, which may result in reduced immunogenicity compared to a circRNA comprising 3′ exon sequences outside of an IRES sequence. In some embodiments, no 5′ exon sequences or fragments thereof and no 3′ exon sequences or fragments thereof from a linear RNA precursor are present in a circRNA outside of an IRES sequence, which may result in reduced immunogenicity compared to a circRNA comprising 5′ and/or 3′ exon sequences outside of an IRES sequence. In some embodiments, no homology arm sequences or fragments thereof from a linear RNA precursor are present in a circRNA outside of an IRES sequence, which may result in reduced immunogenicity compared to a circRNA comprising homology arm sequences outside of an IRES sequence. In some embodiments, no flexible linker sequences or fragments thereof from a linear RNA precursor are present in a circRNA outside of an IRES sequence, which may result in reduced immunogenicity compared to a circRNA comprising flexible linker sequences outside of an IRES sequence. In some embodiments, a circRNA product has no scar sequences outside of an IRES sequence. In some of any of the above embodiments, the circRNA has reduced immunogenicity compared to a circRNA having one or more scar sequences outside of an IRES sequence.

### Effector RNA

The linear RNA precursors and circRNAs described herein comprise an effector RNA sequence (also described herein as a “payload”), which may be a coding RNA sequence or a non-coding RNA sequence. Exemplary non-coding RNAs include, but are not limited to, guide RNAs (gRNA, including single guide RNA or sgRNA), a deaminase-recruiting RNA (dRNA), a small RNA (such as a microRNA, a short hairpin RNA, or a small interfering RNA), or a long intervening non-coding RNA (lincRNA).

In some embodiments, the effector RNA sequence is at least about 50 nt long, such as at least about any one of 100, 150, 200, 300, 600, 900, 1200, 1500, 2000, 3000, 4000, 5000, or more nt long. In some embodiments, the effector RNA sequence is no more than about any one of 5000, 4000, 3000, 2000, 1500, 1200, 900, 600, 300, 200, 150, or 100 nt long. In some embodiments, the effector RNA sequence is about any one of 50-100, 100-500, 500-1000, 1000-2000, 2000-5000, 50-5000, 100-5000, 100-3000, 500-5000, 500-2500, 2500-5000, 1000-5000, 5000-6000, 6000-7000, 7000-8000, 8000-9000, 9000-10000, 10000-11000, 11000-12000, 12000-13000, 13000-14000, 14000-15000, 15000-16000, 16000-17000, 17000-18000, 18000-19000, 19000-20000, 20000-25000, 25000-30000, 30000-50000, 50000-100000, 100000-200000, 200000-500000, or more than 500000 nt long. In some embodiments, the effector RNA sequence is about 4500 nt long. In some embodiments, the effector RNA sequence is more than about 8000 nt long. In some embodiments, the effector RNA sequence is about 10000-14000 nt long, such as, for example, about 12000 nt long. In some embodiments, the effector RNA sequence is more than about 12000 nt long.

In some embodiments, the effector RNA sequence is a coding RNA sequence, which encode any polypeptide of interest. In some embodiments, the polypeptide is at least about 15 amino acids long, such as at least about any one of 20, 50, 100, 200, 300, 400, 500, 600, 700, 800, 900, 1000, 2000, 3000, 4000, 5000, 6000 or more amino acids long. In some embodiments, the polypeptide is no more than about any one of 10000, 9000, 8000, 7000, 6000, 5000, 4000, 3000, 2000, 1000, 900, 800, 700, 600, 500, 400, 300, 200, 100, 50, or 20 amino acids long. In some embodiments, the polypeptide is about any one of 20-50, 50-100, 20-200, 20-500, 20-1000, 50-500, 50-1000, 100-500, 100-1000, 200-1000, 500-1000, 1000-2000, 2000-3000, 3000-4000, 4000-5000, or more than about 5000 amino acids long. In some embodiments, the polypeptide is about 3500-4000 amino acids long. In some embodiments, the polypeptide is about 3600-3700 amino acids long.

In some embodiments, the effector RNA sequence comprises a nucleic acid sequence encoding a sequence of interest. In some embodiments, the sequence of interest codes for a gene of interest (GOI). In some embodiments, the effector RNA sequence comprises a nucleic acid sequence encoding a therapeutic polypeptide. In some embodiments, the therapeutic polypeptide is selected from the group consisting of an antigenic polypeptide, a functional protein, a receptor protein, and a targeting protein.

In some embodiments, the coding RNA sequence encodes an antigenic polypeptide. A circRNA vaccine may be prepared using a linear RNA comprising a coding RNA sequence encoding an antigenic polypeptide. An antigenic polypeptide comprises at least one epitope recognizable by a T cell receptor (TCR). In some embodiments, the antigenic polypeptide is a full-length protein or a fragment thereof, or an antigenic fusion protein that can trigger an immune response in a subject. In some embodiments, the antigenic polypeptide is a short peptide of no more than 100 amino acids long. The antigenic polypeptide can be a naturally derived peptide fragment from a protein antigen containing one or more epitopes, or an artificially designed peptide with one or more natural epitope sequences, wherein a peptide linker may optionally be placed in between adjacent epitope sequences. In some embodiments, the antigenic polypeptide comprises a single epitope of an antigenic protein. In some embodiments, the antigenic polypeptide comprises about any one of 1, 2, 3, 4, 5, 10 or more epitopes from a single antigenic protein. In some embodiments, the antigenic polypeptide comprises epitopes from a plurality (e.g., 2, 3, 4, 5, 10 or more) of different antigenic proteins. In some embodiments, the antigenic polypeptide comprises a Major Histocompatibility Complex (MHC) class I-restricted epitope. In some embodiments, the antigenic polypeptide comprises a MHC class II-restricted epitope. In some embodiments, the antigenic polypeptide comprises both MHC class I-restricted and MHC class II-restricted epitopes.

In some embodiments, the antigenic polypeptide is an antigenic protein or fragment thereof or a variant thereof from a pathogenic agent, such as a bacterium or a virus. In some embodiments, the antigenic polypeptide is an antigenic protein or fragment of a coronavirus, such as SARS-COV2, including variants thereof. In some embodiments, the antigenic polypeptide comprises a Spike(S) protein or a fragment thereof or a variant thereof of a coronavirus, such as SARS-COV, MERS-COV, or SARS-COV-2. CircRNA vaccines have been described, for example, in PCT/CN2021/074998, which is incorporated herein by reference in its entirety. The linear RNAs and constructs described herein may be used to prepare any one of the known circRNA vaccines in the art.

In some embodiments, the antigenic polypeptide is an antigenic protein or fragment thereof or a variant thereof of a self-antigen, such as an antigen involved in a disease or condition. In some embodiments, the antigenic polypeptide is a tumor antigen peptide. Tumor antigen peptide sequences are known in the art and can be found at public databases, such as the Cancer Antigenic Peptide Database (van der Bruggen P et al. (2013) “Peptide database: T cell-defined tumor antigens.” Cancer Immunity. URL: caped.icp.ucl.ac.be). The coding RNA sequence in the linear RNA or circRNA described herein may encode any of the known tumor antigen peptides or combinations thereof. In some embodiments, the antigenic polypeptide comprises an epitope of a tumor associated antigen (TAA). In some embodiments, the antigenic polypeptide comprises an epitope of a tumor specific antigen. In some embodiments, the antigenic polypeptide comprises an epitope of a neoantigen, i.e., newly acquired and expressed antigens present in tumor cells of an individual.

In some embodiments, the amino acid sequences of one or more epitope peptides are predicted based on the sequence of the antigen protein (including neoantigens) using a bioinformatics tool for T cell epitope prediction. Exemplary bioinformatics tools for T cell epitope prediction are known in the art, for example, see Yang X. and Yu X. (2009) “An introduction to epitope prediction methods and software” Rev. Med. Virol. 19 (2): 77-96. In some embodiments, the sequence of the antigen protein is known in the art or available in public databases. In some embodiments, the sequence of the antigen protein (including neoantigens) is determined by sequencing a sample (such as a tumor sample) of the individual being treated.

In some embodiments, the antigenic polypeptide comprises a multimerization domain, such as a dimerization domain, a trimerization domain, or a domain that mediates formation of higher order multimers. In some embodiments, the multimerization domain is a trimerization domain. In non-limiting examples, the multimerization domain comprises a C-terminal Foldon (Fd) domain of a T4 fibritin protein, wherein the C-terminal Foldon domain is the domain that mediates trimerization of the T4 fibritin protein. In another example, the multimerization domain comprises a GCN4-based isoleucine zipper (IZ) domain based on the trimerization domain of the GCN4 transcriptional activator from Saccharomyces cerevisiae. In some embodiments, the GCN4 IZ domain or T4 fibritin Fd domain can be modified to reduce their immunogenicity according to known techniques in the art. For example, the GCN4 IZ domain can be modified with N-linked glycosylation sites to reduce its immunogenicity (Sliepen et al. Immunosilencing a Highly Immunogenic Protein Trimerization Domain. The Journal of Biol. Chem. Vol. 290, No. 12, pp. 7436-7442).

In some embodiments, the antigenic polypeptide further comprises an immunogenic carrier protein. In some embodiments, the antigenic polypeptide comprises an epitope peptide conjugated to an immunogenic carrier protein. Exemplary immunogenic carrier proteins include, but are not limited to, tetanus toxoid (TT), diphtheria toxoid (DT), modified cross-reacting material of diphtheria toxin (CRM197), meningococcal outer membrane protein complex (OMPC), and Hemophilus influenzae protein D (HiD).

In some embodiments, the coding RNA sequence encodes a targeting protein. In some embodiments, the targeting protein is an antibody or an antigen-binding fragment thereof.

In some embodiments, the coding RNA sequence encodes an antibody. In some embodiments, the therapeutic polypeptide is a neutralizing antibody, i.e., an antibody that blocks an interaction between a protein and its binding partner. In some embodiments, the antibody inhibits activity of a protein, e.g., by blocking binding of the protein to a binding partner. In some embodiments, the targeting protein is a therapeutic antibody. In some embodiments, the antibody is a checkpoint inhibitor, e.g., an antibody inhibitor of CTLA-4, PD-1, or PD-L1. In some embodiments, the antibody specifically binds a cell surface antigen, such as a tumor antigen. Exemplary tumor antigens include, but are not limited to, glioma-associated antigen, carcinoembryonic antigen (CEA), β-human chorionic gonadotropin, alphafetoprotein (AFP), lectin-reactive AFP, thyroglobulin, RAGE-1, MN-CAIX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, HER2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and mesothelin. In some embodiments, the antibody specifically binds a target antigen on a pathogenic agent, such as a bacterium or a virus.

The antibody can be an antigen-binding fragment of an antibody, e.g., a portion or fragment of an intact or complete antibody having fewer amino acid residues than the intact or complete antibody, which is capable of binding to an antigen or competing with the intact antibody (i.e., the intact antibody from which the antigen-binding fragment is derived) for binding to an antigen. Antigen-binding fragments can be prepared by recombinant DNA techniques, or by enzymatic or chemical cleavage of intact antibodies. Antigen binding fragments include, but are not limited to, Fab′, F(ab′)2Fv, single chain Fv (scFv), single chain Fab, diabody (diabody), single domain antibody (sdAb, nanobody), camel Ig, Ig NAR, F(ab)′3 Fragment, bis-scFv, (scFv)2 Minibodies, diabodies, triabodies, tetradiabodies, disulfide stabilized Fv proteins (“dsFv”). In some embodiments, the neutralizing antibody can be a genetically engineered antibody, such as a chimeric antibody (e.g., humanized murine antibodies), heteroconjugate antibody (e.g., bispecific antibodies), or antigen-binding fragments thereof.

In some embodiments, the antibody is a neutralizing antibody that binds to a viral protein. In some embodiments, the antibody is a neutralizing antibody that binds to a receptor for a viral protein. In some embodiments, the antibody binds to a receptor that is required for viral entry into a cell (e.g., an ACE2 receptor). In some embodiments, the antibody is a neutralizing antibody (nAb) that binds to the S protein of a coronavirus and prevents or reduces its ability to infect cells. In some embodiments, the coronavirus is SARS-COV-2. In some embodiments, the nAb binds to a S protein comprising one or more mutations. In some embodiments, the nAb binds to a S protein or fragment thereof that comprises at least one point mutation in the S2 region, for example, a K986P, V987P, F817P, A892P, A899P or A942P mutation or combinations thereof. In some embodiments, the nAb binds to a S protein or fragment thereof that comprises at least one point mutation selected from A222V, G339D, S371L, S373P, S375F, E406W, K417N, K417T, N439K, N440K, G446S, L455N, S477N, T478K, E484A, E484K, Q493F, G496S, Q498R, N501Y, Y505H, T547K, A570D, D614G, H655Y, P681H, A701V, T716I, N764K, D796Y, N856K, Q954H, N969K, L981F, S982A, or combinations thereof. In some embodiments the nAb is a monoclonal antibody (mAb), a functional antigen-binding fragment (Fab), a single-chain variable region fragment (scFv), or a single-domain antibody (a VHH or nanobody).

Exemplary nAbs for binding and neutralization of the S protein of SARS-COV-2 have been described, for example, in Barnes, C. O. et al. SARS-COV-2 neutralizing antibody structures inform therapeutic strategies. Nature 588, 682-687 (2020), and Chinese Patent Application No. CN111690058A, the contents of which are herein incorporated by reference in their entirety.

In some embodiments, the coding RNA sequence encodes a targeting protein that is not an antibody. Examples of non-antibody-based targeting proteins include, but are not limited to, a lipocalin, an anticalin (artificial antibody mimetic proteins that are derived from human lipocalins), “T-body”, a peptide (e.g., a BICYCLE™ peptide), an affibody (antibody mimetics composed of alpha helices, e.g. an three-helix bundle), a peptibody (peptide-Fc fusion), a DARPin (designed ankyrin repeat proteins, engineered antibody mimetic proteins consisting repeat motifs), an affimer, an avimer, a knottin (a protein structural motif containing 3 disulfide bridges), a monobody, an affinity clamp, an ectodomain, a receptor ectodomain, a receptor, a cytokine, a ligand, an immunocytokine, and a centryin. See, for example, Vazquez-Lombardi, Rodrigo, et al. Drug discovery today 20.10 (2015): 1271-1283.

In some embodiments, the coding RNA sequence encodes a soluble receptor. Soluble receptors (sometimes referred to as soluble receptor decoys or “traps”) can comprise all or a portion of the extracellular domain of a receptor protein. In some embodiments, a nucleotide sequence encoding all or a portion of the extracellular domain of a receptor protein is operably linked to a signal peptide for secretion from cells.

In some embodiments, the soluble receptor comprises an extracellular domain of a naturally occurring receptor. In some embodiments, the soluble receptor variant comprises an engineered variant of an extracellular domain of a naturally occurring receptor, such as a variant comprising one or more mutations in the extracellular domain. In some embodiments, the soluble receptor comprises one or more mutations that increase the affinity of the soluble receptor for its ligand compared to the affinity of the naturally occurring receptor for its ligand.

In some embodiments, the soluble receptor is a fusion protein comprising one or more additional protein domains operably linked to the extracellular domain of the receptor or a variant thereof. In some embodiments, the soluble receptor comprises an Fc domain of an immunoglobulin (Ig), e.g., a human immunoglobulin. In some embodiments, the soluble receptor comprises an Fc domain of a human IgG1.

In some embodiments, the soluble receptor comprises the extracellular domain of a signaling receptor, and the soluble receptor can reduce or inhibit activity of the signaling pathway by blocking binding between the endogenous receptor and its ligand.

In some embodiments, the soluble receptor is a receptor that binds to a viral protein and/or that mediates viral entry. In some embodiments, soluble receptor is a soluble ACE2 receptor. In some embodiments, the therapeutic polypeptide is a soluble ACE2 receptor variant capable of binding to an S protein of a coronavirus. In some embodiments, the soluble ACE2 receptor variant binds to the receptor binding domain (RBD) of the S protein. In some embodiments, the ACE2 receptor variant is enzymatically active. In other embodiments, the ACE2 receptor variant is enzymatically inactive. In some embodiments, the soluble ACE2 receptor variant comprises the soluble extracellular domain of wild-type (WT) human recombinant ACE2 (APN01). In some embodiments, the soluble ACE2 receptor variant comprises one or more mutations in the extracellular domain of human ACE2. In some embodiments, the soluble ACE2 receptor variant is engineered via affinity maturation to have increased binding affinity to the RBD of the S protein. Soluble ACE2 receptor variants have been described, for example in Haschke M et al., Clin Pharmacokinet. 2013 September; 52 (9): 783-92; Glasgow A et al., Proceedings of the National Academy of Sciences November 2020, 117 (45) 28046-28055; and Higuchi Y. et al., bioRxiv 2020. 09.16.299891, the contents of which are herein incorporated by reference in their entirety. In some embodiments, the soluble ACE2 receptor variant is a fusion protein, e.g., a fusion of the extracellular ACE2 receptor domain to the Fc region of the human IgG1.

In some embodiments, the coding RNA sequence encodes a functional protein. In some embodiments, the coding RNA sequence is capable of being expressed by target cells (e.g., human or mouse cells, or any other organism capable of being transformed) for the production (and in certain instances, the secretion) of a functional enzyme or protein as disclosed, for example, in International Application No. PCT/US2010/058457 and WO2020237227, the contents of which are herein incorporated by reference in their entirety. In some embodiments, the therapeutic polypeptide can be engineered for secretion by operably linking a signal peptide to the amino terminus of the therapeutic polypeptide. For example, in some embodiments, upon the expression of one or more therapeutic polynucleotides by target cells, the production of a functional enzyme or protein in which a subject is deficient (e.g., a urea cycle enzyme or an enzyme associated with a lysosomal storage disorder) may be observed.

In some embodiments, the coding RNA sequence encodes a protein such as IDUA, OTC, FAH, miniDMD, DMD, p53, PTEN, COL3A1, BMPR2, AHI1, FANCC, MYBPC3, ILRG2, or ARG1, wherein deficiency of the functional protein is associated with a disease or disorder. In some embodiments, the coding RNA sequence a protein (e.g., a lysosomal enzyme) wherein deficiency of the protein is associated with a lysosomal storage disorder.

In some embodiments, the coding RNA sequence encodes a protein (e.g., an enzyme), wherein deficiency of the protein is associated with a metabolic disorder. In some embodiments, the therapeutic polypeptide comprises a urea cycle enzyme (e.g., ARG1).

In some embodiments, the coding RNA sequence encodes a protein (e.g., p53 or PTEN), wherein deficiency of the protein is associated with a cancer. In some embodiments, the therapeutic polypeptide comprises a tumor suppressor.

In some embodiments, the coding RNA sequence encodes a reporter protein, such as a fluorescent protein. Fluorescent proteins are well known to those skilled in the art, and include but are not limited to, green fluorescent proteins (GFPs), enhanced green fluorescent proteins (EGFPs), red fluorescent proteins (RFPs), and blue fluorescent proteins (BFPs).

In some embodiments, the coding RNA sequence encodes a site-specific genome modification enzyme, such as an endonuclease, a recombinase, a helicase, or a transposase. Endonucleases are well known to those skilled in the art, and include but are not limited to a meganuclease, a zinc finger nuclease, a transcription activator-like effector nuclease (TALEN), an Argonaute, a DNA-guided recombinase, a DNA-guided endonuclease, an RNA-guided recombinase, an RNA-guided endonuclease, a type I CRISPR-Cas system, a type II CRISPR-Cas system; a type III CRISPR-Cas system, a type IV CRISPR-Cas system, a type V CRISPR-Cas system, a type VI CRISPR-Cas system; Cpf1 (also known as Cas12a), Cas1, Cas1B, Cas2, Cas3, Cas4, Cas5, Cas6, Cas7, Cas8, Cas9 (also known as Csn1 and Csx12), Cas10, Cas12b, Cas12c, Cas13a, Cas13b, Cas13c, Cas13d, Cas14, CasX, CasΦ (CasPhi), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4 nuclease; a dCas9-recombinase fusion protein; a tyrosine recombinase, a serine recombinase, a Cre recombinase, a Flp recombinase, a Tnp1 recombinase, a PhiC31 integrase, an R4 integrase, or a TP-901 integrase. In some embodiments, the RNA sequence encodes a guide RNA (gRNA) that associates with a Cas protein.

In some embodiments, the coding RNA sequence encodes two or more polypeptides, such as two or more therapeutic polypeptides. In some embodiments, the coding RNA sequence encodes a therapeutic polypeptide and a reporter protein.

In some embodiments, the various domains or fragments in the polypeptide encoded by the coding RNA sequence may be fused to each other via a peptide linker. Flexible peptide linkers such as glycine linkers, glycine-serine linkers, and linkers containing other amino acids are known in the art (for example, suitable peptide linkers are described by Chen et al. in Fusion Protein Linkers: Property, Design and Functionality. Adv. Drug Deli Rev. 2013 Oct. 15; 65 (10): 1357-1369). Peptide linkers can also be designed by computation methods. The peptide linker can be of any length from 1 to 10, 10 to 20, 20 to 30, 30 to 40, 40 to 50, or greater than 50 amino acids.

In some embodiments, the coding RNA sequence is codon-optimized. A codon optimized sequence may be one in which codons in a polynucleotide encoding a polypeptide have been substituted in order to increase the expression, stability and/or activity of the polypeptide. Factors that influence codon optimization include, but are not limited to one or more of: (i) variation of codon biases between two or more organisms or genes or synthetically constructed bias tables, (ii) variation in the degree of codon bias within an organism, gene, or set of genes, (iii) systematic variation of codons including context, (iv) variation of codons according to their decoding tRNAs, (v) variation of codons according to GC %, either overall or in one position of the triplet, (vi) variation in degree of similarity to a reference sequence for example a naturally occurring sequence, (vii) variation in the codon frequency cutoff, (viii) structural properties of mRNAs transcribed from the DNA sequence, (ix) prior knowledge about the function of the DNA sequences upon which design of the codon substitution set is to be based, and/or (x) systematic variation of codon sets for each amino acid. In some embodiments, a codon optimized polynucleotide may minimize ribozyme collisions and/or limit structural interference between the expression sequence and the internal ribosomal entry site (IRES).

In some embodiments, the coding RNA sequence may encode or be operably linked to one or more additional elements that facilitate translation of the coding RNA sequence into a functional polypeptide. In some embodiments, the one or more additional elements are useful for monitoring translation of the coding RNA sequence.

In some embodiments, the coding RNA sequence encodes a polypeptide comprising a signal peptide (SP). In non-limiting examples, the signal peptide is the signal sequence and propeptide from human tissue plasminogen activator (tPA), the signal sequence from human IgE Immunoglobulin, or the signal peptide sequence of MHC I. In some embodiments, the signal peptide can facilitate secretion of the polypeptide encoded by the coding RNA sequence.

In some embodiments, the 3′ end of the coding RNA sequence is operably linked to an in-frame 2A peptide coding sequence. In some embodiments, the coding RNA sequence does not comprise a stop codon at the 3′ end. In some embodiments, the in-frame 2A peptide coding sequence replaces the stop codon. In some embodiments, the coding RNA sequence contains no stop codon and the number of nucleotides composing the coding RNA is a multiple of three. In some embodiments, the coding RNA sequence having no stop codon and the number of nucleotides composing the RNA being a multiple of three allow for rolling circle translation of the circRNA prepared using the linear RNA precursor. In some embodiments, the 2A peptide coding sequence allows for rolling circle translation of the circRNA prepared using the linear RNA precursor. In some embodiments, the 2A peptide allows cleavage of a polypeptide generated by rolling circle translation into monomeric polypeptide sequences. In non-limiting examples, the 2A peptide coding sequence encodes a P2A or T2A peptide.

In some embodiments, the coding RNA sequence comprises a nucleotide sequence encoding an affinity or identification tag. Exemplary tags include, but are not limited to, His tag, FLAG tag, SUMO tag, GST tag, and MBP tag.

In some embodiments, the 5′ end of the coding RNA sequence is operably linked to a Kozak sequence or portion thereof. In some embodiments, the Kozak sequence functions as a protein translation initiation site. In some embodiments, the linear RNA comprises from the 5′ end to the 3′ end: a first portion of an RNA element (e.g., IRES), a Kozak sequence, a coding RNA sequence, and a second portion of the RNA element (e.g., IRES). In some embodiments, the first portion of an IRES comprises the sequence set forth in any of SEQ ID NOs: 26 or 28. In some embodiments, the first portion of an IRES comprises the sequence set forth in any of SEQ ID NOs: 27 or 29.

In some embodiments, the effector RNA sequence comprises an internal ribosomal entry site (IRES) sequence or portion thereof.

In some embodiments, the effector RNA sequence comprises an m6A modification motif sequence operably linked to the coding RNA sequence.

In some embodiments, the linear RNA further comprises a polyA or polyAC sequence disposed at the 3′ end of the coding RNA sequence and at the 5′ end of the second portion of the RNA element (e.g., IRES). The internal polyA sequence or polyAC spacer may range from 1 to 500 nucleotides in length (e.g., at least 20, 30, 40, 50, 60, 70, 80, 90, 120, 140, 160, 180, 200, 250, 300, 350, 400, 450, or 500 nucleotides). In some embodiments, the polyA sequence or polyAC sequence may range from 10-70, 20-60, or 30-60 nucleotides in length. In some embodiments, the linear RNA comprises no polyA sequence or polyAC sequence. Without being bound by any theory or hypothesis, an internal polyA sequence or a polyAC spacer added before IRES sequences in a circRNA can help to keep the functional second structure of IRES elements for efficient protein translation initiated by IRES. In some embodiments, the polyA sequence or polyAC spacer increases expression of the coding RNA.

In some embodiments, the effector RNA sequence comprises a nucleic acid sequence comprising a therapeutic RNA. In some embodiments, the therapeutic RNA is an RNA molecule selected from the group consisting of a gRNA, a dRNA, a siRNA, a miRNA, a shRNA, and a lincRNA.

Circularization Methods with Group I Introns

In some embodiments, there is provided a method for producing a circular RNA from a DNA construct encoding a linear RNA precursor, wherein the method comprises in vitro transcription of a DNA template into a linear RNA precursor, followed by DNase I treatment that removes the DNA template, and a separate step to circularize of the linear RNA precursor by activation of self-splicing (for example, in cis by a catalytic Group I intron, or in trans by a 3′ catalytic Group I intron fragment in association with a free 5′ catalytic Group I intron fragment) in a linear RNA precursor through a GTP treatment step. In some embodiments, the method comprises supplementing the reagent composition with new and/or additional reagents. In some embodiments, the method comprises removing one or more starting materials, such as unreacted DNA construct, NTPs, RNA polymerase, etc., from the reaction mixture before circularization of the linear RNA precursor. In some embodiments, the method comprises isolating or purifying the linear RNA precursor prior to circularization of the linear RNA precursor.

In some embodiments comprising a PIE-style linear RNA precursor, such as, e.g., embodiments comprising a free 5′ catalytic Group I intron fragment that is used in combination with a canonical PIE-style linear RNA precursor to facilitate circularization of the canonical PIE-style linear RNA precursor, the method comprises a GTP treatment step. GTP treatment refers to a reaction in which the linear RNA precursor is contacted with one or more reagents to activate Group I intron self-splicing. A GTP treatment step comprises contacting the linear RNA precursor with GTP (e.g., at a final concentration of 2 mM). A GTP treatment step may further comprise contacting the linear RNA precursor with a divalent metal ion, such as Mg²⁺. In some embodiments, a GTP treatment step comprises contacting the linear RNA precursor with GTP and Mg²⁺ at about 55° C. for about 8 minutes.

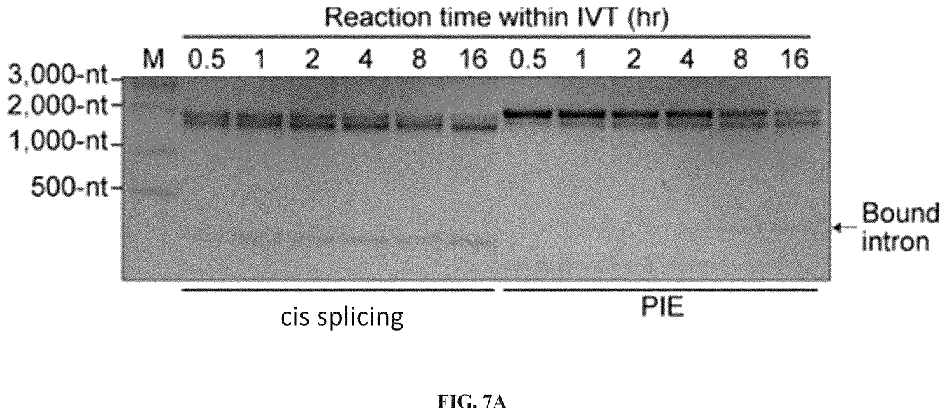

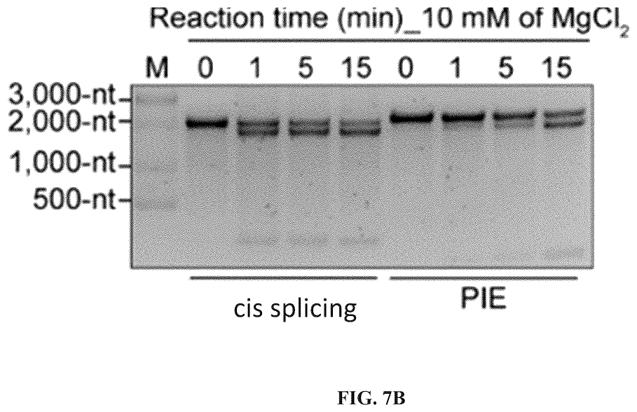

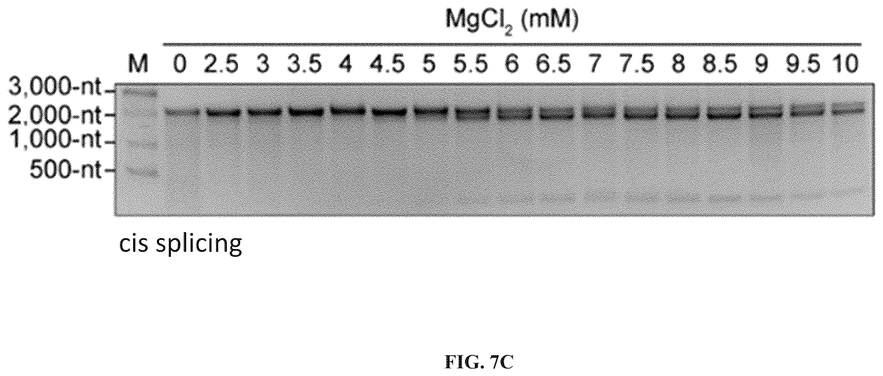

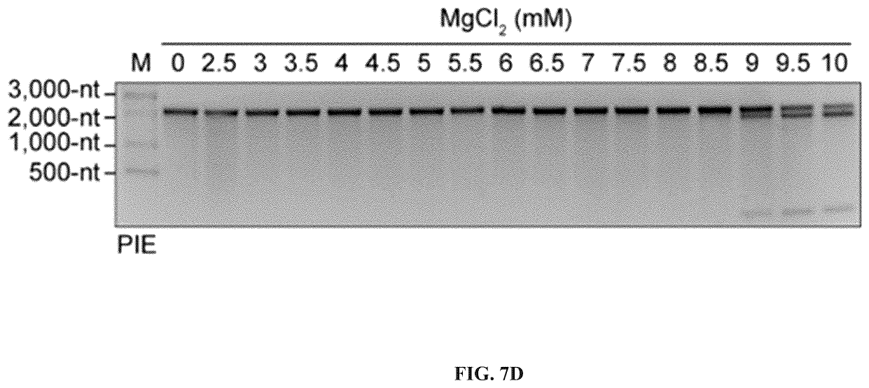

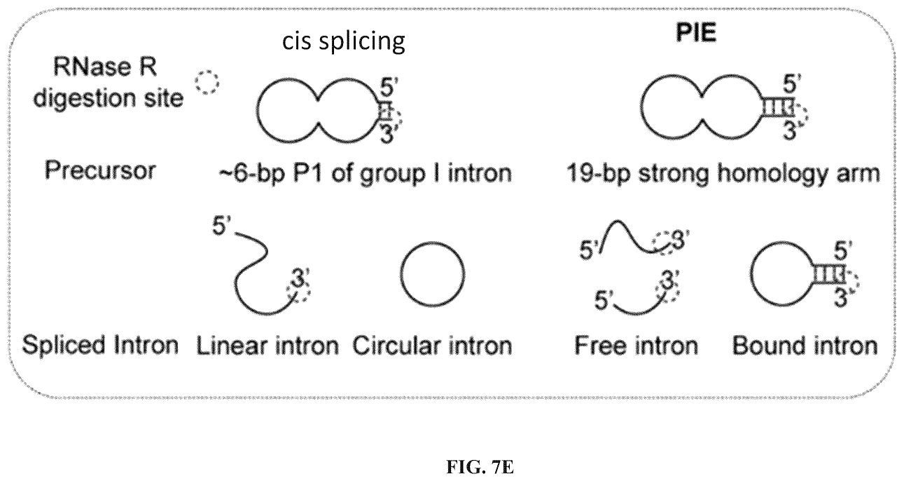

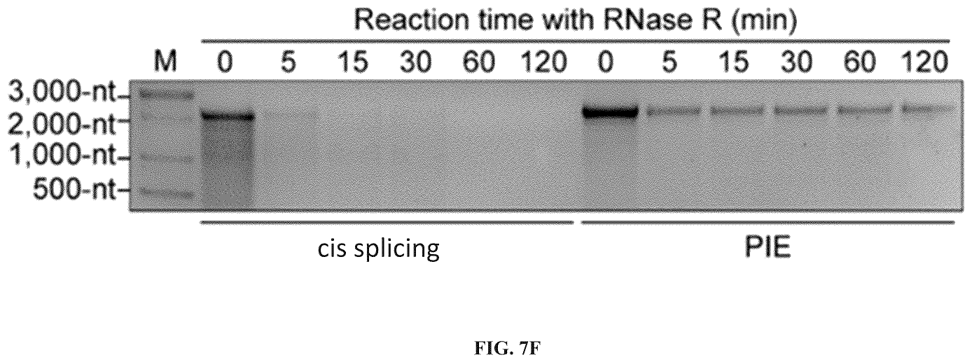

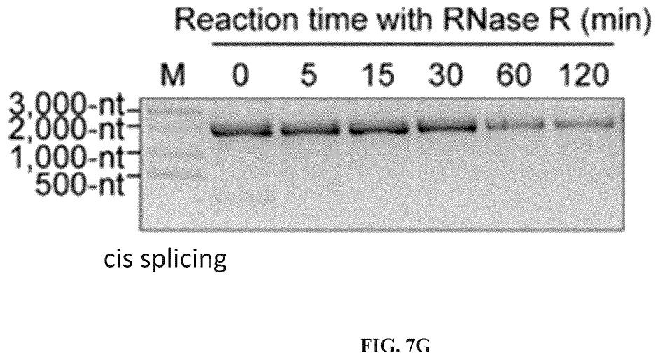

In some embodiments, buffers with reduced, minimal, or no measurable amounts of monovalent cations improve circularization efficiency of RNA by, for example, lowering the requirement for divalent cations such as Mg²⁺. In some embodiments, a decreased amount of a monovalent cation (such as, for example, a decreased amount of Na⁺ and/or K⁺) in the buffer improves circularization efficiency of RNA compared to a control buffer having the same concentration of divalent cation (for example, having the same concentration of Mg²⁺). In some embodiments, a buffer with no detectable concentration monovalent cations improves circularization efficiency of RNA. In some embodiments, the reduction in monovalent cation concentrations lowers the amount of Mg²⁺ required. For example, in some embodiments, adding extra 150 mM NaCl in buffer raises the requirement for Mg²⁺ from ˜0.12 mM to ˜5.5 mM for cis splicing, and from ˜2 mM to ˜9 mM for PIE (see, e.g., FIG. 7C and FIG. 7D). In some embodiments, the monovalent cation content of the buffer is reduced by 0-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 95-100, 100-150, 150-200, 200-250, 250-300, 300-350, 350-400, 400-450, 450-500, or reduced by more than 500 mM compared to a corresponding buffer. In some embodiments, the monovalent cation content of the buffer is reduced by 500-550, 550-600, 600-650, 650-700, 700-750, 750-800, 800-850, 850-900, 900-950, 950-1000 or reduced by more than 1000 mM compared to a corresponding buffer. In some embodiments, the corresponding buffer has the same concentration of divalent cations. In some embodiments, the corresponding buffer has the same concentration of Mg²⁺. In some embodiments, RNA circularization takes place less efficiently in the corresponding buffer.

In some embodiments, the monovalent cation is Na⁺, K⁺, H⁺, Li⁺, Cu⁺, Ag⁺, Cs⁺, or Au⁺. In some embodiments, the monovalent cation is Na. In some embodiments, the monovalent cation is K⁺.

Further description of buffers used in circularization reactions is included in the Examples. In some embodiments, the 1× in vitro reaction buffer comprises 40 mM Tris-HCl, 6 mM MgCl₂, 1 mM DTT, and 2 mM spermidine. In some embodiments, the buffers contain Mg²⁺, 50 mM HEPES, and 150 mM NaCl. In some embodiments, buffers with decreased amounts of monovalent cations have ½, ¼, ⅕, ⅛, 1/10, 1/15, 1/20, 1/30, 1/40, 1/50, 1/60, 1/70, 1/80, 1/90, or less than 1/100 of the amount of monovalent cations in the previously described buffers. In some embodiments, buffers with decreased amounts of monovalent cations have 1/90, 1/91, 1/92, 1/93, 1/94, 1/95, 1/96, 1/97, 1/98, 1/99, or less than 1/100 of the amount of monovalent cations in the previously described buffers. In some embodiments, buffers with decreased amounts of monovalent cations have 1/100, 1/500, or less than 1/1000 of the amount of monovalent cations in the previously described buffers. In some embodiments, buffers with decreased amounts of monovalent cations have undetectable amounts of the monovalent cations when compared to the amounts in the previously described buffers.

In some embodiments, the method comprises supplementing the reagent composition with one or more nucleoside triphosphates prior to the circularization of the linear RNA precursor. In some embodiments, the method comprises supplementing the reagent composition with GTP. In some embodiments, the NTP mixture for in vitro transcription is sufficient to allow activation of self-splicing of the Group I intron fragments in the linear RNA precursor.

In some embodiments, the method comprises supplementing the reaction composition with a divalent metal ion prior to circularization of the linear RNA precursor. In some embodiments, the divalent metal ion is selected from the group consisting of Mg²⁺, Mn²⁺, Ca²⁺, Co²⁺, Be²⁺, Cu²⁺, Fe²⁺, Zn²⁺, Sr²⁺, Ba²⁺, Al²⁺, and Cd²⁺. In some embodiments, the method comprises supplementing the reaction composition with Mg²⁺.

In some embodiments, the method comprises supplementing the reagent composition with GTP or a divalent metal ion such as Mg²⁺.

In some embodiments, the method comprises incubating the linear RNA precursor at about at least 30, 35, 37, 40, 45, 50, 55, 60, 65, or 70° C. In some embodiments, the method comprises incubating the linear RNA precursor at about any one of 30-40, 40-50, 50-60, 60-70, 40-70 or 50-60° C. In some embodiments, the method comprises incubating the linear RNA precursor at about 55° C. In some embodiments, the method comprises a chemical reaction with a pH of 6-9. In some embodiments, the method comprises a chemical reaction with a pH of 6.3, 6.6, 6.9, 7.3, 7.6, 7.9, 8.3, 8.6, 8.9, or 9. In some embodiments, the method comprises a chemical reaction with a pH of 6.8, 7.0, 7.2, 7.3, 7.5, 7.5, or 8.0.

In known methods of producing circularized RNA, the in vitro transcription product from the DNA construct is subject to a number of treatment and/or reaction steps, including DNase I treatment and isolation of the linear RNA precursor prior to circularization by activation of the Group I intron fragments. DNase I treatment removes DNA construct from the reaction mixture after completion of in vitro transcription.

In some embodiments, the method comprises DNase treatment prior to circularization of the linear RNA precursor. In some embodiments, the DNase is DNase I or DNase II. In some embodiments, the DNase is a micrococcal nuclease. In some embodiments, the DNase is a restriction enzyme. In some embodiments, the method comprises DNase I treatment prior to circularization of the linear RNA precursor.

In some embodiments, the method comprises contacting the product of in vitro transcription with a DNase I at 37° C. for about 20 minutes. In some embodiments, the method comprises isolating and/or purifying the linear RNA precursor from the in vitro transcription reaction.

In some embodiments, the RNA polymerase is a T7 RNA polymerase, a T3 RNA polymerase, a SP6 RNA polymerase, or a derivative thereof. In some embodiments, the in vitro transcription is driven by a T7 promoter in the DNA construct, and the RNA polymerase is a T7 RNA polymerase. In some embodiments, the in vitro transcription is driven by a T3 phage promoter in the DNA, and the RNA polymerase is a T3 RNA polymerase. In some embodiments, the in vitro transcription is driven by an SP6 promoter in the DNA construct, and the RNA polymerase is a SP6 RNA polymerase.

In some embodiments, the reagent composition comprises an NTP mixture. In some embodiments, the NTP mixture comprises ATP, UTP, GTP and CTP. In some embodiments, the NTP mixture comprises one or more modified nucleoside 5′ triphosphate. In some embodiments, the NTP mixture does not comprise a modified nucleoside 5′ triphosphate. In some embodiments, the reagent composition comprises an equal concentration for each of ATP, UTP, GTP, and CTP. In some embodiments, the reagent composition comprises an equal concentration for at least two of ATP, UTP, GTP, or CTP. In some embodiments, the reagent composition comprises a different concentration for each of ATP, UTP, GTP, and CTP. In some embodiments, the concentration of GTP in the reagent composition is higher than one or more of the concentrations of ATP, UTP and GTP. In some embodiments, the concentration of a nucleoside 5′ triphosphate (e.g., ATP, UTP, GTP or CTP) in the reagent composition is at least about 0.01, 0.05, 0.1, 0.5, 1, 2, 3, 4, 5, 6, 7, 8, 9, 10, 15, 20, 30, 40, 50 mM, or higher. In some embodiments, the concentration of a nucleoside 5′ triphosphate (e.g., ATP, UTP, GTP or CTP) in the reagent composition is no more than about any one of 50, 40, 30, 20, 15, 10, 9, 8, 7, 6, 5, 4, 3, 2, 1, 0.05, 0.01 mM or less. In some embodiments, the concentration of a nucleoside 5′ triphosphate (e.g., ATP, UTP, GTP or CTP) in the reagent composition is about any one of 0.01-0.05, 0.05-0.1, 0.1-0.5, 0.5-1, 1-2, 2-3, 3-4, 4-5, 5-6, 6-7, 7-8, 8-9, 9-10, 10-15, 15-20, 20-30, 30-40, 40-50, 0.01-10, 0.01-50, 0.1-10, 0.1-50, 10-50, 5-20, 20-40 or 5-25 mM. In some embodiments, the concentration of each of ATP, GTP, CTP and UTP in the reaction composition is about 10 mM. In some embodiments, the concentration of GTP is about 7.5 mM. In some embodiments, the concentration of each of ATP, UTP and CTP is the same, and the concentration of GTP is higher than the concentration of each of ATP, UTP and CTP. In some embodiments, the concentration of GTP is about at least any one of 1.1, 1.2, 1.25, 1.3, 1.4, 1.5, 1.6, 1.7, 1.75, 1.8, 1.9, 2, 2.5, 3, 3.5, 4 times or more than the concentration of ATP, UTP or CTP.

In some embodiments, the DNA construct is contacted with the reagent composition at about at least any one of 30, 31, 32, 33, 34, 35, 36, 37, 38, 39, 40, 41, 42, 43, 44, 45, 46, 47, 48, 49, 50, 51, 52, 53, 54, 55, or 56° C. In some embodiments, the DNA construct is contacted with the reagent composition at no more than about any one of 56, 54, 53, 52, 51, 50, 49, 48, 47, 46, 45, 44, 43, 42, 41, 40, 39, 38, 37, 36, 35, 34, 33, 32, 31, or 30° C. In some embodiments, the DNA construct is contacted with the reagent composition at about any one of 30-31, 31-32, 32-33, 33-34, 34-35, 35-36, 36-37, 37-38, 38-40, 40-42, 42-44, 44-46, 46-48, 48-50, 50-52, 52-54, 54-56, 37-45, 45-56, or 40-50° C. In some embodiments, the DNA construct is contacted with the reagent composition at about 37° C.

In some embodiments, the DNA construct is contacted with the reagent composition for at least about any one of 5, 10, 20, 30, or 40 minutes, or 1, 2, 3, 4, 5, 6, 7, 8, 9, 10, 11, 12, 13, 14, 15, 16, 17, 18, 19, 20, 21, 22, 23, or 24 hours. In some embodiments, the DNA construct is contacted with the reagent composition for no more than about any one of 24, 23, 22, 21, 20, 19, 18, 17, 16, 15, 14, 13, 12, 11, 10, 9, 8, 7, 6, 5, 4, 3, 2, or 1 hour(s), or 40, 30, 20, 10, or 5 minutes. In some embodiments, the DNA construct is contacted with the reagent composition for about any one of 1-2, 2-4, 4-6, 6-8, 8-10, 10-12, 12-16, 16-18, 18-20, 20-22, 22-24, 1-6, 6-12, 12-18, 18-24, 1-12, 12-24, or 6-16 hours, or 20 minutes to 1 hour, or 20 minutes to 2 hours, or 20 minutes to 16 hours, or 20 minutes to 24 hours. In some embodiments, the DNA construct is contacted with the reagent composition for about 16 hours. In some embodiments, the DNA construct is contacted with the reagent composition for about 20 minutes to about 24 hours.

In some embodiments, the method comprises one or more additional steps for obtaining the DNA construct and/or isolating the circular RNA.

Circularization Methods with Group II Introns

In some embodiments, there is provided a method for producing a circular RNA from a DNA construct encoding a linear RNA precursor, wherein the method comprises in vitro transcription of a DNA template into a linear RNA precursor, followed by DNase I treatment that removes the DNA template, and a separate step to circularize of the linear RNA precursor by activation of self-splicing (for example, in cis by a catalytic Group II intron, or in trans by a 3′ catalytic Group II intron fragment in association with a free 5′ catalytic Group II intron fragment) in a linear RNA precursor through a GTP treatment step. In some embodiments, the method comprises supplementing the reagent composition with new and/or additional reagents. In some embodiments, the method comprises removing one or more starting materials, such as unreacted DNA construct, NTPs, RNA polymerase, etc., from the reaction mixture before circularization of the linear RNA precursor. In some embodiments, the method comprises isolating or purifying the linear RNA precursor prior to circularization of the linear RNA precursor.

In some embodiments comprising a PIE-style linear RNA precursor, such as, e.g., embodiments comprising a free 5′ catalytic Group II intron fragment that is used in combination with a canonical PIE-style linear RNA precursor to facilitate circularization of the canonical PIE-style linear RNA precursor, the method comprises a GTP treatment step. GTP treatment refers to a reaction in which the linear RNA precursor is contacted with one or more reagents to activate Group II intron self-splicing. A GTP treatment step comprises contacting the linear RNA precursor with GTP (e.g., at a final concentration of 2 mM). A GTP treatment step may further comprise contacting the linear RNA precursor with a divalent metal ion, such as Mg²⁺. In some embodiments, a GTP treatment step comprises contacting the linear RNA precursor with GTP and Mg²⁺ at about 55° C. for about 8 minutes.

In some embodiments, buffers with reduced, minimal, or no measurable amounts of monovalent cations improve circularization efficiency of RNA by, for example, lowering the requirement for divalent cations such as Mg²⁺. In some embodiments, a decreased amount of a monovalent cation (such as, for example, a decreased amount of Na⁺ and/or K⁺) in the buffer improves circularization efficiency of RNA compared to a control buffer having the same concentration of divalent cation (for example, having the same concentration of Mg²⁺). In some embodiments, a buffer with no detectable concentration monovalent cations improves circularization efficiency of RNA. In some embodiments, the reduction in monovalent cation concentrations lowers the amount of Mg²⁺ required. For example, in some embodiments, adding extra 150 mM NaCl in buffer raises the requirement for Mg²⁺ from ˜0.12 mM to ˜5.5 mM for cis splicing, and from ˜2 mM to ˜9 mM for PIE (see, e.g., FIG. 7C and FIG. 7D). In some embodiments, the monovalent cation content of the buffer is reduced by 0-5, 5-10, 10-15, 15-20, 20-25, 25-30, 30-35, 35-40, 40-45, 45-50, 50-55, 55-60, 60-65, 65-70, 70-75, 75-80, 80-85, 85-90, 90-95, 100-150, 150-200, 200-250, 250-300, 300-350, 350-400, 400-450, 450-500, or reduced by more than 500 mM compared to a corresponding buffer. In some embodiments, the corresponding buffer has the same concentration of divalent cations. In some embodiments, the corresponding buffer has the same concentration of Mg²⁺. In some embodiments, RNA circularization takes place less efficiently in the corresponding buffer.

In some embodiments, the monovalent cation is Na⁺, K⁺, H⁺, Li⁺, Cu⁺, Ag⁺, Cs⁺, or Au⁺. In some embodiments, the monovalent cation is Na. In some embodiments, the monovalent cation is K⁺.

Further description of buffers used in circularization reactions is included in the Examples. In some embodiments, the 1× in vitro reaction buffer comprises 40 mM Tris-HCl, 6 mM MgCl₂, 1 mM DTT, and 2 mM spermidine. In some embodiments, the buffers contain Mg²⁺, 50 mM HEPES, and 150 mM NaCl. In some embodiments, buffers with decreased amounts of monovalent cations have ½, ¼, ⅕, ⅛, 1/10, 1/15, 1/20, 1/30, 1/40, 1/50, 1/60, 1/70, 1/80, 1/90, or less than 1/100 of the amount of monovalent cations in the previously described buffers. In some embodiments, buffers with decreased amounts of monovalent cations have 1/90, 1/91, 1/92, 1/93, 1/94, 1/95, 1/96, 1/97, 1/98, 1/99, or less than 1/100 of the amount of monovalent cations in the previously described buffers. In some embodiments, buffers with decreased amounts of monovalent cations have 1/100, 1/500, or less than 1/1000 of the amount of monovalent cations in the previously described buffers. In some embodiments, buffers with decreased amounts of monovalent cations have undetectable amounts of the monovalent cations when compared to the amounts in the previously described buffers.

In some embodiments, the method comprises supplementing the reagent composition with one or more nucleoside triphosphates prior to the circularization of the linear RNA precursor. In some embodiments, the method comprises supplementing the reagent composition with GTP. In some embodiments, the NTP mixture for in vitro transcription is sufficient to allow activation of self-splicing of the Group II intron fragments in the linear RNA precursor.

In some embodiments, the method comprises supplementing the reaction composition with a divalent metal ion prior to circularization of the linear RNA precursor. In some embodiments, the divalent metal ion is selected from the group consisting of Mg²⁺, Mn²⁺, Ca²⁺, Co²⁺, Be²⁺, Cu²⁺, Fe²⁺, Zn²⁺, Sr²⁺, Ba²⁺, Al²⁺, and Cd²⁺. In some embodiments, the method comprises supplementing the reaction composition with Mg²⁺.

In some embodiments, the method comprises supplementing the reagent composition with GTP or a divalent metal ion such as Mg²⁺.

In some embodiments, the method comprises incubating the linear RNA precursor at about at least 30, 35, 37, 40, 45, 50, 55, 60, 65, or 70° C. In some embodiments, the method comprises incubating the linear RNA precursor at about any one of 30-40, 40-50, 50-60, 60-70, 40-70 or 50-60° C. In some embodiments, the method comprises incubating the linear RNA precursor at about 55° C. In some embodiments, the method comprises a chemical reaction with a pH of 6-9. In some embodiments, the method comprises a chemical reaction with a pH of 6.3, 6.6, 6.9, 7.3, 7.6, 7.9, 8.3, 8.6, 8.9, or 9. In some embodiments, the method comprises a chemical reaction with a pH of 6.8, 7.0, 7.2, 7.3, 7.5, 7.5, or 8.0.

In known methods of producing circularized RNA, the in vitro transcription product from the DNA construct is subject to a number of treatment and/or reaction steps, including DNase I treatment and isolation of the linear RNA precursor prior to circularization by activation of the Group II intron fragments. DNase I treatment removes DNA construct from the reaction mixture after completion of in vitro transcription.

In some embodiments, the method comprises DNase treatment prior to circularization of the linear RNA precursor. In some embodiments, the DNase is DNase I or DNase II. In some embodiments, the DNase is a micrococcal nuclease. In some embodiments, the DNase is a restriction enzyme. In some embodiments, the method comprises DNase I treatment prior to circularization of the linear RNA precursor.

In some embodiments, the method comprises contacting the product of in vitro transcription with a DNase I at 37° C. for about 20 minutes. In some embodiments, the method comprises isolating and/or purifying the linear RNA precursor from the in vitro transcription reaction.

In some embodiments, the RNA polymerase is a T7 RNA polymerase, a T3 RNA polymerase, a SP6 RNA polymerase, or a derivative thereof. In some embodiments, the in vitro transcription is driven by a T7 promoter in the DNA construct, and the RNA polymerase is a T7 RNA polymerase. In some embodiments, the in vitro transcription is driven by a T3 phage promoter in the DNA, and the RNA polymerase is a T3 RNA polymerase. In some embodiments, the in vitro transcription is driven by an SP6 promoter in the DNA construct, and the RNA polymerase is a SP6 RNA polymerase.

In some embodiments, the reagent composition comprises an NTP mixture. In some embodiments, the NTP mixture comprises ATP, UTP, GTP and CTP. In some embodiments, the NTP mixture comprises one or more modified nucleoside 5′ triphosphate. In some embodiments, the NTP mixture does not comprise a modified nucleoside 5′ triphosphate. In some embodiments, the reagent composition comprises an equal concentration for each of ATP, UTP, GTP, and CTP. In some embodiments, the reagent composition comprises an equal concentration for at least two of ATP, UTP, GTP, or CTP. In some embodiments, the reagent composition comprises a different concentration for each of ATP, UTP, GTP, and CTP. In some embodiments, the concentration of GTP in the reagent composition is higher than one or more of the concentrations of ATP, UTP and GTP. In some embodiments, the concentration of a nucleoside 5′ triphosphate (e.g., ATP, UTP, GTP or CTP) in the reagent composition is at least about 0.01, 0.05, 0.1, 0.5, 1, 2, 3, 4, 5, 6, 7, 8, 9, 10, 15, 20, 30, 40, 50 mM, or higher. In some embodiments, the concentration of a nucleoside 5′ triphosphate (e.g., ATP, UTP, GTP or CTP) in the reagent composition is no more than about any one of 50, 40, 30, 20, 15, 10, 9, 8, 7, 6, 5, 4, 3, 2, 1, 0.05, 0.01 mM or less. In some embodiments, the concentration of a nucleoside 5′ triphosphate (e.g., ATP, UTP, GTP or CTP) in the reagent composition is about any one of 0.01-0.05, 0.05-0.1, 0.1-0.5, 0.5-1, 1-2, 2-3, 3-4, 4-5, 5-6, 6-7, 7-8, 8-9, 9-10, 10-15, 15-20, 20-30, 30-40, 40-50, 0.01-10, 0.01-50, 0.1-10, 0.1-50, 10-50, 5-20, 20-40 or 5-25 mM. In some embodiments, the concentration of each of ATP, GTP, CTP and UTP in the reaction composition is about 10 mM. In some embodiments, the concentration of GTP is about 7.5 mM. In some embodiments, the concentration of each of ATP, UTP and CTP is the same, and the concentration of GTP is higher than the concentration of each of ATP, UTP and CTP. In some embodiments, the concentration of GTP is about at least any one of 1.1, 1.2, 1.25, 1.3, 1.4, 1.5, 1.6, 1.7, 1.75, 1.8, 1.9, 2, 2.5, 3, 3.5, 4 times or more than the concentration of ATP, UTP or CTP.

In some embodiments, the DNA construct is contacted with the reagent composition at about at least any one of 30, 31, 32, 33, 34, 35, 36, 37, 38, 39, 40, 41, 42, 43, 44, 45, 46, 47, 48, 49, 50, 51, 52, 53, 54, 55, or 56° C. In some embodiments, the DNA construct is contacted with the reagent composition at no more than about any one of 56, 54, 53, 52, 51, 50, 49, 48, 47, 46, 45, 44, 43, 42, 41, 40, 39, 38, 37, 36, 35, 34, 33, 32, 31, or 30° C. In some embodiments, the DNA construct is contacted with the reagent composition at about any one of 30-31, 31-32, 32-33, 33-34, 34-35, 35-36, 36-37, 37-38, 38-40, 40-42, 42-44, 44-46, 46-48, 48-50, 50-52, 52-54, 54-56, 37-45, 45-56, or 40-50° C. In some embodiments, the DNA construct is contacted with the reagent composition at about 37° C.

In some embodiments, the DNA construct is contacted with the reagent composition for at least about any one of 5, 10, 20, 30, or 40 minutes, or 1, 2, 3, 4, 5, 6, 7, 8, 9, 10, 11, 12, 13, 14, 15, 16, 17, 18, 19, 20, 21, 22, 23, or 24 hours. In some embodiments, the DNA construct is contacted with the reagent composition for no more than about any one of 24, 23, 22, 21, 20, 19, 18, 17, 16, 15, 14, 13, 12, 11, 10, 9, 8, 7, 6, 5, 4, 3, 2, or 1 hour(s), or 40, 30, 20, 10, or 5 minutes. In some embodiments, the DNA construct is contacted with the reagent composition for about any one of 1-2, 2-4, 4-6, 6-8, 8-10, 10-12, 12-16, 16-18, 18-20, 20-22, 22-24, 1-6, 6-12, 12-18, 18-24, 1-12, 12-24, or 6-16 hours, or 20 minutes to 1 hour, or 20 minutes to 2 hours, or 20 minutes to 16 hours, or 20 minutes to 24 hours. In some embodiments, the DNA construct is contacted with the reagent composition for about 16 hours. In some embodiments, the DNA construct is contacted with the reagent composition for about 20 minutes to about 24 hours.

In some embodiments, the method comprises one or more additional steps for obtaining the DNA construct and/or isolating the circular RNA.

### Circularization by Ribozyme Autocatalysis Using Group I Introns

In some embodiments, the linear RNA is circularized in vitro. In some embodiments, the linear RNA is circularized in vivo. In some embodiments, circularization in vivo comprises introducing a construct encoding the linear RNA precursor and/or free 5′ Group I intron fragment into an individual, tissue, or cell, and allowing and allowing the linear RNA precursor and/or free 5′ Group I intron fragment to be expressed and circularized in vivo.

In some embodiments, circularization by ribozyme autocatalysis comprises (a) subjecting the linear RNA to a condition that activates autocatalysis of the catalytic Group I intron (or fragment thereof) to provide a circularized RNA product; and (b) isolating the circularized RNA product, thereby providing the circRNA.

In some embodiments, the method comprises a step of obtaining the linear RNA by first cloning the sequence encoding the linearized RNAs into a plasmid vector, and then linearizing the recombinant plasmids. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter. In some embodiments, the method further comprises purifying the linear RNA transcripts. In some embodiments, the linear RNAs are purified by gel purification.

In some embodiments, the present application provides a method of cyclizing a linear RNA (e.g., purified linear RNA) by ribozyme autocatalysis of the Group I intron or fragments thereof. In some embodiments, during splicing, transesterification occurs between the Group I intron or fragment thereof and an exon, resulting in circularization of the intervening region and excision of the intron. In some embodiments, the condition that activates autocatalysis of the Group I intron or fragments thereof is the addition of GTPs and/or Mg²⁺. In some embodiments, there is provided a step of cyclizing the linear RNAs by adding GTPs and Mg²⁺ at 55° C. for 15 min. In some embodiments, autocatalysis of the Group I intron or fragments thereof does not require the addition of GTPs. In some embodiments, there is provided a step of cyclizing the linear RNAs by adding Mg²⁺ at 55° C. for 15 min. In some embodiments, circularization takes place in less than 1 min, 1-2 min, 2-3 min, 3-4 min, 4-5 min, 5-6 min, 6-7 min, 7-8 min, 8-9 min, 9-10 min, 10-11 min, 11-12 min, 12-13 min, 13-14 min, 14-15 min, 15-16 min, 16-17 min, 17-18 min, 18-19 min, 19-20 min, 20-25 min, 25-30 min, 30-35 min, 35-40 min, 40-45 min, 45-50 min, 50-55 min, 55-60 min, 60-65 min, 65-70 min, 70-75 min, 75-80 min, 80-85 min, 85-90 min, 90-95 min, 95-100, 100-150 min or more than about 150 min. In some embodiments, about half of all linear RNA precursors are circularized within less than 1 min, 1-2 min, 2-3 min, 3-4 min, 4-5 min, 5-6 min, 6-7 min, 7-8 min, 8-9 min, 9-10 min, 10-11 min, 11-12 min, 12-13 min, 13-14 min, 14-15 min, 15-16 min, 16-17 min, 17-18 min, 18-19 min, 19-20 min, 20-25 min, 25-30 min, 30-35 min, 35-40 min, 40-45 min, 45-50 min, 50-55 min, 55-60 min, 60-65 min, 65-70 min, 70-75 min, 75-80 min, 80-85 min, 85-90 min, 90-95 min, 95-100, or within about 150 min. In some embodiments, more than half of all linear RNA precursors are circularized within less than 1 min, 1-2 min, 2-3 min, 3-4 min, 4-5 min, 5-6 min, 6-7 min, 7-8 min, 8-9 min, 9-10 min, 10-11 min, 11-12 min, 12-13 min, 13-14 min, 14-15 min, 15-16 min, 16-17 min, 17-18 min, 18-19 min, 19-20 min, 20-25 min, 25-30 min, 30-35 min, 35-40 min, 40-45 min, 45-50 min, 50-55 min, 55-60 min, 60-65 min, 65-70 min, 70-75 min, 75-80 min, 80-85 min, 85-90 min, 90-95 min, 95-100, or within about 150 min.

In some embodiments, circularization takes place at an Mg²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at an Mg²⁺ concentration of about 0-0.02, 0.02-0.04, 0.04-0.06, 0.06-0.08, 0.08-0.1 or more than about 0.1 mM. In some embodiments, circularization takes place at an Mg²⁺ concentration of about 0.1-0.12, 0.12-0.14, 0.14-0.16, 0.16-0.18, 0.18-0.20, 0.20-0.22, 0.22-0.24, or more than about 0.24 mM. In some embodiments, circularization takes place at an Mg²⁺ concentration of about 0.2-0.4, 0.4-0.6, 0.6-0.8, 0.8-1.0, 1.0-1.2, 1.2-1.4, 1.4-1.6, 1.6-1.8, 1.8-2.0, or more than about 2.0 mM. In some embodiments, circularization takes place at an Mg²⁺ concentration of about 5-15 mM. In some embodiments, circularization takes place at an Mg²⁺ concentration of about 10 mM. In some embodiments, circularization takes place at an Mg²⁺ concentration of less than about 10 mM. In some embodiments, circularization takes place at an Mg²⁺ concentration of about 4-8 mM. In some embodiments, circularization takes place at an Mg²⁺ concentration of about 6 mM. In some embodiments, circularization takes place at an Mg²⁺ concentration of about 0, 2.5, 3, 3.5, 4, 4.5, 5, 5.5, 6, 6.5, 7.5, 8, 8.5, 9, 9.5, or about 10 mM. In some embodiments, circularization takes place at an Mg²⁺ concentration of about 0.1, 0.5, 1, 2.5, 5, 10, 20, or about 40 mM. In some embodiments, circularization takes place at an Mg²⁺ concentration of about 20 mM. In some embodiments, circularization takes place at an Mg²⁺ concentration of about 40 mM. In some embodiments, circularization takes place at an Mg²⁺ concentration of about 0, 0.6, 0.9, 1.2, 1.5, 1.8, 2.1, or about 2.4 mM. In some embodiments, circularization takes place at an Mg²⁺ concentration of 0, 8, 10, 12, 14, 16, 18, or about 20 mM. In some embodiments, circularization takes place at an Mg²⁺ concentration of about 0.625 mM. In some embodiments, circularization takes place at an Mg²⁺ concentration of less than about 0.625 mM. In some embodiments, circularization at an Mg²⁺ concentration of about 4-8 mM, such as, for example about 6 mM, occurs faster via cis splicing compared to via PIE. In some embodiments, circularization at an Mg²⁺ concentration of about 4-8 mM, such as, for example about 6 mM, occurs faster via trans splicing compared to via PIE. In some embodiments, circularization at an Mg²⁺ concentration of about 10 mM occurs faster via cis splicing compared to via PIE. In some embodiments, circularization at an Mg²⁺ concentration of about 10 mM occurs faster via trans splicing compared to via PIE. In some embodiments, circularization takes place at an Mn²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Ca²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Co²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Be²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Cu²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at an Fe²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Zn²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Sr²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Ba²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at an Al²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Cd²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM.

In some embodiments, the method further comprises treating with RNase R to digest the linear RNA transcripts. In some embodiments, the method further comprises isolating the circular RNA (circRNA). In some embodiments, the step of isolating the circRNA comprises gel-purifying the circRNA. In some embodiments, the purified circRNA can be stored at −80° C.

In some embodiments, the circularization has an efficiency of at least 2%, at least 5%, at least 10%, at least 15%, at least 20%, at least 25%, at least 30%, at least 32%, at least 34%, at least 36%, at least 38%, at least 40%, at least 42%, at least 44%, at least 46%, at least 48%, or at least 50%. In some embodiments, the circularization has an efficiency of about 40% to about 50% or more than 50%.

A) Splicing in Cis with a Group I Intron

In some embodiments, Group I intron-mediated circRNA autocatalysis occurs via splicing in cis across one initial piece of linear RNA. In some embodiments, cis splicing occurred via a single splicing step. In some embodiments, Group I intron-mediated circRNA autocatalysis occurs via splicing in cis via only a single transesterification step and a single RNA cleavage step. In some embodiments, cis splicing comprises non-covalent binding between the free 5′ intron fragment and the 3′ intron fragment. In some embodiments, cis splicing comprises covalent binding between the free 5′ intron fragment and the 3′ intron fragment. In some embodiments, cis splicing does not require split sites in the Group I intron sequence. In some embodiments, cis splicing does not require Group I intron fragments. In some embodiments, cis splicing comprises Group I intron fragments. In some embodiments, cis splicing comprises an intact catalytic Group I intron sequence in which the 5′ and 3′ sequences are covalently linked without an intervening sequence.

In some embodiments, there is a single G nucleotide present on the 5′ end of the linear RNA. In some embodiments, there are two G nucleotides present on the 5′ end of the linear RNA. In some embodiments, there are 3, 4, 5, 6, 7, 8, 9, 10, or more than 10 G nucleotides present on the 5′ end of the linear RNA. In some embodiments, the linear RNA comprises an intact Group I intron sequence in its natural form. In some embodiments, the linear RNA comprises a split Group I intron sequence.

In some embodiments, RNA ligation during cis splicing occurs between the 5′ of Exon2 and the 3′ of Exon1. In some embodiments, RNA ligation during cis splicing involves physical contact between the 5′ of Exon2 and the 3′ of Exon1. In some embodiments, the presence of homology arms in the exon sequences facilitates circularization. In some embodiments, extending exons length facilitates circularization. In some embodiments, the absence of homology arms in the intron sequences facilitates circularization.

In some embodiments, there is a U nucleotide on the 3′ end of the linear RNA. In some embodiments, there is an A nucleotide on the 3′ end of the linear RNA. In some embodiments, there is a C nucleotide on the 3′ end of the linear RNA. In some embodiments, there is a G nucleotide on the 3′ end of the linear RNA.

B) Splicing in Trans with a Group I Intron

In some embodiments, Group I intron-mediated circRNA autocatalysis occurs via splicing in trans across two initial pieces of linear RNA. In some embodiments, trans splicing occurred via a single splicing step between two starting pieces of RNA: i) a linear RNA precursor (such as, e.g., SEQ ID NO: 2) that comprises, from its 5′ to 3′ end, a 3′ catalytic Group I intron fragment (such as, e.g., SEQ ID NO: 3), a 3′ exon sequence (such as, e.g., SEQ ID NO: 4), and a 5′ exon sequence (such as, e.g., SEQ ID NO: 6); and ii) a free 5′ catalytic Group I intron fragment (such as, e.g., SEQ ID NO: 1). In some embodiments, Group I intron-mediated circRNA autocatalysis occurs via splicing in trans via only a single transesterification step and a single RNA cleavage step. In some embodiments, trans splicing comprises non-covalent binding between the free 5′ intron fragment and the 3′ intron fragment. In some embodiments, the non-covalent binding between the free 5′ intron fragment and the 3′ intron fragment results in the formation of a canonical Group I intron structure.

In some embodiments, the free 5′ catalytic Group I intron fragment is introduced to the in vitro or in vivo reaction context simultaneously with the linear RNA precursor. In some embodiments, the free 5′ catalytic Group I intron fragment is introduced to the in vitro or in vivo reaction context later than the linear RNA precursor, to trigger the reaction. In some embodiments, the free 5′ catalytic Group I intron fragment is introduced as an RNA fragment. In some embodiments, the free 5′ catalytic Group I intron fragment is introduced via expression from a vector or other polynucleotide construct.

### Circularization by Ribozyme Autocatalysis Using Group II Introns

In some embodiments, the linear RNA is circularized in vitro. In some embodiments, the linear RNA is circularized in vivo. In some embodiments, circularization in vivo comprises introducing a construct encoding the linear RNA precursor and/or free 5′ Group II intron fragment into an individual, tissue, or cell, and allowing and allowing the linear RNA precursor and/or free 5′ Group II intron fragment to be expressed and circularized in vivo.

In some embodiments, circularization by ribozyme autocatalysis comprises (a) subjecting the linear RNA to a condition that activates autocatalysis of the catalytic Group II intron (or fragment thereof) to provide a circularized RNA product; and (b) isolating the circularized RNA product, thereby providing the circRNA.

In some embodiments, the method comprises a step of obtaining the linear RNA by first cloning the sequence encoding the linearized RNAs into a plasmid vector, and then linearizing the recombinant plasmids. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter. In some embodiments, the method further comprises purifying the linear RNA transcripts. In some embodiments, the linear RNAs are purified by gel purification.

In some embodiments, the present application provides a method of cyclizing a linear RNA (e.g., purified linear RNA) by ribozyme autocatalysis of the Group II intron or fragments thereof. In some embodiments, during splicing, transesterification occurs between the Group II intron or fragment thereof and an exon, resulting in circularization of the intervening region and excision of the intron. In some embodiments, the condition that activates autocatalysis of the Group II intron or fragments thereof is the addition of GTPs and/or Mg²⁺. In some embodiments, there is provided a step of cyclizing the linear RNAs by adding GTPs and Mg²⁺ at 55° C. for 15 min. In some embodiments, autocatalysis of the Group II intron or fragments thereof does not require the addition of GTPs. In some embodiments, there is provided a step of cyclizing the linear RNAs by adding Mg²⁺ at 55° C. for 15 min. In some embodiments, circularization takes place in less than 1 min, 1-2 min, 2-3 min, 3-4 min, 4-5 min, 5-6 min, 6-7 min, 7-8 min, 8-9 min, 9-10 min, 10-11 min, 11-12 min, 12-13 min, 13-14 min, 14-15 min, 15-16 min, 16-17 min, 17-18 min, 18-19 min, 19-20 min, 20-25 min, 25-30 min, 30-35 min, 35-40 min, 40-45 min, 45-50 min, 50-55 min, 55-60 min, 60-65 min, 65-70 min, 70-75 min, 75-80 min, 80-85 min, 85-90 min, 90-95 min, 95-100, 100-150 min or more than about 150 min. In some embodiments, about half of all linear RNA precursors are circularized within less than 1 min, 1-2 min, 2-3 min, 3-4 min, 4-5 min, 5-6 min, 6-7 min, 7-8 min, 8-9 min, 9-10 min, 10-11 min, 11-12 min, 12-13 min, 13-14 min, 14-15 min, 15-16 min, 16-17 min, 17-18 min, 18-19 min, 19-20 min, 20-25 min, 25-30 min, 30-35 min, 35-40 min, 40-45 min, 45-50 min, 50-55 min, 55-60 min, 60-65 min, 65-70 min, 70-75 min, 75-80 min, 80-85 min, 85-90 min, 90-95 min, 95-100, or within about 150 min. In some embodiments, more than half of all linear RNA precursors are circularized within less than 1 min, 1-2 min, 2-3 min, 3-4 min, 4-5 min, 5-6 min, 6-7 min, 7-8 min, 8-9 min, 9-10 min, 10-11 min, 11-12 min, 12-13 min, 13-14 min, 14-15 min, 15-16 min, 16-17 min, 17-18 min, 18-19 min, 19-20 min, 20-25 min, 25-30 min, 30-35 min, 35-40 min, 40-45 min, 45-50 min, 50-55 min, 55-60 min, 60-65 min, 65-70 min, 70-75 min, 75-80 min, 80-85 min, 85-90 min, 90-95 min, 95-100, or within about 150 min.

In some embodiments, circularization takes place at an Mg²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at an Mg²⁺ concentration of about 0-0.02, 0.02-0.04, 0.04-0.06, 0.06-0.08, 0.08-0.1 or more than about 0.1 mM. In some embodiments, circularization takes place at an Mg²⁺ concentration of about 0.1-0.12, 0.12-0.14, 0.14-0.16, 0.16-0.18, 0.18-0.20, 0.20-0.22, 0.22-0.24, or more than about 0.24 mM. In some embodiments, circularization takes place at an Mg²⁺ concentration of about 0.2-0.4, 0.4-0.6, 0.6-0.8, 0.8-1.0, 1.0-1.2, 1.2-1.4, 1.4-1.6, 1.6-1.8, 1.8-2.0, or more than about 2.0 mM. In some embodiments, circularization takes place at an Mg²⁺ concentration of about 5-15 mM. In some embodiments, circularization takes place at an Mg²⁺ concentration of about 10 mM. In some embodiments, circularization takes place at an Mg²⁺ concentration of less than about 10 mM. In some embodiments, circularization takes place at an Mg²⁺ concentration of about 4-8 mM. In some embodiments, circularization takes place at an Mg²⁺ concentration of about 6 mM. In some embodiments, circularization takes place at an Mg²⁺ concentration of about 0, 2.5, 3, 3.5, 4, 4.5, 5, 5.5, 6, 6.5, 7.5, 8, 8.5, 9, 9.5, or about 10 mM. In some embodiments, circularization takes place at an Mg²⁺ concentration of about 0.1, 0.5, 1, 2.5, 5, 10, 20, or about 40 mM. In some embodiments, circularization takes place at an Mg²⁺ concentration of about 20 mM. In some embodiments, circularization takes place at an Mg²⁺ concentration of about 40 mM. In some embodiments, circularization takes place at an Mg²⁺ concentration of about 0, 0.6, 0.9, 1.2, 1.5, 1.8, 2.1, or about 2.4 mM. In some embodiments, circularization takes place at an Mg²⁺ concentration of 0, 8, 10, 12, 14, 16, 18, or about 20 mM. In some embodiments, circularization takes place at an Mg²⁺ concentration of about 0.625 mM. In some embodiments, circularization takes place at an Mg²⁺ concentration of less than about 0.625 mM. In some embodiments, circularization at an Mg²⁺ concentration of about 4-8 mM, such as, for example about 6 mM, occurs faster via cis splicing compared to via PIE. In some embodiments, circularization at an Mg²⁺ concentration of about 4-8 mM, such as, for example about 6 mM, occurs faster via trans splicing compared to via PIE. In some embodiments, circularization at an Mg²⁺ concentration of about 10 mM occurs faster via cis splicing compared to via PIE. In some embodiments, circularization at an Mg²⁺ concentration of about 10 mM occurs faster via trans splicing compared to via PIE. In some embodiments, circularization takes place at an Mn²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Ca²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Co²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Be²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Cu²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at an Fe²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Zn²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Sr²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Ba²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at an Al²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM. In some embodiments, circularization takes place at a Cd²⁺ concentration of about 0-1, 1-2, 2-3, 3-4, 4-5, 5-6, 6-7, 7-8, 8-9, 9-10, 10-11, 11-12, 12-13, 13-14, 14-15, 15-16, 16-17, 17-18, 18-19, 19-20, 20-25, 25-30, 30-35, 35-40, 40-45, 45-50, 50-55, 55-60, 60-65, 65-70, 70-75, 75-80, 80-85, 85-90, 90-95, 95-100, or more than about 100 mM.

In some embodiments, the method further comprises treating with RNase R to digest the linear RNA transcripts. In some embodiments, the method further comprises isolating the circular RNA (circRNA). In some embodiments, the step of isolating the circRNA comprises gel-purifying the circRNA. In some embodiments, the purified circRNA can be stored at −80° C.

In some embodiments, the circularization has an efficiency of at least 2%, at least 5%, at least 10%, at least 15%, at least 20%, at least 25%, at least 30%, at least 32%, at least 34%, at least 36%, at least 38%, at least 40%, at least 42%, at least 44%, at least 46%, at least 48%, or at least 50%. In some embodiments, the circularization has an efficiency of about 40% to about 50% or more than 50%.

A) Splicing in Cis with a Group II Intron

In some embodiments, Group II intron-mediated circRNA autocatalysis occurs via splicing in cis across one initial piece of linear RNA. In some embodiments, cis splicing occurred via a single splicing step. In some embodiments, Group II intron-mediated circRNA autocatalysis occurs via splicing in cis via only a single transesterification step and a single RNA cleavage step. In some embodiments, cis splicing comprises non-covalent binding between the free 5′ intron fragment and the 3′ intron fragment. In some embodiments, cis splicing comprises covalent binding between the free 5′ intron fragment and the 3′ intron fragment. In some embodiments, cis splicing does not require split sites in the Group II intron sequence. In some embodiments, cis splicing does not require Group II intron fragments. In some embodiments, cis splicing comprises Group II intron fragments. In some embodiments, cis splicing comprises an intact catalytic Group II intron sequence in which the 5′ and 3′ sequences are covalently linked without an intervening sequence.

In some embodiments, there is a single G nucleotide present on the 5′ end of the linear RNA. In some embodiments, there are two G nucleotides present on the 5′ end of the linear RNA. In some embodiments, there are 3, 4, 5, 6, 7, 8, 9, 10, or more than 10 G nucleotides present on the 5′ end of the linear RNA. In some embodiments, the linear RNA comprises an intact Group II intron sequence in its natural form. In some embodiments, the linear RNA comprises a split Group II intron sequence.

In some embodiments, RNA ligation during cis splicing occurs between the 5′ of Exon2 and the 3′ of Exon1. In some embodiments, RNA ligation during cis splicing involves physical contact between the 5′ of Exon2 and the 3′ of Exon1. In some embodiments, the presence of homology arms in the exon sequences facilitates circularization. In some embodiments, extending exons length facilitates circularization. In some embodiments, the absence of homology arms in the intron sequences facilitates circularization.

In some embodiments, there is a U nucleotide on the 3′ end of the linear RNA. In some embodiments, there is an A nucleotide on the 3′ end of the linear RNA. In some embodiments, there is a C nucleotide on the 3′ end of the linear RNA. In some embodiments, there is a G nucleotide on the 3′ end of the linear RNA.

B) Splicing in Trans with a Group II Intron

In some embodiments, Group II intron-mediated circRNA autocatalysis occurs via splicing in trans across two initial pieces of linear RNA. In some embodiments, trans splicing occurred via a single splicing step between two starting pieces of RNA: i) a linear RNA precursor (such as, e.g., SEQ ID NO: 2) that comprises, from its 5′ to 3′ end, a 3′ catalytic Group II intron fragment (such as, e.g., SEQ ID NO: 3), a 3′ exon sequence (such as, e.g., SEQ ID NO: 4), and a 5′ exon sequence (such as, e.g., SEQ ID NO: 6); and ii) a free 5′ catalytic Group II intron fragment (such as, e.g., SEQ ID NO: 1). In some embodiments, Group II intron-mediated circRNA autocatalysis occurs via splicing in trans via only a single transesterification step and a single RNA cleavage step. In some embodiments, trans splicing comprises non-covalent binding between the free 5′ intron fragment and the 3′ intron fragment. In some embodiments, the non-covalent binding between the free 5′ intron fragment and the 3′ intron fragment results in the formation of a canonical Group II intron structure.

In some embodiments, the free 5′ catalytic Group II intron fragment is introduced to the in vitro or in vivo reaction context simultaneously with the linear RNA precursor. In some embodiments, the free 5′ catalytic Group II intron fragment is introduced to the in vitro or in vivo reaction context later than the linear RNA precursor, to trigger the reaction. In some embodiments, the free 5′ catalytic Group II intron fragment is introduced as an RNA fragment. In some embodiments, the free 5′ catalytic Group II intron fragment is introduced via expression from a vector or other polynucleotide construct.

### Plasmids

In some embodiments, the present application provides plasmids comprising the nucleotide sequences described herein. In some embodiments, the plasmids are obtained by cloning the sequence encoding the linearized RNAs into a plasmid vector. Plasmids can be generated by techniques known in the art, such as Gibson cloning or cloning using restriction enzymes. In some embodiments, the plasmid vector includes an antibiotic expression cassette allowing antibiotic selection of bacteria expressing the plasmid. In some embodiments, the plasmids provided can be purified from bacteria and used for production of the linear circRNA constructs. In some embodiments, the plasmids provided can be delivered to a host cell and transcribed in vivo therein. Any plasmid vector suitable for in vitro or in vivo transcription of the linear RNA may be used.

In some embodiments, the plasmids are linearized prior to in vitro transcription of the linear RNA. In some embodiments, the recombinant plasmids are linearized by restriction enzyme digestion. In some embodiments, the recombinant plasmids are linearized by PCR amplification. In some embodiments, the method further comprises performing in vitro transcription with the linearized plasmid template. In some embodiments, the in vitro transcription is driven by a T7 promoter.

In some embodiments, the method comprises producing the circular RNA from a DNA construct encoding a linear RNA precursor. In some embodiments, the DNA construct is a plasmid. In some embodiments, the method comprises linearizing the plasmid. In some embodiments, the plasmid is linearized by restriction enzyme digestion. In some embodiments, the plasmid is linearized by PCR amplification. In some embodiments, a linearized PCR product comprises one or more modification, which can be generated from, for example, use of a modified primer during the PCR amplification. In some embodiments, the modification is a chemical modification, such as, for instance, methylation. In some embodiments, the methylation is 2′-OMe.

In some embodiments, the method comprises treating the product of the circularization reaction with RNase R to digest the linear RNA precursor molecules that are not circularized. In some embodiments, the method does not comprise treating the product of the circularization reaction with RNase R to digest the linear RNA precursor molecules that are not circularized.

In some embodiments, the method further comprises a step of purifying the circularized RNA product. In non-limiting examples, the circRNA is purified by gel-purification or by high-performance liquid chromatography (HPLC). In some embodiments, agarose gel electrophoresis allows for simple and effective separation of circular splicing products from linear precursor molecules, nicked circles, splicing intermediates, and excised introns. In some embodiments, the method comprises purifying the circular dRNA by chromatography, such as HPLC. In some embodiments, the purified circular dRNA can be stored at −80° C.

In some embodiments, the purification comprises removing impurities, such as the spliced intron and linear precursor products of the circularization reaction. In some embodiments, impurities are removed by treatment with oligo dT beads.

Purification of circRNA

In some embodiments, the method provided herein of producing a circRNA further comprises a step of purifying the circularized RNA product. In non-limiting examples, the circRNA is purified by gel-purification or by high-performance liquid chromatography (HPLC). In some embodiments, agarose gel electrophoresis allows for simple and effective separation of circular splicing products from linear precursor molecules, nicked circles, splicing intermediates, and excised introns. In some embodiments, the method comprises purifying the circular RNA by chromatography, such as HPLC. In some embodiments, the method comprises purifying the circular RNA by separating impurities comprising a heterologous sequence, for instance, using the methods described in the Group I intron or Group II intron sections above regarding separation of reaction products comprising heterologous sequences. In some embodiments, the purified circular RNA can be stored at −80° C.

### Pharmaceutical Compositions, Kits and Articles of Manufacture

Further provided by the present application are pharmaceutical compositions comprising any one of circRNAs described herein, and a pharmaceutically acceptable carrier. Pharmaceutical compositions can be prepared by mixing the therapeutic agents described herein having the desired degree of purity with optional pharmaceutically acceptable carriers, excipients or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers, antioxidants including ascorbic acid, methionine, Vitamin E, sodium metabisulfite; preservatives, isotonicifiers (e.g. sodium chloride), stabilizers, metal complexes (e.g. Zn-protein complexes); chelating agents such as EDTA and/or non-ionic surfactants.

In some embodiments, the pharmaceutical composition is contained in a single-use vial, such as a single-use sealed vial. In some embodiments, the pharmaceutical composition is contained in a multi-use vial. In some embodiments, the pharmaceutical composition is contained in bulk in a container. In some embodiments, the pharmaceutical composition is cryopreserved.

The present application further provides kits and articles of manufacture for use in any embodiment of the treatment methods described herein. The kits and articles of manufacture may comprise any one of the formulations and pharmaceutical compositions described herein.

In some embodiments, there is provided a kit comprising any one of the circRNAs described herein and instructions for treating or preventing a disease or condition (e.g., coronavirus infection).

In some embodiments, there is provided a kit comprising any one of the circRNA described herein and instructions for treating or preventing a coronavirus infection.

In some embodiments, there is provided a kit comprising any one of the plasmids or linear RNAs described herein, and instructions for preparing any one of the circRNAs. In some embodiments, there is provided a kit comprising any one of the plasmids, linear RNAs, or circRNAs described herein, and instructions for administering the circRNA.

The kits of the invention are in suitable packaging. Suitable packaging includes, but is not limited to, vials, bottles, jars, flexible packaging (e.g., sealed Mylar or plastic bags), and the like. Kits may optionally provide additional components such as buffers and interpretative information. The present application thus also provides articles of manufacture, which include vials (such as sealed vials), bottles, jars, flexible packaging, and the like.

The instructions relating to the use of the compositions generally include information as to dosage, dosing schedule, and route of administration for the intended treatment. The containers may be unit doses, bulk packages (e.g., multi-dose packages) or sub-unit doses. For example, kits may be provided that contain sufficient dosages of the circRNA as disclosed herein to provide effective treatment of an individual or of many individuals. Additionally, kits may be provided that contain sufficient dosages of the circRNA to allow for multiple administrations to an individual (e.g., initial vaccine administration and subsequent booster administration, in the case of a circRNA vaccine). Kits may also include multiple unit doses of the pharmaceutical compositions and instructions for use and packaged in quantities sufficient for storage and use in pharmacies, for example, hospital pharmacies and compounding pharmacies.

In some embodiments, the kit comprises a delivery system. The delivery system may be a unit dose delivery system. The volume of solution or suspension delivered per dose can be anywhere from about 5 to about 2000 microliters, from about 10 to about 1000 microliters, or from about 50 to about 500 microliters. Delivery systems for these various dosage forms can be syringes, dropper bottles, plastic squeeze units, atomizers, nebulizers or pharmaceutical aerosols in either unit dose or multiple dose packages. In some embodiments, there is provided a delivery system of any one of the circRNAs described herein, comprising the circRNA and a device for delivering the circRNA.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

### III. Circular RNAs and Methods of Use

The present application further provides circRNAs and compositions prepared using any one of the methods of preparation described herein.

In some embodiments, the circRNA comprises an effector RNA sequence. In some embodiments, the effector RNA sequence is a coding RNA. In some embodiments, the effector RNA sequence comprises a nucleic acid sequence encoding a therapeutic polypeptide. In some embodiments, the therapeutic polypeptide is selected from the group consisting of an antigenic polypeptide, a functional protein, a receptor protein, and a targeting protein.

In some embodiments, the effector RNA sequence comprises a Kozak sequence operably linked to the to the nucleic acid sequence encoding the therapeutic polypeptide.

In some embodiments, the effector RNA sequence comprises an in-frame 2A peptide coding sequence operably linked to the 3′ end of the nucleic acid sequence encoding the therapeutic polypeptide.

In some embodiments, the effector RNA sequence comprises an internal ribosomal entry site (IRES) sequence operably linked to the nucleic acid sequence encoding the therapeutic polypeptide.

In some embodiments, the effector RNA sequence comprises an m6A modification motif sequence operably linked to the nucleic acid sequence encoding the therapeutic polypeptide.

In some embodiments, the effector RNA sequence comprises a nucleic acid sequence comprising a therapeutic RNA. In some embodiments, the therapeutic RNA is an RNA molecule selected from the group consisting of a gRNA, a dRNA, a siRNA, a miRNA, a shRNA, and a lincRNA.

In some embodiments, there is provided a cocktail composition comprising a plurality of circRNAs each comprising a coding RNA sequence encoding an antigenic polypeptide, a receptor protein of an infectious agent, or a targeting protein (e.g., an antibody such as a neutralizing antibody). In some embodiments, the plurality of circRNA encode antigenic polypeptides that are different with respect to each other, such as different mutants of an antigenic polypeptide (e.g., S protein or fragment thereof). In some embodiments, the plurality of circRNA encode receptor proteins that are different with respect to each other, such as different mutants of a receptor protein (e.g., ACE2). In some embodiments, the plurality of circRNA encode targeting proteins that are different with respect to each other, such as different antibodies (e.g., neutralizing antibodies).

The circRNAs described herein may be used to treat or prevent a disease or condition in an individual, including, but not limited to genetic diseases (e.g., hereditary genetic diseases, metabolic diseases and cancer), and infections (e.g., viral infections such as coronavirus infections). In some embodiments, the circRNA is subject to rolling circle translation by a ribosome in the individual.

In some embodiments, there is provided a method of treating or preventing a disease or condition in an individual, comprising administering to the individual an effective amount of a circRNA prepared using any one of the methods described herein. In some embodiments, the circRNA comprises a coding RNA sequence encoding a functional protein. In some embodiments, the functional protein is an enzyme, a receptor, a ligand, a signaling molecule, or a transcription factor. In some embodiments, the disease or condition is a metabolic disease. In some embodiments, the disease or condition is a lysosomal storage disorder. In some embodiments, the disease or condition is a cancer.

The circRNAs described herein may be used for treating a genetic disease or condition that is associated with a mutation or deficiency in a naturally-occurring protein corresponding to the therapeutic polypeptide encoded by the circRNA. In some embodiments, the disease or condition is a disease or condition associated with insufficient levels and/or activity of a naturally-occurring protein corresponding to the therapeutic polypeptide. In some embodiments, the disease or condition is a hereditary genetic disease associated with one or more mutations in naturally-occurring protein corresponding to the therapeutic polypeptide. In some embodiments, the therapeutic polypeptide is a wildtype protein, or a functional variant thereof (e.g., a functional fragment, fusion protein, or mutant).

In some embodiments, the therapeutic polypeptide can be any polypeptide that is capable of being expressed by target cells (e.g., human or mouse cells) for the production (and in certain instances, the excretion) of a functional enzyme or protein as disclosed, for example, in International Application No. PCT/US2010/058457. In some embodiments, the therapeutic polypeptide can be engineered for secretion by operably linking a signal peptide to the amino terminus of the therapeutic polypeptide. For example, in some embodiments, upon the expression of one or more therapeutic polynucleotides by target cells, the production of a functional enzyme or protein in which a subject is deficient (e.g., a urea cycle enzyme or an enzyme associated with a lysosomal storage disorder) may be observed.

Examples of disease-associated mutations that may be treated by the methods of the present application include, but are not limited to, TP53^{W53X} (e.g., 158G>A) associated with cancer, IDUA^{W402X} (e.g., TGG>TAG mutation in exon 9) associated with Mucopolysaccharidosis type I (MPS I), COL3A1^{W1278X} (e.g., 3833G>A mutation) associated with Ehlers-Danlos syndrome, BMPR2^{W298X} (e.g., 893G>A) associated with primary pulmonary hypertension, AHI1^{W725X} (e.g., 2174G>A) associated with Joubert syndrome, FANCC^{W506X} (e.g., 1517G>A) associated with Fanconi anemia, MYBPC3^{W1098X} (e.g., 3293G>A) associated with primary familial hypertrophic cardiomyopathy, and IL2RG^{W237X} (e.g., 710G>A) associated with X-linked severe combined immunodeficiency. In some embodiments, the disease or condition is a cancer. In some embodiments, the disease or condition is a monogenetic disease. In some embodiments, the disease or condition is a polygenetic disease.

In some embodiments, the circRNA has a functional half-life of at least or at least about 20 hours, 24 hours, 30 hours, or 36 hours. In some embodiments, the circRNA has a duration of therapeutic effect in a human cell of at least or at least about 20 hours, 24 hours, 30 hours, or 36 hours. In some embodiments, the circRNA has a duration of therapeutic effect in a human cell greater than or equal to that of an equivalent linear RNA comprising the same expression sequence. In some embodiments, the circRNA has a functional half-life in a human cell greater than or equal to that of an equivalent linear RNA comprising the same expression sequence.

In some embodiments, the present application provides circRNAs for use in treating or preventing a disease or condition in an individual.

In some embodiments, the present application provides use of a circRNA comprising a nucleic acid sequence encoding a therapeutic polypeptide for the manufacture of a medicament for treating or preventing a disease or condition in an individual.

In some embodiments, the circRNA is administered as naked circRNA, or as a pharmaceutical composition comprising a transfection agent. In non-limiting examples, the transfection agent is polyethylenimine (PEI) or a lipid nanoparticle (LNP). Other examples of lipidosomes that can be used to administer the circRNA composition for administration (e.g., circRNA vaccine or pharmaceutical composition) include protamines, cationic nanoemulsions, modified dendrimer nanoparticles, protamine liposomes, cationic polymers, cationic polymer liposomes, polysaccharide particles, cationic lipid nanoparticles, cationic lipid-cholesterol nanoparticles, cationic lipid-cholesterol PEG nanoparticle, cationic lipid transfection reagents sold under the trademark LIPOFECTAMINE, nonliposomal transfection reagents sold under the trademark FUGENE, or any combination thereof can be used as the transfection agent.

In some embodiments, the liposome formulation may be influenced by, but not limited to, the selection of the cationic lipid component, the degree of cationic lipid saturation, the nature of the PEGylation, ratio of all components and biophysical parameters such as size. In some embodiments, the liposome formulation comprises a cationic lipid, a cholesterol and a PEGylated lipid. For example, a liposome formulation may comprise a cationic lipid, dipalmitoylphosphatidylcholine, cholesterol, and PEG-c-DMA. See, for example, Semple et al. Nature Biotech. 2010 28:172-176, herein incorporated by reference in its entirety. In some embodiments, liposome formulations may comprise from about 35 to about 45% cationic lipid, from about 40% to about 50% cationic lipid, from about 50% to about 60% cationic lipid and/or from about 55% to about 65% cationic lipid. In some embodiments, the ratio of lipid to RNA in liposomes may be from about 5:1 to about 20:1, from about 10:1 to about 25:1, from about 15:1 to about 30:1 and/or at least 30:1. Suitable liposome formulations have been described, for example, in WO2020237227, the contents of which are herein incorporated by reference in their entirety.

In some embodiments, the circRNA is not formulated with a transfection reagent. In some embodiments, the circRNA is delivered as naked RNA. In some embodiments, the circRNA is delivered by gene gun or by electroporation.

The circRNA composition for administration (e.g., circRNA vaccine or pharmaceutical composition) can be administered to a subject by systemic injection into the vasculature, systemic injection into the lymph nodes, subcutaneous injection or depots, or by local injection.

In some embodiments, the circRNA may be formulated in a lipid nanoparticle such as those described in International Publication No. WO2012170930, herein incorporated by reference in its entirety.

In some embodiments, the synthetic nanocarriers may be formulated for controlled and/or sustained release of the circRNA described herein. As a non-limiting example, the synthetic nanocarriers for sustained release may be formulated by methods known in the art, described herein and/or as described in International Pub No. WO2010138192 and US Pub No. 20100303850, each of which is herein incorporated by reference in their entirety.

In some embodiments, the circRNA may be formulated for controlled and/or sustained release wherein the formulation comprises at least one polymer that is a crystalline side chain (CYSC) polymer. CYSC polymers are described in U.S. Pat. No. 8,399,007, herein incorporated by reference in its entirety.

In some embodiments, the synthetic nanocarrier may be formulated for use as a vaccine. In some embodiments, the synthetic nanocarrier may encapsulate at least one circRNA, which encode at least one antigen. As a nonlimiting example, the synthetic nanocarrier may include at least one antigen and an excipient for a vaccine dosage form (see International Pub No. WO2011150264 and US Pub No. US201 10293723, each of which is herein incorporated by reference in their entirety). As another non-limiting example, a vaccine dosage form may include at least two synthetic nanocarriers with the same or different antigens and an excipient (see International Pub No. WO201 1150249 and US Pub No. US201 10293701, each of which is herein incorporated by reference in their entirety). The vaccine dosage form may be selected by methods described herein, known in the art and/or described in International Pub No. WO2011150258 and US Pub No. US20120027806, each of which is herein incorporated by reference in their entirety).

In some embodiments, the synthetic nanocarrier may comprise at least one circRNA, which encodes at least one adjuvant. As non-limiting example, the adjuvant may comprise dimethyldioctadecylammonium-bromide, dimethyldioctadecylammoniumchloride, dimethyldioctadecylammonium-phosphate or dimethyldioctadecylammoniumacetate (DDA) and an apolar fraction or part of said apolar fraction of a total lipid extract of a mycobacterium (See e.g., U.S. Pat. No. 8,241,610; herein incorporated by reference in its entirety). In another embodiment, the synthetic nanocarrier may comprise at least one circRNA and an adjuvant. As a non-limiting example, the synthetic nanocarrier comprising and adjuvant may be formulated by the methods described in International Pub No. WO2011150240 and US Pub No. US20110293700, each of which is herein incorporated by reference in its entirety.

In some embodiments, the circRNA functions as an adjuvant. As an example, RNA-sensing in the cytoplasm can trigger innate immunity, and innate immune signaling is known to contribute to adaptive immunity by diverse routes. Thus, the circRNA encoding the antigenic polypeptide or a second circRNA (e.g., a circRNA that does not encode a polypeptide) can be used as an adjuvant for boosting the adaptive immune response to the antigenic polypeptide.

In some embodiments, the circRNA compositions of the present application may be administrated with other prophylactic or therapeutic compounds. As a non-limiting example, the prophylactic or therapeutic compound may be an adjuvant or a booster. As used herein, when referring to a prophylactic composition, such as a vaccine, the term “booster” refers to an extra administration of the prophylactic composition. A booster (or booster vaccine) may be given after an earlier administration of the prophylactic composition. The time of administration between the initial administration of the prophylactic composition and the booster may be, but is not limited to, 1 minute, 2 minutes, 3 minutes, 4 minutes, 5 minutes, 6 minutes, 7 minutes, 8 minutes, 9 minutes, 10 minutes, 15 minutes, 20 minutes 35 minutes, 40 minutes, 45 minutes, 50 minutes, 55 minutes, 1 hour, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 1 day, 36 hours, 2 days, 3 days, 4 days, 5 days, 6 days, 1 week, 10 days, 2 weeks, 3 weeks, 1 month, 2 months, 3 months, 4 months, 5 months, 6 months, 7 months, 8 months, 9 months, 10 months, 11 months, 1 year, 18 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 25 years, 30 years, 35 years, 40 years, 45 years, 50 years, 55 years, 60 years, 65 years, 70 years, 75 years, 80 years, 85 years, 90 years, 95 years or more than 99 years.

In some embodiments, the circRNA composition for administration (e.g., circRNA vaccine or pharmaceutical composition) may be administered intranasally. For example, circRNA vaccines may be administered intranasally similar to the administration of live vaccines. In some embodiments, the circRNA may be administered intramuscularly or intradermally similarly to the administration of inactivated vaccines known in the art.

In some embodiments, the circRNA vaccine comprises an adjuvant, which may enable the vaccine to elicit a higher immune response. As a non-limiting example, the adjuvant could be a sub-micron oil-in-water emulsion, which can elicit a higher immune response in human pediatric populations (see e.g., the adjuvant-containing vaccines described in US Patent Publication No. US20120027813 and US Patent No. U.S. Pat. No. 8,506,966, the contents of each of which are herein incorporated by reference in its entirety).

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended embodiments.

### Example 1: Group I Intron-Mediated RNA Circularization with Trans-Splicing

This example demonstrates that circRNA can be generated by trans splicing between i) a linear RNA precursor comprising a 3′ intron fragment on the 5′ end but lacking a 5′ intron fragment on the 3′ end, and ii) a free 5′ intron fragment with a G on its 5′ end.

### Background

Canonical Group I autocatalysis and PIE circularization are understood to require a linear RNA precursor that contains, in a single piece of RNA, at least an exon sequence and 5′ and 3′ Group I intron sequences (FIG. 1A). Group I introns work as ribozymes to mediate RNA splicing without protein via a two-step transesterification reaction requiring a guanosine cofactor. The PIE method involves both steps of this transesterification reaction to accomplish canonical RNA circularization, using a single linear RNA precursor that has a 3′ Group I intron fragment on its 5′ end, exon and Gene of Interest (GOI) sequences in the middle, and a 5′ Group I intron fragment on its 3′ end (FIG. 1A, right). In the 1st step of the PIE method, an exogenous guanosine (exoG) attacks the boundary between the exon and the 5′ intron fragment on the linear RNA precursor, mediating RNA cleavage and the addition of a single G nucleotide on the 5′ end of the 5′ intron fragment (FIG. 1A, top right). The 3′ Group I intron fragment is then cleaved from the linear RNA intermediate in the 2^{nd} step of the PIE method, resulting in a free linear 3′ intron fragment, a free circRNA (containing the exon and GOI sequences), and a free linear 5′ intron fragment with a single G nucleotide on its 5′ end. The PIE method is the state of the art for Group I intron-mediated circRNA autocatalysis.

Surprisingly, the experiments presented herein demonstrate that Group I intron-mediated circRNA autocatalysis can also take place in trans by providing two initial pieces of linear RNA rather than a single linear RNA precursor. In the trans splicing method described herein, circularization occurred via a single splicing step between two starting pieces of RNA: i) a linear RNA precursor (SEQ ID NO: 2) that comprises, from its 5′ to 3′ end, a 3′ catalytic Group I intron fragment (SEQ ID NO: 3), a 3′ exon sequence (SEQ ID NO: 4), an effector RNA sequence (SEQ ID NO: 5) comprising an IRES and EGFP, and a 5′ exon sequence (SEQ ID NO: 6); and ii) a free 5′ catalytic Group I intron fragment (SEQ ID NO: 1) (FIG. 1B). Without wishing to be bound by theory, this trans splicing may be able to occur by effectively starting from an artificial recapitulation of the results of the 1^{st} step of the PIE method. Surprisingly, this artificial trans arrangement was able to achieve the “2^{nd}” step of splicing without a “1^{st}” splicing having occurred. Having the Group I intron fragments present on separate starting pieces of RNA allows for the option of adjusting the ratio between the 3′ and 5′ intron fragments, which is not available under the PIE method. Surprisingly, the results presented herein demonstrate that adjusting the ratio between the 3′ and 5′ intron fragments can increase splicing efficiency.

### Methods

A linear 1,820-nt linear RNA precursor (SEQ ID NO: 2) and a 155-nt free 5′ catalytic Group I intron fragment (SEQ ID NO: 1) were generated as shown in FIG. 1B (top left) and placed in a 1× in vitro reaction buffer comprising 40 mM Tris-HCl, 6 mM MgCl₂, 1 mM DTT, and 2 mM spermidine unless otherwise noted. DNA sequences used in the experiments described herein are listed in the Exemplary Sequences section below.

For the reaction described in FIG. 1D, a (⅕)× buffer was used. CircRNA transfection into HEK293T cells was conducted as follows: 3×10⁵ cells per well were seeded in 12-well plates. 1-5 micrograms of circRNA was transfected into HEK293T cells using Lipofectamine MessengerMax (Invitrogen, LMRNA003) according to the manufacturer's instructions. GFP detection was conducted as follows: 24-48 hr after transfection, the cells were collected for subsequent detection. Samples were washed and images were acquired on an LSRFortessa (BD Biosciences). Analysis was performed using FlowJo software.

### Results

CircRNA was generated by trans splicing in vitro in the buffered conditions described above (FIG. 1B). Using 5-fold molar ratio of 5′ intron, increasing the reaction time dramatically increased the efficiency of circularization (FIGS. 1D-1E). Production of circRNA was confirmed by RNase R assay (FIG. 1E). Further, increasing the molar ratio of 5′ intron increased the efficiency of circularization (FIGS. 1C, 1F, and 1G). The circRNA was validated by transfecting to HEK293T cells, which would express more protein than its linear precursor in cells (FIG. 1G).

RNA production by in vitro transcription (IVT) using the T7 promoter requires G nucleotides as the first several bases of transcripts. Thus, using only a single G may impair the yield. Therefore, “1G” and “2G” versions—with one or two G nucleotides, respectively, present at the 5′ end of the free 5′ intron fragment-were designed and tested. Surprisingly, the 1G version performed better in RNA circularization (FIG. 1H).

### Discussion

Without wishing to be bound by theory, it is believed that the trans splicing described in this Example achieved RNA circularization via the following mechanism (FIG. 1I): the free 5′ intron bound the 3′ intron via non-covalent interactions to form a structure approximating the “correct” Group I intron structure, and then mediate the “second” step of transesterification reaction. Without wishing to be bound by theory, it is believed that the binding of the 5′ intron and 3′ intron in trans splicing thus simulated a complete intron structure.

### Example 2: Group I Intron-Mediated RNA Circularization with Cis-Splicing

This example demonstrates that circRNA can be generated by cis splicing within a linear RNA precursor comprising, from the 5′ end to the 3′ end: a catalytic Group I intron, a 3′ exon sequence, an effector RNA sequence, and a 5′ exon sequence, and lacking a 5′ intron fragment on the 3′ end.

### Background

As described in Example 1, canonical Group I autocatalysis and PIE circularization are understood to require a linear RNA precursor that has a 3′ Group I intron fragment on its 5′ end and a 5′ Group I intron fragment on its 3′ end. In the experiments described in this Example, a different design of a linear RNA precursor was tested in which there is a complete catalytic Group I intron sequence on the 5′ end and no intron sequence on the 3′ end (FIG. 2A). Surprisingly, the results presented herein demonstrated that such a structure can still generate circRNA. Without wishing to be bound by theory, it is believed that the complete Group I intron mediates circularization by splicing in cis.

### Methods

The experiments were run according to the methods described in Example 1 unless otherwise noted. The data shown in FIGS. 2B, 2G, 2H, and 2I are reaction products after 16 hours of IVT reaction. The data shown in FIG. 2E are from reactions run with ⅕× buffer. DNA sequences used in the experiments described herein are listed in the Exemplary Sequences section below. The sequences used for the experiments shown in FIG. 2H are provided in Table 1. The PIE reactions described in FIG. 2M were conducted in an IVT reaction using 1× buffer for varying lengths of time as indicated in FIG. 2M.

TABLE 1
Sequences used in FIG. 2H

| SEQ ID NO | Description | Sequence |
|---|---|---|
| SEQ ID NO: 63 | Exon1_Ana_3 nt | CTT |
| SEQ ID NO: 64 | Exon1_Ana_15 nt | AGACGCTACGGACTT |
| SEQ ID NO: 65 | Exon1_Ana_30 nt | GTGTGGCGGAATGGTAGACGCTACGGACTT |
| SEQ ID NO: 66 | Exon1_Ana_60 nt | AATATAGCAAGCTCCCAAGACAACGGGGGGG |
| | | TGTGGCGGAATGGTAGACGCTACGGACTT |
| SEQ ID NO: 67 | Exon2_Ana_15 nt | AAATCCGTTGACCTT |
| SEQ ID NO: 30 | Exon2_Ana_30 nt | AAATCCGTTGACCTTAAACGGTCGTGTGGG |
| SEQ ID NO: 31 | Exon2_Ana_60 nt | AAATCCGTTGACCTTAAACGGTCGTGTGGG |
| | | TTCAAGTCCCTCCACCCCCATAAGAGCAAG |
| SEQ ID NO: 32 | Intron_Co | ggGAACGATGACGCATCAACGGGGTCAGTAGC |
| | | GGTCAGCGTGCCGCTAGTCCAGTCGGCCA |
| | | GCATCTGTGGGTGGCCACGGGCGAGACAA |
| | | CCTGGTACGGGGGAGCCTACGGGGGAGGA |
| | | CTCGTCCTCCCTACGGTAATCCCGTGGCGA |
| | | CCTTTCCACTGGACTTCCACTGGAGAGGCG |
| | | TCGTAACGCGCGGAAAGGTGTCGGTTGGC |
| | | GGTCGCGAGGCCGCCGGCTTAAGGGACGT |
| | | GCTAAACCCTGGCGAAAGCCAGCCCGCCG |
| | | ACGGAGCGCCCCCAGCGCAAAGTCGTCGG |
| | | GGGTCGTACTTACACCACGCCTGGGAGGA |
| | | AATGCCCTGGCGGAGACCGGTAGCCTCTG |
| | | CTGGCCTGCAAAGGCCGCGGGGGAATTG |
| SEQ ID NO: 33 | Exon1_Co_6 nt | gaccct |
| SEQ ID NO: 34 | Exon1_Co_15 nt | gggaaagaagaccct |
| SEQ ID NO: 35 | Exon1_Co_30 nt | ttggcagaatcagcggggaaagaagaccct |
| SEQ ID NO: 36 | Exon1_Co_60 nt | tctagcgaaaccacagccaagggaatgggcttggcagaatcagcgggga |
| | | aagaagaccct |
| SEQ ID NO: 37 | Exon2_Co_7 nt | gttgagc |
| SEQ ID NO: 38 | Exon2_Co_15 nt | gttgagcttgactct |
| SEQ ID NO: 39 | Exon2_Co_30 nt | gttgagcttgactctagtttgacattgtga |
| SEQ ID NO: 40 | Exon2_Co_60 nt | gttgagcttgactctagtttgacattgtgaaaagacataggaggtgtagaata |
| | | ggtggga |
| SEQ ID NO: 41 | Exon1_Gvi_6 nt | tctggt |
| SEQ ID NO: 42 | Exon1_Gvi_15 nt | gagggcaagtctggt |
| SEQ ID NO: 43 | Exon1_Gvi_30 nt | aacgaggaacaattggagggcaagtctggt |
| SEQ ID NO: 44 | Exon1_Gvi_60 nt | taattggaatgagaacaatctaaatcccttaacgaggaacaattggagggca |
| | | agtctggt |
| SEQ ID NO: 45 | Exon2_Gvi_7 nt | ccagcag |
| SEQ ID NO: 46 | Exon2_Gvi_15 nt | ccagcagccgcggta |
| SEQ ID NO: 47 | Exon2_Gvi_30 nt | ccagcagccgcggtaattccagctccaata |
| SEQ ID NO: 48 | Exon2_Gvi_60 nt | ccagcagccgcggtaattccagctccaatagcgtatattaaagttgttgcagt |
| | | taaaaag |

### Results

The number of G nucleotides on the 5′ end of the 5′ intron sequence were tested by conducting the cis splicing method in “1G” or “2G” versions, with either one or two G nucleotides present on the 5′ end, respectively (FIG. 2B, right). The 2G version worked well in circularization efficiency and yielded more RNA in the IVT reaction than did the 1G version (FIG. 2B, left). Thus, the remaining experiments were conducted with at least 2Gs at the 5′ end of the intron.

The canonical PIE method requires a specific split site in the Group I intron and additional engineering, which restricts the applicability of the canonical PIE method to only several group I introns, limiting the application of other Group I introns in RNA circularization. Because the cis method does not split the Group I intron, it presents an opportunity to expand the palette of Group I introns used to generate circRNA beyond the limited set amenable to PIE. Thus, additional Group I introns were tested in the cis splicing method (FIGS. 2C-2D). First, the Ana intron was tested, as it is used in the canonical PIE circularization method. The Ctu, Par, and Gvi introns, which are not used in the PIE method, were chosen from a Group I intron database and tested in addition to the Ana intron. In its natural form, the tested Ctu intron sequence showed no activity in the IVT buffer, while the Par and Gvi introns showed successful splicing under the same conditions (FIGS. 2C-2D). Thus, the tested Ctu intron sequence was used as a negative control.

The Pob, Tpa, Co, Pte, and Cpro introns were also tested for the ability to induce self-splicing, as they are in the same 1E2 intron family as, e.g., Gvi and Par. Each of these introns demonstrated ability to induce self-splicing (FIG. 2L), indicating that they and other Group I introns with similar structures or sequences are likely amenable to the cis splicing method.

According to the mechanism of canonical intron splicing, there are some restrictions in the sequence of the exon-intron boundary, such that the sequence of 3′ end may affect splicing. Effects of the sequence of the exon-intron boundary were thus tested in the context of cis splicing. An additional 7-nt sequence (GGGTCGG; SEQ ID NO: 12) was added to the 3′ end of exon1. The Ana intron was greatly affected by the additional 7-nt, but the Par or Gvi introns were less affected (FIG. 2C). These RNA were then transfected into HEK293T cells to validate RNA circularization (FIG. 2D).

The reaction conditions were then modified to see if circularization efficiency could be further improved. By extending the reaction time to 16 hrs at 50° C. under ⅕× buffer, only the Ana intron achieved higher circularization efficiency with RNA degradation (FIG. 2E). The resulting RNA were then transfected and the EGFP expression detected (FIG. 2F). Although there was a large difference in circularization efficiency with or without 16 hrs treatment, a similar expression pattern was observed with the Ana intron, indicating the possibility of in vivo circularization in mammalian cells (FIG. 2F).

Without wishing to be bound by theory, it is believed that the RNA ligation during cis splicing happens between the 5′ of exon2 and the 3′ of exon1, and thus requires physical contact between these sites. It is thus possible that adding homology arms or extending exons length may help to bring the two sites nearby and thus facilitate circularization. The effects of homology arms (FIG. 2G) and extension of exons (FIG. 2H) were therefore tested in different Group I introns. In different group I introns, the presence of homology arms or exon extensions resulted in a difference in circularization efficiency: for the Ana intron, adding a homology arm or extending exon length increased the circularization efficiency, but this effect was not observed for the Gvi intron (FIGS. 2G-2H). The presence of a homology arm in site 8 of the IRES showed different effects in different introns. Site 3 of the IRES served as a control for no homology (FIG. 2G).

3′ end heterogenicity has been reported to happen regularly in IVT reactions under the traditional PIE method. Potential effects of 3′ end heterogenicity on cis splicing efficiency were examined by changing the 3′ end base of the linear RNA precursor. The results showed no obvious effects on circularization efficiency in our cis-splicing method (FIG. 2I)

Obtaining high purity circRNA (lacking the spliced out intron sequences and remaining precursors, which are viewed as impurities alongside the desired circRNA product) via the PIE method is time-consuming and not suitable for industrial production, as it traditionally requires HPLC and RNase R treatment, which are difficult to achieve on a large scale. Different lengths of A nucleotide repeats were added to the Group I introns at different sites in order to test whether the cis splicing linear RNA design may be amenable to such additions and still successfully mediate circularization, with the hypothesis being that the addition of sufficient A nucleotides to the impurities would allow the impurities to be removed using oligo dTs beads, which could bind to the impurities and be pulled down, leaving the circRNA in the supernatant. In order to test this, different lengths of A repeats (0 As, 15 As, 25 As, and 35 As) were added in Group I introns at two different sites (FIGS. 2J-2K). The addition of As at site 2 resulted in only minor damage on the efficiency of circularization (FIG. 2J). Thus, RNA with insertions at site 2 was treated with oligo dT beads. The results showed that only impurities with the 35-A insertion were enriched on beads, increasing the ratio of circRNA in supernatant (FIG. 2K). Finally, a comparison was made between the cis reaction and the canonical PIE method. It was found that the cis method performed better than the PIE method in both speed and circularization efficiency when varying the time of the IVT (FIG. 2M).

### Example 3: Efficient In Vitro RNA Circularization with Cis Splicing and Trans Splicing

This example demonstrates the high efficiency of RNA circularization achieved by using two novel methods.

Circular RNA (circRNA) has emerged as a promising candidate in RNA therapeutics due to its extended half-life and ability to encode proteins. However, existing tools for in vitro RNA circularization have their limitations. Here, we introduce innovative methods for in vitro RNA circularization, referred to herein as trans splicing and cis splicing. Trans splicing initiates from the second step of group I intron splicing, offering an alternative approach for efficient RNA circularization. Cis splicing effectively facilitates the circularization of RNA synthesized through in vitro transcription (IVT), relying on intact group I introns without the need for fragmentation. Cis splicing aids in circRNA purification through RNase R or the use of oligo(dT) beads. Notably, cis splicing has lower Mg²⁺ requirements and enables faster circularization under mild conditions compared to PIE, benefiting circRNA integrity. Additionally, we demonstrate that cis splicing can utilize various natural group I introns, opening up possibilities for discovering superior introns for RNA circularization.

Trans splicing presents an alternative approach for RNA circularization. Furthermore, by adjusting the component ratios within trans splicing, we attained highly efficient circularization. Notably, cis splicing excels in achieving RNA circularization by effectively utilizing intact group I intron, opening up possibilities for the identification of superior introns. Moreover, cis splicing streamlines circRNA purification through the use of RNase R or oligo(dT) beads. Compared to PIE, cis splicing reduces the Mg²⁺ requirements and facilitates swift RNA circularization, thereby promoting RNA integrity and offering potential for the circularization of large RNA.

### Methods

### Plasmids Construction

The sequences involved PCR amplification of sequences from our lab's plasmids or sequences synthesized by Tsingke Biotech. Cloning was performed using Gibson assembly or enzyme digestion and ligation. Gibson assembly was conducted with Gibson Assembly Master Mix (NEB, E2611L) or 2× MultiF Seamless Assembly Mix (Abclonal, RK21020) according to the manufacturer's instructions. Restriction enzymes from NEB and the T4 DNA ligase (NEB, M0202L) were used for digestion and ligation.

### DNA Template Generation

Linearized plasmids were produced using endonuclease such as BbsI-HF (NEB, R3539L) for cis splicing and Pmel (NEB, R0560L) for PIE. Primers with 2′ OMe modification were obtained from Tsingke Biotech. PCR products were amplified using specific primers with PrimeSTAR GXL Premix (Takara, R051A) and purified by gel recovery using Zymoclean Gel DNA Recovery Kit (ZYMO, D4008).

### In Vitro Transcription

In vitro transcriptions were conducted using the HiScribe™ T7 High Yield RNA Synthesis Kit (NEB, E2040S). A homemade 5×IVT buffer with 400 mM HEPES (pH=6.8), 90 mM MgCl₂, 38 mM DTT, and 10 mM spermidine was used for short-time IVT reactions to prevent intron splicing.

### Purification of CircRNA

Purification of circRNA involved treating IVT products with DNase I (NEB, M0303L), followed by column purification using the Monarch RNA Cleanup Kit (NEB, T2040L). Optional circularization was performed in a buffer containing MgCl₂, 150 mM NaCl, and 50 mM HEPES (pH=6.8). CircRNAs were finally column purified and concentrated with the RNA Clean & Concentrator Kit (ZYMO, R1018) for agarose gel electrophoresis analysis.

### Cell Culture

HEK293T was maintained in Gibco BASIC DMEM, High Glucose (Gibco, C11965500BT), supplemented with 10% fetal bovine serum (FBS) (Biological Industrials, C04001-500), 1% penicillin-streptomycin, 1% GlutaMAX (Gibco, 35050061), and 1% NEAA (Non-Essential Amino Acids Solution) (Gibco, 11140050) in a 5% CO2 incubator at 37° C.

### CircRNA Transfection

For circRNA transfection, HEK293T cells were seeded at approximately 1.5×10⁵ cells per well in 24-well plates. After 24 hr, 500 ng to 1 μg of purified circRNA was transfected into the cells using Lipofectamine MessengerMax (Invitrogen, LMRNA008). 24 or 48 hr post transfection, the cells were collected for the subsequent detections.

### RNase R Treatment

RNase R treatment involved heating RNA at 65° C. for 5 min, followed by cooling on ice. Reaction buffer and RNase R (mostly 0.2 U/μg) were added and mixed (Epicentre, RNR07250), and the reaction was conducted at 37° C. RNA was then purified and concentrated with the RNA Clean & Concentrator Kit (ZYMO, R1018)

### Poly(A) Tailing of RNA

RNA was heated at 65° C. for 5 min, followed by cooling on ice. Reaction buffer, ATP, polymerase, and appropriate RNAs were mixed according to the manufacturer's instruction (NEB, M0276L).

### RNA Purification by Oligo(dT) Magnetic Beads

VAHTS mRNA Capture Beads (Vazyme, N401-02) was used for circRNA purification by depleting poly(A) containing byproducts. In brief, 2 μg RNA was incubated with 50 μL beads for one round, and the supernatant after incubation was used for downstream analysis or another round of incubation.

### RNA Agarose Gel Electrophoresis

RNA agarose gel electrophoresis was performed using TAE for agarose gel prepared in-house, with 2% agarose gel and 0.5× or 1×TAE used for RNA electrophoresis.

RT-PCR Detection of the circRNA Junction

The circRNAs were reverse transcribed into DNA using the AMV First Strand cDNA Synthesis Kit (Sangon, B532445). Targeted sequences were then PCR amplified with corresponding primers, and the PCR products were cloned into T vectors using the Zero TOPO-TA/Blunt Cloning Kit (ABclonal, RK30130). Single clones were selected for Sanger sequencing.

### FACS

The cells were treated with trypsin and resuspended in PBS containing 2% FBS. Samples were acquired and recorded by LSRFortessa (BD Biosciences). Analysis was performed using FlowJo software.

### Luciferase Activity Detection

Gaussia luciferase activity was measured using Gaussia Luciferase Reporter Gene Assay Kit (Beyotime, RG021M) according to the instructions. Data were collected using Infinite M200 (TECAN).

### Selection of Group I Introns

Group I introns from the reported database (Zhou, Y. et al. GISSD: Group I Intron Sequence and Structure Database. Nucleic Acids Res 36, D31-37 (2008)) were selected based on certain sequence information, ranked by MFE (Minimal Free Energy), and normalized with sequence length. The top-ranking sequences were tested for in vitro activity, and introns in the same family as the active group I intron were selected.

### Results

PIE can Start from the Second Step of Transesterification

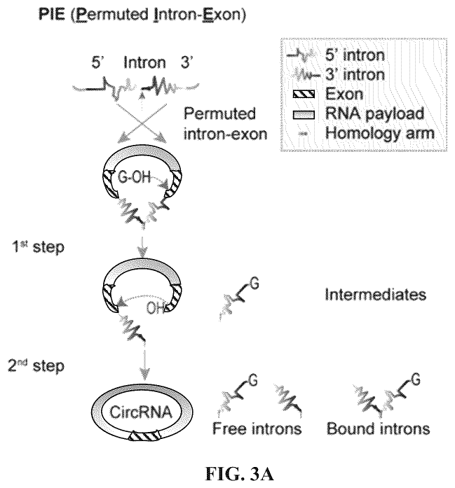

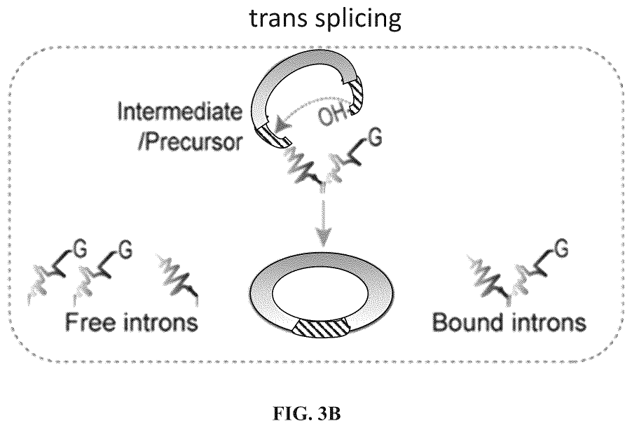

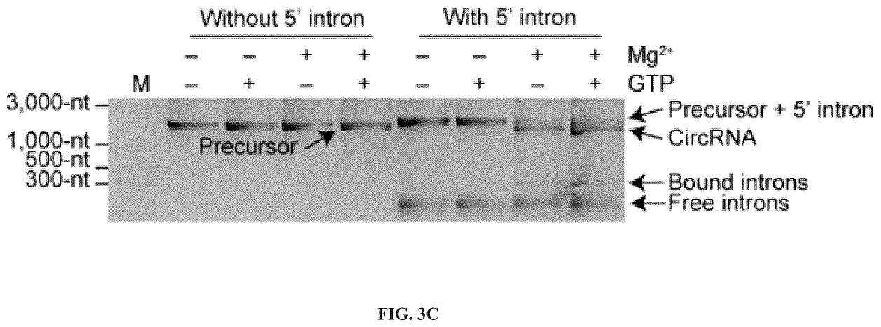

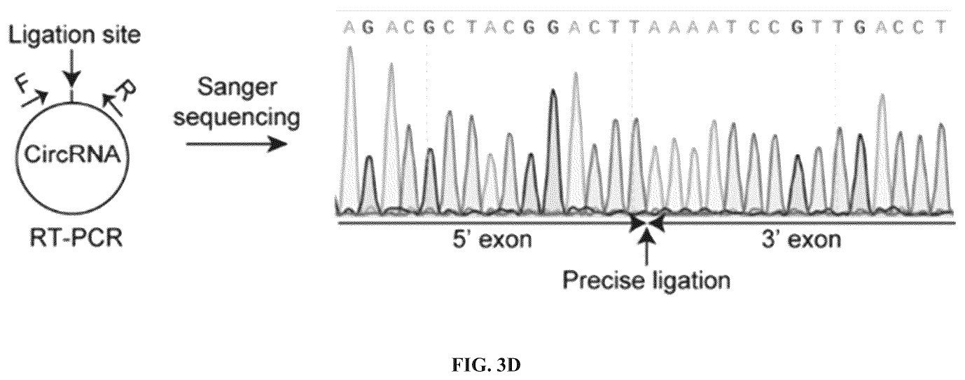

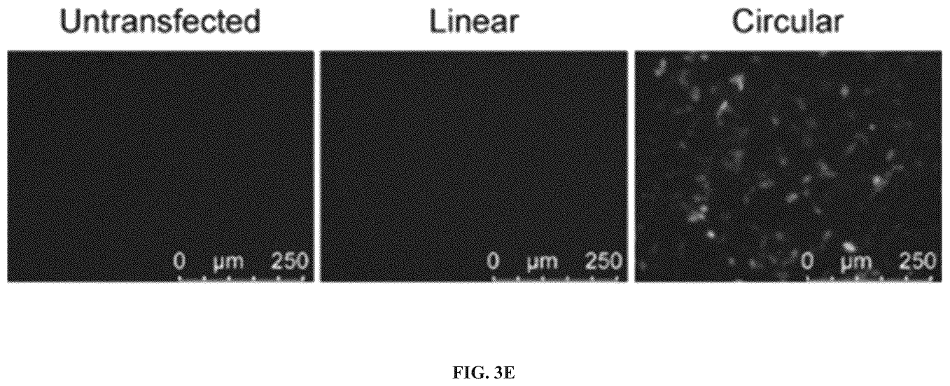

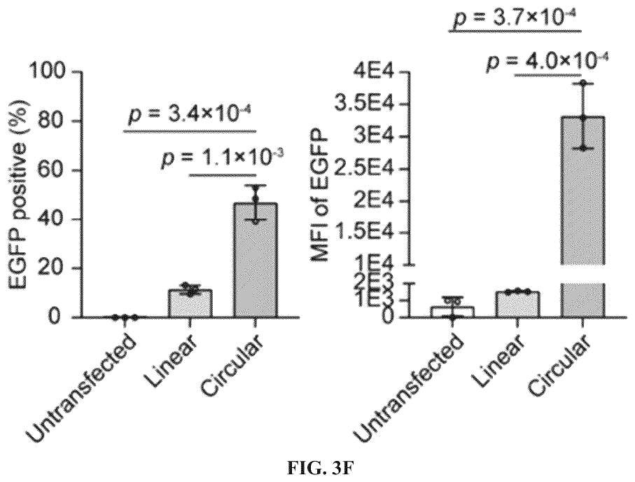

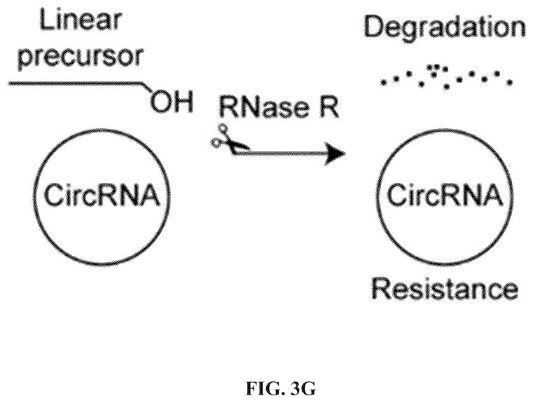

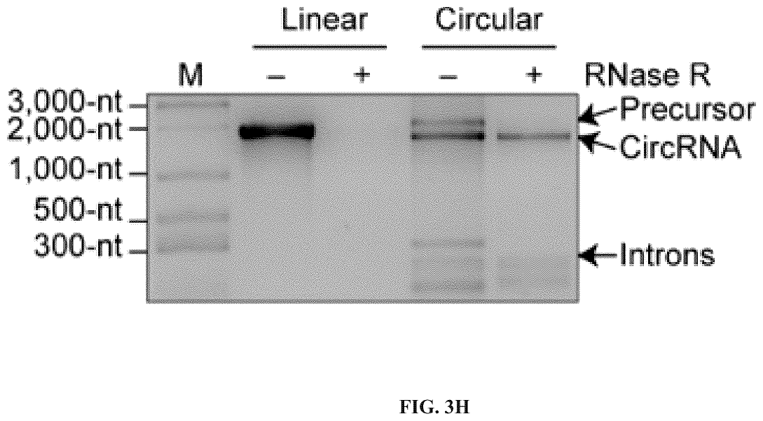

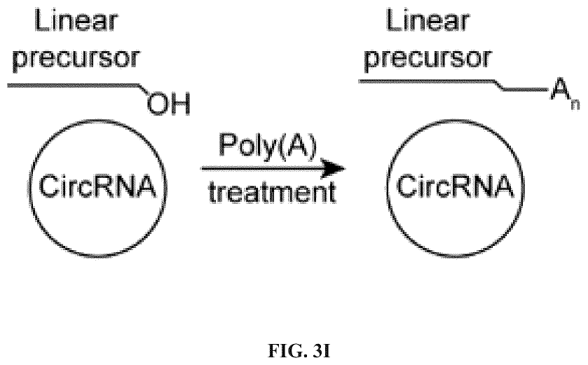

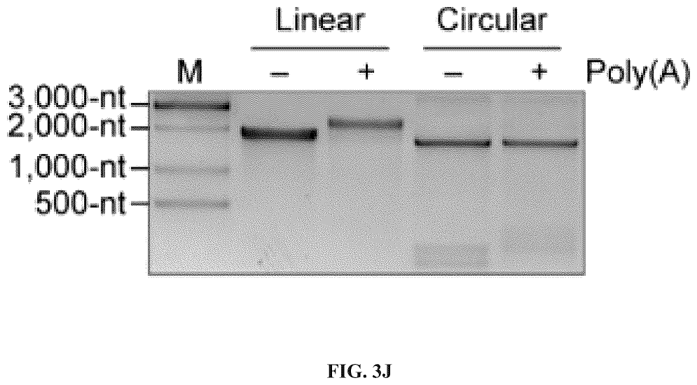

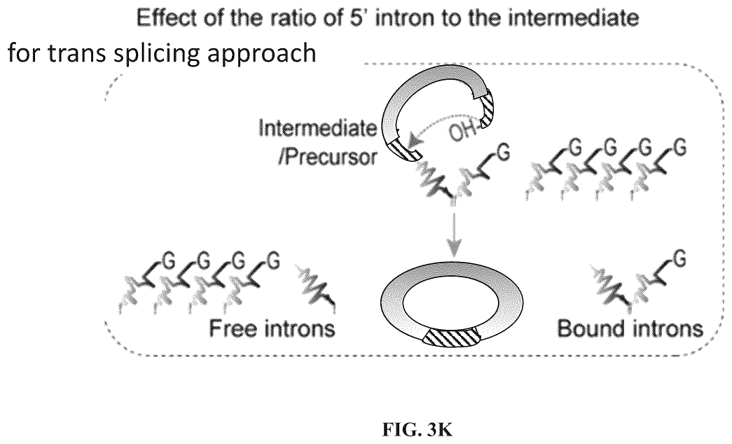

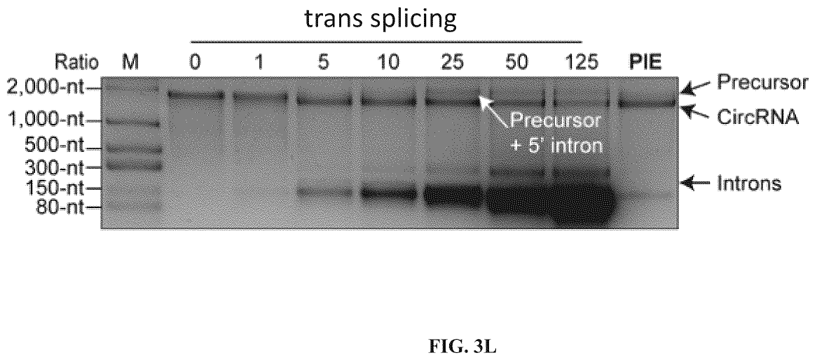

As self-splicing ribozymes, group I introns mediate RNA splicing without protein assistance (Kruger, K. et al. Self-splicing RNA: autoexcision and autocyclization of the ribosomal RNA intervening sequence of Tetrahymena. Cell 31, 147-157 (1982)). Group I introns function through a two-step transesterification reaction, requiring guanosine as a cofactor. Canonical RNA circularization mediated by group I introns has been achieved through PIE (Wesselhoeft, R. A., Kowalski, P. S. & Anderson, D. G. Engineering circular RNA for potent and stable translation in eukaryotic cells. Nat Commun 9, 2629 (2018)), successfully completing both steps of the transesterification reaction (FIG. 3A). During the first step of group I intron splicing, exoG initiates an attack on the junction between the 5′ exon and 5′ intron. This results in RNA cleavage and the incorporation of a single G at the 5′ end of the 5′ intron. Here, we tested whether omitting the initial step of group I intron splicing would produce the outcomes of the first step splicing—namely, the intermediate and 5′ intron—and assessed their capacity to autonomously accomplish the second step of splicing, separate from the first step (FIG. 3B). This method is referred to herein as trans splicing. Intron splicing was observed under different conditions, with 5′ intron and Mg²⁺ as limiting factors, while GTP was not required (FIG. 3C). To confirm circRNA generation, RT-PCR demonstrated precise ligation of exons (FIG. 3D). RNase R assay further confirmed circRNA production (FIGS. 3G, 3H). The product post RNase R treatment was subjected to poly(A) polymerase (FIG. 3I), revealing that the length of circRNA remained unchanged (FIG. 3J). Overall, these findings indicate that trans splicing generated circRNA successfully.

### Enhancing the Circularization Efficiency was Achieved by Introducing Additional 5′ Half Introns

Given that trans splicing operates as a two-component circularization system, we tested whether adjusting the ratio of 5′ intron to intermediate would lead to an increase in circularization efficiency (FIG. 3K). Through varying the ratio from 0 to 125, RNAs exhibited different circularization efficiencies, with the highest efficiency observed at a ratio of 5 to 10 (FIG. 3L). However, at high ratios, the 5′ intron bound some intermediates without mediating circularization, resulting in larger sizes in the agarose gel and lower circRNA generation efficiency (FIG. 3L).

Utilizing a Complete Intron is a Viable Approach for circRNA Generation

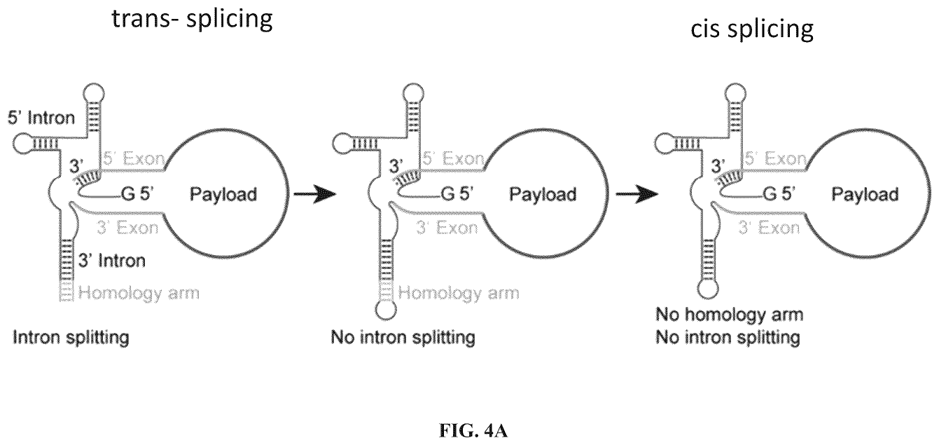

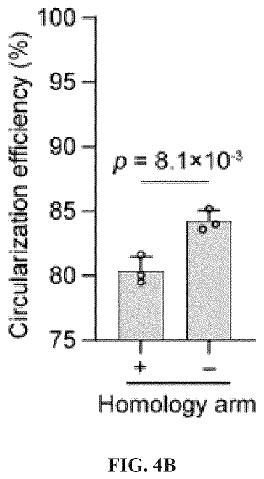

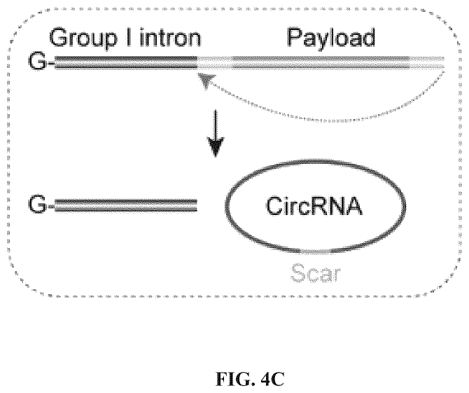

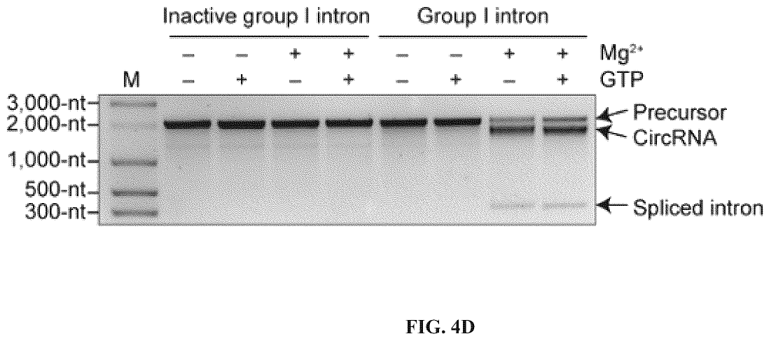

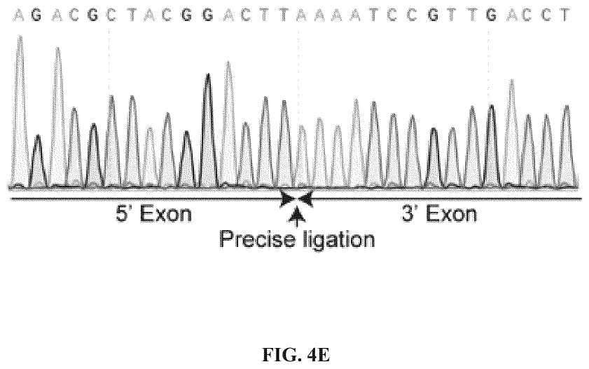

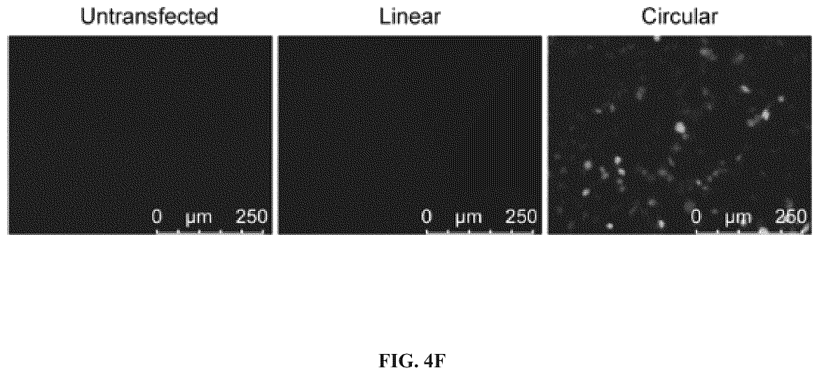

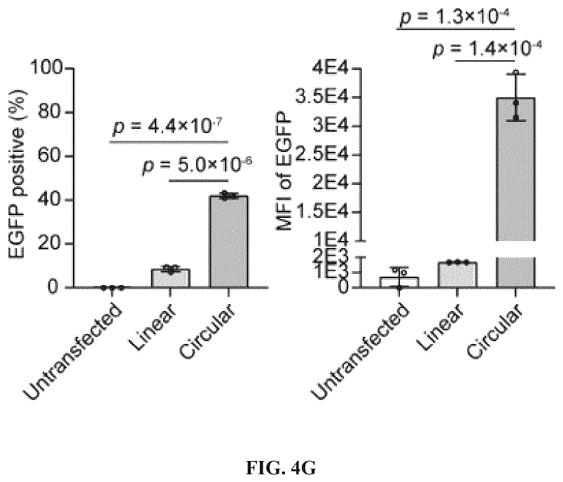

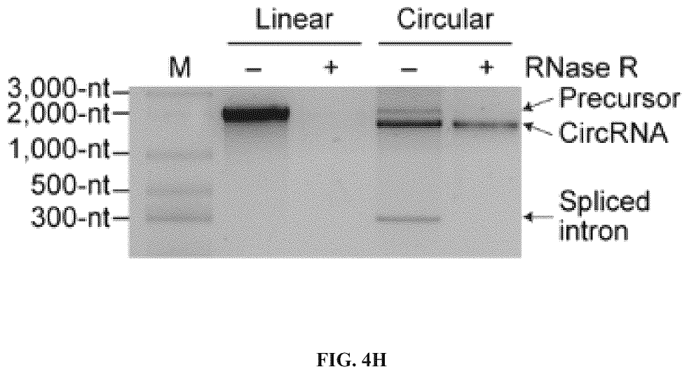

Given that trans splicing effectively circularized RNA through a split intron in the second step transesterification reaction, we tested whether a complete intron without intron splitting could also function for RNA circularization with a similar design (FIG. 4A). We further tested whether the homology arm, which is essential in PIE, could be eliminated (FIG. 4A). Surprisingly, by removing the homology arm, circularization efficiency was significantly improved (FIG. 4B). Consequently, the construct was designed as G-complete intron-3′ exon-payload-5′ exon (FIG. 4C). We tested intron splicing under varied conditions, and identified Mg²⁺ as a limiting factor, while GTP was not required (FIG. 4D). This observation suggested that this method was independent of the first step of group I intron splicing. The RT-PCR assay confirmed the precise ligation of exons (FIG. 4E), and the RNase R assay further indicated the production of circRNA (FIG. 4H). Poly(A) were introduced to the product after RNase R treatment, and the length of circRNA remained unchanged (FIG. 4I). These findings collectively verified the efficacy of RNA circularization. This new RNA circularization method is referred to herein as cis splicing.

Functional Proteins can be Produced from circRNA Generated Through Trans Splicing and Cis Splicing

To further substantiate the generation of circular RNAs and their capacity to encode proteins, circRNAs harboring an IRES-EGFP reporter that were produced by trans splicing or cis splicing were transfected into HEK293T cells. Our findings revealed that circRNAs generated from both methods efficiently translated abundant EGFP proteins (FIG. 3E and FIG. 4F). Moreover, when compared to their linear precursors, circRNAs exhibited a higher expression of proteins (FIG. 3F and FIG. 4G). These results provided compelling evidence that trans splicing and cis splicing could generate protein-coding circRNAs effectively.

### The Quantity of Gs at the 5′ End Affects RNA Yield but not Circularization

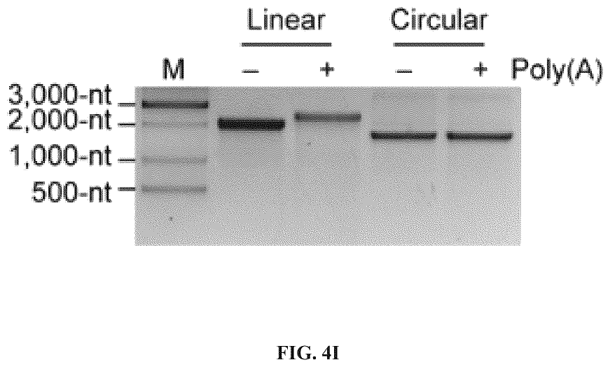

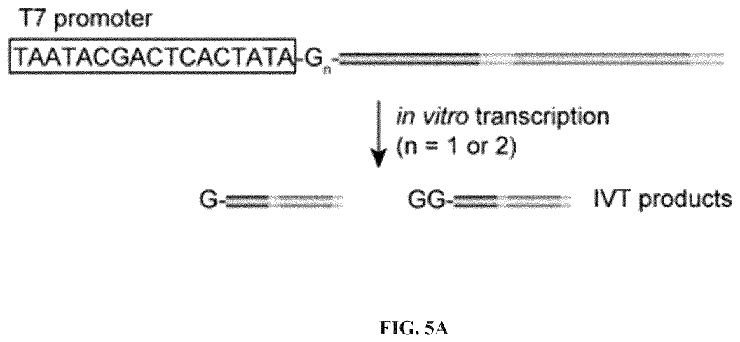

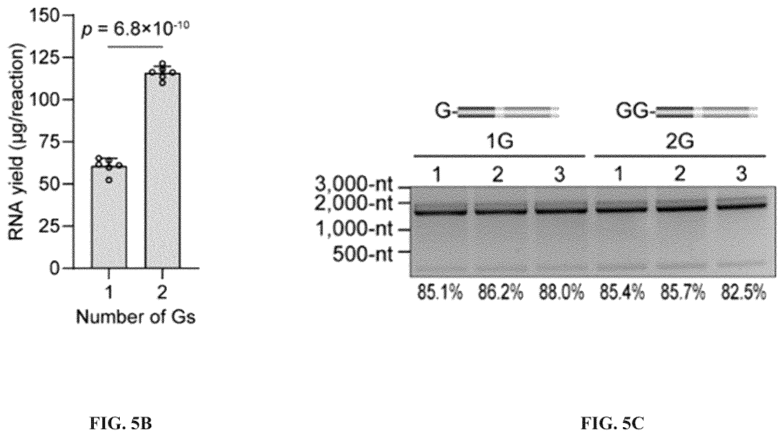

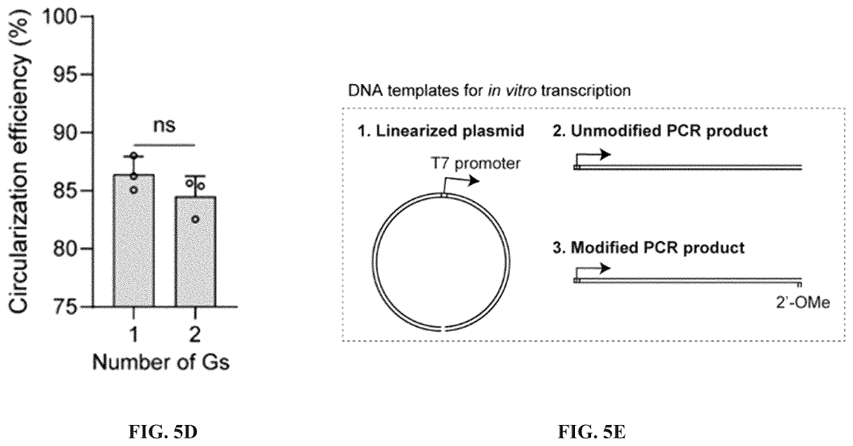

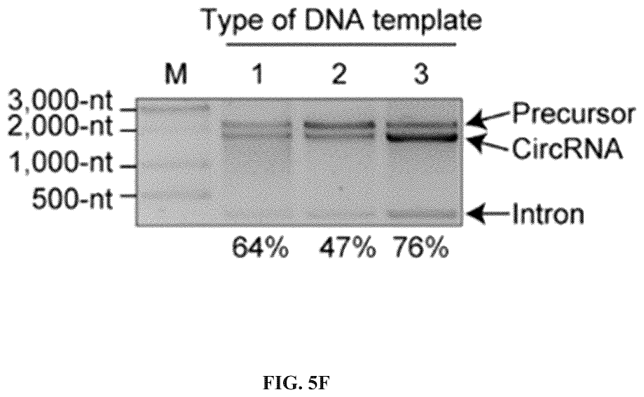

In the cis splicing IVT product, initiation occurred from G at the 5′ end (FIG. 4C). For introns with a non-G first base at the 5′ end, only one G was present at the 5′ end of RNA. However, T7 polymerase could not efficiently transcribe RNA with fewer than 2 Gs at the 5′ end of RNA (Komura, R., Aoki, W., Motone, K., Satomura, A. & Ueda, M. High-throughput evaluation of T7 promoter variants using biased randomization and DNA barcoding. PLOS One 13, e0196905 (2018); Conrad, T., Plumbom, I., Alcobendas, M., Vidal, R. & Sauer, S. Maximizing transcription of nucleic acids with efficient T7 promoters. Commun Biol 3, 439 (2020)). Given that the initial base of Ana intron was A, we investigated the impact of adding G at the 5′ end on RNA yield and circularization. Initially, to assess whether additional G affected transcription and circularization, RNAs with 1G or 2G extension at the 5′ end were used for evaluation of RNA yield and circularization efficiency (FIG. 5A). The 2G sample exhibited higher RNA yield compared to the 1G sample (FIG. 5B). While the 1G sample demonstrated slightly better circularization efficiency than the 2G sample, the difference was not statistically significant (FIGS. 5C, 5D). For a balance between RNA yield and circularization efficiency, a minimum of 2 Gs at the 5′ end of the RNA was employed for all RNAs in this study.

### The Heterogenicity of the 3′ End of RNA Poses a Challenge to RNA Circularization Through Cis Splicing

Upon successfully achieving circularization through cis splicing, we sought to identify the critical limiting factors for RNA circularization. T7 polymerase-driven IVT tends to generate a heterogenous 3′ end (Milligan, J. F. & Uhlenbeck, O. C. Synthesis of small RNAs using T7 RNA polymerase. Methods Enzymol 180, 51-62 (1989); Triana-Alonso, F. J., Dabrowski, M., Wadzack, J. & Nierhaus, K. H. Self-coded 3′-extension of run-off transcripts produces aberrant products during in vitro transcription with T7 RNA polymerase. J Biol Chem 270, 6298-6307 (1995); Zaher, H. S. & Unrau, P. J. T7 RNA polymerase mediates fast promoter-independent extension of unstable nucleic acid complexes. Biochemistry 43, 7873-7880 (2004); Gholamalipour, Y., Karunanayake Mudiyanselage, A. & Martin, C. T. 3′ end additions by T7 RNA polymerase are RNA self-templated, distributive and diverse in character-RNA-Seq analyses. Nucleic Acids Res 46, 9253-9263 (2018)). Considering that the last base in the precursor of cis splicing corresponds to the splicing site, we speculated that the homogeneity of the 3′ end would affect circularization a lot. It has been reported that using DNA templates with certain modifications such as 2′-OMe could partially alleviate this effect (Kao, C., Zheng, M. & Rudisser, S. A simple and efficient method to reduce nontemplated nucleotide addition at the 3 terminus of RNAs transcribed by T7 RNA polymerase. RNA 5, 1268-1272 (1999)). We compared the circularization efficiency of RNAs generated from linearized plasmid, unmodified PCR product, and modified PCR product (FIG. 5E). The RNA generated from the modified primer exhibited the highest circularization efficiency (FIG. 5F). Consequently, we opted for a modified DNA template for IVT for the majority of RNAs used in subsequent experiments.

### Sequence Optimization for Enhanced Cis Splicing Efficiency

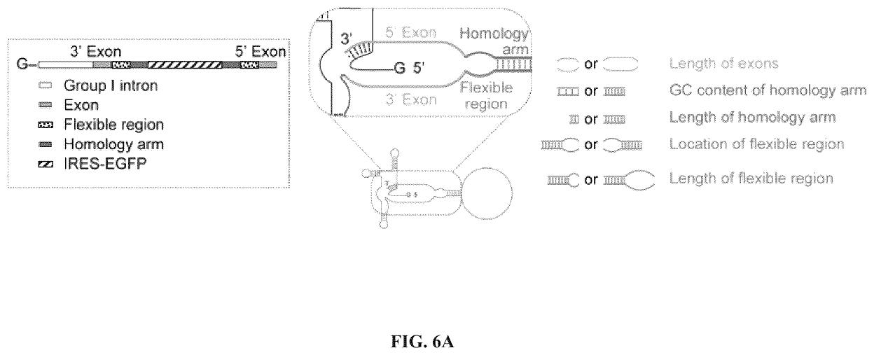

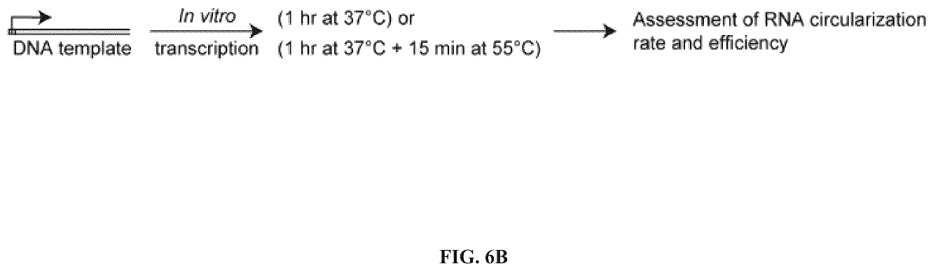

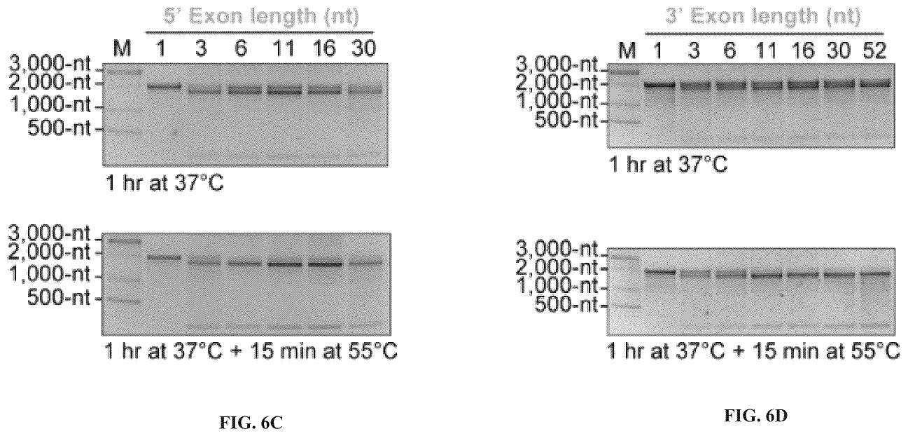

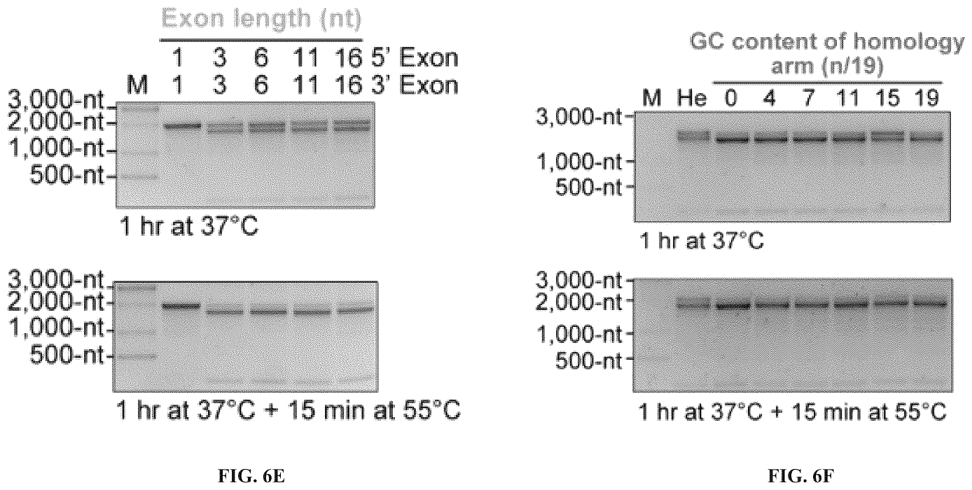

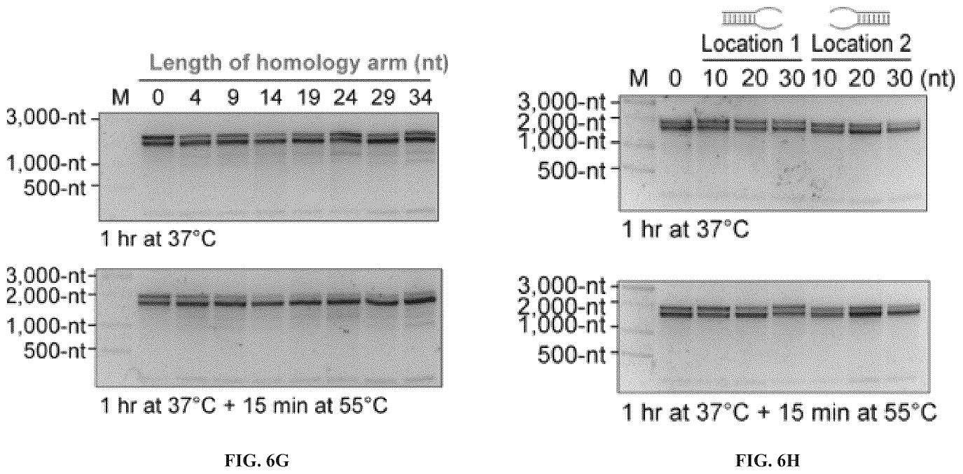

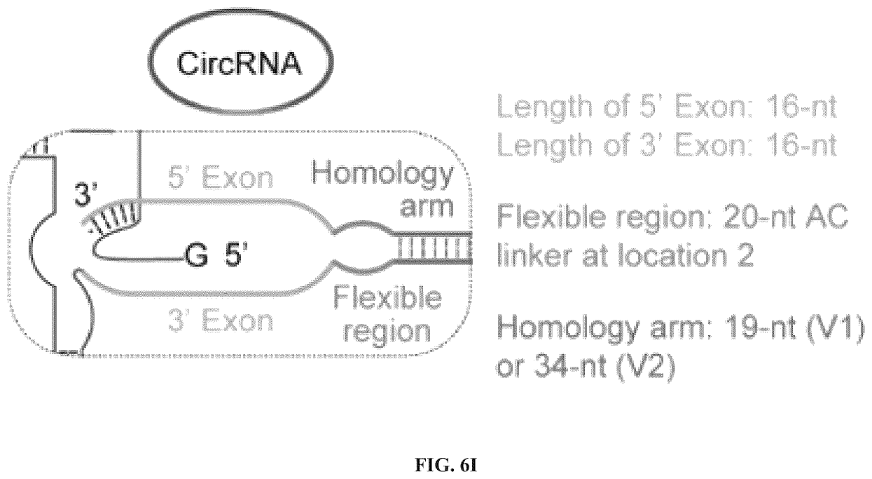

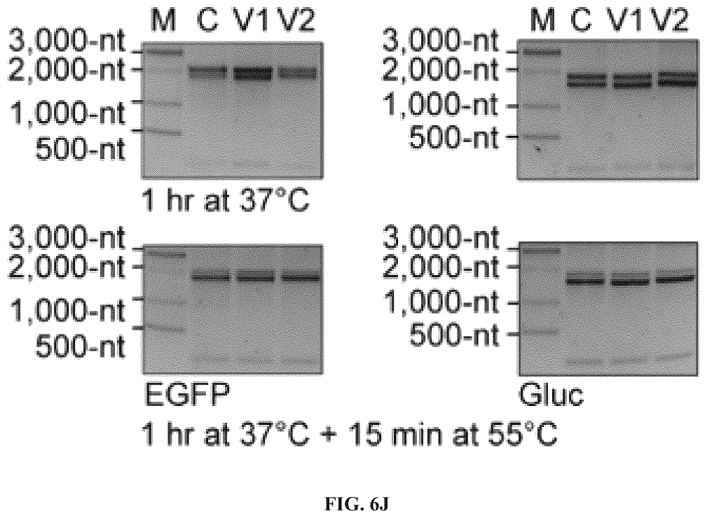

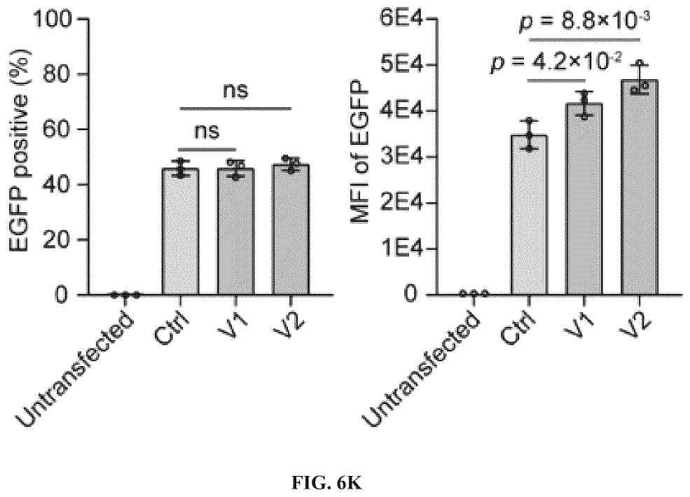

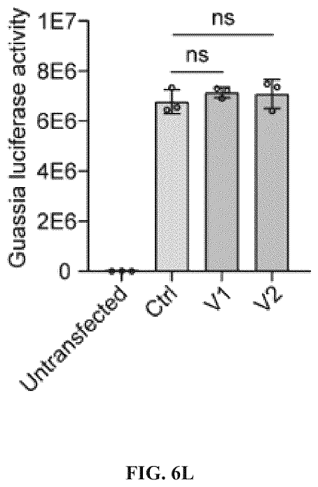

Key considerations for sequence optimization included the scrutiny and enhancement of exons, homology arm, and the flexible region sequences, all considered indispensable for the circularization process (FIG. 6A). Additionally, the circularization rate was a significant factor that needed evaluation because rapid circularization was beneficial for maintaining RNA integrity. The circularization rate and efficiency were assessed under two different conditions (FIG. 6B). The original sequence, derived from a preceding PIE report, included a 16-nt 5′ exon, a 52-nt 3′ exon, a 19-nt homology arm with 11/19 GC content, and a flexible region of 20-nt length AC linker situated in location 1 (Wesselhoeft, R. A., Kowalski, P. S. & Anderson, D. G. Engineering circular RNA for potent and stable translation in eukaryotic cells. Nat Commun 9, 2629 (2018)). Initially, we investigated the length requirements for exons in circularization. The 5′ exon's length varied from 1-nt to 30-nt, and the 3′ exon's length varied from 1-nt to 52-nt. Our findings revealed that a minimum of 3-nt for both the 5′ exon and 3′ exon was essential for achieving circularization (FIGS. 6C-6E). Moreover, when the 3′ exon length was fixed at 52-nt, a 3-nt 5′ exon was sufficient for efficient circularization (FIG. 6C). With the 5′ exon length set at 16-nt, efficient circularization required the 3′ exon to be at least 11-nt (FIG. 6D). However, when both exons had equal lengths, adequate circularization was observed with only 3-nt for each exon, potentially benefiting the generation of short scar circRNA (FIG. 6E).

Next, we explored the impact of the homology arm tightness (quantified by GC content) and length on circularization. The GC content of homology arm did not significantly affect circularization efficiency or rate (FIG. 6F). However, the length of the homology arm proved to be crucial for circularization, which mainly affected the circularization rate (FIG. 6G). Considering the RNA secondary structure, we hypothesized that the flexible region between the exons and the homology arms could mitigate the adverse effects of surrounding sequences on the circularization process. To evaluate the hypothesis, AC linkers with different lengths were set in two locations to circularize the same payload. AC linkers between exons and homology arms increased circularization efficiency, with location 2 being more effective than location 1 (FIG. 6H).

### Sequence Optimization for a Balanced Protein Expression and RNA Circularization

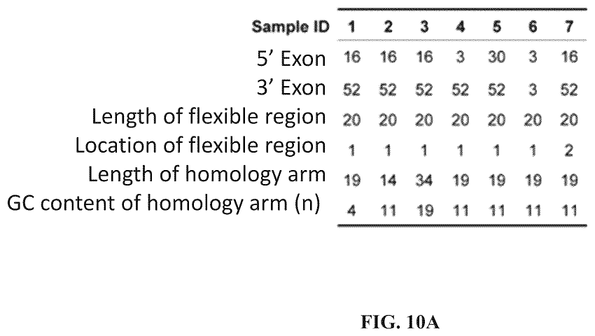

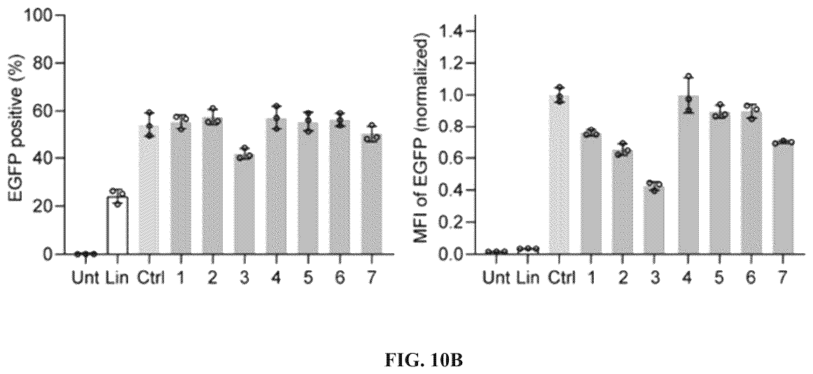

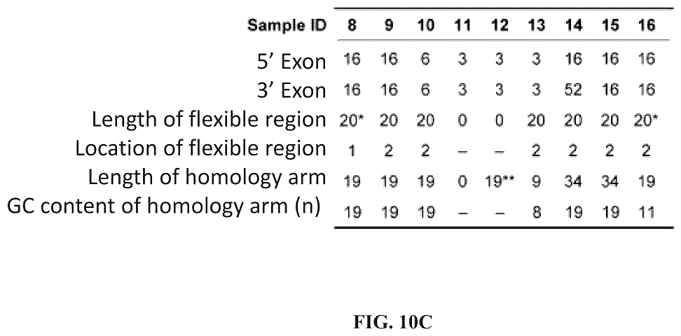

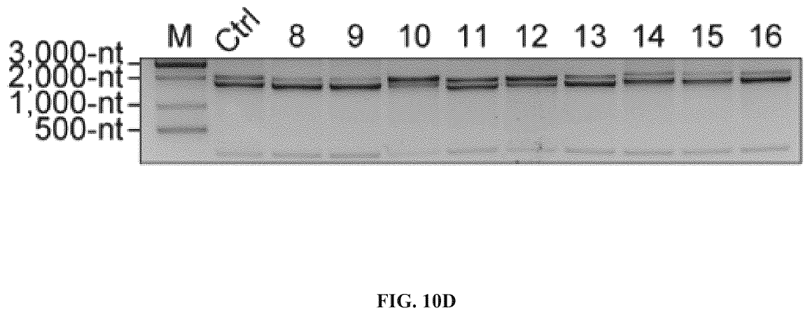

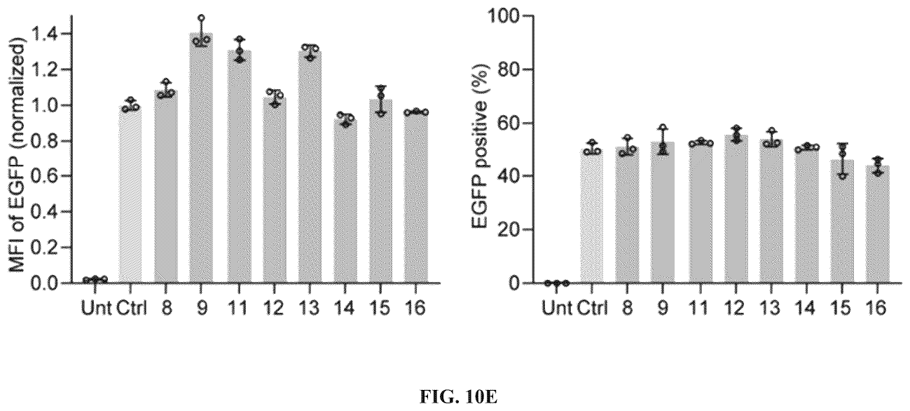

Recognizing that sequence alterations would be retained in the final product of circRNA, potentially affecting its protein expression capability, circRNAs with high circularization efficiency underwent treatment with RNase R and then transfected to HEK293T cells for quantification of the protein expression level (FIG. 10A). We observed that an extended arm might compromise protein expression ability (FIG. 10B). Introducing an increased spacer before IRES might potentially alleviate this impairment. Subsequently, more various sequences were designed to assess circularization efficiency, protein expression ability, and scar length (FIG. 10C). The results depicting circularization efficiency and protein expression levels are presented (Supplementary FIGS. 10D, 10E).

Ultimately, the final version of cis splicing was designed, featuring homology arm lengths of 19-bp, 34-bp, denoted cis splicing-VI, cis splicing-V2. Both 5′ exon and 3′ exon lengths were set at 16-nt, with the flexible region located at position 2, utilizing a 20-nt AC linker (FIG. 6I). cis splicing-V1, cis splicing-V2 exhibited faster RNA circularization while maintaining comparably high efficiency on two different sequences (FIG. 6J). Besides, the ability to express protein was evaluated. We observed that cis splicing-VI or cis splicing-V2 did not impair protein expression and exhibited higher protein expression in the case of Gaussia luciferase (Gluc) (FIGS. 6K, 6L).

### Condition Optimization for Cis Splicing

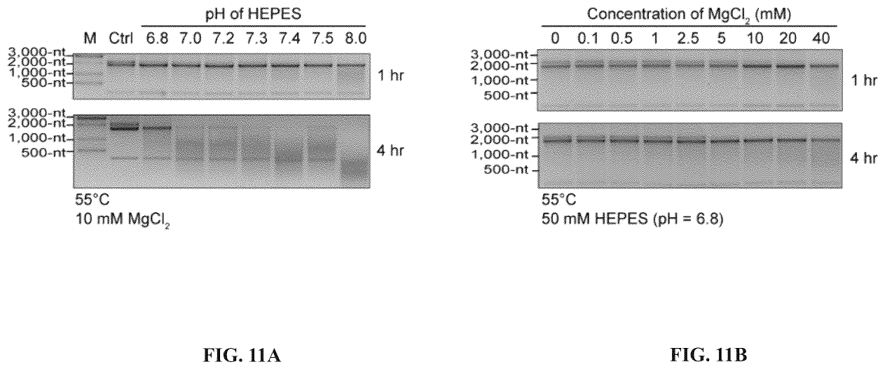

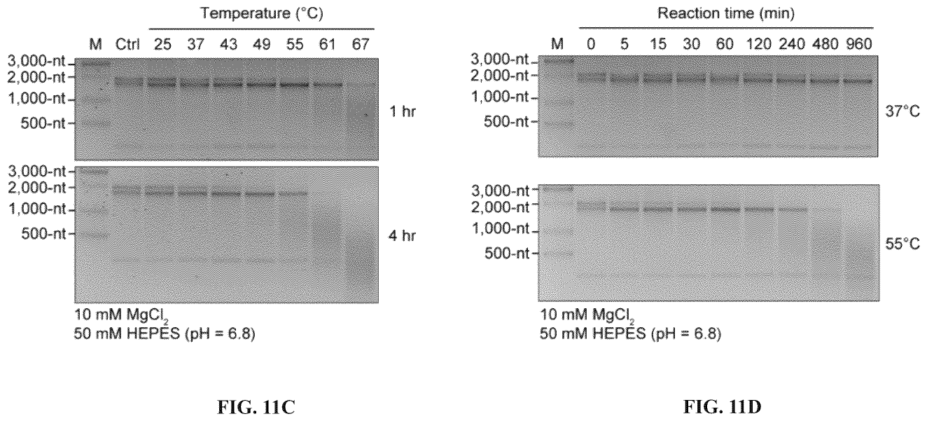

The successful circularization through cis splicing relied on the self-splicing activity of group I intron. The activity of group I intron required a suitable concentration of Mg²⁺, optimal temperature, and ample reaction time. Yet, these factors might foster non-enzymatic degradation of RNA, particularly in settings with high Mg²⁺ concentration, increased temperature, and prolonged incubation periods (Chheda, U. et al. Factors Affecting Stability of RNA-Temperature, Length, Concentration, pH, and Buffering Species. J Pharm Sci 113, 377-385 (2024); Fabre, A. L., Colotte, M., Luis, A., Tuffet, S. & Bonnet, J. An efficient method for long-term room temperature storage of RNA. Eur J Hum Genet 22, 379-385 (2014)). Additionally, the pH of the reaction condition also influenced RNA integrity (Chheda, U. et al. Factors Affecting Stability of RNA-Temperature, Length, Concentration, pH, and Buffering Species. J Pharm Sci 113, 377-385 (2024)). To strike a balance between RNA integrity and circularization efficiency, these factors underwent testing. The objective was to achieve satisfactory RNA circularization under relatively mild conditions. Buffer pH, MgCl₂ concentration, temperature, and reaction time were all assessed (FIGS. 11A-11D). In most instances, the conditions of 55° C. for 1 hour in 50 mM HEPES (pH=6.8) and 10 mM MgCl₂ were determined to be optimal for RNA circularization with minimal damage on RNA integrity.

### Cis Splicing Outperforms PIE at Low Mg²⁺ Concentration

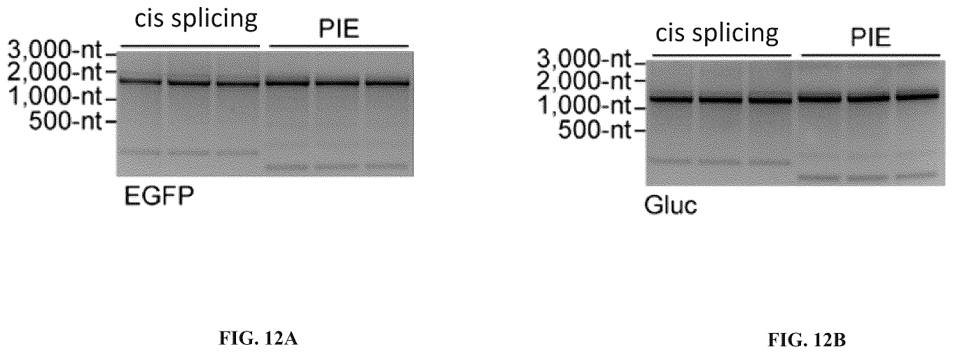

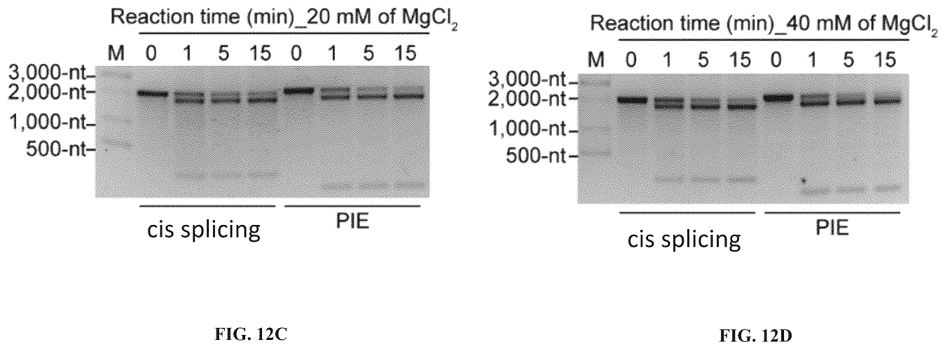

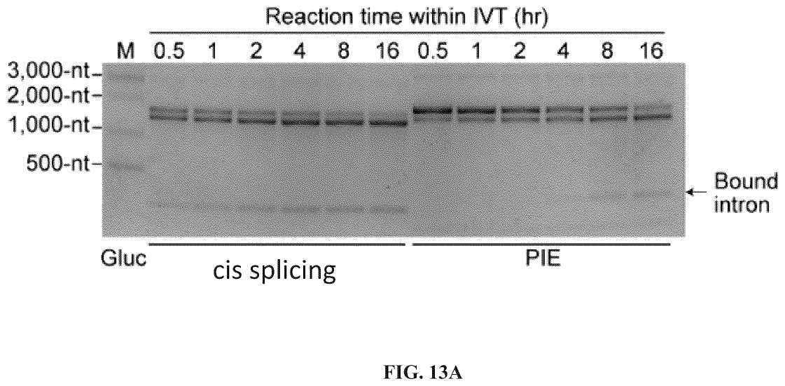

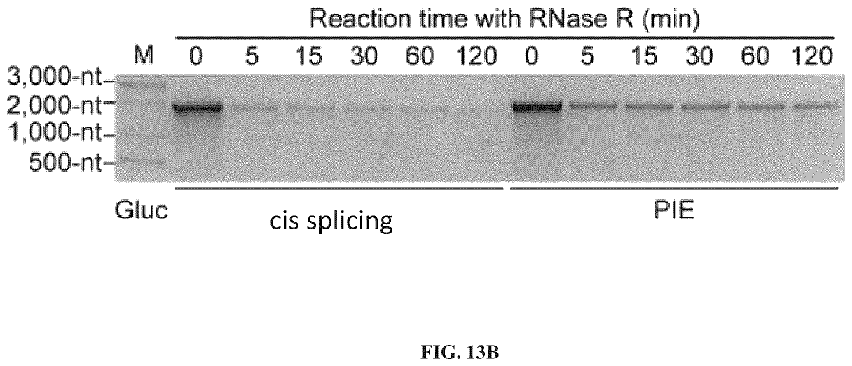

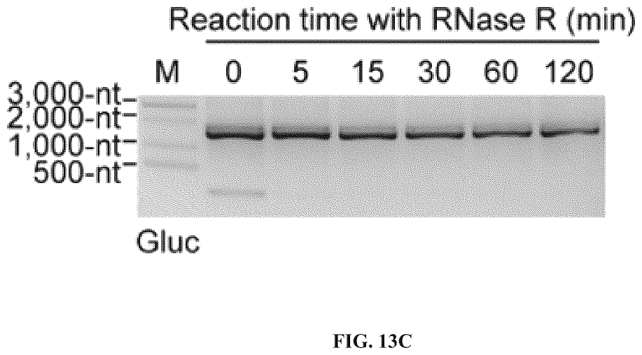

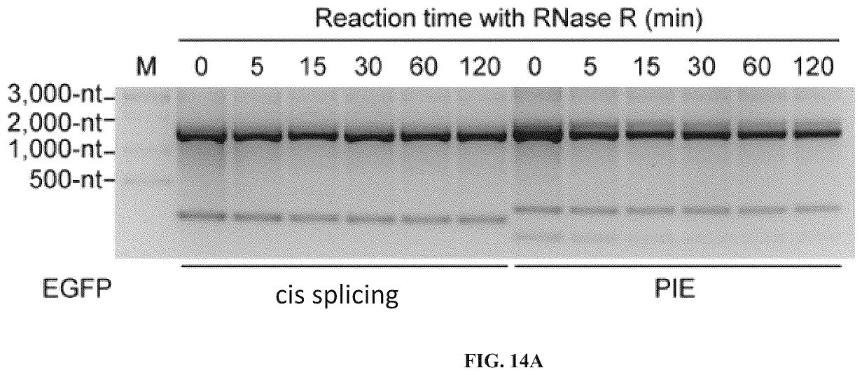

In an effort to identify unique features and potential advantages of cis splicing, we compared the circularization efficiency of cis splicing and PIE using the same payload. Under optimized condition with sufficient reaction time, both methods achieved high-efficiency RNA circularization (FIGS. 12A, 12B). To assess circularization efficiency during IVT reactions at different time points, we found that cis splicing achieved faster RNA circularization than PIE under IVT conditions (FIG. 7A and FIG. 13A). For instance, cis splicing at 37° C. for 1-2 hours achieved similar circularization efficiency to PIE at 37° C. for 16 hours (FIG. 7A and FIG. 13A).

Furthermore, RNA circularization was conducted at different concentrations of Mg²⁺ using precursors. At 10 mM Mg²⁺, cis splicing exhibited a higher circularization rate than PIE (FIG. 7B). When the Mg²⁺ concentration was increased to 20 mM, no significant difference was observed between cis splicing and PIE (FIG. 12C). However, at a Mg²⁺ concentration of 40 mM, slight inhibition was noted in the second step of the transesterification reaction of group I intron splicing, resulting in slower circularization with intermediate generation than in PIE (FIG. 12D). It is worth noting that high Mg²⁺ concentrations were considered harmful to RNA integrity (Li, Y. F. & Breaker, R. R. Kinetics of RNA degradation by specific base catalysis of transesterification involving the 2′-hydroxyl group. J Am Chem Soc 121, 5364-5372 (1999); Guth-Metzler, R. et al. Cutting in-line with iron: ribosomal function and non-oxidative RNA cleavage. Nucleic Acids Res 48, 8663-8674 (2020); Guth-Metzler, R. et al. Goldilocks and RNA: where Mg2+ concentration is just right. Nucleic Acids Res 51, 3529-3539 (2023)).

Next, we investigated the Mg²⁺ requirements for both approaches. We observed that cis splicing could effectively circularize RNA at lower Mg²⁺ concentration, approximately around 6 mM (FIG. 7C). In contrast, PIE required higher Mg²⁺ concentration for RNA circularization, approximately around 9 mM (FIG. 5D). Collectively, these distinctive features could be advantageous for circRNA integrity and large RNA circularization.

Cis Splicing Streamlines circRNA Purification with RNase R

In contrast to linear mRNA production, self-splicing intron-generated circRNA introduces additional byproducts such as linear precursors and spliced introns. Achieving high circRNA purity conveniently is crucial for advancing circRNA-based therapeutics. In laboratory settings, RNase R has been widely employed for circRNA purification due to its convenience. In comparing the two methods, particularly examining the resistance of spliced introns and linear precursors to RNase R, distinct features were observed in PIE, the 3′ end of the linear precursor was embedded in the homology arm, essential for RNA circularization (Wesselhoeft, R. A., Kowalski, P. S. & Anderson, D. G. Engineering circular RNA for potent and stable translation in eukaryotic cells. Nat Commun 9, 2629 (2018)), but this also led to increased resistance to RNase R digestion (FIG. 7E). Conversely, in cis splicing, the 3′ end of precursor was 5′ exon of group I intron, forming an ˜6 bp paired structure in the P1 domain in group I intron, suggesting a potential for easier digestion of linear precursor by RNase R (FIG. 7E).

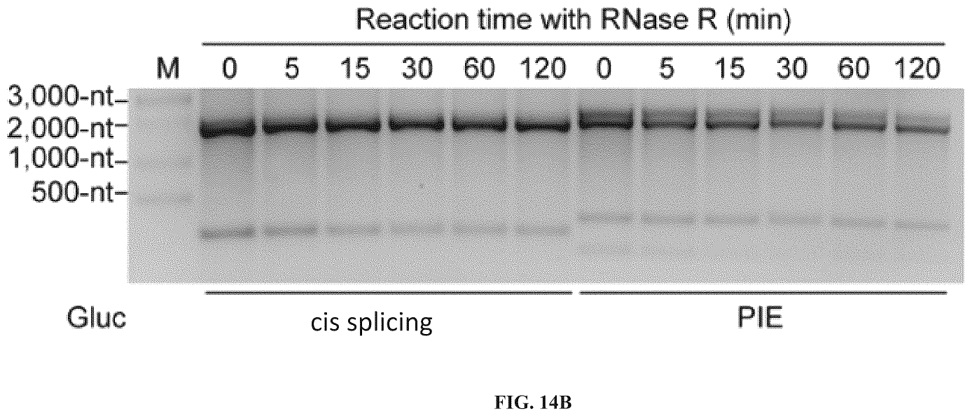

RNase R treatment of precursors in methods over different durations revealed that the precursor in PIE exhibited greater resistance to RNase R (FIG. 7F and FIG. 13B). It has been documented that the Ana intron exhibits self-ligase activity, implying that the intron's end can undergo ligation (Vicens, Q. & Cech, T. R. A natural ribozyme with 3′,5′ RNA ligase activity. Nat Chem Biol 5, 97-99 (2009)). According to our observations, the 5′ and 3′ intron ends were ligated during prolonged IVT reactions or RNA circularization, resulting in the formation of a circular intron in cis splicing and bound intron in PIE, both of which exhibited resistance to RNase R. RNase R treatment to these samples with prolonged incubation within IVT confirmed the resistance of the complete intron and bound intron to RNase R (FIGS. 14A, 14B). Thus, preventing intron ligation while achieving RNA circularization was identified as a straightforward method to enhance the efficacy of RNase R.

Notably, in cis splicing, RNA could be rapidly circularized without intron ligation. To facilitate RNase R digestion, we selected RNA samples after a short time of circularization by cis splicing (FIG. 7G and FIG. 13C). In summary, the expedited circularization process in cis splicing facilitated the prevention of intron ligation by shortening the reaction time. Overall, cis splicing demonstrated greater suitability for streamlined RNA purification through RNase R treatment, offering enhanced convenience for obtaining highly pure circRNA. However, the susceptibility of the cis splicing precursor to RNase R varied depending on sequence differences (FIG. 7F and FIG. 13B).

Cis Splicing Facilitates circRNA Purification Through Poly(A) Insertion

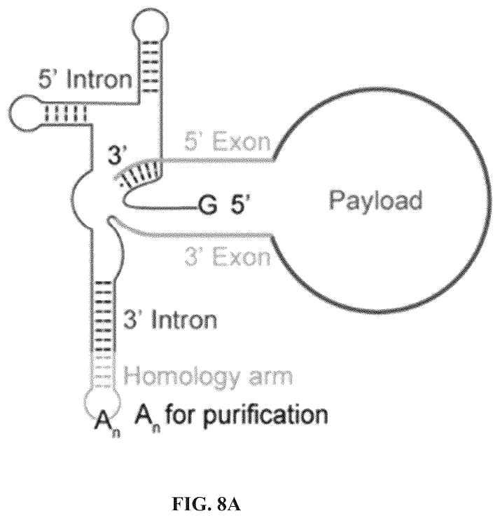

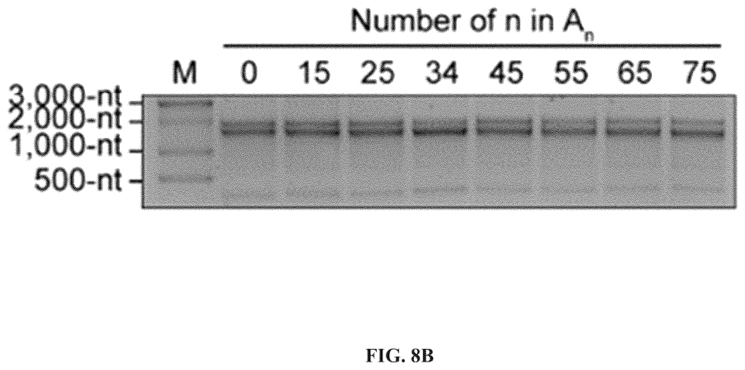

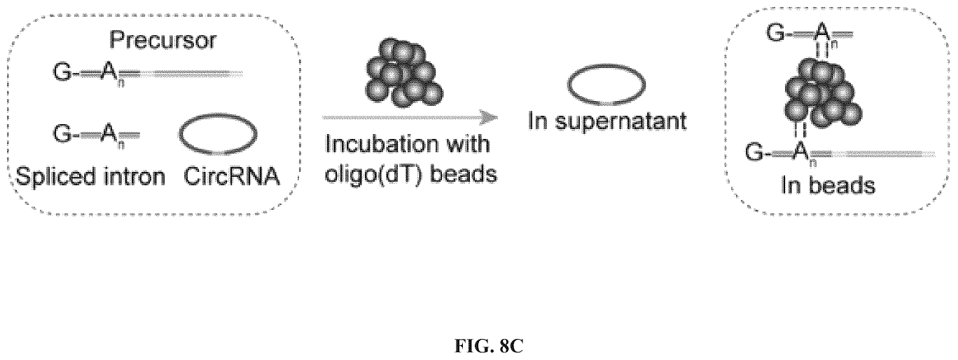

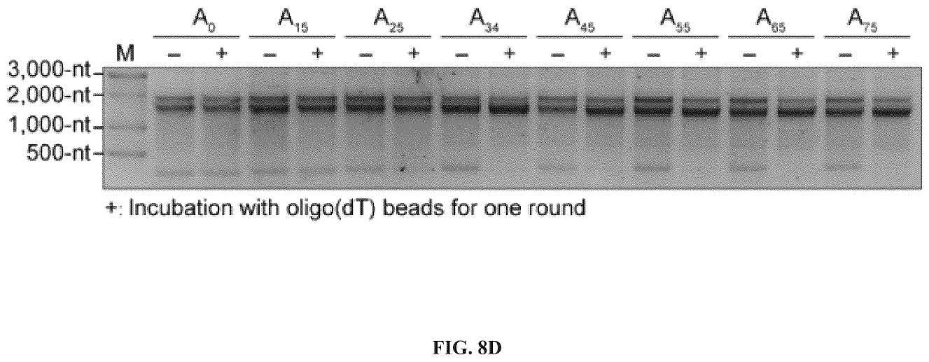

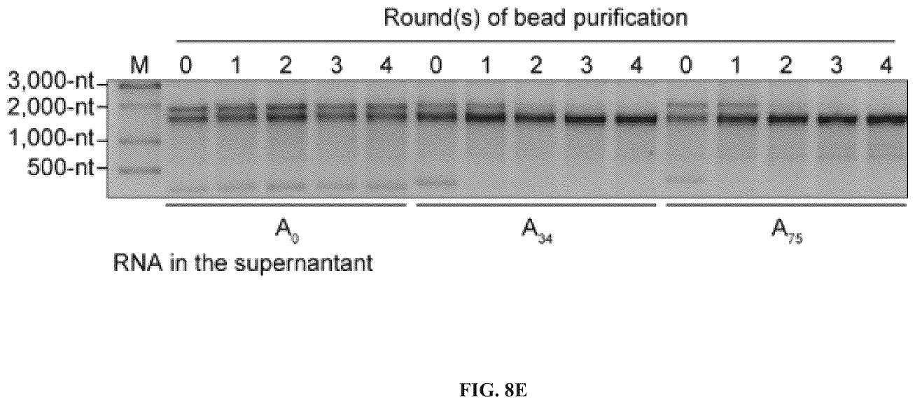

Oligo(dT) columns are commonly employed for mRNA purification by binding the poly(A) tails of mRNAs (Corbett, K. S. et al. SARS-COV-2 mRNA vaccine design enabled by prototype pathogen preparedness. Nature 586, 567-571 (2020); Mencin, N. et al. Development and scale-up of oligo-dT monolithic chromatographic column for mRNA capture through understanding of base-pairing interactions. Sep Purif Technol 304 (2023); Cui, T. et al. Comprehensive studies on building a scalable downstream process for mRNAs to enable mRNA therapeutics. Biotechnol Prog 39, e3301 (2023)). Considering that the byproducts in the cis splicing all contain intron sequences, the insertion of poly(A) sequences in the intron was explored to facilitate the removal of byproducts using oligo(dT) column or beads. Various lengths of poly(A) sequences were inserted into the P6 domain of Ana group I intron (FIG. 8A). To demonstrate the robustness of this method, precursors with only 3-nt for both exons, regardless of other assistant sequences, were used to reduce the circularization efficiency. Initially, the effect of A insertion on group I intron activity was tested, revealing that 0-75 A insertions did not affect intron splicing (FIG. 8B). Subsequently, a mix of circRNA and byproducts was incubated with oligo(dT) magnetic beads, and the supernatant was recovered to assess circRNA enrichment (FIG. 8C). After one round of incubation, an obvious enrichment of circRNA in supernatant was observed when the number of A insertions exceeded 25 (FIG. 8D). However, the precursor was more challenging to remove by beads than the intron due to its large size. To effectively eliminate precursors, more rounds of incubation with beads were performed, revealing that three rounds of incubation could effectively remove the precursor and intron in this case when the number of A insertions was 34 and 75 (FIG. 8E).

### Cis Splicing can Leverage Other Group I Introns for RNA Circularization

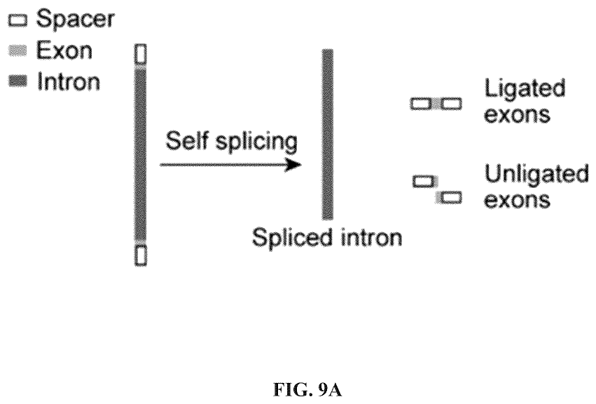

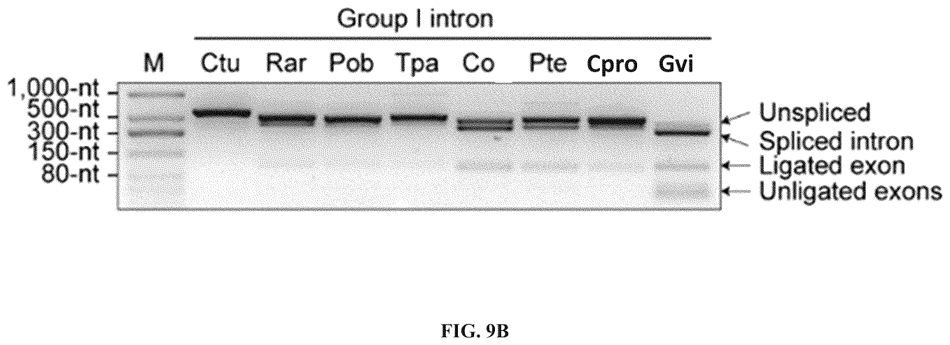

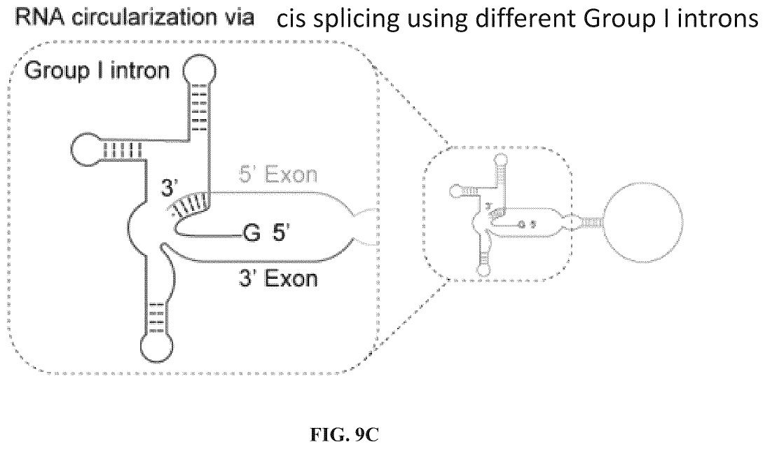

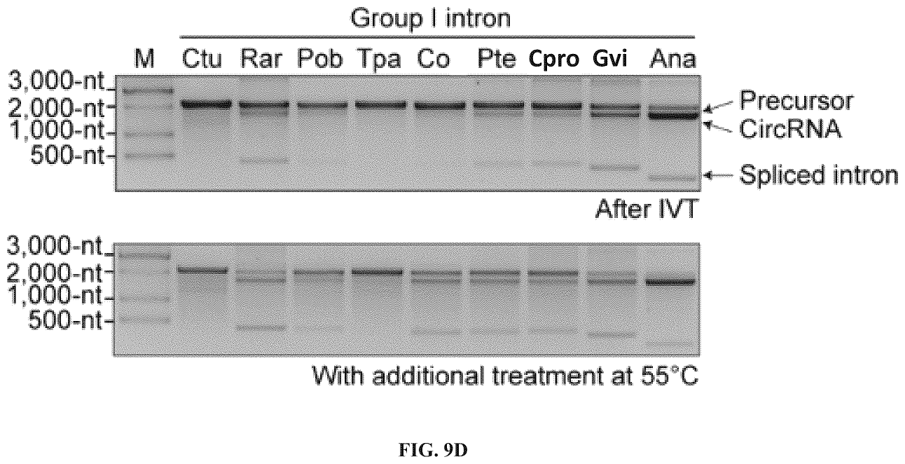

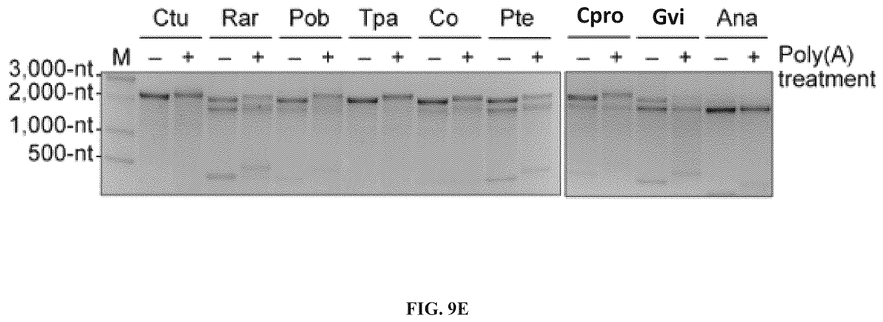

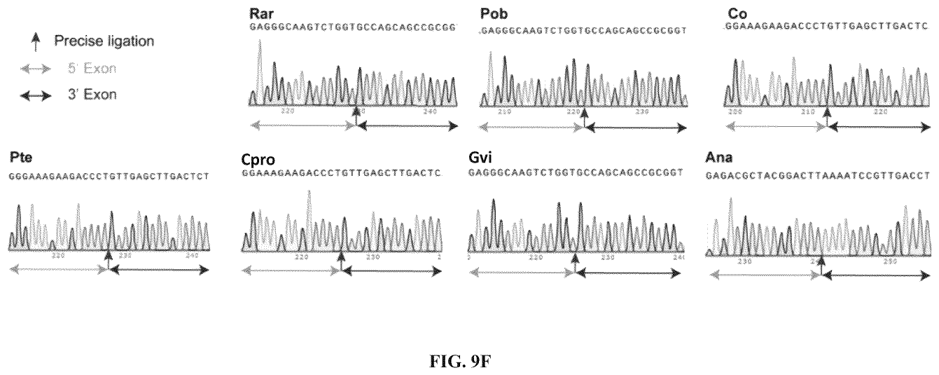

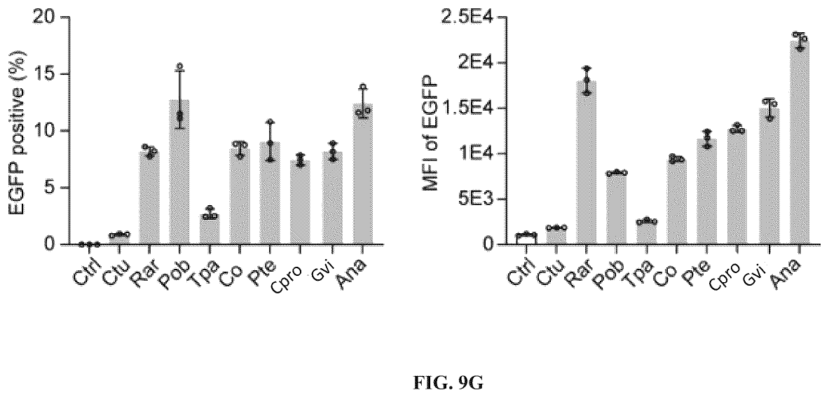

Since cis splicing can utilize complete introns for RNA circularization (FIG. 4), any group I introns with in vitro activity could be engineered to circularize RNA using cis splicing. Initially, various group I introns were selected from a reported database (Zhou, Y. et al. GISSD: Group I Intron Sequence and Structure Database. Nucleic Acids Res 36, D31-37 (2008)). Selected group I introns were tested for the in vitro activity (FIG. 9A). Great in vitro activity was observed in several introns (FIG. 9B), which were engineered for circRNA generation. The linear precursor was designed as G-intron-3′ exon-payload-5′ exon, with changes in the sequence of the intron and corresponding exons (FIG. 9C). Intron splicing was achieved within both IVT and after additional 55° C. treatment, resulting in the observation of spliced group I introns, except from introns of Closterium tumidum (Ctu) and Trichocoma paradoxa (Tpa) (FIG. 9D). Validation of the putative circRNAs were conducted by poly(A) assays, RT-PCR assays, and the detection of protein expression through transfecting RNA into cells. CircRNA generation was successful using the introns from Rasamsonia argillacea (Par), Penicillium oblatum (Pob), Cordyceps sp. 97009 (Co), Paecilomyces tenuipes (Pte), Polycephalomyces prolificus (Cpro), Talaromyces viridulus (Gvi) and Ana (FIG. 9E). Precise ligation of exons was confirmed through Sanger sequencing (FIG. 9F). Transfecting RNAs without purification into HEK293T cells revealed higher protein expression in circRNA compared to linear RNA without a cap and poly(A), with the exception of Ctu and Tpa (FIG. 9G). In conclusion, circRNA was successfully generated using various group I introns and could be engineered to produce protein-coding circRNA.

### Conclusions

This study introduces two innovative in vitro RNA circularization systems: trans splicing and cis splicing. Trans splicing capitalizes on the second step of group I intron splicing, providing an alternative method for RNA circularization. By adjusting the ratio of 5′ intron to intermediate, trans splicing holds potential to achieve higher circularization efficiency compared to PIE (FIGS. 3K, 3L). Notably, cis splicing, not constrained by intron splitting, can leverage various natural group I introns for RNA circularization, offering the potential to discover superior introns (FIG. 9).

In contrast to PIE (Wesselhoeft, R. A., Kowalski, P. S. & Anderson, D. G. Engineering circular RNA for potent and stable translation in eukaryotic cells. Nat Commun 9, 2629 (2018)), cis splicing offers several advantages. Firstly, cis splicing requires lower Mg²⁺ concentrations and achieves faster circularization under mild conditions or within IVT reactions compared to PIE, benefiting RNA integrity (FIGS. 7A-D). Secondly, cis splicing is more amenable to RNase R-mediated purification due to reduced generation of ligated introns during rapid circularization and the more flexible 3′ end of the precursor (FIGS. 7E-G). Additionally, cis splicing demonstrates the ability to tolerate poly(A) insertions with intron, enabling the use of oligo(dT) column/beads for circRNA purification (FIG. 8).

Currently, effective in vitro generation of circRNA with a payload exceeding 5,000 nucleotides is challenging (Wesselhoeft, R. A., Kowalski, P. S. & Anderson, D. G. Engineering circular RNA for potent and stable translation in eukaryotic cells. Nat Commun 9, 2629 (2018)), limiting the broader applications of the circRNA platform in large protein expression. Challenges in generating large-sized circRNA stem from conditions such as high concentrations of Mg²⁺, elevated temperatures, and extended reaction times, all of which can be detrimental to RNA integrity. Cis splicing may address these limitations by reducing the requirements for Mg²⁺ and time, offering the potential for rapid circularization of large RNA with minimal damage to RNA integrity.

Furthermore, group II introns have been reported to be used for RNA circularization by PIE (Chen, C. et al. A flexible, efficient, and scalable platform to produce circular RNAs as new therapeutics. BioRxiv (2022)). In general, both trans splicing and cis splicing hold potential to utilize group II introns due to the capability of group II introns to achieve self-splicing without proteins in vitro and undergo a two-step transesterification reaction. However, challenges arise from the branch-point pathway and lariat generation in the second step of splicing for group II introns in trans splicing and cis splicing. Nevertheless, reports suggest that group II introns can achieve splicing without lariat generation through a hydrolytic pathway under certain conditions (Daniels, D. L., Michels, W. J., Jr. & Pyle, A. M. Two competing pathways for self-splicing by group II introns: a quantitative analysis of in vitro reaction rates and products. J Mol Biol 256, 31-49 (1996)). Therefore, it is plausible that these group II introns can accomplish RNA circularization through trans splicing and cis splicing as described herein by utilizing the hydrolytic pathway.

Moreover, current ribozymes-mediated RNA circularization results in undesired scar sequences in the final circRNA, mainly consisting of exons sequence and other surrounding sequences assisted in circularization, such as homology arms. The scar was previously considered highly immunogenic and make it challenging to generate endogenous circRNA with the same sequences (Liu, C. X. & Chen, L. L. Circular RNAs: Characterization, cellular roles, and applications. Cell 185, 2016-2034 (2022)). However, in cis splicing, modest RNA circularization is achieved with only 3-nt for each exon (6-nt in total for both exons), regardless of any additional sequences (FIG. 10A and FIG. 10C), offering the potential to lower the immunogenicity of circRNAs by shortening the scar. Such a short scar has been hidden to achieve scarless results in PIE using distinct group I or group II introns (Wesselhoeft, R. A., Kowalski, P. S. & Anderson, D. G. Engineering circular RNA for potent and stable translation in eukaryotic cells. Nat Commun 9, 2629 (2018); Qiu, Z. et al. Clean-PIE: a novel strategy for efficiently constructing precise circRNA with thoroughly minimized immunogenicity to direct potent and durable protein expression. BioRxiv (2022)). Therefore, only 6-nt of exons sequence reserved in cis splicing holds the potential to be hidden in a similar approach, offering the possibility of generating exactly the same endogenous circRNA.

In conclusion, trans splicing and cis splicing present novel, efficient, and convenient methods for in vitro circRNA generation.

### Example 4: Efficient In Vitro Circularization of Large RNA Sequences

This example demonstrates the optimization of RNA circularization methods to enable cis splicing and subsequent circularization of large RNA sequences.

Presently, the use of common circularization methods to efficiently generate circRNAs spanning kilobases is challenging. Improved methods of circularization of RNA, particularly for large RNA sequences are needed. In this example, a reaction composition that improves RNA circularization, including large RNA sequences, was identified.

### Methods

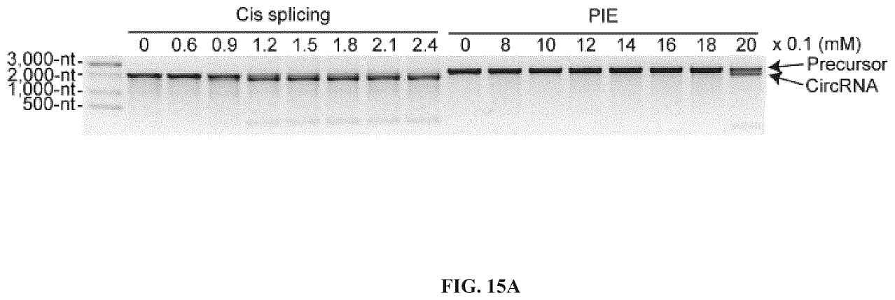

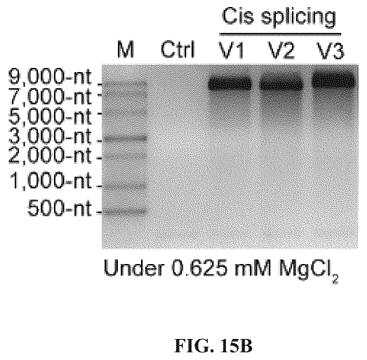

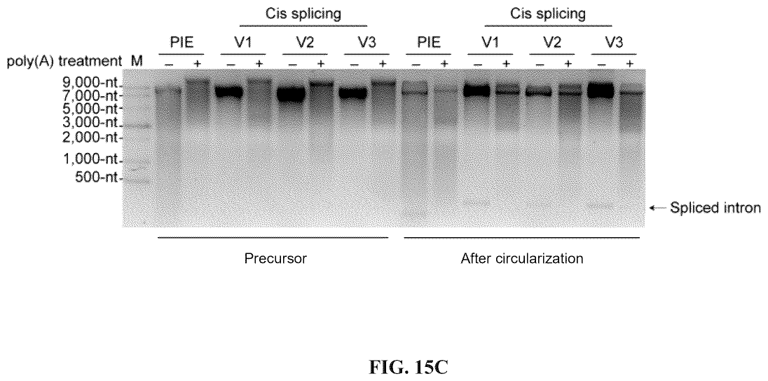

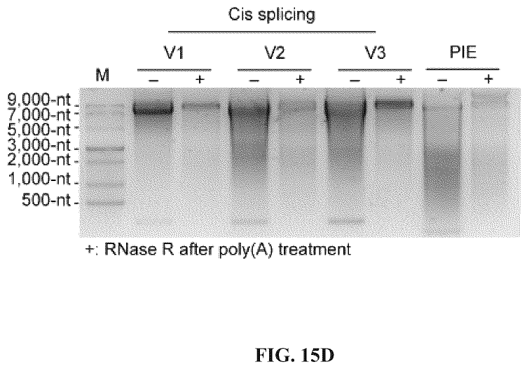

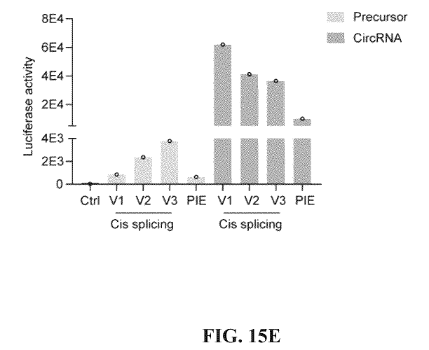

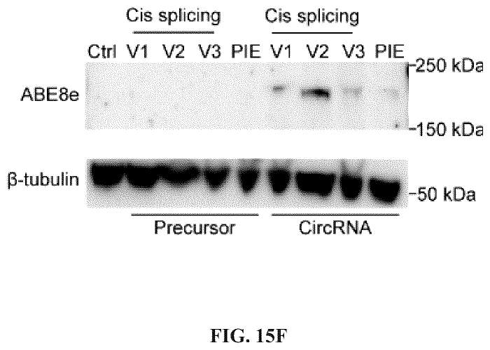

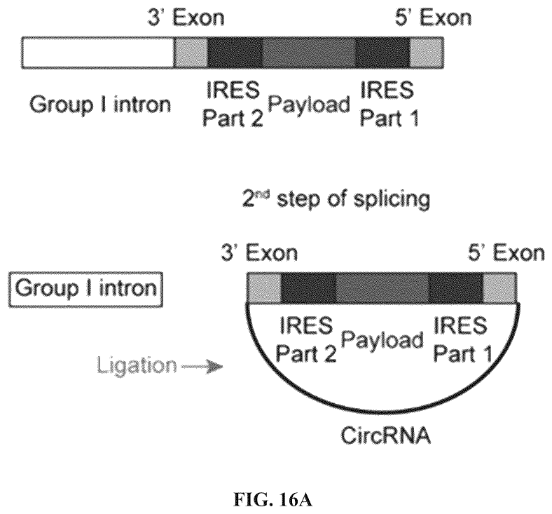

As described in previous Examples, the reactions required to circularize RNA comprise various components. Reaction components included Tris-HCl, MgCl₂, DTT, spermidine, monovalent cations (e.g., Na⁺, K⁺), MgCl₂, Mg²⁺, and others. A precursor (˜2,000 nt) was circularized by using cis splicing and PIE methods described in the aforementioned Examples. Further, the monovalent cations, Na⁺ and K⁺, were removed from the buffers. Various concentrations of MgCl₂ were tested for both cis splicing and PIE under buffer conditions lacking the monovalent cations. The tested MgCl₂ concentrations include 0, 0.06, 0.09, 0.12, 0.15, 0.18, 0.21, and 0.24 mM for cis splicing and 0, 0.8, 1.0, 1.2, 1.4, 1.6, 1.8, and 2.0 mM for PIE (FIG. 15A).

The cis splicing circularization of the ABE8e-2A-Fluc-EGFP reporter encoding an RNA over 8,000-nt was tested under these conditions, using under 0.625 mM of MgCl₂ (FIG. 15B). A control of equal molarity intron (that is, spliced intron produced in vitro and loaded in equal molarity to the precursors from lanes V1-V3 in order to help resolve the bands of precursor and circular RNA) was included in the lane labeled “Ctrl” to determine relative splicing efficiencies of the circularization reactions from the other lanes. The precursor and the products are circularization were treated with poly(A) polymerase and then RNase R, as previously described in the above Examples (FIG. 15C and FIG. 15D). The final circularized products were transfected in HEK293T cells to detect protein expression of ABE8e and luciferase (FIG. 15E and FIG. 15F). Results

After testing multiple reaction buffers in RNA circularization reactions, the buffer conditions lacking monovalent cations (such as Na⁺ or K⁺) further lowered the requirement for Mg²⁺ for both cis splicing and PIE methods (FIG. 15A). After, MgCl₂ was supplied at different concentrations for both methods, in which it was seen that the requirement for Mg²⁺ was lower in cis splicing than in previously observed buffer conditions with the monovalent cations included. Cis splicing was effective with as little as about 0.12 mM Mg²⁺, while PIE splicing required much higher Mg²⁺ concentrations (FIG. 15A).

Further, the circularization of the ABE8e-2A-Fluc-EGFP reporter (SEQ ID NO: 199) encoding RNA over 8,000-nt was tested, as a large improvement in RNA circularization efficiency was observed with a precursor of ˜2,000 nt as described above. “V1”, “V2”, and “V3”, which correspond to three reporter versions tested. Each version has a different homology arm length; “V1”: 19-nt, “V2”: 34-nt, and “V3”: 151-nt. The results showed that cis splicing effectively circularized RNA with little damage to the RNA integrity (FIG. 15B). RNA after circularization treatment as well as their precursors were treated with poly(A) polymerase and then RNase R. Both of these treatments verified the production of circRNA (FIG. 15C and FIG. 15D). The expression of the final product was detected after transfection in HEK293 cell. Detection of both luciferase and ABE8e indicated that RNA circularization of the reporter was achieved (FIG. 15E and FIG. 15F).

In conclusion, an improvement in circularization by cis splicing was observed with the use of buffers lacking monovalent cations, allowing for a decrease in the concentration of MgCl₂. Additionally, the circularization of large RNA sequences by cis splicing was shown to be efficient with the use of this buffer composition.

### Example 5: Circular RNAs with Reduced Immunogenicity

As previously reported (Liu et al., RNA circles with minimized immunogenicity as potent PKR inhibitors. 2021, Molecular cell; Guo et al., Therapeutic application of circular RNA aptamers in a mouse model of psoriasis, 2024, Nature Biotechnology), the “scar” sequence comprised of exon sequences or other assistant sequences found in the final circRNA product can alter the immunogenicity associated with in vitro synthesized circRNA.

This example demonstrates the optimization of sequences used for cis splicing to circularize RNA and produce circRNAs with smaller scars and/or scars hidden in an IRES sequence. The circRNAs with smaller and/or hidden scars display decreased immunogenicity compared to their longer and/or non-hidden counterparts.

Group I intron-mediated RNA circularization methods produce circularized RNA that include the exon sequences used to initiate splicing. These one or more exon sequences and/or other sequences not present in the effector sequence can comprise homology arms, and linker sequences. The inclusion of these sequences not originally present in the effector sequence in the final product is known as ‘a scar’. This scar may produce immunogenic effects in downstream applications using the circularized RNA. This example describes hiding the scar in regions such as an IRES sequence to reduce immunogenicity associated with circRNAs, especially when compared to circRNAs with corresponding non-hidden scars. Further, this example uses a split site design of the minimal motif for Group I Ana intron to produce circRNA with minimal scars.

### Methods

The linear RNA schematics described in FIG. 16A and FIG. 16B contain a split IRES design. Specifically, the CVB3 IRES was split into part 1 and part 2 as shown in FIG. 16B. The plasmids encoding these components (e.g., from 5′ to 3′: Group I intron, 3′ Exon, IRES Part 2, payload, IRES Part 1, and 5′ exon) were constructed following the cis splicing methods described in Example 3. Cis splicing effectively facilitated the circularization of RNA synthesized through in vitro transcription (IVT), relying on Group I introns without the need for fragmentation. After in vitro transcription of the RNA, the products were purified to obtain circRNAs.

Following the procedure described in FIG. 16C, the purified circRNAs were transfected to A549 cells (i.e., adenocarcinoma human alveolar basal epithelial cells). For circRNA transfection, cells were seeded and after 24 hr, purified circRNA was transfected using Lipofectamine MessengerMax (Invitrogen, LMRNA008). 6-hr post transfection, the cells were collected for the subsequent analysis. Poly(I:C) was used as positive control (Sigma Cat. No. P9582). The controls “linear 5′-3P” and “TNRNL2” both encode SEQ ID NO: 203. “TNRNL2” is a circular RNA generated by T4 RNA Ligase 2. The RNA was extracted from harvested cells and the circRNAs were reverse transcribed into DNA using the AMV First Strand cDNA Synthesis Kit (Sangon, B532445). Samples were dephosphorylated to reduce measuring an immune response associated with phosphates in contaminated RNA or nicked circRNA. qPCR was performed to evaluate the gene expression level of innate immune genes RIG-I, TNF-alpha, and IFN-beta (FIG. 16D). The fold change was relative to untransfected cells.

The minimal sequence identity requirements for the Group I Ana intron to undergo cis splicing using the split site vector design was also tested (FIG. 16D). The “NNUA” motif was identified to allow Group I Ana intron to produce circRNAs by cis splicing. Several motif “NNU|A” split sites were tested, including split sites 1, 2, and 3 for IRES-EGFP (SEQ ID NO: 5) and split sites 1 and 2 for POLR2A (SEQ ID NO: 204). The plasmids encoding the aforementioned sequences with the split site were cloned and circularized in vitro by cis splicing according to the methods provided herein. The circularized products were transfected into cells to isolate RNA and validate their expression in cells (FIG. 16E).

### Results

In this example, the immunogenicity of Group I intron-mediated scarless circRNA produced by cis splicing was evaluated. By hiding the non-effector sequences in the IRES, the circRNA displayed decreased immunogenic effects. Additionally, the Group I Ana intron produced circRNA by cis splicing through various tested split site designs.

Overall, a longer scar produced higher immunogenicity and shorter or hidden scars produced lower immunogenic effects. The 3′ exon and 5′ exon sequences were incorporated in the IRES by splitting the IRES into two parts, which allowed for the production of circRNAs with scars “hidden” within the IRES, thus decreasing the immunogenicity associated with circRNAs (FIG. 16A and FIG. 16B). The split site was between the 381^{st} and 382^{nd} nucleotides of CVB3 IRES (FIG. 16B). The gene expression levels of innate immune genes, RIG-I, TNF-alpha, and IFN-beta was measured to evaluate immunogenicity of the various circRNAs. The positive controls used are classical pathogenic mimics like poly (I:C) and 5′-triphosphate-containing linear RNAs (“linear 5′-3P”), which showed a high fold change in expression of these measured immune genes. The negative control (T4RNL2) produced minimal to no changes in gene expression levels (FIG. 16D). The circRNAs with hidden scars (i.e., samples labeled with “hi” in FIG. 16D) or corresponding circRNAs with scars found in the effector RNA region were both tested. The scar length “6” or “30” is labeled in the x-axis of FIG. 16D. The samples with circRNAs containing hidden scars produced smaller changes in gene expression levels for RIG-I, TNF-alpha, and IFN-beta than the corresponding circRNA with non-hidden scars (FIG. 16D). Minimal changes in gene expression levels were also observed in dephosphorylated samples. Further, the dephosphorylated samples with a hidden scar produced a more minimal change in expression levels than dephosphorylated samples with non-hidden scars. These data show the immunogenic benefits of hiding the scar sequence in the IRES.

Furthermore, the split site design used to produce circRNAs with hidden scars could also be applied to the Group I Ana intron when used to circularized RNA by cis splicing. For the Group I Ana intron, the sequence “NNUA” was the minimal motif that produced circRNAs by cis splicing (FIG. 16E). By splitting the motif, “scarless” circRNA were produced. The motif “NNUA” was split (|) as “NNU|A”, and the sequence “GNN” was included in the Group I intron to base pair with the “NNU” sequence downstream as shown in FIG. 16E. RNA circularization by cis splicing of IRES-EGFP and POLR2A is shown (FIG. 16F). The agarose gel electrophoresis results show the precursor, the scarless circRNA, and the spliced intron were produced in each of the split site designs and in both of the tested sequence contexts (FIG. 16F).

### Conclusion

These data demonstrate the production of circRNAs with hidden scars by incorporating a split site design in the sequence constructs used for RNA circularization. The circRNAs with hidden scars displayed decreased immunogenicity when compared to the circRNA comprising a corresponding non-hidden scar. Additionally, the identification of the Group I Ana intron “NNUA” motif and incorporation of the split site design shows an additional method that can be employed to produce scarless circRNA.

### Example 6: Novel Group II Introns for Cis Splicing Mediated Circularization of RNA

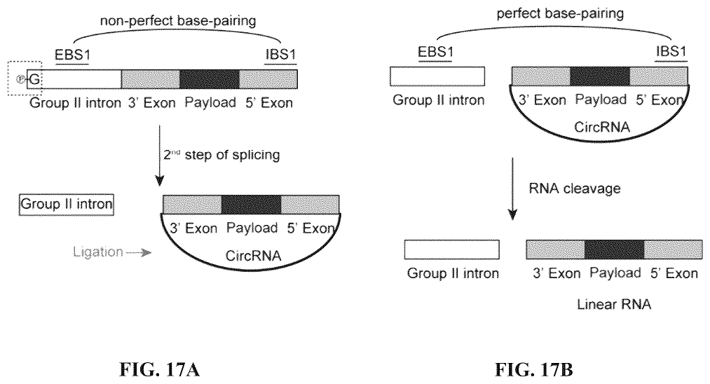

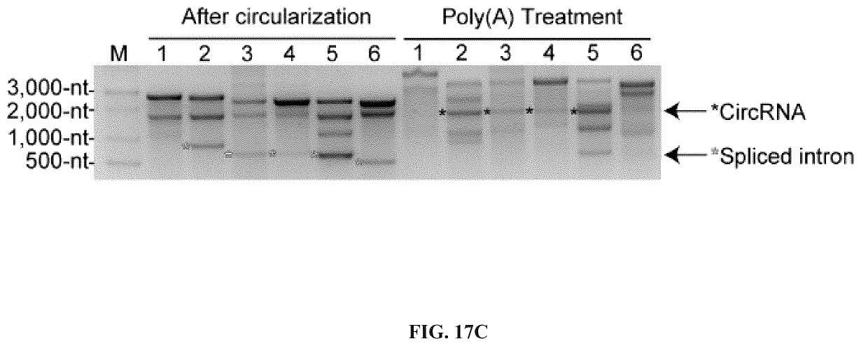

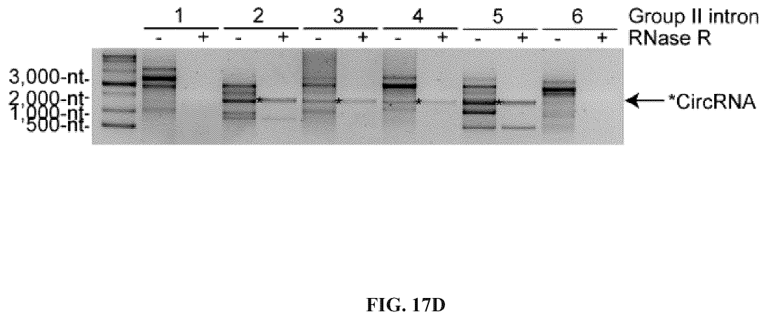

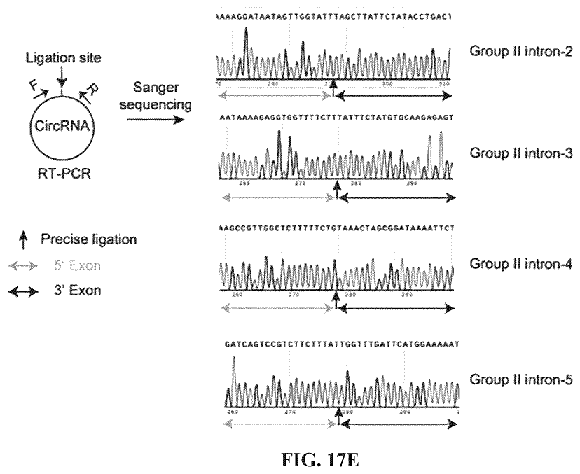

This example demonstrates the use of novel Group II introns for the circularization of RNA by cis splicing. This example shows that circRNAs were generated by cis splicing within a linear RNA precursor comprising, from the 5′ end to the 3′ end: a Group II intron, a 3′ exon sequence, an effector RNA sequence (i.e., payload), and a 5′ exon sequence, and lacking an intron fragment on the 3′ end (FIG. 17A).

### Methods

The methods described herein can apply to a catalytic Group II intron that undergoes the hydrolysis pathway for splicing (i.e., is self-splicing). For example, the same principles described herein in previous examples for cis splicing in the context of a catalytic Group I intron are also applicable for a catalytic Group II intron, as long as at least the following conditions are met: 1) there are no additional G residues introduced to the 5′ end of the linear RNA precursor (that is, no additional G residue should be added on the 5′ end of the linear RNA precursor); 2) there is no more than one phosphate group (i.e., 0 phosphate groups or 1 phosphate group) on the first G residue in the linear RNA precursor (i.e., on the G residue on or closest to the 5′ end of the linear RNA precursor); and 3) there is not perfect base pairing between the first exon binding sequence (EBS1) and the first intron binding sequence (IBS1) in the linear RNA precursor, as it may lead to cleavage activity near the circRNA ligation site (FIG. 17B).

The linear RNA schematics described in this example contain Group II introns with non-perfect base pairing (FIG. 17A) and Group II introns with perfect base pairing (FIG. 17B). The plasmids encoding these components (e.g., Group II intron, 3′ Exon, payload, and 5′ exon) were constructed following previously described cloning strategies, such as in Example 3. Cis splicing effectively facilitated the circularization of RNA synthesized through in vitro transcription (IVT). The construct with perfect base pairing underwent RNA cleavage. After in vitro transcription of the RNA, the circular products were validated by poly(A) and RNase R treatments. RNA was reverse transcribed and amplified using primers near the ligation site to confirm the results by PCR and Sanger sequencing (FIG. 17E).

### Results

The circularity of the products produced from constructs encoding Group II introns was validated by poly(A) (FIG. 17C) and RNase R (FIG. 17D) treatments. Precise ligation was detected using RT-PCR and Sanger sequencing (FIG. 17E).

Four Group II introns were identified to be compatible with in vitro RNA circularization by cis splicing. The circularization efficiency of samples with Group II introns by cis splicing was tested and circRNA products were confirmed after treatment of the transcribed products with poly (A) treatment (FIG. 17C). The constructs comprising Group II introns were reacted at 55° C. for circularization. Circularized products were then treated with poly(A) treatment, which only adds a poly(A) tail to non-circular products, such that circular products remained the same size. The spliced introns (white asterisk) and circularized RNAs (circRNAs, black asterisk) are shown in FIG. 17C, in which lane labels 1-6 represent constructs encoding linear RNA depicted in FIG. 17A, each of which has one of six different Group II intron sequences: Group II intron-1, Group II intron-2, Group II intron-3, Group II intron-4, Group II intron-5, or Group II intron-6. The sequence information of the intron, exon1, and exon2 sequences for each Group II intron is listed in Table 7. As indicated by the asterisks in FIG. 17C, Group II intron-2, Group II intron-3, Group II intron-4, and Group II intron-5 produced circRNAs. Samples were treated with RNase R to confirm production of circRNAs from Group II intron-mediated circularization by cis splicing. As indicated by the asterisks in FIG. 17D, Group II intron-2, Group II intron-3, Group II intron-4, and Group II intron-5 produced circRNAs, as a band remained after RNase R treatment. The circRNAs as validated by poly(A) and RNase R treatments were found in Group II intron-2, Group II intron-3, Group II intron-4, and Group II intron-5 and further processed for Sanger sequencing validation. The reverse transcribed (RT)-PCR products from circRNAs were amplified to confirm the sequence covering the ligation site. Sequencing data corresponding to the circRNA RT-PCR products derived from linear RNAs that encoded Group II intron-2, Group II intron-3, Group II intron-4, or Group II intron-5 confirmed the correct ligation site (FIG. 17E).

Four new Group II introns were identified herein to be compatible with in vitro RNA circularization by cis splicing. The molecular validation and sequencing confirmation demonstrated the applicability of these Group II introns in producing circRNAs by cis splicing.

TABLE 7
Group II intron sequences

| | Source | | Exon1 | Exon2 |
|---|---|---|---|---|
| Group II Intron No. | Organism | Intron SEQ ID NO. | SEQ ID NO. | SEQ ID NO. |
| 1 | Bacillus | 175 | 181 | 187 |
| | thuringiensis | | | |
| 2 | Clostridium | 176 | 182 | 188 |
| | perfringens | | | |
| 3 | Anoxybacillus | 177 | 183 | 189 |
| | pushchinoensis | | | |
| 4 | Desulforamulus | 178 | 184 | 190 |
| | ferrireducens | | | |
| 5 | Bacillus smithii | 179 | 185 | 191 |
| 6 | Oceanobacillus | 180 | 186 | 192 |
| | iheyensis | | | |

Table 7 above lists the SEQ ID NOs of the intronic and exonic sequences of the six Group II introns tested in Example 6.

### Example 7: Novel Group I Introns for Cis Splicing Mediated Circularization of RNA

This example demonstrates the use of novel Group I introns for the circularization of RNA by cis splicing. This example shows that circRNAs were generated by cis splicing within a linear RNA precursor comprising, from the 5′ end to the 3′ end: a Group I intron, a 3′ exon sequence, an effector RNA sequence (i.e., payload), and a 5′ exon sequence, and lacking an intron fragment on the 3′ end.

### Methods

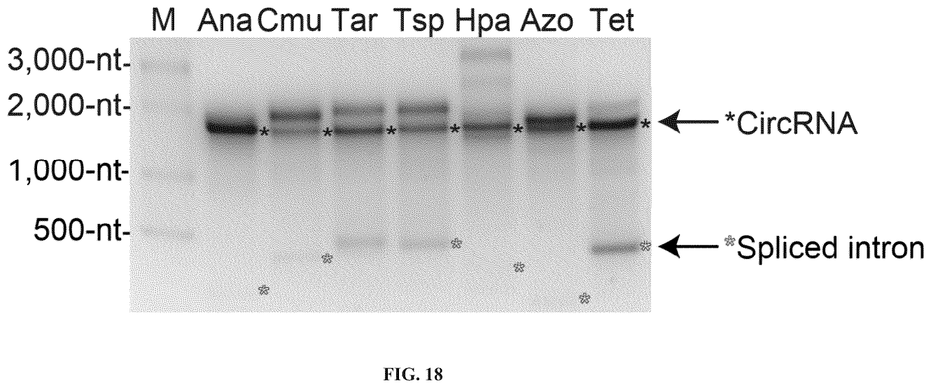

The plasmids encoding these components (e.g., Group I intron, 3′ Exon, payload, and 5′ exon) were constructed following previously described cloning strategies, such as in Example 3. Cis splicing effectively facilitated the circularization of RNA synthesized through in vitro transcription (IVT). RNA circularization was conducted under 6 mM MgCl₂ and 50 mM HEPES buffer (pH=6.8) without Na⁺ or K⁺ at 55° C. for 15 min. After in vitro transcription of the RNA, the circular products were validated by agarose gel electrophoresis to separate the spliced intron and circRNA (FIG. 18).

### Results

The circularity of the products produced from constructs encoding Group I introns was validated by visualizing the spliced introns and circRNA on an agarose gel. Six Group I introns were identified to be compatible with in vitro RNA circularization by cis splicing. The spliced introns (“Spliced intron”, white asterisks) and circularized RNAs (“CircRNA”, black asterisks) are indicated in FIG. 18. Lane label “M” indicates the molecular ladder. Lane labels indicate the following origin species for the Group I intron used in each linear precursor RNA construct, from left to right: “Ana”=Group I intron sequence from Anabaena (provided in SEQ ID NO: 9); “Cmu”=Group I intron sequence from Coelastrella multistriata (provided in SEQ ID NO: 154); “Tar”=Group I intron sequence from Trebouxia arboricola (provided in SEQ ID NO: 155); “Tsp”=Group I intron sequence from Trebouxia sp. (provided in SEQ ID NO: 156); “Hpa”=Group I intron sequence from Hypocrea pallida (provided in SEQ ID NO: 157); “Azo”=Group I intron sequence from Azoarcus olearius (provided in SEQ ID NO: 159); and “Tet”=Group I intron sequence from Tetrahymena thermophila (provided in SEQ ID NO: 158). The sequence information for each Group I intron and corresponding Exon1 and Exon2 is listed in Table 8.

Six new Group I introns were identified herein to be compatible with in vitro RNA circularization by cis splicing. The molecular validation demonstrated the applicability of these Group I introns in producing circRNAs by cis splicing.

TABLE 8
Group I intron sequences

| | | | Exon1 | Exon2 |
|---|---|---|---|---|
| Group I Intron No. | Organism | Intron SEQ ID NO. | SEQ ID NO. | SEQ ID NO. |
| 1 | Coelastrella multistriata | 154 | 160 | — |
| 2 | Trebouxia arboricola | 155 | 161 | 166 |
| 3 | Trebouxia sp. | 156 | 162 | 167 |
| 4 | Hypocrea pallida | 157 | 163 | 168 |
| 5 | Azoarcus olearius | 159 | 165 | 170 |
| 6 | Tetrahymena thermophila | 158 | 164 | 169 |

Table 8 above lists the SEQ ID NOs of the intronic and exonic sequences of the six Group I introns tested in Example 7.

### Exemplary Sequences

Exemplary sequences for Group I intron-mediated RNA circularization with trans-splicing can be found in the proceeding sequences. SEQ ID NO: 1: 5′ intron used in FIG. 1B. SEQ ID NO: 2: “Intermediate” linear RNA precursor used in FIG. 1B. SEQ ID NO: 3: 3′ intron used in FIG. 1B. SEQ ID NO: 4: Exon2 used in FIG. 1B. SEQ ID NO: 5: IRES-EGFP (GOI) used in FIG. 1B. SEQ ID NO: 6: Exon1 used in FIG. 1B.

Exemplary sequences for Group I intron-mediated RNA circularization with cis-splicing can be found in the proceeding sequences. SEQ ID NO: 7: Ana-1G used in FIG. 2B. SEQ ID NO: 8: Ana-2G used in FIG. 2B. SEQ ID NO: 9: Ana intron (engineered; containing homology arm introduced when using PIE method) used in FIG. 2B. SEQ ID NO: 10: Exon2 used in FIG. 2B. SEQ ID NO: 11: Exon1 used in FIG. 2B. SEQ ID NO: 12: 7-nt extension in FIG. 2C. SEQ ID NO: 13: Full length Ctu construct in FIG. 2C. SEQ ID NO: 14: Full length Par construct in FIG. 2C. SEQ ID NO: 15: Full length Gvi construct in FIG. 2C. SEQ ID NO: 16: Ctu intron in FIG. 2C. SEQ ID NO: 17: Par intron in FIG. 2C. SEQ ID NO: 18: Gvi intron in FIG. 2C. SEQ ID NO: 19: Ctu Exon2 in FIG. 2C. SEQ ID NO: 20: Par Exon2 and Gvi Exon2 in FIG. 2C. SEQ ID NO: 21: Ctu Exon1 in FIG. 2C. SEQ ID NO: 22: Par Exon1 and Gvi Exon1 in FIG. 2C. SEQ ID NO: 23: Full length Ana_no arm construct in FIG. 2G. SEQ ID NO: 24: Exon2 of Ana_no arm construct in FIG. 2G. SEQ ID NO: 25: Exon1 of Ana no arm construct in FIG. 2G. SEQ ID NO: 26: Part 1 of IRES8 in FIG. 2G; the split site is between Part 1 and Part 2. SEQ ID NO: 27: Part 2 of IRES8 in FIG. 2G; the split site is between Part 1 and Part 2. SEQ ID NO: 28: Part 1 of IRES3 in FIG. 2G; the split site is between Part 1 and Part 2. SEQ ID NO: 29: Part 2 of IRES3 in FIG. 2G; the split site is between Part 1 and Part 2. SEQ ID NO: 49: Ana Intron A insertion site 1: 15A in FIG. 2J. SEQ ID NO: 50: Ana Intron A insertion site 1: 25A in FIG. 2J. SEQ ID NO: 51: Ana Intron A insertion site 1: 35A in FIG. 2J. SEQ ID NO: 52: Ana Intron A insertion site 2: 15A in FIG. 2J. SEQ ID NO: 53: Ana Intron A insertion site 2: 25A in FIG. 2J. SEQ ID NO: 54: Ana Intron A insertion site 2: 35A in FIG. 2J.

Exemplary sequences for Group I intron-mediated RNA circularization can be found in the proceeding sequences. SEQ ID NO: 55: IC1_Ctu. S1506 (from Closterium tumidum), containing, from 5′ to 3′: a 15-nt exon 1 sequence, an IC1 Group I intron sequence, and a 15-nt exon 2 sequence. SEQ ID NO: 56: IE2_Par.S516-1 (from Rasamsonia argillacea), containing, from 5′ to 3′: a 15-nt exon 1 sequence, an IE2 Group I intron sequence, and a 15-nt exon 2 sequence. SEQ ID NO: 57: IE2_Cpro.L2066 (from Polycephalomyces prolificus), containing, from 5′ to 3′: a 15-nt exon 1 sequence, an IE2 Group I intron sequence, and a 15-nt exon 2 sequence. SEQ ID NO: 58: IE2_Pob. S516 (from Penicillium oblatum), containing, from 5′ to 3′: a 15-nt exon 1 sequence, an IE2 Group I intron sequence, and a 15-nt exon 2 sequence. SEQ ID NO: 59: IE2_Pte.L2066 (from Cordyceps tenuipes), containing, from 5′ to 3′: a 15-nt exon 1 sequence, an IE2 Group I intron sequence, and a 15-nt exon 2 sequence. SEQ ID NO: 60: IE2_Gvi.S516 (from Talaromyces viridulus), containing, from 5′ to 3′: a 15-nt exon 1 sequence, an IE2 Group I intron sequence, and a 15-nt exon 2 sequence. SEQ ID NO: 61: IE2_Co_sp-3.L2066 (from Cordyceps sp. 97009), containing, from 5′ to 3′: a 15-nt exon 1 sequence, an IE2 Group I intron sequence, and a 15-nt exon 2 sequence. SEQ ID NO: 62: IE2_Tpa.S516-1 (from Trichocoma paradoxa), containing, from 5′ to 3′: a 15-nt exon 1 sequence, an IE2 Group I intron sequence, and a 15-nt exon 2 sequence.

Exemplary sequences for Group I intron-mediated RNA circularization with cis-splicing and trans-splicing can be found in the proceeding sequences. SEQ ID NO: 68: FIG. 3: Trans splicing intermediate/precursor. SEQ ID NO: 69: FIG. 3: Trans splicing 5′ intron. SEQ ID NO: 70: FIG. 4B: Cis splicing without homology arm. SEQ ID NO: 71: Cis splicing with homology arm. FIG. 4D: Cis splicing group I intron=Cis splicing with homology arm (used in FIG. 4B). SEQ ID NO: 72: Cis splicing inactive group I intron. FIG. 6: Original sequence (before sequence optimization)=Cis splicing without homology arm (used in FIG. 4B). SEQ ID NO: 73: Intron-3′ Exon (52 nt)-5′ Homology arm-5′ Spacer-AC30-IRES-EGFP-3′ Spacer-3′ Homology arm-5′ Exon (16 nt): Intron. SEQ ID NO: 4: 3′ Exon (52 nt). SEQ ID NO: 6: 5′ Exon (16 nt). SEQ ID NO: 74: 5′ Homology arm. SEQ ID NO: 75: 3′ Homology arm. FIG. 6E: Same as in FIG. 6C and FIG. 6D. FIG. 6J: Lane EGFP C=Cis splicing without homology arm. SEQ ID NO: 111: Lane EGFP VI. SEQ ID NO: 112: Lane EGFP V2. SEQ ID NO: 113: Lane Gluc C. SEQ ID NO: 114: Lane Gluc V1. SEQ ID NO: 115: Lane Gluc V2. FIG. 8: no homology arms and spacers, with only 3 nt for both exons; FIG. 8: Intron sequences with inserted As. SEQ ID NO: 116: Intron_0A=Intron with homology arm. SEQ ID NO: 52: Intron_15A. SEQ ID NO: 53: Intron_25A. SEQ ID NO: 117: Intron 34A. SEQ ID NO: 118: Intron_45A. SEQ ID NO: 119: Intron_55A. SEQ ID NO: 120: Intron_65A. SEQ ID NO: 121: Intron_75A. FIG. 8: Full sequences: SEQ ID NO: 122: Intron_0A=Intron with homology arm; SEQ ID NO: 123: Intron_15A. SEQ ID NO: 124: Intron_25A. SEQ ID NO: 125: Intron_34A. SEQ ID NO: 126: Intron_45A. SEQ ID NO: 127: Intron_55A. SEQ ID NO: 128: Intron 65A. SEQ ID NO: 129: Intron_75A. FIG. 9D: Group I intron full sequences are included: SEQ ID NO: 130: Ctu; SEQ ID NO: 131: Rar (Par); SEQ ID NO: 132: Pob; SEQ ID NO: 133: Tpa; SEQ ID NO: 134: Cor (Co); SEQ ID NO: 135: Pte; SEQ ID NO: 136: Ppr (Cpro); SEQ ID NO: 137: Tvi (Gvi); SEQ ID NO: 138: Ana. FIG. 10: Sequences of Each Sample ID: Ctrl=Cis splicing without homology arm (as in FIG. 4B) Samples 1-7=sequences for each component disclosed as FIG. 6 sequences above (and Tables 2-6); each sample's combination of components disclosed in FIG. 10A. SEQ ID NO: 139: Sample 8. SEQ ID NO: 140: Sample 9. SEQ ID NO: 141: Sample 10. SEQ ID NO: 142: Sample 11. SEQ ID NO: 143: Sample 12. SEQ ID NO: 144: Sample 13. SEQ ID NO: 145: Sample 14. SEQ ID NO: 146: Sample 15. SEQ ID NO: 147: Sample 16. Payload for each sequence=IRES-EGFP (except for FIG. 6J, half of which uses Gluc): Intron Construct Sequences. SEQ ID NO: 148: Ctu. SEQ ID NO: 17: Rar (Par). SEQ ID NO: 149: Pob. SEQ ID NO: 150: Tpa. SEQ ID NO: 151: Cor (Co). SEQ ID NO: 152: Pte. SEQ ID NO.: 153: Ppr (Cpro). SEQ ID NO: 18: Tvi (Gvi). SEQ ID NO: 73: Ana without homology arm. SEQ ID NO: 116: Ana with homology arm.

TABLE 2
5′ Exon sequences in FIG. 6C

| Lane | 5′ Exon | SEQ ID NO. |
|---|---|---|
| 5′ Exon 1 nt | t | — |
| 5′ Exon 3 nt | ctt | — |
| 5′ Exon 6 nt | ggactt | — |
| 5′ Exon 11 nt | gctacggactt | 76 |
| 5′ Exon 16 nt | gagacgctacggactt | 6 |
| 5′ Exon 30 nt | gtgtggcggaatggga | 77 |
| | gacgctacggactt | |

TABLE 3
3′ Exon sequences in FIG. 6D

| | | SEQ |
|---|---|---|
| | | ID |
| Lane | 3′ Exon | NO. |
| 3′ Exon 1 nt | a | — |
| 3′ Exon 3 nt | aaa | — |
| 3′ Exon 6 nt | aaaatc | — |
| 3′ Exon 11 nt | aaaatccgttg | 78 |
| 3′ Exon 16 nt | aaaatccgttgacctt | 79 |
| 3′ Exon 30 nt | aaaatccgttgacct | 80 |
| | taaacggtcgtgtgg | |
| 3′ Exon 52 nt | aaaatccgttgacctt | 4 |
| | aaacggtcgtgtgggt | |
| | tcaagtccctccaccc | |
| | ccac | |

TABLE 4
Homology arm sequences in FIG. 6F

| | | SEQ | | SEQ |
|---|---|---|---|---|
| | | ID | | ID |
| Lane | 5′ Homology arm | NO. | 3′ Homology arm | NO. |
| He | TAAGCACAGGAGTG | 81 | TTACTGCAGGGATC | 88 |
| | TCATG | | CAAGT | |
| 0 | TTTAATTTAATAAA | 82 | AAAATTTTATTAAA | 89 |
| | ATTTT | | TTAAA | |
| 4 | GCCGATATAATTTA | 83 | TTTAATAAATTATA | 90 |
| | TTAAA | | TCGGC | |
| 7 | GCCGTAATAATTAA | 84 | CGGTATTAATTATT | 91 |
| | TACCG | | ACGGC | |
| 11 | GCCGTGTATAGTGA | 85 | CGGGTTCACTATAC | 92 |
| | ACCCG | | ACGGC | |
| 15 | GCCGAGCAAGCGCT | 86 | CGGCCAGCGCTTGC | 93 |
| | GGCCG | | TCGGC | |
| 19 | GCCGGCCCGGGGGG | 87 | CGGCCCCGCCCGGG | 94 |
| | GCCG | | CCGGC | |

TABLE 5
Homology arm sequences in FIG. 6G

| | | SEQ | | SEQ |
|---|---|---|---|---|
| | | ID | | ID |
| Lane | 5′ Homology arm | NO. | 3′ Homology arm | NO. |
| 4 | GCCG | — | CGGC | — |
| 9 | GCCGACCCG | — | CGGGTCGGC | — |
| 14 | GCCGAAGGCTGCCG | 95 | CGGCAGCCTTCGGC | 100 |
| 19 | GCCGAAGGCCTAGA | 96 | CGGAGTCTAGGCCTT | 101 |
| | CTCCG | | CGGC | |
| 24 | GCCGACGAAAGCCA | 97 | CGGGAGGCTTTGGCT | 102 |
| | AAGCCTCCCG | | TTCGTCGGC | |
| 29 | GCCGTGAAGTCGAGA | 98 | CGGCTGTGGTTAAGT | 103 |
| | CTTAACCACAGCCG | | CTCGACTTCACGGC | |
| 34 | GCCGTCAATATTGGA | 99 | CGGCACGTCGGTCAC | 104 |
| | GTTCGTGACCGACGT | | GAACTCCAATATTGA | |
| | GCCG | | CGGC | |

TABLE 6
sequences in FIG. 6H

| | | SEQ | | SEQ |
|---|---|---|---|---|
| | | ID | | ID |
| FIG. 6H | 5′ Spacer | NO. | 3′ Spacer | NO. |
| 10 | AAAAACAAAA | 105 | AAAAAACAAA | 108 |
| 20 | AAAAACAAAA | 106 | AAAAAACAAA | 109 |
| | AACAAAAAAA | | AAACAAAACA | |
| 30 | AAAAACAAAA | 107 | AAAAAACAAA | 110 |
| | AACAAAAAAA | | AAACAAAACA | |
| | AACAAAAAAA | | AACAAAAAAA | |

Exemplary sequences for circularization of large RNA sequences can be found in the proceeding sequences. SEQ ID NO: 199: ABE8e-2A-Fluc-EGFP reporter.

Exemplary sequences for circular RNAs with reduced immunogenicity can be found in the proceeding sequences. SEQ ID NO: 203: encodes controls “linear 5′-3P” and “TNRNL2”.

Additional exemplary sequences for Group I introns can be found in the proceeding sequences. SEQ ID NO: 154: Cmu intron sequence. SEQ ID NO: 155: Tar intron sequence. SEQ ID NO: 156: Tsp intron sequence. SEQ ID NO: 157: Hpa intron sequence. SEQ ID NO: 158: Tetrahymena intron sequence. SEQ ID NO: 159: Azoarcus intron sequence. SEQ ID NO: 160: Cmu Exon1 sequence. SEQ ID NO: 161: Tar Exon1 sequence. SEQ ID NO: 162: Tsp Exon1 sequence. SEQ ID NO: 163: Hpa Exon1 sequence. SEQ ID NO: 164: Tetrahymena Exon1 sequence. SEQ ID NO: 165: Azoarcus Exon1 sequence. SEQ ID NO: 166: Tar Exon2 sequence. SEQ ID NO: 167: Tsp Exon2 sequence. SEQ ID NO: 168: Hpa Exon2 sequence. SEQ ID NO: 169: Tetrahymena Exon2 sequence. SEQ ID NO: 170: Azoarcus Exon2 sequence.

Exemplary sequences for group II introns and associated exon sequences for use in the methods provided herein can be found in the proceeding sequences. SEQ ID NO: 175: Group II Intron-1. SEQ ID NO: 181: Group II Intron-1-Exon1 and SEQ ID NO: 187: Group II Intron-1-Exon2.SEQ ID NO: 176: Group II Intron-2. SEQ ID NO: 182: Group II Intron-2-Exon1 and SEQ ID NO: 188: Group II Intron-2-Exon2. SEQ ID NO: 177: Group II Intron-3. SEQ ID NO: 183: Group II Intron-3-Exon1 and SEQ ID NO: 189: Group II Intron-3-Exon2. SEQ ID NO: 178: Group II Intron-4. SEQ ID NO: 184: Group II Intron-4-Exon1 and SEQ ID NO: 190: Group II Intron-4-Exon2. SEQ ID NO: 179: Group II Intron-5. SEQ ID NO: 185: Group II Intron-5-Exon1 and SEQ ID NO: 191: Group II Intron-5-Exon2. SEQ ID NO: 180: Group II Intron-6. SEQ ID NO: 186: Group II Intron-6-Exon1 and SEQ ID NO: 192: Group II Intron-6-Exon2.

### Exemplary Embodiments

Embodiment 1. A linear RNA precursor, comprising from the 5′ end to the 3′ end:
   (a) a catalytic Group I intron;
   (b) a 3′ exon sequence;
   (c) an effector RNA sequence, and
   (d) a 5′ exon sequence,
   wherein the catalytic Group I intron is capable of splicing the 3′ exon sequence and the 5′ exon sequence from the linear RNA precursor, thereby forming a circular RNA (“circRNA”) comprising the effector RNA.
Embodiment 2. The linear RNA precursor of Embodiment 1, wherein the catalytic Group I intron is derived from a naturally occurring intron selected from the group consisting of: a member of the IC3 family of Group I introns and a member of the IE2 family of Group I introns.
Embodiment 3. The linear RNA precursor of Embodiment 1 or Embodiment 2, wherein the 3′ exon sequence or 5′ exon sequence is no more than about 60 nucleotides long.
Embodiment 4. The linear RNA precursor of Embodiment 3, wherein the 3′ exon sequence or the 5′ exon sequence is no more than about 10 nucleotides long.
Embodiment 5. The linear RNA precursor of any one of Embodiments 1-4, wherein the catalytic Group I intron comprises a heterologous sequence.
Embodiment 6. The linear RNA precursor of Embodiment 5, wherein the heterologous sequence is inserted in a loop region of the Group I intron.
Embodiment 7. The linear RNA precursor of Embodiment 5, wherein the heterologous sequence is inserted in a stem region of the Group I intron.
Embodiment 8. The linear RNA precursor of any one of Embodiments 1-7, wherein the effector RNA sequence comprises a coding RNA sequence.
Embodiment 9. The linear RNA precursor of Embodiment 8, wherein the coding RNA sequence encodes a therapeutic polypeptide.
Embodiment 10. The linear RNA precursor of Embodiment 9, wherein the therapeutic polypeptide is selected from the group consisting of an antigenic polypeptide, a functional protein, a receptor protein, and a targeting protein.
Embodiment 11. The linear RNA precursor of any one of Embodiments 8-10, wherein the RNA precursor further comprises a Kozak sequence, an internal ribosomal entry site (IRES) sequence, or a portion thereof operably linked to the coding RNA sequence.
Embodiment 12. The linear RNA precursor of any one of Embodiments 1-7, wherein the effector RNA sequence is a sequence of a non-coding RNA selected from the group consisting of a guide RNA (gRNA), a deaminase-recruiting RNA (dRNA), a siRNA, a miRNA, a shRNA, and a long intervening non-coding (line) RNA.
Embodiment 13. The linear RNA precursor of any one of Embodiments 1-12, wherein the effector RNA sequence is about 50 to about 25000 nucleotides (nt) long.
Embodiment 14. A DNA construct comprising a coding DNA sequence encoding the linear RNA precursor of any one of Embodiments 1-13.
Embodiment 15. The DNA construct of Embodiment 14, further comprising a promoter operably linked to the coding DNA sequence.
Embodiment 16. The DNA construct of Embodiment 15, wherein the promoter is a T7 promoter.
Embodiment 17. The DNA construct of any one of Embodiments 14-16, wherein the construct is a viral vector or a plasmid.
Embodiment 18. A method of preparing a circRNA, comprising a) providing the linear RNA precursor of any one of Embodiments 1-13; and b) activating the catalytic Group I intron in the linear RNA precursor, wherein the activation of the catalytic Group I intron results in splicing of the 3′ exon sequence and the 5′ exon sequence from the linear RNA precursor, thereby forming the circRNA comprising the effector RNA.
Embodiment 19. The method of Embodiment 18, wherein the linear RNA precursor is provided by in vitro transcription from a DNA construct encoding the linear RNA precursor.
Embodiment 20. The method of Embodiment 18 or 19, wherein the activation of the catalytic Group I intron comprises incubating the linear RNA precursor in a reaction medium.
Embodiment 21. The method of Embodiment 20, wherein the reaction medium comprises a divalent metal ion.
Embodiment 22. The method of Embodiment 21, wherein the divalent metal ion is Mg²⁺.
Embodiment 23. The method of Embodiment 20 or 21, wherein the reaction medium comprises spermidine.
Embodiment 24. The method of any of Embodiments 20-23, wherein the incubation is carried out at a temperature of about 30-60° C.
Embodiment 25. The method of any one of Embodiments 19-24, further comprising isolating the circRNA.
Embodiment 26. The method of Embodiment 18, wherein the linear RNA precursor is provided by introducing a linear RNA precursor of any one of Embodiments 1-17 or a DNA construct encoding the linear RNA precursor to an individual.
Embodiment 27. A circular RNA prepared using the method of any one of Embodiments 18-26.
Embodiment 28. A RNA circularization system comprising:
   (i) a linear RNA precursor, comprising from the 5′ end to the 3′ end:
      (a) a 3′ catalytic Group I intron fragment;
      (b) a 3′ exon sequence;
      (c) an effector RNA sequence, and
      (d) a 5′ exon sequence;
   and
   (ii) a free 5′ catalytic Group I intron fragment,
   wherein the 3′ catalytic Group I intron fragment and the 5′ catalytic Group I intron fragment are capable of splicing the 3′ exon sequence and the 5′ exon sequence from the linear RNA precursor, thereby forming a circular RNA (“circRNA”) comprising the effector RNA.
Embodiment 29. The RNA circularization system of Embodiment 28, wherein the molar ratio of the 5′ Group I intron fragment and the linear RNA precursor is at least about 2:1.
Embodiment 30. The RNA circularization system of Embodiment 28 or Embodiment 29, wherein the catalytic Group I intron is derived from a naturally occurring intron selected from the group consisting of: a member of the IC3 family of Group I introns and a member of the IE2 family of Group I introns.
Embodiment 31. The RNA circularization system of any of Embodiments 28-30, wherein the 3′ exon sequence or 5′ exon sequence is no more than about 60 nucleotides long.
Embodiment 32. The RNA circularization system of Embodiment 31, wherein the 3′ exon sequence or the 5′ exon sequence is no more than about 10 nucleotides long.
Embodiment 33. The RNA circularization system of any one of Embodiments 28-32, wherein the 3′ catalytic Group I intron fragment comprises a heterologous sequence.
Embodiment 34. The RNA circularization system of Embodiment 33, wherein the heterologous sequence is inserted in a loop region of the 3′ catalytic Group I intron fragment.
Embodiment 35. The RNA circularization system of Embodiment 33, wherein the heterologous sequence is inserted in a stem region of the 3′ catalytic Group I intron fragment.
Embodiment 36. The RNA circularization system of any one of Embodiments 28-35, wherein the effector RNA sequence comprises a coding RNA sequence.
Embodiment 37. The RNA circularization system of Embodiment 36, wherein the coding RNA sequence encodes a therapeutic polypeptide.
Embodiment 38. The RNA circularization system of Embodiment 37, wherein the therapeutic polypeptide is selected from the group consisting of an antigenic polypeptide, a functional protein, a receptor protein, and a targeting protein.
Embodiment 39. The RNA circularization system of any one of Embodiments 36-38, wherein the RNA precursor further comprises a Kozak sequence, an internal ribosomal entry site (IRES) sequence, or a portion thereof operably linked to the coding RNA sequence.
Embodiment 40. The RNA circularization system of any one of Embodiments 28-35, wherein the effector RNA sequence is a sequence of a non-coding RNA selected from the group consisting of a guide RNA (gRNA), a deaminase-recruiting RNA (dRNA), a siRNA, a miRNA, a shRNA, and a long intervening non-coding (line) RNA.
Embodiment 41. The RNA circularization system of any one of Embodiments 28-40, wherein the effector RNA sequence is about 50 to about 25000 nucleotides (nt) long.
Embodiment 42. A DNA construct comprising a coding DNA sequence encoding the linear RNA precursor of any one of Embodiments 28-41.
Embodiment 43. The DNA construct of Embodiment 42, further comprising a promoter operably linked to the coding DNA sequence.
Embodiment 44. The DNA construct of Embodiment 43, wherein the promoter is a T7 promoter.
Embodiment 45. The DNA construct of any one of Embodiments 42-44, wherein the construct is a viral vector or a plasmid.
Embodiment 46. A method of preparing a circRNA, comprising a) providing the RNA circularization system of any one of Embodiments 28-41; and b) activating the 3′ catalytic Group I intron fragment in the linear RNA precursor and the free 5′ catalytic Group I intron fragment, wherein the activation of the catalytic Group I intron results in splicing of the 3′ exon sequence and the 5′ exon sequence from the linear RNA precursor, thereby forming the circRNA comprising the effector RNA.
Embodiment 47. The method of preparing a circRNA of Embodiment 46, wherein the linear RNA precursor and the free 5′ Group I intron fragment are provided sequentially.
Embodiment 48. The method of preparing a circRNA of Embodiment 46, wherein the linear RNA precursor and the free 5′ Group I intron fragment are provided simultaneously.
Embodiment 49. The method of preparing a circRNA of Embodiment 46, wherein the linear RNA precursor and the free 5′ Group I intron fragment are provided in a single composition.
Embodiment 50. The method of Embodiment 46, wherein the linear RNA precursor is provided by in vitro transcription from a DNA construct encoding the linear RNA precursor.
Embodiment 51. The method of any of Embodiments 46-50, wherein the activation of the 3′ catalytic Group I intron fragment and the 5′ Group I intron fragment comprises incubating the linear RNA precursor and the free 5′ catalytic Group I intron fragment in a reaction medium.
Embodiment 52. The method of Embodiment 51, wherein the reaction medium comprises a divalent metal ion.
Embodiment 53. The method of Embodiment 52, wherein the divalent metal ion is Mg²⁺.
Embodiment 54. The method of Embodiment 52 or 53, wherein the reaction medium comprises spermidine.
Embodiment 55. The method of any of Embodiments 51-54, wherein the incubation is carried out at a temperature of about 30-60° C.
Embodiment 56. The method of any one of Embodiments 51-55, further comprising isolating the circRNA.
Embodiment 57. The method of Embodiment 46, wherein the RNA circularization system is provided by introducing the RNA circularization system of any one of Embodiments 28-45 or a DNA construct encoding the linear RNA precursor and/or the free 5′ Group I intron fragment to an individual.
Embodiment 58. The linear RNA precursor of any one of Embodiments 1-13, wherein the catalytic Group I intron comprises a heterologous sequence that tunes the catalytic activity of the catalytic Group I intron.
Embodiment 59. The RNA circularization system of any one of Embodiments 28-41, wherein the 3′ catalytic Group I intron fragment comprises a heterologous sequence that tunes the catalytic activity of the 3′ catalytic Group I intron fragment.
Embodiment 60. The method of any one of Embodiments 18-26, wherein the catalytic Group I intron comprises a heterologous sequence that tunes the catalytic activity of the catalytic Group I intron.
Embodiment 61. The method of any one of Embodiments 46-57, wherein the 3′ catalytic Group I intron fragment comprises a heterologous sequence that tunes the catalytic activity of the 3′ catalytic Group I intron fragment.
Embodiment 62. A circular RNA prepared using the method of any one of Embodiments 46-57 or 61.
Embodiment 63. The method of any one of Embodiments 18-26 and 60, the method of any one of Embodiments 46-57 and 61, the circular RNA of Embodiments 27, and/or the circular RNA of Embodiment 62, wherein the circular RNA comprises a short scar sequence.
Embodiment 64. The method of any one of Embodiments 18-26 and 60, the method of any one of Embodiments 46-57 and 61, the circular RNA of Embodiments 27, and/or the circular RNA of Embodiment 62, wherein the circular RNA comprises a hidden scar sequence
Embodiment 65. The method or circular RNA of Embodiment 63 or Embodiment 64, wherein the circular RNA exhibits reduced immunogenicity compared to a corresponding circular RNA comprising a longer scar sequence and/or wherein the circular RNA exhibits reduced immunogenicity compared to a corresponding circular RNA comprising a scar sequence that is not hidden.
Embodiment 66. The method or circular RNA of Embodiment 63 or Embodiment 64, wherein the short scar sequence or the hidden scar sequence is in an IRES sequence or in a UTR sequence.
Embodiment 67. The method or circular RNA of Embodiment 66, wherein the short scar sequence or the hidden scar sequence is in an IRES sequence.
Embodiment 68. The method or circular RNA of any one of Embodiments 63-67, wherein the scar sequence comprises an NNUA motif.
Embodiment 69. The linear RNA precursor of any one of Embodiments 1-13 and 28-41, wherein the 3′ exon sequence comprises a 3′ A base, and wherein the 5′ exon sequence comprises a 5′ NNU motif.
Embodiment 70. The method of any one of Embodiments 18-26, 60-61, and 63-68, further comprising a dephosphorylation step after the formation of the circRNA.
Embodiment 71. The method or circular RNA of Embodiment 66, wherein the circular RNA exhibits reduced immunogenicity compared to a corresponding circular RNA comprising a scar sequence.
Embodiment 72. The method of any one of Embodiments 18-26 and 60 or the method of any one of Embodiments 46-57 and 61, wherein the activating occurs in a buffer that lacks Na⁺, that lacks K⁺, and/or that lacks other monovalent cations.
Embodiment 73. The method of any one of Embodiments 18-26 and 60, the method of any one of Embodiments 46-57 and 61, or the method of Embodiment 72, wherein the splicing occurs in a buffer comprising less than 1 mM Mg²⁺.
Embodiment 74. The method of Embodiment 73, wherein the splicing occurs in a buffer comprising less than 0.5 mM Mg²⁺.
Embodiment 75. A linear RNA precursor, comprising from the 5′ end to the 3′ end:
   (a) a catalytic Group II intron;
   (b) a 3′ exon sequence;
   (c) an effector RNA sequence, and
   (d) a 5′ exon sequence,
   wherein the catalytic Group II intron is capable of splicing the 3′ exon sequence and the 5′ exon sequence from the linear RNA precursor, thereby forming a circular RNA (“circRNA”) comprising the effector RNA.
Embodiment 76. The linear RNA precursor of Embodiment 75, wherein the catalytic Group II intron is derived from a naturally occurring intron of a species selected from the group consisting of: Bacillus thuringiensis, Clostridium perfringens, Anoxybacillus pushchinoensis, Desulforamulus ferrireducens, Bacillus smithii, and Oceanobacillus iheyensis.
Embodiment 77. The linear RNA precursor of Embodiment 75 or Embodiment 76, wherein the 3′ exon sequence or 5′ exon sequence is no more than about 60 nucleotides long.
Embodiment 78. The linear RNA precursor of Embodiment 77, wherein the 3′ exon sequence or the 5′ exon sequence is no more than about 10 nucleotides long.
Embodiment 79. The linear RNA precursor of any one of Embodiments 75-78, wherein the catalytic Group II intron comprises a heterologous sequence.
Embodiment 80. The linear RNA precursor of Embodiment 79, wherein the heterologous sequence is inserted in a loop region of the Group II intron.
Embodiment 81. The linear RNA precursor of Embodiment 80, wherein the heterologous sequence is inserted in a stem region of the Group II intron.
Embodiment 82. The linear RNA precursor of any one of Embodiments 75-81, wherein the effector RNA sequence comprises a coding RNA sequence.
Embodiment 83. The linear RNA precursor of Embodiment 82, wherein the coding RNA sequence encodes a therapeutic polypeptide.
Embodiment 84. The linear RNA precursor of Embodiment 83, wherein the therapeutic polypeptide is selected from the group consisting of an antigenic polypeptide, a functional protein, a receptor protein, and a targeting protein.
Embodiment 85. The linear RNA precursor of any one of Embodiments 82-84, wherein the RNA precursor further comprises a Kozak sequence, an internal ribosomal entry site (IRES) sequence, or a portion thereof operably linked to the coding RNA sequence.
Embodiment 86. The linear RNA precursor of any one of Embodiments 75-81, wherein the effector RNA sequence is a sequence of a non-coding RNA selected from the group consisting of a guide RNA (gRNA), a deaminase-recruiting RNA (dRNA), a siRNA, a miRNA, a shRNA, and a long intervening non-coding (line) RNA.
Embodiment 87. The linear RNA precursor of any one of Embodiments 75-86, wherein the effector RNA sequence is about 50 to about 25000 nucleotides (nt) long.
Embodiment 88. A DNA construct comprising a coding DNA sequence encoding the linear RNA precursor of any one of Embodiments 75-87.
Embodiment 89. The DNA construct of Embodiment 88, further comprising a promoter operably linked to the coding DNA sequence.
Embodiment 90. The DNA construct of Embodiment 89, wherein the promoter is a T7 promoter.
Embodiment 91. The DNA construct of any one of Embodiments 88-90, wherein the construct is a viral vector or a plasmid.
Embodiment 92. A method of preparing a circRNA, comprising a) providing the linear RNA precursor of any one of Embodiments 75-87; and b) activating the catalytic Group II intron in the linear RNA precursor, wherein the activation of the catalytic Group II intron results in splicing of the 3′ exon sequence and the 5′ exon sequence from the linear RNA precursor, thereby forming the circRNA comprising the effector RNA.
Embodiment 93. The method of Embodiment 92, wherein the linear RNA precursor is provided by in vitro transcription from a DNA construct encoding the linear RNA precursor.
Embodiment 94. The method of Embodiment 92 or 93, wherein the activation of the catalytic Group II intron comprises incubating the linear RNA precursor in a reaction medium.
Embodiment 95. The method of Embodiment 94, wherein the reaction medium comprises a divalent metal ion.
Embodiment 96. The method of Embodiment 95, wherein the divalent metal ion is Mg²⁺.
Embodiment 97. The method of Embodiment 94 or 95, wherein the reaction medium comprises spermidine.
Embodiment 98. The method of any of Embodiments 94-97, wherein the incubation is carried out at a temperature of about 30-60° C.
Embodiment 99. The method of any one of Embodiments 93-98, further comprising isolating the circRNA.
Embodiment 100. The method of Embodiment 92, wherein the linear RNA precursor is provided by introducing a linear RNA precursor of any one of Embodiments 75-91 or a DNA construct encoding the linear RNA precursor to an individual.
Embodiment 101. A circular RNA prepared using the method of any one of Embodiments 92-100.
Embodiment 102. A RNA circularization system comprising:
   (i) a linear RNA precursor, comprising from the 5′ end to the 3′ end:
      (a) a 3′ catalytic Group II intron fragment;
      (b) a 3′ exon sequence;
      (c) an effector RNA sequence, and
      (d) a 5′ exon sequence;
   and
   (ii) a free 5′ catalytic Group II intron fragment,
   wherein the 3′ catalytic Group II intron fragment and the 5′ catalytic Group II intron fragment are capable of splicing the 3′ exon sequence and the 5′ exon sequence from the linear RNA precursor, thereby forming a circular RNA (“circRNA”) comprising the effector RNA.
Embodiment 103. The RNA circularization system of Embodiment 102, wherein the molar ratio of the 5′ Group II intron fragment and the linear RNA precursor is at least about 2:1.
Embodiment 104. The RNA circularization system of Embodiment 102 or Embodiment 103, wherein the catalytic Group II intron is derived from a naturally occurring intron of a species selected from the group consisting of: Bacillus thuringiensis, Clostridium perfringens, Anoxybacillus pushchinoensis, Desulforamulus ferrireducens, Bacillus smithii, and Oceanobacillus iheyensis.
Embodiment 105. The RNA circularization system of any of Embodiments 102-104, wherein the 3′ exon sequence or 5′ exon sequence is no more than about 60 nucleotides long.
Embodiment 106. The RNA circularization system of Embodiment 105, wherein the 3′ exon sequence or the 5′ exon sequence is no more than about 10 nucleotides long.
Embodiment 107. The RNA circularization system of any one of Embodiments 102-106, wherein the 3′ catalytic Group II intron fragment comprises a heterologous sequence.
Embodiment 108. The RNA circularization system of Embodiment 107, wherein the heterologous sequence is inserted in a loop region of the 3′ catalytic Group II intron fragment.
Embodiment 109. The RNA circularization system of Embodiment 107, wherein the heterologous sequence is inserted in a stem region of the 3′ catalytic Group II intron fragment.
Embodiment 110. The RNA circularization system of any one of Embodiments 102-109, wherein the effector RNA sequence comprises a coding RNA sequence.
Embodiment 111. The RNA circularization system of Embodiment 110, wherein the coding RNA sequence encodes a therapeutic polypeptide.
Embodiment 112. The RNA circularization system of Embodiment 111, wherein the therapeutic polypeptide is selected from the group consisting of an antigenic polypeptide, a functional protein, a receptor protein, and a targeting protein.
Embodiment 113. The RNA circularization system of any one of Embodiments 110-112, wherein the RNA precursor further comprises a Kozak sequence, an internal ribosomal entry site (IRES) sequence, or a portion thereof operably linked to the coding RNA sequence.
Embodiment 114. The RNA circularization system of any one of Embodiments 102-109, wherein the effector RNA sequence is a sequence of a non-coding RNA selected from the group consisting of a guide RNA (gRNA), a deaminase-recruiting RNA (dRNA), a siRNA, a miRNA, a shRNA, and a long intervening non-coding (line) RNA.
Embodiment 115. The RNA circularization system of any one of Embodiments 102-114, wherein the effector RNA sequence is about 50 to about 25000 nucleotides (nt) long.
Embodiment 116. A DNA construct comprising a coding DNA sequence encoding the linear RNA precursor of any one of Embodiments 102-115.
Embodiment 117. The DNA construct of Embodiment 116, further comprising a promoter operably linked to the coding DNA sequence.
Embodiment 118. The DNA construct of Embodiment 117, wherein the promoter is a T7 promoter.
Embodiment 119. The DNA construct of any one of Embodiments 116-118, wherein the construct is a viral vector or a plasmid.
Embodiment 120. A method of preparing a circRNA, comprising a) providing the RNA circularization system of any one of Embodiments 102-115; and b) activating the 3′ catalytic Group II intron fragment in the linear RNA precursor and the free 5′ catalytic Group II intron fragment, wherein the activation of the catalytic Group II intron results in splicing of the 3′ exon sequence and the 5′ exon sequence from the linear RNA precursor, thereby forming the circRNA comprising the effector RNA.
Embodiment 121. The method of preparing a circRNA of Embodiment 120, wherein the linear RNA precursor and the free 5′ Group II intron fragment are provided sequentially.
Embodiment 122. The method of preparing a circRNA of Embodiment 120, wherein the linear RNA precursor and the free 5′ Group II intron fragment are provided simultaneously.
Embodiment 123. The method of preparing a circRNA of Embodiment 120, wherein the linear RNA precursor and the free 5′ Group II intron fragment are provided in a single composition.
Embodiment 124. The method of Embodiment 120, wherein the linear RNA precursor is provided by in vitro transcription from a DNA construct encoding the linear RNA precursor.
Embodiment 125. The method of any of Embodiments 120-124, wherein the activation of the 3′ catalytic Group II intron fragment and the 5′ Group II intron fragment comprises incubating the linear RNA precursor and the free 5′ catalytic Group II intron fragment in a reaction medium.
Embodiment 126. The method of Embodiment 125, wherein the reaction medium comprises a divalent metal ion.
Embodiment 127. The method of Embodiment 126, wherein the divalent metal ion is Mg²⁺.
Embodiment 128. The method of Embodiment 126 or 127, wherein the reaction medium comprises spermidine.
Embodiment 129. The method of any of Embodiments 125-128, wherein the incubation is carried out at a temperature of about 30-60° C.
Embodiment 130. The method of any one of Embodiments 125-129, further comprising isolating the circRNA.
Embodiment 131. The method of Embodiment 120, wherein the RNA circularization system is provided by introducing the RNA circularization system of any one of Embodiments 102-119 or a DNA construct encoding the linear RNA precursor and/or the free 5′ Group II intron fragment to an individual.
Embodiment 132. The linear RNA precursor of any one of Embodiments 75-87, wherein the catalytic Group II intron comprises a heterologous sequence that tunes the catalytic activity of the catalytic Group II intron.
Embodiment 133. The RNA circularization system of any one of Embodiments 102-115, wherein the 3′ catalytic Group II intron fragment comprises a heterologous sequence that tunes the catalytic activity of the 3′ catalytic Group II intron fragment.
Embodiment 134. The method of any one of Embodiments 92-100, wherein the catalytic Group II intron comprises a heterologous sequence that tunes the catalytic activity of the catalytic Group II intron.
Embodiment 135. The method of any one of Embodiments 120-131, wherein the 3′ catalytic Group II intron fragment comprises a heterologous sequence that tunes the catalytic activity of the 3′ catalytic Group II intron fragment.
Embodiment 136. A circular RNA prepared using the method of any one of Embodiments 120-131 or 135.
Embodiment 137. The method of any one of Embodiments 92-100 and 134, the method of any one of Embodiments 120-131 and 135, the circular RNA of Embodiment 101, and/or the circular RNA of Embodiment 136, wherein the circular RNA comprises a short scar sequence.
Embodiment 138. The method of any one of Embodiments 92-100 and 134, the method of any one of Embodiments 120-131 and 135, the circular RNA of Embodiment 101, and/or the circular RNA of Embodiment 136, wherein the circular RNA comprises a hidden scar sequence.
Embodiment 139. The method or circular RNA of Embodiment 137 or Embodiment 138, wherein the circular RNA exhibits reduced immunogenicity compared to a corresponding circular RNA comprising a longer scar sequence and/or wherein the circular RNA exhibits reduced immunogenicity compared to a corresponding circular RNA comprising a scar sequence that is not hidden.
Embodiment 140. The method or circular RNA of Embodiment 137 or Embodiment 138, wherein the short scar sequence or the hidden scar sequence is in an IRES sequence or in a UTR sequence.
Embodiment 141. The method or circular RNA of Embodiment 140, wherein the short scar sequence or the hidden scar sequence is in an IRES sequence.
Embodiment 142. The method or circular RNA of any one of Embodiments 137-141, wherein the scar sequence comprises an NNUA motif.
Embodiment 143. The linear RNA precursor of any one of Embodiments 75-87 and 102-115, wherein the 3′ exon sequence comprises a 3′ A base, and wherein the 5′ exon sequence comprises a 5′ NNU motif.
Embodiment 144. The method of any one of Embodiments 92-100, 134-135, and 137-142, further comprising a dephosphorylation step after the formation of the circRNA.
Embodiment 145. The method or circular RNA of Embodiment 140, wherein the circular RNA exhibits reduced immunogenicity compared to a corresponding circular RNA comprising a scar sequence.
Embodiment 146. The method of any one of Embodiments 92-100 and 134 or the method of any one of Embodiments 120-131 and 135, wherein the activating occurs in a buffer that lacks Na⁺, that lacks K⁺, and/or that lacks other monovalent cations.
Embodiment 147. The method of any one of Embodiments 92-100 and 134, the method of any one of Embodiments 120-131 and 135, or the method of Embodiment 146, wherein the splicing occurs in a buffer comprising less than 1 mM Mg²⁺.
Embodiment 148. The method of Embodiment 147, wherein the splicing occurs in a buffer comprising less than 0.5 mM Mg²⁺.

1. A linear RNA precursor, comprising from the 5′ end to the 3′ end:
(a) a catalytic intron;
(b) a 3′ exon sequence;
(c) an effector RNA sequence, and
(d) a 5′ exon sequence,
wherein the catalytic intron is capable of splicing the 3′ exon sequence and the 5′ exon sequence from the linear RNA precursor, thereby forming a circular RNA (“circRNA”) comprising the effector RNA.

2. A RNA circularization system comprising:
(i) a linear RNA precursor, comprising from the 5′ end to the 3′ end:
(a) a 3′ catalytic intron fragment;
(b) a 3′ exon sequence;
(c) an effector RNA sequence, and
(d) a 5′ exon sequence;
and
(ii) a free 5′ catalytic intron fragment,
wherein the 3′ catalytic fragment and the 5′ catalytic intron fragment are capable of splicing the 3′ exon sequence and the 5′ exon sequence from the linear RNA precursor, thereby forming a circular RNA (“circRNA”) comprising the effector RNA.

3. The linear RNA precursor of claim 1, wherein the catalytic intron is a catalytic Group I intron.

4. The linear RNA precursor of claim 3, wherein the catalytic Group I intron is derived from a naturally occurring intron selected from the group consisting of: a member of the IC3 family of Group I introns and a member of the IE2 family of Group I introns.

5. The linear RNA precursor of claim 1, wherein the catalytic intron is a catalytic Group II intron.

6. The linear RNA precursor of claim 5, wherein the catalytic intron is a catalytic Group II intron derived from a naturally occurring intron of a species selected from the group consisting of: Bacillus thuringiensis, Clostridium perfringens, Anoxybacillus pushchinoensis, Desulforamulus ferrireducens, Bacillus smithii, and Oceanobacillus iheyensis.

7. The linear RNA precursor of claim 1, wherein the 3′ exon sequence or 5′ exon sequence is no more than about 60 nucleotides long.

8. The linear RNA precursor of claim 1, wherein the 3′ exon sequence or the 5′ exon sequence is no more than about 10 nucleotides long.

9. The linear RNA precursor of claim 1, wherein the catalytic intron comprises a heterologous sequence.

10. The linear RNA precursor of claim 9, wherein the heterologous sequence is inserted in a loop region of the catalytic intron, the 3′ catalytic intron fragment, or the 5′ catalytic intron fragment.

11. The linear RNA precursor of claim 9, wherein the heterologous sequence is inserted in a stem region of the catalytic intron, the 3′ catalytic intron fragment, or the 5′ catalytic intron fragment.

12. The linear RNA precursor of claim 1, wherein the effector RNA sequence comprises a coding RNA sequence.

13. The linear RNA precursor of claim 12, wherein the coding RNA sequence encodes a therapeutic polypeptide.

14. The linear RNA precursor of claim 13, wherein the therapeutic polypeptide is selected from the group consisting of an antigenic polypeptide, a functional protein, a receptor protein, and a targeting protein.

15. The linear RNA precursor of claim 1, wherein the RNA precursor further comprises a Kozak sequence, an internal ribosomal entry site (IRES) sequence, or a portion thereof operably linked to the coding RNA sequence.

16. The linear RNA precursor of claim 1, wherein the effector RNA sequence is a sequence of a non-coding RNA selected from the group consisting of a guide RNA (gRNA), a deaminase-recruiting RNA (dRNA), a siRNA, a miRNA, a shRNA, and a long intervening non-coding (line) RNA.

17. The linear RNA precursor of claim 1, wherein the effector RNA sequence is about 50 to about 25000 nucleotides (nt) long.

18. The linear RNA precursor of claim 1, wherein the catalytic intron comprises a heterologous sequence that tunes the catalytic activity of the catalytic intron.

19. A method of preparing a circRNA, comprising a) providing the linear RNA precursor of claim 1; and b) activating the catalytic intron in the linear RNA precursor, wherein the activation of the catalytic intron results in splicing of the 3′ exon sequence and the 5′ exon sequence from the linear RNA precursor, thereby forming the circRNA comprising the effector RNA.

20. A method of preparing a circRNA, comprising a) providing the RNA circularization system of claim 2; and b) activating the 3′ catalytic intron fragment in the linear RNA precursor and the free 5′ catalytic intron fragment, wherein the activation of the catalytic intron results in splicing of the 3′ exon sequence and the 5′ exon sequence from the linear RNA precursor, thereby forming the circRNA comprising the effector RNA.


SEQUENCE LISTING (204 sequences)

SEQ ID NO: 1 - DNA, 155 bp, synthetic construct
  source 1..155: mol_type = other DNA
gaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagttt

SEQ ID NO: 2 - DNA, 1820 bp, synthetic construct
  source 1..1820: mol_type = other DNA
ggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtaaaaaacaaaaaacaaaacggctattatgcgttaccggcgagacgctacggactt

SEQ ID NO: 3 - DNA, 165 bp, synthetic construct
  source 1..165: mol_type = other DNA
ggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 4 - DNA, 52 bp, synthetic construct
  source 1..52: mol_type = other DNA
aaaatccgttgaccttaaacggtcgtgtgggttcaagtccctccacccccac

SEQ ID NO: 5 - DNA, 1474 bp, synthetic construct
  source 1..1474: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 6 - DNA, 16 bp, synthetic construct
  source 1..16: mol_type = other DNA
gagacgctacggactt

SEQ ID NO: 7 - DNA, 1866 bp, synthetic construct
  source 1..1866: mol_type = other DNA
gaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgagacgctacggactt

SEQ ID NO: 8 - DNA, 1867 bp, synthetic construct
  source 1..1867: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgagacgctacggactt

SEQ ID NO: 9 - DNA, 319 bp, synthetic construct
  source 1..319: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatga

SEQ ID NO: 10 - DNA, 51 bp, synthetic construct
  source 1..51: mol_type = other DNA
aaatccgttgaccttaaacggtcgtgtgggttcaagtccctccacccccac

SEQ ID NO: 11 - DNA, 16 bp, synthetic construct
  source 1..16: mol_type = other DNA
gagacgctacggactt

SEQ ID NO: 12 - skipped

SEQ ID NO: 13 - DNA, 478 bp, synthetic construct
  source 1..478: mol_type = other DNA
ggtgcctgtcgatgagcacacacgggagagccagccccttccgcagccaccgacacgggagcctgagcgggcccaccgggctgggcccgctggtccgacgacgcgcaaggggccggggtacggcgggctcgcgcccgtcctccccggcgccgccgtcgggcgacaccgtccaagtgcggagaccttcccagggcacacgctaccaagcgggagaggcgaggggagcggtcggctctccccgtcctccccgtggcccggccggaacgaggccggggtacggtcacaacgcgtgcgccgagctcaggggaacgatccgcagccaacccgggaccggccgccctcggccggtcgcggggcagtccacagactgaacgacggtgggtcggccgctcgagcccgagcggccggcctaagatgcagtcggcgagcgggggagacctcgctccgtccgcatagcagcacgcagcgcttcaccgtt

SEQ ID NO: 14 - DNA, 1920 bp, synthetic construct
  source 1..1920: mol_type = other DNA
gggaaccatcagttctctgctgacgccagagatagtagggtcccccccggaggaccgggggggattacgcctgctagtcgagcactcagtgtggcgatgggtgagtgccggcgaggtgacctggtacggggaagcctaagggttgtcggccgcgaggggggcccgttcgacctatggtaatcccgtggcgagctccagccgggcggaagcccgctggggccgtcgtaacgcgcggaaaggcaccggctgccacctccgggtggtggctcaagggacgtgctgtcccatccgaaaggatgccctcaactggagcacccatcgtgtaaaggttgaggggggcacgcggcgcggcgccgtgtgcccgggagaccctccccccgcgggttcccgcgggcgggggggctgttaacggccagcagccgcggtttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgagggcaagtctggt

SEQ ID NO: 15 - DNA, 1859 bp, synthetic construct
  source 1..1859: mol_type = other DNA
ggggacattcagttccaacatgctgacgccagagatagtgggcttcacgattccccccctggggaggtgaagtacgcccgctagtcgagcactcgcagtcgcgatgggtgagtgccggcgaggtgacctggtacggggaagcctacggcctgatgtctcaggctatggtaatcccgtggcgagcccccttccggggccgtcgtaacgcgcgcaaaggcaccggtcgtccgcccccgggcgacgactcaagggacgtgctgtcccatccgaaaggatgcccgccaccggaacacccatcgtgtaaaggtgacggggggcgtggggaagcccacgcccgggtgatgaaaacggccagcagccgcggtttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgagggcaagtctggt

SEQ ID NO: 16 - DNA, 476 bp, synthetic construct
  source 1..476: mol_type = other DNA
tgcctgtcgatgagcacacacgggagagccagccccttccgcagccaccgacacgggagcctgagcgggcccaccgggctgggcccgctggtccgacgacgcgcaaggggccggggtacggcgggctcgcgcccgtcctccccggcgccgccgtcgggcgacaccgtccaagtgcggagaccttcccagggcacacgctaccaagcgggagaggcgaggggagcggtcggctctccccgtcctccccgtggcccggccggaacgaggccggggtacggtcacaacgcgtgcgccgagctcaggggaacgatccgcagccaacccgggaccggccgccctcggccggtcgcggggcagtccacagactgaacgacggtgggtcggccgctcgagcccgagcggccggcctaagatgcagtcggcgagcgggggagacctcgctccgtccgcatagcagcacgcagcgcttcaccgtt

SEQ ID NO: 17 - DNA, 409 bp, synthetic construct
  source 1..409: mol_type = other DNA
gaaccatcagttctctgctgacgccagagatagtagggtcccccccggaggaccgggggggattacgcctgctagtcgagcactcagtgtggcgatgggtgagtgccggcgaggtgacctggtacggggaagcctaagggttgtcggccgcgaggggggcccgttcgacctatggtaatcccgtggcgagctccagccgggcggaagcccgctggggccgtcgtaacgcgcggaaaggcaccggctgccacctccgggtggtggctcaagggacgtgctgtcccatccgaaaggatgccctcaactggagcacccatcgtgtaaaggttgaggggggcacgcggcgcggcgccgtgtgcccgggagaccctccccccgcgggttcccgcgggcgggggggctgttaacg

SEQ ID NO: 18 - DNA, 348 bp, synthetic construct
  source 1..348: mol_type = other DNA
ggacattcagttccaacatgctgacgccagagatagtgggcttcacgattccccccctggggaggtgaagtacgcccgctagtcgagcactcgcagtcgcgatgggtgagtgccggcgaggtgacctggtacggggaagcctacggcctgatgtctcaggctatggtaatcccgtggcgagcccccttccggggccgtcgtaacgcgcgcaaaggcaccggtcgtccgcccccgggcgacgactcaagggacgtgctgtcccatccgaaaggatgcccgccaccggaacacccatcgtgtaaaggtgacggggggcgtggggaagcccacgcccgggtgatgaaaacg

SEQ ID NO: 19 - DNA, 15 bp, synthetic construct
  source 1..15: mol_type = other DNA
ttccgtaggtgaacc

SEQ ID NO: 20 - DNA, 15 bp, synthetic construct
  source 1..15: mol_type = other DNA
gccagcagccgcggt

SEQ ID NO: 21 - DNA, 15 bp, synthetic construct
  source 1..15: mol_type = other DNA
aagtcgtaacaaggt

SEQ ID NO: 22 - DNA, 15 bp, synthetic construct
  source 1..15: mol_type = other DNA
gagggcaagtctggt

SEQ ID NO: 23 - DNA, 1829 bp, synthetic construct
  source 1..1829: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgacctttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtagacgctacggactt

SEQ ID NO: 24 - DNA, 15 bp, synthetic construct
  source 1..15: mol_type = other DNA
aaaatccgttgacct

SEQ ID NO: 25 - DNA, 15 bp, synthetic construct
  source 1..15: mol_type = other DNA
agacgctacggactt

SEQ ID NO: 26 - DNA, 384 bp, synthetic construct
  source 1..384: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggg

SEQ ID NO: 27 - DNA, 357 bp, synthetic construct
  source 1..357: mol_type = other DNA
gaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 28 - DNA, 181 bp, synthetic construct
  source 1..181: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttacc

SEQ ID NO: 29 - DNA, 560 bp, synthetic construct
  source 1..560: mol_type = other DNA
ccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaa

SEQ ID NO: 30 - DNA, 30 bp, synthetic construct
  source 1..30: mol_type = other DNA
aaatccgttgaccttaaacggtcgtgtggg

SEQ ID NO: 31 - DNA, 60 bp, synthetic construct
  source 1..60: mol_type = other DNA
aaatccgttgaccttaaacggtcgtgtgggttcaagtccctccacccccataagagcaag

SEQ ID NO: 32 - DNA, 381 bp, synthetic construct
  source 1..381: mol_type = other DNA
gggaacgatgacgcatcaacggggtcagtagcggtcagcgtgccgctagtccagtcggccagcatctgtgggtggccacgggcgagacaacctggtacgggggagcctacgggggaggactcgtcctccctacggtaatcccgtggcgacctttccactggacttccactggagaggcgtcgtaacgcgcggaaaggtgtcggttggcggtcgcgaggccgccggcttaagggacgtgctaaaccctggcgaaagccagcccgccgacggagcgcccccagcgcaaagtcgtcgggggtcgtacttacaccacgcctgggaggaaatgccctggcggagaccggtagcctctgctggcctgcaaaggccgcgggggaattg

SEQ ID NO: 33 - skipped

SEQ ID NO: 34 - DNA, 15 bp, synthetic construct
  source 1..15: mol_type = other DNA
gggaaagaagaccct

SEQ ID NO: 35 - DNA, 30 bp, synthetic construct
  source 1..30: mol_type = other DNA
ttggcagaatcagcggggaaagaagaccct

SEQ ID NO: 36 - DNA, 60 bp, synthetic construct
  source 1..60: mol_type = other DNA
tctagcgaaaccacagccaagggaatgggcttggcagaatcagcggggaaagaagaccct

SEQ ID NO: 37 - skipped

SEQ ID NO: 38 - DNA, 15 bp, synthetic construct
  source 1..15: mol_type = other DNA
gttgagcttgactct

SEQ ID NO: 39 - DNA, 30 bp, synthetic construct
  source 1..30: mol_type = other DNA
gttgagcttgactctagtttgacattgtga

SEQ ID NO: 40 - DNA, 60 bp, synthetic construct
  source 1..60: mol_type = other DNA
gttgagcttgactctagtttgacattgtgaaaagacataggaggtgtagaataggtggga

SEQ ID NO: 41 - skipped

SEQ ID NO: 42 - DNA, 15 bp, synthetic construct
  source 1..15: mol_type = other DNA
gagggcaagtctggt

SEQ ID NO: 43 - DNA, 30 bp, synthetic construct
  source 1..30: mol_type = other DNA
aacgaggaacaattggagggcaagtctggt

SEQ ID NO: 44 - DNA, 60 bp, synthetic construct
  source 1..60: mol_type = other DNA
taattggaatgagaacaatctaaatcccttaacgaggaacaattggagggcaagtctggt

SEQ ID NO: 45 - skipped

SEQ ID NO: 46 - DNA, 15 bp, synthetic construct
  source 1..15: mol_type = other DNA
ccagcagccgcggta

SEQ ID NO: 47 - DNA, 30 bp, synthetic construct
  source 1..30: mol_type = other DNA
ccagcagccgcggtaattccagctccaata

SEQ ID NO: 48 - DNA, 60 bp, synthetic construct
  source 1..60: mol_type = other DNA
ccagcagccgcggtaattccagctccaatagcgtatattaaagttgttgcagttaaaaag

SEQ ID NO: 49 - DNA, 333 bp, synthetic construct
  source 1..333: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataaaaaaaaaaaaaaaaacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 50 - DNA, 343 bp, synthetic construct
  source 1..343: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataaaaaaaaaaaaaaaaaaaaaaaaaaacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 51 - DNA, 353 bp, synthetic construct
  source 1..353: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataaaaaaaaaaaaaaaaaaaaaaaaaaaaaaaaaaaaacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 52 - DNA, 333 bp, synthetic construct
  source 1..333: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 53 - DNA, 343 bp, synthetic construct
  source 1..343: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 54 - DNA, 353 bp, synthetic construct
  source 1..353: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 55 - RNA, 534 nt, synthetic construct
  source 1..534: mol_type = other RNA
aagtcgtaacaaggttgcctgtcgatgagcacacacgggagagccagccccttccgcagccaccgacacgggagcctgagcgggcccaccgggctgggcccgctggtccgacgacgcgcaaggggccggggtacggcgggctcgcgcccgtcctccccggcgccgccgtcgggcgacaccgtccaagtgcggagaccttcccagggcacacgctaccaagcgggagaggcgaggggagcggtcggctctccccgtcctccccgtggcccggccggaacgaggccggggtacggtcacaacgcgtgcgccgagctcaggggaacgatccgcagccaacccgggaccggccgccctcggccggtcgcggggcagtccacagactgaacgacggtgggtcggccgctcgagcccgagcggccggcctaagatgcagtcggcgagcgggggagacctcgctccgtccgcatagcagcacgcagcgcttcaccgttccgtaggtgaaccacaaaaaaaaaaccaaaaaaacaaaacaca

SEQ ID NO: 56 - RNA, 469 nt, synthetic construct
  source 1..469: mol_type = other RNA
gagggcaagtctggtgaaccatcagttctctgctgacgccagagatagtagggtcccccccggaggaccgggggggattacgcctgctagtcgagcactcagtgtggcgatgggtgagtgccggcgaggtgacctggtacggggaagcctaagggttgtcggccgcgaggggggcccgttcgacctatggtaatcccgtggcgagctccagccgggcggaagcccgctggggccgtcgtaacgcgcggaaaggcaccggctgccacctccgggtggtggctcaagggacgtgctgtcccatccgaaaggatgccctcaactggagcacccatcgtgtaaaggttgaggggggcacgcggcgcggcgccgtgtgcccgggagaccctccccccgcgggttcccgcgggcgggggggctgttaacggccagcagccgcggtacaaaaaaaaaaccaaaaaaacaaaacaca

SEQ ID NO: 57 - RNA, 411 nt, synthetic construct
  source 1..411: mol_type = other RNA
ggaaagaagaccctgagagcagctcaacggggtccaaaatagcgcacatcgctattagtccagcggccgaccatctgcaggtggccgcgggcgagacaacctggtacggggaagcctaagggcgccgagaaggcgcctacggtgatcccgtggcgagccccctccttgcactacagtgcagcgggggccgtcgtaacgcgcggtaaggtgtcggtcgcgggggtgggggagttcgcccagcgaggcccccccagcccctccggcttaaggtacgtgctaaacccaggcgaaagcctgcgcccagtcggagtgcctgcagcacgaactctgggcgggtggccggggcgatgcgtcgatcgatcgagagagcgcggccgcccgccggtagttgaaaatggttgagcttgactc

SEQ ID NO: 58 - RNA, 425 nt, synthetic construct
  source 1..425: mol_type = other RNA
gagggcaagtctggtggagaatcagtttcccaaactgcttgacgccagagatagtagggcccttccccggcgggaagggttaagcctgctagtcgagcactcagcgtggcgatgggtgagtgccggcgaggtacctggtacggggaagcctaagggttcggtcgcgagggaggccggtgtaacctatggtaatcccgtggcgagctccggggggcgtaggcccgctggggccgtcgtaacgcgcggaaaggcaccggccaccacctccggggtggtggctcaagggacgtgctgtcccatccgaaaggatgcccctaactggaacacccatcgtgcaaaggttgggggaggtttggccgcccgtccgggcggtcatgcctgggtgactcactaaagtgatttttctaacggccagcagccgcggt

SEQ ID NO: 59 - RNA, 430 nt, synthetic construct
  source 1..430: mol_type = other RNA
gggaaagaagaccctgaacgcgactgcggttcgttccacgacaagcatcgacggggtcaagagcggtcacggactgagcctcagtccgggtgccgttagtccaatgcggccgcagcatctgtgggtggccgcgggcgagacaacctggaacgggggagcctgacggctggagcctgcgggcccagcacggtaatcccgtggcgagtccctctcggggggccgtcgtaacgcgcgccaaggtgtcggttggtggcctcccggccgccggcttaaggtacgtgctaatcccaggcgaaagcctgcccggagctggagcgcccacagcgcaaaggcgtcgggagccgcaagtgacgtctaggaggaaatgcctggacggaggctggtaggggccgtcgcgcgcccgcgcgacggcatggttgagcttgactct

SEQ ID NO: 60 - RNA, 378 nt, synthetic construct
  source 1..378: mol_type = other RNA
gagggcaagtctggtggacattcagttccaacatgctgacgccagagatagtgggcttcacgattccccccctggggaggtgaagtacgcccgctagtcgagcactcgcagtcgcgatgggtgagtgccggcgaggtgacctggtacggggaagcctacggcctgatgtctcaggctatggtaatcccgtggcgagcccccttccggggccgtcgtaacgcgcgcaaaggcaccggtcgtccgcccccgggcgacgactcaagggacgtgctgtcccatccgaaaggatgcccgccaccggaacacccatcgtgtaaaggtgacggggggcgtggggaagcccacgcccgggtgatgaaaacggccagcagccgcggt

SEQ ID NO: 61 - RNA, 407 nt, synthetic construct
  source 1..407: mol_type = other RNA
ggaaagaagaccctgaacgatgacgcatcaacggggtcagtagcggtcagcgtgccgctagtccagtcggccagcatctgtgggtggccacgggcgagacaacctggtacgggggagcctacgggggaggactcgtcctccctacggtaatcccgtggcgacctttccactggacttccactggagaggcgtcgtaacgcgcggaaaggtgtcggttggcggtcgcgaggccgccggcttaagggacgtgctaaaccctggcgaaagccagcccgccgacggagcgcccccagcgcaaagtcgtcgggggtcgtacttacaccacgcctgggaggaaatgccctggcggagaccggtagcctctgctggcctgcaaaggccgcgggggaattggttgagcttgactc

SEQ ID NO: 62 - RNA, 450 nt, synthetic construct
  source 1..450: mol_type = other RNA
gagggcaagtctggtgaaccatcagttccacaatgctgacgccagagatagtagggccctctctccggggagggttatgcctgctagtcgagcactcgcaatcgcgatgggtgagtgccggggaggtgacctggtacggggaagcctaagggtgcggcccacaagggaggccagtgacatctatggtaatcccgtggcgagcctgtttgcaggccgtcgtaacgcgcggaaaggcaccggtcactacaggttagtggctcaagggacgtgctgtcccatccgaaaggatgccttcaactggagcacccatcgtgtaaaggttgaaggaggcttacagcaggccgctgtaagcctgggagttcttattgtggcatggtgaagtccactcggcattcataatgtcgtccccatgtcagtcgatgagtgtataaaacggccagcagccgcggt

SEQ ID NO: 63 - skipped

SEQ ID NO: 64 - DNA, 15 bp, synthetic construct
  source 1..15: mol_type = other DNA
agacgctacggactt

SEQ ID NO: 65 - DNA, 30 bp, synthetic construct
  source 1..30: mol_type = other DNA
gtgtggcggaatggtagacgctacggactt

SEQ ID NO: 66 - DNA, 60 bp, synthetic construct
  source 1..60: mol_type = other DNA
aatatagcaagctcccaagacaacgggggggtgtggcggaatggtagacgctacggactt

SEQ ID NO: 67 - DNA, 15 bp, synthetic construct
  source 1..15: mol_type = other DNA
aaatccgttgacctt

SEQ ID NO: 68 - DNA, 1821 bp, synthetic construct
  source 1..1821: mol_type = other DNA
gggggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtaaaaaacaaaaaacaaaacggctattatgcgttaccggcgagacgctacggactt

SEQ ID NO: 69 - DNA, 156 bp, synthetic construct
  source 1..156: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagttt

SEQ ID NO: 70 - DNA, 1906 bp, synthetic construct
  source 1..1906: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtaaaaaacaaaaaacaaaacggctattatgcgttaccggcgagacgctacggactt

SEQ ID NO: 71 - DNA, 1975 bp, synthetic construct
  source 1..1975: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtaaaaaacaaaaaacaaaacggctattatgcgttaccggcgagacgctacggactt

SEQ ID NO: 72 - DNA, 1975 bp, synthetic construct
  source 1..1975: mol_type = other DNA
gggaagcggtgcatcccactaaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtaaaaaacaaaaaacaaaacggctattatgcgttaccggcgagacgctacggactt

SEQ ID NO: 73 - DNA, 249 bp, synthetic construct
  source 1..249: mol_type = other DNA
aaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 74 - DNA, 19 bp, synthetic construct
  source 1..19: mol_type = other DNA
gccggaaacgcaatagccg

SEQ ID NO: 75 - DNA, 19 bp, synthetic construct
  source 1..19: mol_type = other DNA
gctattatgcgttaccggc

SEQ ID NO: 76 - DNA, 11 bp, synthetic construct
  source 1..11: mol_type = other DNA
gctacggactt

SEQ ID NO: 77 - DNA, 30 bp, synthetic construct
  source 1..30: mol_type = other DNA
gtgtggcggaatgggagacgctacggactt

SEQ ID NO: 78 - DNA, 11 bp, synthetic construct
  source 1..11: mol_type = other DNA
aaaatccgttg

SEQ ID NO: 79 - DNA, 16 bp, synthetic construct
  source 1..16: mol_type = other DNA
aaaatccgttgacctt

SEQ ID NO: 80 - DNA, 30 bp, synthetic construct
  source 1..30: mol_type = other DNA
aaaatccgttgaccttaaacggtcgtgtgg

SEQ ID NO: 81 - DNA, 19 bp, synthetic construct
  source 1..19: mol_type = other DNA
taagcacaggagtgtcatg

SEQ ID NO: 82 - DNA, 19 bp, synthetic construct
  source 1..19: mol_type = other DNA
tttaatttaataaaatttt

SEQ ID NO: 83 - DNA, 19 bp, synthetic construct
  source 1..19: mol_type = other DNA
gccgatataatttattaaa

SEQ ID NO: 84 - DNA, 19 bp, synthetic construct
  source 1..19: mol_type = other DNA
gccgtaataattaataccg

SEQ ID NO: 85 - DNA, 19 bp, synthetic construct
  source 1..19: mol_type = other DNA
gccgtgtatagtgaacccg

SEQ ID NO: 86 - DNA, 19 bp, synthetic construct
  source 1..19: mol_type = other DNA
gccgagcaagcgctggccg

SEQ ID NO: 87 - DNA, 19 bp, synthetic construct
  source 1..19: mol_type = other DNA
gccggcccgggcggggccg

SEQ ID NO: 88 - DNA, 19 bp, synthetic construct
  source 1..19: mol_type = other DNA
ttactgcagggatccaagt

SEQ ID NO: 89 - DNA, 19 bp, synthetic construct
  source 1..19: mol_type = other DNA
aaaattttattaaattaaa

SEQ ID NO: 90 - DNA, 19 bp, synthetic construct
  source 1..19: mol_type = other DNA
tttaataaattatatcggc

SEQ ID NO: 91 - DNA, 19 bp, synthetic construct
  source 1..19: mol_type = other DNA
cggtattaattattacggc

SEQ ID NO: 92 - DNA, 19 bp, synthetic construct
  source 1..19: mol_type = other DNA
cgggttcactatacacggc

SEQ ID NO: 93 - DNA, 19 bp, synthetic construct
  source 1..19: mol_type = other DNA
cggccagcgcttgctcggc

SEQ ID NO: 94 - DNA, 19 bp, synthetic construct
  source 1..19: mol_type = other DNA
cggccccgcccgggccggc

SEQ ID NO: 95 - DNA, 14 bp, synthetic construct
  source 1..14: mol_type = other DNA
gccgaaggctgccg

SEQ ID NO: 96 - DNA, 19 bp, synthetic construct
  source 1..19: mol_type = other DNA
gccgaaggcctagactccg

SEQ ID NO: 97 - DNA, 24 bp, synthetic construct
  source 1..24: mol_type = other DNA
gccgacgaaagccaaagcctcccg

SEQ ID NO: 98 - DNA, 29 bp, synthetic construct
  source 1..29: mol_type = other DNA
gccgtgaagtcgagacttaaccacagccg

SEQ ID NO: 99 - DNA, 34 bp, synthetic construct
  source 1..34: mol_type = other DNA
gccgtcaatattggagttcgtgaccgacgtgccg

SEQ ID NO: 100 - DNA, 14 bp, synthetic construct
  source 1..14: mol_type = other DNA
cggcagccttcggc

SEQ ID NO: 101 - DNA, 19 bp, synthetic construct
  source 1..19: mol_type = other DNA
cggagtctaggccttcggc

SEQ ID NO: 102 - DNA, 24 bp, synthetic construct
  source 1..24: mol_type = other DNA
cgggaggctttggctttcgtcggc

SEQ ID NO: 103 - DNA, 29 bp, synthetic construct
  source 1..29: mol_type = other DNA
cggctgtggttaagtctcgacttcacggc

SEQ ID NO: 104 - DNA, 34 bp, synthetic construct
  source 1..34: mol_type = other DNA
cggcacgtcggtcacgaactccaatattgacggc

SEQ ID NO: 105 - DNA, 10 bp, synthetic construct
  source 1..10: mol_type = other DNA
aaaaacaaaa

SEQ ID NO: 106 - DNA, 20 bp, synthetic construct
  source 1..20: mol_type = other DNA
aaaaacaaaaaacaaaaaaa

SEQ ID NO: 107 - DNA, 30 bp, synthetic construct
  source 1..30: mol_type = other DNA
aaaaacaaaaaacaaaaaaaaacaaaaaaa

SEQ ID NO: 108 - DNA, 10 bp, synthetic construct
  source 1..10: mol_type = other DNA
aaaaaacaaa

SEQ ID NO: 109 - DNA, 20 bp, synthetic construct
  source 1..20: mol_type = other DNA
aaaaaacaaaaaacaaaaca

SEQ ID NO: 110 - DNA, 30 bp, synthetic construct
  source 1..30: mol_type = other DNA
aaaaaacaaaaaacaaaacaaacaaaaaaa

SEQ ID NO: 111 - DNA, 1969 bp, synthetic construct
  source 1..1969: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaagagacgctacggactt

SEQ ID NO: 112 - DNA, 1999 bp, synthetic construct
  source 1..1999: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtcaatattggagttcgtgaccgacgtgccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacggcacgtcggtcacgaactccaatattgacggcaaaaaacaaaaaacaaaagagacgctacggactt

SEQ ID NO: 113 - DNA, 1839 bp, synthetic construct
  source 1..1839: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacgccggaaacgcaatagccgaaaaacaaaaaacaaaaaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaccggtgctggagcctcggtggccatgcttcttgccccttgggcctccccccagcccctcctccccttcctgcacccgtacccccgtggtctttgaataaagtctgaaaaaaacaaaaaacaaaacggctattatgcgttaccggcgagacgctacggactt

SEQ ID NO: 114 - DNA, 1796 bp, synthetic construct
  source 1..1796: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaagccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaagctggagcctcggtggccatgcttcttgccccttgggcctccccccagcccctcctccccttcctgcacccgtacccccgtggtctttgaataaagtctgacgggttcactatacacggcaaaaaacaaaaaacaaaagagacgctacggactt

SEQ ID NO: 115 - DNA, 1837 bp, synthetic construct
  source 1..1837: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtcaatattggagttcgtgaccgacgtgccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatgggagtcaaagttctgtttgccctgatctgcatcgctgtggccgaggccaagcccaccgagaacaacgaagacttcaacatcgtggccgtggccagcaacttcgcgaccacggatctcgatgctgaccgcgggaagttgcccggcaagaagctgccgctggaggtgctcaaagagatggaagccaatgcccggaaagctggctgcaccaggggctgtctgatctgcctgtcccacatcaagtgcacgcccaagatgaagaagttcatcccaggacgctgccacacctacgaaggcgacaaagagtccgcacagggcggcataggcgaggcgatcgtcgacattcctgagattcctgggttcaaggacttggagcccatggagcagttcatcgcacaggtcgatctgtgtgtggactgcacaactggctgcctcaaagggcttgccaacgtgcagtgttctgacctgctcaagaagtggctgccgcaacgctgtgcgacctttgccagcaagatccagggccaggtggacaagatcaagggggccggtggtgactaaccggtgctggagcctcggtggccatgcttcttgccccttgggcctccccccagcccctcctccccttcctgcacccgtacccccgtggtctttgaataaagtctgacggcacgtcggtcacgaactccaatattgacggcaaaaaacaaaaaacaaaagagacgctacggactt

SEQ ID NO: 116 - DNA, 318 bp, synthetic construct
  source 1..318: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 117 - DNA, 352 bp, synthetic construct
  source 1..352: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 118 - DNA, 363 bp, synthetic construct
  source 1..363: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 119 - DNA, 373 bp, synthetic construct
  source 1..373: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 120 - DNA, 383 bp, synthetic construct
  source 1..383: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 121 - DNA, 393 bp, synthetic construct
  source 1..393: mol_type = other DNA
aaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatg

SEQ ID NO: 122 - DNA, 1805 bp, synthetic construct
  source 1..1805: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtttgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 123 - DNA, 1820 bp, synthetic construct
  source 1..1820: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 124 - DNA, 1830 bp, synthetic construct
  source 1..1830: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 125 - DNA, 1839 bp, synthetic construct
  source 1..1839: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 126 - DNA, 1850 bp, synthetic construct
  source 1..1850: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 127 - DNA, 1860 bp, synthetic construct
  source 1..1860: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 128 - DNA, 1870 bp, synthetic construct
  source 1..1870: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 129 - DNA, 1880 bp, synthetic construct
  source 1..1880: mol_type = other DNA
ggaaataattgagccttaaagaagaaattctttaagtggatgctctcaaactcagggaaacctaaatctagttatagacaaggcaatcctgagccaagccgaagtagtaattagtaagaccagtggacaatcgacggataacagcatatctagtaaaaaaaaaaaaaaaaaaaaaaaaaaaaaaaaaaaaaaaaaaaaaaaaaaaaaaaaaaaaaaaaaaaaaaaaaaattgggagaccctcgaccgtcgattgtccactggtcaacaatagatgacttacaactaatcggaaggtgcagagactcgacgggagctaccctaacgtcaagacgagggtaaagagagagtccaattctcaaagccaataggcagtagcgaaagctgcaagagaatgaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 130 - DNA, 2113 bp, synthetic construct
  source 1..2113: mol_type = other DNA
ggtgcctgtcgatgagcacacacgggagagccagccccttccgcagccaccgacacgggagcctgagcgggcccaccgggctgggcccgctggtccgacgacgcgcaaggggccggggtacggcgggctcgcgcccgtcctccccggcgccgccgtcgggcgacaccgtccaagtgcggagaccttcccagggcacacgctaccaagcgggagaggcgaggggagcggtcggctctccccgtcctccccgtggcccggccggaacgaggccggggtacggtcacaacgcgtgcgccgagctcaggggaacgatccgcagccaacccgggaccggccgccctcggccggtcgcggggcagtccacagactgaacgacggtgggtcggccgctcgagcccgagcggccggcctaagatgcagtcggcgagcgggggagacctcgctccgtccgcatagcagcacgcagcgcttcaccgttccgtaggtgaaccaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaaaagtcgtaacaaggt

SEQ ID NO: 131 - DNA, 2047 bp, synthetic construct
  source 1..2047: mol_type = other DNA
ggaaccatcagttctctgctgacgccagagatagtagggtcccccccggaggaccgggggggattacgcctgctagtcgagcactcagtgtggcgatgggtgagtgccggcgaggtgacctggtacggggaagcctaagggttgtcggccgcgaggggggcccgttcgacctatggtaatcccgtggcgagctccagccgggcggaagcccgctggggccgtcgtaacgcgcggaaaggcaccggctgccacctccgggtggtggctcaagggacgtgctgtcccatccgaaaggatgccctcaactggagcacccatcgtgtaaaggttgaggggggcacgcggcgcggcgccgtgtgcccgggagaccctccccccgcgggttcccgcgggcgggggggctgttaacggccagcagccgcggtaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaagagggcaagtctggt

SEQ ID NO: 132 - DNA, 2032 bp, synthetic construct
  source 1..2032: mol_type = other DNA
ggagaatcagtttcccaaactgcttgacgccagagatagtagggcccttccccggcgggaagggttaagcctgctagtcgagcactcagcgtggcgatgggtgagtgccggcgaggtacctggtacggggaagcctaagggttcggtcgcgagggaggccggtgtaacctatggtaatcccgtggcgagctccggggggcgtaggcccgctggggccgtcgtaacgcgcggaaaggcaccggccaccacctccggggtggtggctcaagggacgtgctgtcccatccgaaaggatgcccctaactggaacacccatcgtgcaaaggttgggggaggtttggccgcccgtccgggcggtcatgcctgggtgactcactaaagtgatttttctaacggccagcagccgcggtaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaagagggcaagtctggt

SEQ ID NO: 133 - DNA, 2058 bp, synthetic construct
  source 1..2058: mol_type = other DNA
ggaaccatcagttccacaatgctgacgccagagatagtagggccctctctccggggagggttatgcctgctagtcgagcactcgcaatcgcgatgggtgagtgccggggaggtgacctggtacggggaagcctaagggtgcggcccacaagggaggccagtgacatctatggtaatcccgtggcgagcctgtttgcaggccgtcgtaacgcgcggaaaggcaccggtcactacaggttagtggctcaagggacgtgctgtcccatccgaaaggatgccttcaactggagcacccatcgtgtaaaggttgaaggaggcttacagcaggccgctgtaagcctgggagttcttattgtggcatggtgaagtccactcggcattcataatgtcgtccccatgtcagtcgatgagtgtataaaacggccagcagccgcggtaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaagagggcaagtctggt

SEQ ID NO: 134 - DNA, 2015 bp, synthetic construct
  source 1..2015: mol_type = other DNA
ggaacgatgacgcatcaacggggtcagtagcggtcagcgtgccgctagtccagtcggccagcatctgtgggtggccacgggcgagacaacctggtacgggggagcctacgggggaggactcgtcctccctacggtaatcccgtggcgacctttccactggacttccactggagaggcgtcgtaacgcgcggaaaggtgtcggttggcggtcgcgaggccgccggcttaagggacgtgctaaaccctggcgaaagccagcccgccgacggagcgcccccagcgcaaagtcgtcgggggtcgtacttacaccacgcctgggaggaaatgccctggcggagaccggtagcctctgctggcctgcaaaggccgcgggggaattggttgagcttgactcaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaaggaaagaagaccct

SEQ ID NO: 135 - DNA, 2038 bp, synthetic construct
  source 1..2038: mol_type = other DNA
ggaacgcgactgcggttcgttccacgacaagcatcgacggggtcaagagcggtcacggactgagcctcagtccgggtgccgttagtccaatgcggccgcagcatctgtgggtggccgcgggcgagacaacctggaacgggggagcctgacggctggagcctgcgggcccagcacggtaatcccgtggcgagtccctctcggggggccgtcgtaacgcgcgccaaggtgtcggttggtggcctcccggccgccggcttaaggtacgtgctaatcccaggcgaaagcctgcccggagctggagcgcccacagcgcaaaggcgtcgggagccgcaagtgacgtctaggaggaaatgcctggacggaggctggtaggggccgtcgcgcgcccgcgcgacggcatggttgagcttgactctaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaagggaaagaagaccct

SEQ ID NO: 136 - DNA, 2019 bp, synthetic construct
  source 1..2019: mol_type = other DNA
ggagagcagctcaacggggtccaaaatagcgcacatcgctattagtccagcggccgaccatctgcaggtggccgcgggcgagacaacctggtacggggaagcctaagggcgccgagaaggcgcctacggtgatcccgtggcgagccccctccttgcactacagtgcagcgggggccgtcgtaacgcgcggtaaggtgtcggtcgcgggggtgggggagttcgcccagcgaggcccccccagcccctccggcttaaggtacgtgctaaacccaggcgaaagcctgcgcccagtcggagtgcctgcagcacgaactctgggcgggtggccggggcgatgcgtcgatcgatcgagagagcgcggccgcccgccggtagttgaaaatggttgagcttgactcaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaaggaaagaagaccct

SEQ ID NO: 137 - DNA, 1986 bp, synthetic construct
  source 1..1986: mol_type = other DNA
gggacattcagttccaacatgctgacgccagagatagtgggcttcacgattccccccctggggaggtgaagtacgcccgctagtcgagcactcgcagtcgcgatgggtgagtgccggcgaggtgacctggtacggggaagcctacggcctgatgtctcaggctatggtaatcccgtggcgagcccccttccggggccgtcgtaacgcgcgcaaaggcaccggtcgtccgcccccgggcgacgactcaagggacgtgctgtcccatccgaaaggatgcccgccaccggaacacccatcgtgtaaaggtgacggggggcgtggggaagcccacgcccgggtgatgaaaacggccagcagccgcggtaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaagagggcaagtctggt

SEQ ID NO: 138 - DNA, 1888 bp, synthetic construct
  source 1..1888: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgacctaaaaacaaaaaacaaaaaaagccggcccgggcggggccgaaaaaaaaaaaaccaaaaaaaaaaaacaaaaaaaaaaaataattgactaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaacaaaaaacaaaaaaaagacgctacggactt

SEQ ID NO: 139 - DNA, 1868 bp, synthetic construct
  source 1..1868: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgacctttaagcacaggagtgtcatggccggcccgggcggggccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcttactgcagggatccaagtgagacgctacggactt

SEQ ID NO: 140 - DNA, 1868 bp, synthetic construct
  source 1..1868: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccggcccgggcggggccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaaacaaaaaacaaaagagacgctacggactt

SEQ ID NO: 141 - DNA, 1848 bp, synthetic construct
  source 1..1848: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatcaaaaacaaaaaacaaaaaaagccggcccgggcggggccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggccccgcccgggccggcaaaaaacaaaaaacaaaaggactt

SEQ ID NO: 142 - DNA, 1766 bp, synthetic construct
  source 1..1766: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtctt

SEQ ID NO: 143 - DNA, 1804 bp, synthetic construct
  source 1..1804: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaataagcacaggagtgtcatgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtttactgcagggatccaagtctt

SEQ ID NO: 144 - DNA, 1822 bp, synthetic construct
  source 1..1822: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaaaaaacaaaaaacaaaaaaagccgacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcgggtcggcaaaaaacaaaaaacaaaactt

SEQ ID NO: 145 - DNA, 1935 bp, synthetic construct
  source 1..1935: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaacggtcgtgtgggttcaagtccctccacccccacaaaaacaaaaaacaaaaaaagccgtcaatattggagttcgtgaccgacgtgccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggcacgtcggtcacgaactccaatattgacggcaaaaaacaaaaaacaaaacgagacgctacggactt

SEQ ID NO: 146 - DNA, 1899 bp, synthetic construct
  source 1..1899: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtcaatattggagttcgtgaccgacgtgccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggcacgtcggtcacgaactccaatattgacggcaaaaaacaaaaaacaaaacgagacgctacggactt

SEQ ID NO: 147 - DNA, 1898 bp, synthetic construct
  source 1..1898: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgacctttaagcacaggagtgtcatggccgtcaatattggagttcgtgaccgacgtgccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtcggcacgtcggtcacgaactccaatattgacggcttactgcagggatccaagtgagacgctacggactt

SEQ ID NO: 148 - DNA, 474 bp, synthetic construct
  source 1..474: mol_type = other DNA
tgcctgtcgatgagcacacacgggagagccagccccttccgcagccaccgacacgggagcctgagcgggcccaccgggctgggcccgctggtccgacgacgcgcaaggggccggggtacggcgggctcgcgcccgtcctccccggcgccgccgtcgggcgacaccgtccaagtgcggagaccttcccagggcacacgctaccaagcgggagaggcgaggggagcggtcggctctccccgtcctccccgtggcccggccggaacgaggccggggtacggtcacaacgcgtgcgccgagctcaggggaacgatccgcagccaacccgggaccggccgccctcggccggtcgcggggcagtccacagactgaacgacggtgggtcggccgctcgagcccgagcggccggcctaagatgcagtcggcgagcgggggagacctcgctccgtccgcatagcagcacgcagcgcttcaccg

SEQ ID NO: 149 - DNA, 395 bp, synthetic construct
  source 1..395: mol_type = other DNA
ggagaatcagtttcccaaactgcttgacgccagagatagtagggcccttccccggcgggaagggttaagcctgctagtcgagcactcagcgtggcgatgggtgagtgccggcgaggtacctggtacggggaagcctaagggttcggtcgcgagggaggccggtgtaacctatggtaatcccgtggcgagctccggggggcgtaggcccgctggggccgtcgtaacgcgcggaaaggcaccggccaccacctccggggtggtggctcaagggacgtgctgtcccatccgaaaggatgcccctaactggaacacccatcgtgcaaaggttgggggaggtttggccgcccgtccgggcggtcatgcctgggtgactcactaaagtgatttttctaacg

SEQ ID NO: 150 - DNA, 420 bp, synthetic construct
  source 1..420: mol_type = other DNA
gaaccatcagttccacaatgctgacgccagagatagtagggccctctctccggggagggttatgcctgctagtcgagcactcgcaatcgcgatgggtgagtgccggggaggtgacctggtacggggaagcctaagggtgcggcccacaagggaggccagtgacatctatggtaatcccgtggcgagcctgtttgcaggccgtcgtaacgcgcggaaaggcaccggtcactacaggttagtggctcaagggacgtgctgtcccatccgaaaggatgccttcaactggagcacccatcgtgtaaaggttgaaggaggcttacagcaggccgctgtaagcctgggagttcttattgtggcatggtgaagtccactcggcattcataatgtcgtccccatgtcagtcgatgagtgtataaaacg

SEQ ID NO: 151 - DNA, 379 bp, synthetic construct
  source 1..379: mol_type = other DNA
gaacgatgacgcatcaacggggtcagtagcggtcagcgtgccgctagtccagtcggccagcatctgtgggtggccacgggcgagacaacctggtacgggggagcctacgggggaggactcgtcctccctacggtaatcccgtggcgacctttccactggacttccactggagaggcgtcgtaacgcgcggaaaggtgtcggttggcggtcgcgaggccgccggcttaagggacgtgctaaaccctggcgaaagccagcccgccgacggagcgcccccagcgcaaagtcgtcgggggtcgtacttacaccacgcctgggaggaaatgccctggcggagaccggtagcctctgctggcctgcaaaggccgcgggggaattg

SEQ ID NO: 152 - DNA, 401 bp, synthetic construct
  source 1..401: mol_type = other DNA
ggaacgcgactgcggttcgttccacgacaagcatcgacggggtcaagagcggtcacggactgagcctcagtccgggtgccgttagtccaatgcggccgcagcatctgtgggtggccgcgggcgagacaacctggaacgggggagcctgacggctggagcctgcgggcccagcacggtaatcccgtggcgagtccctctcggggggccgtcgtaacgcgcgccaaggtgtcggttggtggcctcccggccgccggcttaaggtacgtgctaatcccaggcgaaagcctgcccggagctggagcgcccacagcgcaaaggcgtcgggagccgcaagtgacgtctaggaggaaatgcctggacggaggctggtaggggccgtcgcgcgcccgcgcgacggcatg

SEQ ID NO: 153 - DNA, 383 bp, synthetic construct
  source 1..383: mol_type = other DNA
gagagcagctcaacggggtccaaaatagcgcacatcgctattagtccagcggccgaccatctgcaggtggccgcgggcgagacaacctggtacggggaagcctaagggcgccgagaaggcgcctacggtgatcccgtggcgagccccctccttgcactacagtgcagcgggggccgtcgtaacgcgcggtaaggtgtcggtcgcgggggtgggggagttcgcccagcgaggcccccccagcccctccggcttaaggtacgtgctaaacccaggcgaaagcctgcgcccagtcggagtgcctgcagcacgaactctgggcgggtggccggggcgatgcgtcgatcgatcgagagagcgcggccgcccgccggtagttgaaaatg

SEQ ID NO: 154 - DNA, 395 bp, Coelastrella multistriata
  source 1..395: mol_type = unassigned DNA
gacattcaacccatgctgttgacgccagagataagtagggcagttgctttttttggcaattgttatgcctgctagtcgagcaatcattcaattgaacgggctgattgccggcaagacaacctggtacggggaaggccttcacaactatggggctaatcccgtggcgagcttgcaaagggtgacttttgtaaagccgtcgtaacgcacgctaaggtgtcggctgactcagtcagagttggcttaagggacgtgctaaccccacccgatgataaagggtgtccaatgcaatagcacccgttctgcaaaggcattggagggctacagtgtaaggaggaaatgccgtacactgcccggtacaaatcaactgggattccatgtcgaacccagtgaaagtg

SEQ ID NO: 155 - DNA, 463 bp, Trebouxia arboricola
  source 1..463: mol_type = unassigned DNA
gaggtgacatttactatgcggataaaagcatgccttaaaagggtagtgctagtacttatgtctgtcactagactgagtgcgacaccgtcaaactgcctgaaagtcctgttaagcctctgataccgcacagcatgggaaactatgttgctgcacctcggtaaaagtcgagggcatggtaaaaactcagaaggatagggataatgggcagccaagtgctaaaggaatcatttccaatgcatgcagttcacagactaaatggcggtgggttggaatgcaaaatcaaaaatacacagaggattttgccttttggcttaagatatagtcggtcctcgccgaaaggcggcctgcaggaggaatttccagtcggaaaggagagcctgcaggagtttggataatactctgcattgcaaaatgtggtagggcttgcatcaatttgattcgagcctctaaactggaataaacg

SEQ ID NO: 156 - DNA, 453 bp, Trebouxia sp.
  source 1..453: mol_type = unassigned DNA
gaggtactttattcactatgcggacaaaagcaggcctgaaaagggttccctgctagtgcttgcctttccagcagtcaagcgcgacatcgtcaaattgcctgaaagtcctgctaagcctctgataccgcactgcaagggaaaccttgtctttgcactccggtgaaagtcgggggtatggtaaaaattcagttggatagggataatgggcagccaagtgctaaaggaaattacttcccatgcatgcagttcacagactaaatggcgatgggttgaaatggtgatcactattgattgccttttggcttaagatatagtcggtcctcgctgaaaaggcgacctgtgggaggatttccctttcgggaaggagagcttacaggagtttagataatactcttcattgtaaaatgttagagggcttgaccagtggtttttgcctctaaactggattaaacg

SEQ ID NO: 157 - DNA, 332 bp, Hypocrea pallida
  source 1..332: mol_type = unassigned DNA
gaaacgttcgctggtgccagaaatagccagccctgtaaaatggctagtcgagtgctcgatctgtagcgggtgagcaccggcgagacaacctggatcggggaagcctaagggacctgtcacagggtcctatggtgatcccgagcagagccgcaaggcctgtgtagagcacgctaaggtgtcggtcccgtcctctgcggcggggcttaaggtacgtgccaatcccctgcgaaagctgggccctagggagtagcacccgctgtgcgaagctctaggggacctgtgcccgccgtggcgcaggccgtccgacagtcgtcagactgctcactataatg

SEQ ID NO: 158 - DNA, 413 bp, Tetrahymena thermophila
  source 1..413: mol_type = unassigned DNA
aaatagcaatatttacctttggagggaaaagttatcaggcatgcacctggtagctagtctttaaaccaatagattgcatcggtttaaaaggcaagaccgtcaaattgcgggaaaggggtcaacagccgttcagtaccaagtctcaggggaaactttgagatggccttgcaaagggtatggtaataagctgacggacatggtcctaaccacgcagccaagtcctaagtcaacagatcttctgttgatatggatgcagttcacagactaaatgtcggtcggggaagatgtattcttctcataagatatagtcggacctctccttaatgggagctagcggatgaagtgatgcaacactggagccgctgggaactaatttgtatgcgaaagtatattgattagttttggagtactcg

SEQ ID NO: 159 - DNA, 205 bp, Azoarcus olearius
  source 1..205: mol_type = unassigned DNA
atttcgatgtgccttgcgccgggaaaccacgcaagggatggtgtcaaattcggcgaaacctaagcgcccgcccgggcgtatggcaacgccgagccaagcttcggcgcctgcgccgatgaaggtgtagagactagacggcacccacctaaggcaaacgctatggtgaaggcatagtccagggagtggcgaaagtcacacaaaccgg

SEQ ID NO: 160 - DNA, 14 bp, Coelastrella multistriata
  source 1..14: mol_type = unassigned DNA
agggcaagtctggt

SEQ ID NO: 161 - DNA, 15 bp, Trebouxia arboricola
  source 1..15: mol_type = unassigned DNA
aaacaaggtttccgt

SEQ ID NO: 162 - DNA, 15 bp, Trebouxia sp.
  source 1..15: mol_type = unassigned DNA
aaacaaggtttccgt

SEQ ID NO: 163 - DNA, 14 bp, Hypocrea pallida
  source 1..14: mol_type = unassigned DNA
agggcaagtctggt

SEQ ID NO: 164 - DNA, 15 bp, Tetrahymena thermophila
  source 1..15: mol_type = unassigned DNA
taactatgactctct

SEQ ID NO: 165 - DNA, 15 bp, Azoarcus olearius
  source 1..15: mol_type = unassigned DNA
gagcagcggactcat

SEQ ID NO: 166 - DNA, 10 bp, Trebouxia arboricola
  source 1..10: mol_type = unassigned DNA
agaaactgcg

SEQ ID NO: 167 - DNA, 11 bp, Trebouxia sp.
  source 1..11: mol_type = unassigned DNA
aggtgaacctg

SEQ ID NO: 168 - DNA, 12 bp, Hypocrea pallida
  source 1..12: mol_type = unassigned DNA
gccagcagccgc

SEQ ID NO: 169 - DNA, 15 bp, Tetrahymena thermophila
  source 1..15: mol_type = unassigned DNA
taaggtagccaaatg

SEQ ID NO: 170 - DNA, 15 bp, Azoarcus olearius
  source 1..15: mol_type = unassigned DNA
aatccgttggtgctg

SEQ ID NO: 171 - DNA, 2103 bp, Coelastrella multistriata
  source 1..2103: mol_type = unassigned DNA
ggacattcaacccatgctgttgacgccagagataagtagggcagttgctttttttggcaattgttatgcctgctagtcgagcaatcattcaattgaacgggctgattgccggcaagacaacctggtacggggaaggccttcacaactatggggctaatcccgtggcgagcttgcaaagggtgacttttgtaaagccgtcgtaacgcacgctaaggtgtcggctgactcagtcagagttggcttaagggacgtgctaaccccacccgatgataaagggtgtccaatgcaatagcacccgttctgcaaaggcattggagggctacagtgtaaggaggaaatgccgtacactgcccggtacaaatcaactgggattccatgtcgaacccagtgaaagtggccagcaaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaaagggcaagtctggt

SEQ ID NO: 172 - DNA, 2175 bp, Trebouxia arboricola
  source 1..2175: mol_type = unassigned DNA
ggaggtgacatttactatgcggataaaagcatgccttaaaagggtagtgctagtacttatgtctgtcactagactgagtgcgacaccgtcaaactgcctgaaagtcctgttaagcctctgataccgcacagcatgggaaactatgttgctgcacctcggtaaaagtcgagggcatggtaaaaactcagaaggatagggataatgggcagccaagtgctaaaggaatcatttccaatgcatgcagttcacagactaaatggcggtgggttggaatgcaaaatcaaaaatacacagaggattttgccttttggcttaagatatagtcggtcctcgccgaaaggcggcctgcaggaggaatttccagtcggaaaggagagcctgcaggagtttggataatactctgcattgcaaaatgtggtagggcttgcatcaatttgattcgagcctctaaactggaataaacgagaaactgcgaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaaaaacaaggtttccgt

SEQ ID NO: 173 - DNA, 2166 bp, Trebouxia sp.
  source 1..2166: mol_type = unassigned DNA
ggaggtactttattcactatgcggacaaaagcaggcctgaaaagggttccctgctagtgcttgcctttccagcagtcaagcgcgacatcgtcaaattgcctgaaagtcctgctaagcctctgataccgcactgcaagggaaaccttgtctttgcactccggtgaaagtcgggggtatggtaaaaattcagttggatagggataatgggcagccaagtgctaaaggaaattacttcccatgcatgcagttcacagactaaatggcgatgggttgaaatggtgatcactattgattgccttttggcttaagatatagtcggtcctcgctgaaaaggcgacctgtgggaggatttccctttcgggaaggagagcttacaggagtttagataatactcttcattgtaaaatgttagagggcttgaccagtggtttttgcctctaaactggattaaacgaggtgaacctgaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaaaaacaaggtttccgt

SEQ ID NO: 174 - DNA, 2045 bp, Hypocrea pallida
  source 1..2045: mol_type = unassigned DNA
ggaaacgttcgctggtgccagaaatagccagccctgtaaaatggctagtcgagtgctcgatctgtagcgggtgagcaccggcgagacaacctggatcggggaagcctaagggacctgtcacagggtcctatggtgatcccgagcagagccgcaaggcctgtgtagagcacgctaaggtgtcggtcccgtcctctgcggcggggcttaaggtacgtgccaatcccctgcgaaagctgggccctagggagtagcacccgctgtgcgaagctctaggggacctgtgcccgccgtggcgcaggccgtccgacagtcgtcagactgctcactataatggccagcagccgcaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaaagggcaagtctggt

SEQ ID NO: 175 - DNA, 949 bp, Bacillus thuringiensis
  source 1..949: mol_type = unassigned DNA
gcgcgacacgttcctaagtgaaaagcttaggcactgtcgaactcaacagttcagcagtgaactgtcattctaagaagtcaaatgaaggagtaacgtctggaagggcttcccttaatcctccgacatgcaggaaagtaggcaagtactgaactgtgtgaagctcggtgaagtcggttgaaggttaccgtaaattagtatctctaatacgaaagctatccagcggtggatggtgtaactgatagaccggaggtctataaaacactcaaggttaggatgcgcgatgaactagaggcgatcgctagtaagcgcagacgaatccctgatggtacgggtctatatcgggagggaatcgaaaggttctctgacacaaataagtgtcgctactgtgggtgagtaaaactctcctttatgaaagcccatatatcgttacaggcgttattaaggtagcaggctcataggggaaacctaaaagtgtatgtacagataagaatgacggaacgtggtaagctgccgacatggagggcttgttctctttgaagtgttgccaaggaaagtcacaatgagattagttgtcgatataacttggtttaacggcagtgaaagtggtggcacagtaccgatgaaacgtgtaatgaacgtggagggatagccactagtcgattgaagattgaaggttactattggttaacatggtttcgagtaagactaagagatgtaatgctccaaagtaataaggaggttacagcccatgttaaagaaaaccaagctaagacataacgaatattatgatacacaaaaaaagtgtatgacaatttatactcgaacagtcttaacggtaacaatttctttcaattggaaacgatggaacgccgtatgcccggaaacgggcgcgtacggtgtggagtgggggaaaagctggagataatctcaaaggcttacctatcactat

SEQ ID NO: 176 - DNA, 834 bp, Clostridium perfringens
  source 1..834: mol_type = unassigned DNA
gtgcgagacgttcatagttgaaaagactatgcacattctaaatataagagaatggagaactcttaacttgaaaagtggaatgatagggtaacgccttgaaacgctttctctaatactccgacatgcagtaactatgtgtgaaagttattgtgtgaagctcggtgaagtcggcagaaaattaccgtaatagataatattatctaacgaaagtgttctagaggtagaatatgtagttgatatgccgggagtcgttaaaaacagaatatggtgagaatgttccaataggaactgacgaactggcgaaggtacgctcctaagctataaatatagaaatatattaataaattggtttgaggattagtaagaatagtacccatgaaaatccttgatatgttataggcatataataagccaatggggtaaagtcagcacctaagttctagtattgataaagttaagggaacgtcgaaagctaaagacgtggaggatacacacctatgaagcgaagaaagggaatacataacctttcataatctttagtgagagtagtggcatgactgatgaagattttgtaatgaaattggaggaatagccacaagtcaattatttattaaaagaaacttaatcaattcatggattctagtaagataaaaaaggtcactgaatatagaaaattagtgatagaccatggcaagatatttatattaatacttgagattaagaaaaatgaatttaatatatacttgatggaacgccgtatgcggtgaaagtcgcacgtacggtgtggagcgggggaaaaattggagataacatcaaagatttacctatcgctat

SEQ ID NO: 177 - DNA, 633 bp, Anoxybacillus pushchinoensis
  source 1..633: mol_type = unassigned DNA
gtgcgctcggcatgggtgcaatctctagggtgaaagtcccgaactgcgaaggcagaagtagcagttagcttaacgcaagggtgtccgtggtgacgcggaatctgaaggaagcgggcggcaaacttccggtctgaggaacacgaacttcatataaggctaggtatcattggatgagtttgcaagacaaaacaaagtcctttctgccgaaggtgatacagagtaaatgaagcagatagatggaaggaaagattgtactcttacccgaggaggtctgatggatacgtgaagtgcgcttcataacctacttagtgataagtaactgaaccatcagaagtcagcagaggtcatagtacgaatcggtctagaacgattcggaaggactgaacaatcaagagaaaatagcccttggcattcagtacgtcatgatgaacacagaaaacatggtacctcccaagagaaaggaaacggtgaatcccgtgggaatcttttggagggtggagtgacgactggcataagaagatcagctatttacggaaggaagcttgcgtcattatcttgattgaaccgccgtatacggaaccgtacgtacggtggtgtgagaggacggaggttaatcacctcctcctactcgat

SEQ ID NO: 178 - DNA, 647 bp, Desulforamulus ferrireducens
  source 1..647: mol_type = unassigned DNA
gtatgcccggcatgggcgatagctttagggtgaaagtcccgaacaggggttggcaacaccaactgttagccaagagcaagggtgtccatcgtgaggtggaatctgaaagaagctggaggcaaattcccgacctgaggtacacgaatcacattcgaggctgttatattcgggcaagactgccagacaagtcaaagccctatgttgccaagggatatagcagtaaatgtggcaggtacatgggatgaaagttatcgctcttatccggggaggtctgtcgatccggtaatggtaaggaagtccttaacaataccaacccgcatcgtgaggtgtcggctgagtcggcagaagtcagcagaggccatagtactcctaacgaggaagggccgaaggtcagggcagactaagaacggtgcgttcgagagaatttgctgatagcagttgtctctgaaacagagacctatctataagagggagtggtgaagccacaatggcttatagaagggctgagaattctccggcacaatacccgaaaatcttaatcttaatcgctgagtaatcagttaaccctggccaaccgccggatgcggacccgcatgtccggtggtgtgggagggtcggcggagattatctccgccccctatcccaat

SEQ ID NO: 179 - DNA, 640 bp, Bacillus smithii
  source 1..640: mol_type = unassigned DNA
gtgcgcccggcatgtacatgaactatagggtgtaagtcccgaaccccgaagacagaagtagaggttagccaagagcaagggtgtccgtggtgacgcggaatctgaaggaagctggaggcaaaacaccggtccgaggaacacgaacctcatataaggctaggtatgattgagtgagtttgcataacaaaacaaagctctttctgtcgaaggtcatatcgagtaaatgaggcggatagatggtgtgaaagtgcatgtacttacccggggaggtctggcggatatgtgaagtactcttcataacctacttagtgataagtagctgaaccgtcagaagtcagcagaggtcatagtattagttggtctagaacaactaagaaggaccgaacaattaagagagaatagcccttggtattcagtgagtcatgatgaacacagaaaacgtagtacctcacttgagggaggaagcggtgaatcccgtgggagacctcttggagggtggagtgaccactggcataaagagaacagctattcacggaagttataaagacttgcgtcaattatcttaattgaaccgccgtatacggaaccgtacgtacggtggtgtgagaggacgggagttaatcgctccctcctactcgat

SEQ ID NO: 180 - DNA, 479 bp, Oceanobacillus iheyensis
  source 1..479: mol_type = unassigned DNA
gtgtgcccggcatgggtgcagtctatagggtgagagtcccgaactgtgaaggcagaagtaacagttagcctaacgcaagggtgtccgtggcgacatggaatctgaaggaagcggacggcaaaccttcggtctgaggaacacgaacttcatatgaggctaggtatcaatggatgagtttgcataacaaaacaaagtcctttctgccaaagttggtacagagtaaatgaagcagattgatgaagggaaagactgcattcttacccggggaggtctgatcgaaacgccaagcactcttggtaacccattcagcaatggatggctgaacggtcagaagtcagcagaagtcatagtaccctgcatactcgagaatgtaaggggaaggacggaacaattaagttcgcttaattgaaccgccgtataccgaacggtacgtacggtggtgtgagaggacgggggttagtcgctcccttctactctat

SEQ ID NO: 181 - DNA, 20 bp, Bacillus thuringiensis
  source 1..20: mol_type = unassigned DNA
caaaggcttacctatcacta

SEQ ID NO: 182 - DNA, 20 bp, Clostridium perfringens
  source 1..20: mol_type = unassigned DNA
aaaggataatagttggtatt

SEQ ID NO: 183 - DNA, 20 bp, Anoxybacillus pushchinoensis
  source 1..20: mol_type = unassigned DNA
ataaaagaggtggttttctt

SEQ ID NO: 184 - DNA, 20 bp, Desulforamulus ferrireducens
  source 1..20: mol_type = unassigned DNA
agccgttggctctttttctg

SEQ ID NO: 185 - DNA, 20 bp, Bacillus smithii
  source 1..20: mol_type = unassigned DNA
gatcagtccgtcttctttat

SEQ ID NO: 186 - DNA, 20 bp, Oceanobacillus iheyensis
  source 1..20: mol_type = unassigned DNA
aaaaccttccctttttttat

SEQ ID NO: 187 - DNA, 20 bp, Bacillus thuringiensis
  source 1..20: mol_type = unassigned DNA
cgcgacacgttcctaagtga

SEQ ID NO: 188 - DNA, 20 bp, Clostridium perfringens
  source 1..20: mol_type = unassigned DNA
tagcttattctatacctgac

SEQ ID NO: 189 - DNA, 20 bp, Anoxybacillus pushchinoensis
  source 1..20: mol_type = unassigned DNA
tatttctatgtgcaagagag

SEQ ID NO: 190 - DNA, 20 bp, Desulforamulus ferrireducens
  source 1..20: mol_type = unassigned DNA
taaactagcggataaaattc

SEQ ID NO: 191 - DNA, 20 bp, Bacillus smithii
  source 1..20: mol_type = unassigned DNA
tggtttgattcatggaaaaa

SEQ ID NO: 192 - DNA, 20 bp, Oceanobacillus iheyensis
  source 1..20: mol_type = unassigned DNA
tatttatcatttataaaaat

SEQ ID NO: 193 - DNA, 2569 bp, Bacillus thuringiensis
  source 1..2569: mol_type = unassigned DNA
gcgcgacacgttcctaagtgaaaagcttaggcactgtcgaactcaacagttcagcagtgaactgtcattctaagaagtcaaatgaaggagtaacgtctggaagggcttcccttaatcctccgacatgcaggaaagtaggcaagtactgaactgtgtgaagctcggtgaagtcggttgaaggttaccgtaaattagtatctctaatacgaaagctatccagcggtggatggtgtaactgatagaccggaggtctataaaacactcaaggttaggatgcgcgatgaactagaggcgatcgctagtaagcgcagacgaatccctgatggtacgggtctatatcgggagggaatcgaaaggttctctgacacaaataagtgtcgctactgtgggtgagtaaaactctcctttatgaaagcccatatatcgttacaggcgttattaaggtagcaggctcataggggaaacctaaaagtgtatgtacagataagaatgacggaacgtggtaagctgccgacatggagggcttgttctctttgaagtgttgccaaggaaagtcacaatgagattagttgtcgatataacttggtttaacggcagtgaaagtggtggcacagtaccgatgaaacgtgtaatgaacgtggagggatagccactagtcgattgaagattgaaggttactattggttaacatggtttcgagtaagactaagagatgtaatgctccaaagtaataaggaggttacagcccatgttaaagaaaaccaagctaagacataacgaatattatgatacacaaaaaaagtgtatgacaatttatactcgaacagtcttaacggtaacaatttctttcaattggaaacgatggaacgccgtatgcccggaaacgggcgcgtacggtgtggagtgggggaaaagctggagataatctcaaaggcttacctatcactatcgcgacacgttcctaagtgattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacaaaggcttacctatcacta

SEQ ID NO: 194 - DNA, 2454 bp, Clostridium perfringens
  source 1..2454: mol_type = unassigned DNA
gtgcgagacgttcatagttgaaaagactatgcacattctaaatataagagaatggagaactcttaacttgaaaagtggaatgatagggtaacgccttgaaacgctttctctaatactccgacatgcagtaactatgtgtgaaagttattgtgtgaagctcggtgaagtcggcagaaaattaccgtaatagataatattatctaacgaaagtgttctagaggtagaatatgtagttgatatgccgggagtcgttaaaaacagaatatggtgagaatgttccaataggaactgacgaactggcgaaggtacgctcctaagctataaatatagaaatatattaataaattggtttgaggattagtaagaatagtacccatgaaaatccttgatatgttataggcatataataagccaatggggtaaagtcagcacctaagttctagtattgataaagttaagggaacgtcgaaagctaaagacgtggaggatacacacctatgaagcgaagaaagggaatacataacctttcataatctttagtgagagtagtggcatgactgatgaagattttgtaatgaaattggaggaatagccacaagtcaattatttattaaaagaaacttaatcaattcatggattctagtaagataaaaaaggtcactgaatatagaaaattagtgatagaccatggcaagatatttatattaatacttgagattaagaaaaatgaatttaatatatacttgatggaacgccgtatgcggtgaaagtcgcacgtacggtgtggagcgggggaaaaattggagataacatcaaagatttacctatcgctattagcttattctatacctgacttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgaaaaggataatagttggtatt

SEQ ID NO: 195 - DNA, 2253 bp, Anoxybacillus pushchinoensis
  source 1..2253: mol_type = unassigned DNA
gtgcgctcggcatgggtgcaatctctagggtgaaagtcccgaactgcgaaggcagaagtagcagttagcttaacgcaagggtgtccgtggtgacgcggaatctgaaggaagcgggcggcaaacttccggtctgaggaacacgaacttcatataaggctaggtatcattggatgagtttgcaagacaaaacaaagtcctttctgccgaaggtgatacagagtaaatgaagcagatagatggaaggaaagattgtactcttacccgaggaggtctgatggatacgtgaagtgcgcttcataacctacttagtgataagtaactgaaccatcagaagtcagcagaggtcatagtacgaatcggtctagaacgattcggaaggactgaacaatcaagagaaaatagcccttggcattcagtacgtcatgatgaacacagaaaacatggtacctcccaagagaaaggaaacggtgaatcccgtgggaatcttttggagggtggagtgacgactggcataagaagatcagctatttacggaaggaagcttgcgtcattatcttgattgaaccgccgtatacggaaccgtacgtacggtggtgtgagaggacggaggttaatcacctcctcctactcgattatttctatgtgcaagagagttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgaataaaagaggtggttttctt

SEQ ID NO: 196 - DNA, 2267 bp, Desulforamulus ferrireducens
  source 1..2267: mol_type = unassigned DNA
gtatgcccggcatgggcgatagctttagggtgaaagtcccgaacaggggttggcaacaccaactgttagccaagagcaagggtgtccatcgtgaggtggaatctgaaagaagctggaggcaaattcccgacctgaggtacacgaatcacattcgaggctgttatattcgggcaagactgccagacaagtcaaagccctatgttgccaagggatatagcagtaaatgtggcaggtacatgggatgaaagttatcgctcttatccggggaggtctgtcgatccggtaatggtaaggaagtccttaacaataccaacccgcatcgtgaggtgtcggctgagtcggcagaagtcagcagaggccatagtactcctaacgaggaagggccgaaggtcagggcagactaagaacggtgcgttcgagagaatttgctgatagcagttgtctctgaaacagagacctatctataagagggagtggtgaagccacaatggcttatagaagggctgagaattctccggcacaatacccgaaaatcttaatcttaatcgctgagtaatcagttaaccctggccaaccgccggatgcggacccgcatgtccggtggtgtgggagggtcggcggagattatctccgccccctatcccaattaaactagcggataaaattcttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgaagccgttggctctttttctg

SEQ ID NO: 197 - DNA, 2260 bp, Bacillus smithii
  source 1..2260: mol_type = unassigned DNA
gtgcgcccggcatgtacatgaactatagggtgtaagtcccgaaccccgaagacagaagtagaggttagccaagagcaagggtgtccgtggtgacgcggaatctgaaggaagctggaggcaaaacaccggtccgaggaacacgaacctcatataaggctaggtatgattgagtgagtttgcataacaaaacaaagctctttctgtcgaaggtcatatcgagtaaatgaggcggatagatggtgtgaaagtgcatgtacttacccggggaggtctggcggatatgtgaagtactcttcataacctacttagtgataagtagctgaaccgtcagaagtcagcagaggtcatagtattagttggtctagaacaactaagaaggaccgaacaattaagagagaatagcccttggtattcagtgagtcatgatgaacacagaaaacgtagtacctcacttgagggaggaagcggtgaatcccgtgggagacctcttggagggtggagtgaccactggcataaagagaacagctattcacggaagttataaagacttgcgtcaattatcttaattgaaccgccgtatacggaaccgtacgtacggtggtgtgagaggacgggagttaatcgctccctcctactcgattggtttgattcatggaaaaattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgagatcagtccgtcttctttat

SEQ ID NO: 198 - DNA, 2099 bp, Oceanobacillus iheyensis
  source 1..2099: mol_type = unassigned DNA
gtgtgcccggcatgggtgcagtctatagggtgagagtcccgaactgtgaaggcagaagtaacagttagcctaacgcaagggtgtccgtggcgacatggaatctgaaggaagcggacggcaaaccttcggtctgaggaacacgaacttcatatgaggctaggtatcaatggatgagtttgcataacaaaacaaagtcctttctgccaaagttggtacagagtaaatgaagcagattgatgaagggaaagactgcattcttacccggggaggtctgatcgaaacgccaagcactcttggtaacccattcagcaatggatggctgaacggtcagaagtcagcagaagtcatagtaccctgcatactcgagaatgtaaggggaaggacggaacaattaagttcgcttaattgaaccgccgtataccgaacggtacgtacggtggtgtgagaggacgggggttagtcgctcccttctactctattatttatcatttataaaaatttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaaactagtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgaaaaaccttccctttttttat

SEQ ID NO: 199 - protein, 2425 aa, synthetic construct
MKRTADGSEFESPKKKRKVSEVEFSHEYWMRHALTLAKRARDEREVPVGAVLVLNNRVIGEGWNRAIGLHDPTAHAEIMALRQGGLVMQNYRLIDATLYVTFEPCVMCAGAMIHSRIGRVVFGVRNSKRGAAGSLMNVLNYPGMNHRVEITEGILADECAALLCDFYRMPRQVFNAQKKAQSSINSGGSSGGSSGSETPGTSESATPESSGGSSGGSDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGDSGGSKRTADGSEFEPKKKRKVGSGATNFSLLKQAGDVEENPGPMEDAKNIKKGPAPFYPLEDGTAGEQLHKAMKRYALVPGTIAFTDAHIEVDITYAEYFEMSVRLAEAMKRYGLNTNHRIVVCSENSLQFFMPVLGALFIGVAVAPANDIYNERELLNSMGISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKIAVSGGGSGGGSMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 200 - DNA, 8526 bp, synthetic construct
  source 1..8526: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaactagtgccaccatgaaacggacagccgacggaagcgagttcgagtcaccaaagaagaagcggaaagtctctgaggtggagttttcccacgagtactggatgagacatgccctgaccctggccaagagggcacgggatgagagggaggtgcctgtgggagccgtgctggtgctgaacaatagagtgatcggcgagggctggaacagagccatcggcctgcacgacccaacagcccatgccgaaattatggccctgagacagggcggcctggtcatgcagaactacagactgattgacgccaccctgtacgtgacattcgagccttgcgtgatgtgcgccggcgccatgatccactctaggatcggccgcgtggtgtttggcgtgaggaactcaaaaagaggcgccgcaggctccctgatgaacgtgctgaactaccccggcatgaatcaccgcgtcgaaattaccgagggaatcctggcagatgaatgtgccgccctgctgtgcgatttctatcggatgcctagacaggtgttcaatgctcagaagaaggcccagagctccatcaactccggaggatctagcggaggctcctctggctctgagacacctggcacaagcgagagcgcaacacctgaaagcagcgggggcagcagcggggggtcagacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggagccctgctgttcgacagcggcgaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggaaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacattgacggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgcttccgcccagagcttcatcgagcggatgaccaacttcgataagaacctgcccaacgagaaggtgctgcccaagcacagcctgctgtacgagtacttcaccgtgtataacgagctgaccaaagtgaaatacgtgaccgagggaatgagaaagcccgccttcctgagcggcgagcagaaaaaggccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggaccatatcgtgcctcagagctttctgaaggacgactccatcgacaacaaggtgctgaccagaagcgacaagaaccggggcaagagcgacaacgtgccctccgaagaggtcgtgaagaagatgaagaactactggcggcagctgctgaacgccaagctgattacccagagaaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaatgacaagctgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaaccggggagatcgtgtgggataagggccgggattttgccaccgtgcggaaagtgctgagcatgccccaagtgaatatcgtgaaaaagaccgaggtgcagacaggcggcttcagcaaagagtctatcctgcccaagaggaacagcgataagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaagcactacctggacgagatcatcgagcagatcagcgagttctccaagagagtgatcctggccgacgctaatctggacaaagtgctgtccgcctacaacaagcaccgggataagcccatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggtgactctggcggctcaaaaagaaccgccgacggcagcgaattcgagcccaagaagaagaggaaagtcggaagcggagctactaacttcagcctgctgaagcaggctggagacgtggaggagaaccctggacctatggaggacgccaagaacatcaagaagggccccgcccccttctaccccctggaggacggcaccgccggcgagcagctgcacaaggccatgaagcggtacgccctggtgcccggcaccatcgccttcaccgacgcccacatcgaggtggacatcacctacgccgagtacttcgagatgagcgtgcggctggccgaggccatgaagcggtacggcctgaacaccaaccaccggatcgtggtgtgcagcgagaacagcctgcagttcttcatgcccgtgctgggcgccctgttcatcggcgtggccgtggcccccgccaacgacatctacaacgagcgggagctgctgaacagcatgggcatcagccagcccaccgtggtgttcgtgagcaagaagggcctgcagaagatcctgaacgtgcagaagaagctgcccatcatccagaagatcatcatcatggacagcaagaccgactaccagggcttccagagcatgtacaccttcgtgaccagccacctgccccccggcttcaacgagtacgacttcgtgcccgagagcttcgaccgggacaagaccatcgccctgatcatgaacagcagcggcagcaccggcctgcccaagggcgtggccctgccccaccggaccgcctgcgtgcggttcagccacgcccgggaccccatcttcggcaaccagatcatccccgacaccgccatcctgagcgtggtgcccttccaccacggcttcggcatgttcaccaccctgggctacctgatctgcggcttccgggtggtgctgatgtaccggttcgaggaggagctgttcctgcggagcctgcaggactacaagatccagagcgccctgctggtgcccaccctgttcagcttcttcgccaagagcaccctgatcgacaagtacgacctgagcaacctgcacgagatcgccagcggcggcgcccccctgagcaaggaggtgggcgaggccgtggccaagcggttccacctgcccggcatccggcagggctacggcctgaccgagaccaccagcgccatcctgatcacccccgagggcgacgacaagcccggcgccgtgggcaaggtggtgcccttcttcgaggccaaggtggtggacctggacaccggcaagaccctgggcgtgaaccagcggggcgagctgtgcgtgcggggccccatgatcatgagcggctacgtgaacaaccccgaggccaccaacgccctgatcgacaaggacggctggctgcacagcggcgacatcgcctactgggacgaggacgagcacttcttcatcgtggaccggctgaagagcctgatcaagtacaagggctaccaggtggcccccgccgagctggagagcatcctgctgcagcaccccaacatcttcgacgccggcgtggccggcctgcccgacgacgacgccggcgagctgcccgccgccgtggtggtgctggagcacggcaagaccatgaccgagaaggagatcgtggactacgtggccagccaggtgaccaccgccaagaagctgcggggcggcgtggtgttcgtggacgaggtgcccaagggcctgaccggcaagctggacgcccggaagatccgggagatcctgatcaaggccaagaagggcggcaagatcgccgtgtctggtggcggatcaggtggcggatccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaagagacgctacggactt

SEQ ID NO: 201 - DNA, 8526 bp, synthetic construct
  source 1..8526: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaactagtgccaccatgaaacgtaccgccgatggcagcgaattcgagagtcctaaaaagaaaagaaaagtgagcgaggtggagttcagccacgagtattggatgcgccacgctctgacactggcaaagcgcgcgagagacgaacgggaagtgccagtgggcgcagtgctggtgttaaacaacagagtcatcggcgagggatggaacagagccatcggtctgcatgaccctactgcccatgccgaaatcatggctctgcgtcagggcggactggtgatgcagaactaccgcttaatcgacgccaccctgtatgtgacctttgagccttgtgtcatgtgtgctggcgcgatgatccactctagaataggtcgcgtcgtgttcggtgttcgcaatagcaagagaggagcagctggctctctgatgaacgtactgaactaccctggtatgaatcaccgcgtggaaatcaccgagggcatcctggccgacgagtgcgccgccttgctgtgtgatttctacaggatgcctagacaggtgttcaacgcgcagaagaaagcccagtcaagtattaactctgggggctcctccggcggcagtagcggctctgagacgcccggcacttccgaatcggctactcctgaaagcagcggtggatcaagcggaggttccgacaagaagtactctatcggcctcgccatagggaccaacagcgtgggttgggccgtgatcaccgacgagtacaaggttccctctaagaagttcaaggtgctgggaaatacggacagacactctatcaagaagaacctgatcggcgccctgctgttcgactccggcgagacagctgaggccacccgcctgaaaagaacagctagaagaagatacaccagaaggaagaacaggatctgctaccttcaggagattttctcaaacgagatggccaaagtggatgacagcttcttccacagactggaggagtctttcctggtggaggaggacaagaagcatgaacgtcaccccatcttcggcaacatcgtcgatgaggtcgcctaccacgagaagtaccccaccatctaccacctgcgaaagaagctggtggactccaccgacaaagcggatctgagactgatctacctggccttagctcatatgatcaagttccgcggccactttctcattgagggcgacctgaaccccgacaacagcgatgtggataaactgttcatccagctggtgcaaacatacaaccagctattcgaggaaaaccccatcaatgctagcggagtggacgccaaggctatccttagcgccagactgtccaagagccggaggttggaaaacctgatcgcccagttgcctggggaaaagaaaaatggcttattcggtaacttaatcgccctctccctgggcctcacccctaacttcaagagcaacttcgacctggccgaggacgccaagctgcagctctctaaggacacctacgacgacgatcttgacaacctgctggctcaaatcggcgaccagtacgccgatctcttcctggccgcgaagaacctgagcgatgcgatcctgctgagcgatatcctgcgggtgaatactgagatcaccaaggcccccctgtcagcttctatgatcaaacgttacgacgagcaccaccaggatctgacattgctgaaggctctggtgagacaacagctgcccgagaaatacaaagaaatattcttcgaccagtccaagaacggctacgctggctacattgacggtggggcgtctcaggaggagttctacaaatttatcaagcctattctggaaaagatggatggcaccgaagaactgttagtcaagctgaaccgcgaggacctgctgaggaagcagcggaccttcgacaacggcagtatccctcaccagatccatctgggcgaactgcacgcaattctgcgccgccaggaggacttctacccctttctgaaggacaaccgtgagaagattgaaaagatcctgacgttcagaatcccctattacgtgggccctctggctaggggtaattcgagatttgcttggatgacccggaaatccgaagaaaccattacgccatggaacttcgaagaagtagttgacaaaggcgcctcggcccagtccttcatcgagaggatgactaacttcgacaaaaatttgcctaacgagaaagtgctgcccaagcacagcctgctctacgagtactttactgtgtacaacgagctgacgaaagtgaaatacgtgacagaaggtatgagaaagcccgccttcctcagcggcgagcagaagaaggccattgtcgacctgctgttcaagacgaaccgcaaggtcaccgtgaaacagctgaaagaggattatttcaagaagatcgaatgctttgattccgtggagatcagcggtgttgaggacagattcaatgcctcgctgggcacctaccacgacctgctgaagattatcaaggataaggacttcctggacaatgaggaaaacgaagatatcctggaggacatcgttttaacattaaccctcttcgaggaccgggagatgatcgaggaacgtctgaaaacctacgcccatttattcgacgacaaggtgatgaagcagctaaagagacgccgctacaccggctggggccggctgagccggaagctaatcaacggcatcagagataagcagtctggcaagaccattctggacttcttgaagtctgacggcttcgccaatagaaacttcatgcagctgatccacgacgactctctcacattcaaggaagatatccagaaggctcaggtgtccggccagggcgacagtctgcacgagcacatcgccaacctggcaggctctccagccatcaagaagggtatcctgcagaccgtgaaggtcgtggacgagctggtgaaggtcatgggacgtcacaagccagagaacatcgttattgagatggcgagagagaaccagacgacccagaagggccagaagaacagccgggagcgcatgaagagaatcgaggaaggcatcaaagaactcggcagccagatactgaaggaacaccctgtcgagaatacccagctgcagaacgagaagctatacctgtactacctccagaacggtcgcgacatgtacgtagaccaggagctagacatcaacaggttaagcgattacgatgttgatcacatcgtccctcagtccttcctcaaggacgatagcatcgacaacaaggtcctcacacgaagcgataagaatcgtgggaagtccgacaatgtgcctagcgaggaagtggtcaagaagatgaagaattactggcggcagcttcttaatgcaaagctgataacacagagaaaatttgacaacctaactaaggccgagagaggaggactgagcgagctggacaaggcaggattcatcaaaagacaactggttgaaaccaggcaaattaccaagcacgtggcccagatcctggacagcaggatgaacacgaagtacgacgagaacgacaaactaatccgggaagtaaaggtgatcaccctgaagtccaagctcgtgtccgacttcaggaaggacttccaattctacaaggttcgggagattaacaactaccaccacgcgcatgacgcatacctgaatgccgtagtaggcaccgcgctgataaagaagtacccaaagctggagtcagagttcgtgtacggagactacaaagtgtacgacgtgagaaaaatgatcgcaaagagcgagcaggagatcggcaaagcgaccgcaaaatacttcttttactccaacataatgaactttttcaaaacagagatcaccctcgcaaatggcgagatcagaaagcggcccctgatcgagaccaacggagaaactggtgaaattgtatgggataaaggcagagatttcgccaccgtccgcaaggtcctgtccatgcctcaggttaacatcgtgaaaaagaccgaagttcagacgggcggcttctctaaggaaagtatcctgcccaaacggaactctgacaagttaatagcccgcaaaaaagactgggacccaaaaaagtacggcggcttcgacagccctaccgtggcctattccgtcctggttgtggctaaggtggagaagggcaaatcgaagaagctgaagtcggttaaagagctgctaggcatcaccatcatggagcgaagttcctttgagaagaaccctatcgattttctggaagccaagggatacaaggaggtgaagaaagacctgattatcaaactgcccaagtactccctgtttgagctagagaatggccggaagagaatgctggcctcggcaggggaattgcaaaagggcaacgagctcgccctacctagcaagtatgtcaacttcctgtacctggctagccattacgagaagctgaagggaagcccagaggataacgagcaaaaacagctctttgtcgaacagcacaaacactacttggacgagatcatcgagcagatctctgagtttagcaagcgcgtgatcctagcggacgccaacctggacaaggtgctgagcgcctacaataagcaccgtgacaagcccatccgcgagcaggccgagaacattatccacctgttcaccctgaccaacctgggtgcacccgccgcttttaagtacttcgacaccactattgacagaaagcgctacacgtcgaccaaggaggtgctggacgctacattgatacaccagagcatcaccggcctttacgagacaagaattgacctgtctcagctgggtggggactctggcggttctaagcggaccgccgatgggtcggagttcgagcccaagaagaagcggaaggttggttccggcgccaccaacttcagcctgctcaagcaggccggcgacgtggaggagaatcctggcccgatggaggacgccaagaacatcaagaaaggtcctgctcccttctaccctctagaggacggcaccgcaggcgagcaattgcacaaagccatgaagcggtacgcgttagtgccggggaccatcgccttcaccgacgctcacatcgaggtcgatatcacctatgccgagtacttcgagatgagcgtgcggctggccgaggccatgaagaggtacggcctgaacaccaaccaccggatcgtcgtgtgctctgaaaacagcttacagttcttcatgcccgtcctgggagcgctgttcatcggggtggccgtagctcctgccaacgacatctacaacgagcgggagctgctgaacagcatgggcatctctcagccaaccgtggtgttcgtgtcgaagaagggactacagaagattctgaacgtgcagaagaaactgcccattatccagaagatcataatcatggattccaaaacagactaccaaggctttcagagcatgtataccttcgtcaccagccacctgcccccagggttcaacgagtacgactttgtgccagagagctttgatagagacaagaccatcgctctgatcatgaactccagcgggagcaccggcctgccaaagggcgttgccctgcctcatagaaccgcctgcgtgcgcttcagccacgcccgtgatcctatcttcggcaaccagatcatccctgatacagctatcctgagcgtggtccccttccaccatggattcggaatgtttaccaccctcggctacctgatttgcgggttccgagtagtcctcatgtatagattcgaggaggagctgtttctcaggagcctccaggactacaagatccagtccgctcttctcgtgcctacccttttcagcttcttcgctaagagtacgttaatcgacaagtatgacttgagtaacctgcacgaaattgcgagcggaggcgccccgctgtccaaagaagtgggcgaggcagtggctaagagattccacttacccggaatccgtcagggctacggcctgacagaaactaccagcgccattctcattactccggagggcgacgacaagcccggcgccgtggggaaggtggtgcctttcttcgaggccaaggtggtggacttagacaccggcaaaaccctaggcgtcaaccagagaggagagctgtgcgtgcggggccctatgataatgtctggctacgttaacaacccagaggctaccaacgctctgatcgacaaggacggctggctgcacagcggcgacatcgcctattgggatgaggacgaacacttcttcatcgtggaccgtcttaagtctctgataaagtacaaaggctaccaggtcgcccctgctgagctggagtctatccttttgcagcaccctaatatttttgacgccggcgtggctggattacccgacgatgacgcgggcgagctgcctgccgccgtggtagttctcgaacacggtaagaccatgaccgaaaaggagatcgtggattacgtggcaagccaagtgaccaccgccaagaagctgcggggaggcgtagtatttgtcgacgaggtaccgaagggcctgactggtaagctggacgcccgcaaaatacgggagattctgatcaaggctaagaagggcggcaagatcgccgtgtccggcggtggatccggcggcggatccatggtgtccaaaggagaggagctgtttacaggcgtggtcccgatcctggtggagctggatggagatgtgaatggccacaaattttccgtgtctggggagggcgagggggacgcgacctatggcaagctgacactgaaatttatctgcactacaggcaagctacctgtgccgtggcctacgctggtgaccactctgacctacggcgtgcagtgttttagccgataccccgaccacatgaagcagcatgacttttttaaatcagccatgccagaaggctatgtgcaggagcgtacaatattcttcaaagacgacggcaactacaagaccagagccgaagtcaagttcgagggcgacaccctggtgaacagaatcgagctgaagggtatcgatttcaaagaggatggcaacattctgggccacaaattggagtacaactacaacagtcacaacgtatacatcatggcggacaagcagaagaatggtatcaaggtgaattttaagatcagacacaacattgaggatgggagcgttcagctggctgaccattaccagcagaacacccctatcggagatggacctgtgctcctgccagataaccactacctgtccacccagtccgctctgagcaaggaccccaatgagaagcgagatcacatggtgttgctggagttcgtgaccgccgccggcatcaccctgggcatggacgagctgtacaagtgaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaagagacgctacggactt

SEQ ID NO: 202 - DNA, 8526 bp, synthetic construct
  source 1..8526: mol_type = other DNA
ggaaataattgagccttaaagaagaaattctttaagtggatgctctcaaactcagggaaacctaaatctagttatagacaaggcaatcctgagccaagccgaagtagtaattagtaagttaacaatagatgacttacaactaatcggaaggtgcagagactcgacgggagctaccctaacgtcaagacgagggtaaagagagagtccaattctcaaagccaataggcagtagcgaaagctgcaagagaatgaaaatccgttgaccttaaaaacaaaaaacaaaaaaagccgtgtatagtgaacccgacaaaaaaaaaaccaaaaaaacaaaacacattaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaactagtgccaccatgaagcgcaccgccgacgggtctgagtttgaatcccctaagaagaaaagaaaggtcagcgaagtggagttcagccacgagtattggatgcggcatgccctgaccctcgctaagcgagccagagacgagagagaagtacccgtgggcgcggtgctggttctgaataaccgggtgatcggcgaaggatggaacagagccatcggcctccatgatccaaccgcccatgccgagatcatggccctgagacagggcggcctggttatgcagaactaccgcttgatagacgccacactgtacgttacattcgaaccgtgcgtgatgtgtgccggcgccatgatacacagcagaatcggcagagtggtgtttggagtgcggaactccaagcggggagctgccggaagcctgatgaacgtgttgaactaccctggaatgaaccacagagtggagatcaccgagggcatcctggctgatgagtgcgccgctctgctctgtgatttctacagaatgcctagacaggtgttcaatgcccagaagaaggcccagagcagcatcaacagcggcggcagctctggtggctctagcggctctgagacacctggcactagcgagagcgccacccctgagagctccggtggctccagcggcggctcggataagaaatatagcatcggcctggcaatcggcacaaacagcgtgggctgggccgtgatcaccgatgagtacaaggtgccttctaaaaagttcaaggtgctgggcaataccgacagacacagcatcaagaagaacctgatcggcgctctgctgttcgactccggcgagactgccgaggccactaggctgaagaggacagcccgtcgtagatataccagaagaaagaaccggatctgctacctgcaggagatcttcagcaacgagatggccaaagtggacgactcattcttccaccggttggaagaaagtttcctggtggaggaagataagaaacacgagagacatccaatcttcggcaacattgtcgacgaggttgcctaccatgagaaataccctaccatctaccacctgcggaagaagctggtggattctacagataaggccgatctgcggctgatctacctggcgctggcccacatgatcaagttcagaggacacttcctcatcgaaggcgatctgaaccccgataacagcgacgtggacaagctgttcatccagctggtgcagacctacaaccagctgtttgaagaaaatcctatcaacgccagcggcgttgacgccaaggccatattgagcgcccggctgagcaagagtagacggctggagaacctgatcgcccagctgcccggcgagaagaagaacggcctgtttggcaacctgatcgccctgagcttgggcctgacccctaacttcaagagcaacttcgacctagccgaggacgccaagctgcagctgagcaaggacacctacgatgatgacctggacaacctcctggcccagattggggatcaatacgccgacctgtttctggcggctaaaaacttgtctgacgccatcctgctgagcgacatcctgcgggtgaacaccgagatcacaaaggcccctctgtcagccagcatgatcaagagatacgacgagcaccaccaggacctgaccctgctgaaagctctggtgcggcagcagctgcccgagaagtacaaggaaatcttcttcgaccaaagcaaaaatggctacgccggctacatcgatggcggcgctagccaggaagagttctataagtttattaagcctatcctggaaaaaatggacggcaccgaggaactgctggtgaagctgaaccgggaggatctgctgcggaagcagagaacattcgacaacggcagcatccctcaccagattcacctgggagagctgcacgccattctgcgtagacaagaggacttctaccccttcttaaaggacaaccgcgagaaaatcgaaaagatcttaacatttcggatcccctactacgtgggccctctggcacggggaaattctagattcgcctggatgaccagaaagtctgaagaaaccataacaccttggaacttcgaggaggtggtggataagggcgccagcgctcagagcttcatcgaacggatgaccaacttcgacaagaacctgccaaacgagaaggtgctgcccaagcacagcctgctgtatgaatacttcaccgtgtacaatgagctgacaaaagtgaagtacgtgacagaaggcatgcggaagcccgccttcttgtcaggcgagcaaaaaaaggcaatcgtggatctgctgtttaagacaaacagaaaggtgaccgtgaagcagctcaaggaggactacttcaaaaagatcgaatgcttcgacagcgtcgagatatctggcgtggaggaccggttcaacgctagcctgggcacctaccacgacctgctgaagatcatcaaggacaaggatttcctggacaatgaagaaaatgaagatatcctggaggacatcgttctgacactgaccctgtttgaggatagagaaatgatcgaagaacggctaaaaacctatgcccacctgttcgatgacaaagtgatgaagcagctgaagcgaagaagatacacaggctggggccgtctaagcagaaagctgatcaacggtatccgggacaagcaatctggcaagaccatcctcgattttctgaagagtgacggcttcgccaatagaaactttatgcagctgatccacgatgatagcctgaccttcaaagaggacatccagaaggcgcaggtgagcggccagggggactctctgcacgagcacatcgccaacctggccggcagccctgccatcaagaagggaattctgcaaaccgtgaaggtggtagacgaactggtgaaggtgatgggcagacacaagcctgagaatatcgtgatcgagatggccagagagaaccagaccacacagaagggccagaaaaattctcgggaaagaatgaaaagaatcgaggagggcattaaagaactggggagccagatccttaaggagcaccccgttgaaaacacccagttacagaacgagaagctgtacctgtattacctgcaaaacggcagagatatgtacgtggaccaggagctggacatcaacagactgtctgactatgacgtggaccacatcgtgccccaaagtttcctgaaggacgacagcatcgataacaaggtgctgaccagaagcgataagaacagaggcaagagcgacaacgtgcctagcgaggaagtggtgaaaaagatgaagaactactggcggcaactgctcaatgccaaactgatcacccagagaaagttcgataacctcaccaaggccgagaggggcggcctgagcgaactggacaaggccggcttcatcaaaagacaactggtggaaaccagacagatcaccaagcacgtggcccagatcctggacagcagaatgaacacaaagtacgatgagaacgataagttaatcagagaggtgaaggtaatcacactgaagagcaagctggtgtccgatttcagaaaggatttccagttctacaaggtcagagaaatcaataactaccaccacgcccacgacgcctacctgaacgccgtggtcggcaccgccctgatcaagaagtaccctaaactggagagcgagttcgtgtacggcgactacaaggtttacgacgtgcggaagatgatcgccaagagcgagcaggagattggaaaggctacagccaaatacttcttctacagcaacatcatgaatttctttaagaccgagatcaccctggccaacggtgaaatccggaagagacccctgattgagaccaatggagagactggcgagatcgtgtgggataaaggcagagatttcgccaccgtgcggaaagtgctgtctatgcctcaggtgaacatcgtgaagaaaaccgaggtccagaccggcggattcagcaaggagtctatcctgcctaagaggaattccgacaagttaatcgctagaaagaaggactgggaccctaagaaatacggcgggttcgacagtcctaccgtcgcctattcggtgctggtggtggccaaagtggaaaaaggaaaatcaaagaagctgaaaagcgtgaaggagctgctgggaatcaccatcatggaacggagctctttcgagaagaaccccatcgatttcctggaagccaagggctacaaggaagtgaagaaagatctgatcatcaagctgcctaaatactctctgttcgaactggaaaatggccggaagagaatgctggcgagcgccggagagttgcagaaggggaatgagctggcactgccaagcaagtacgtgaacttcctgtacctggcctctcactacgagaagctgaagggcagcccagaggacaacgagcagaagcagttgttcgtggagcagcacaagcactacctggacgagatcatcgagcagatcagcgaattctccaagcgggtgatcctggccgatgccaacctggacaaggtgctgagcgcttacaataagcacagagataagcctatcagagagcaggccgaaaacatcatccacctgtttaccctcacaaacctcggcgcccctgctgcttttaagtactttgatacaaccatcgacagaaagaggtacaccagcaccaaggaggtgctggacgccacactgatccaccagtccatcacaggcctgtacgagacaagaatagatctgtcccagctggggggcgacagcggaggatccaagcggaccgctgacggcagcgagttcgagccaaagaaaaagcggaaggtgggttccggcgccaccaacttcagcctgctcaagcaggccggcgacgtggaggagaatcctggcccgatggaggacgccaagaacatcaagaaaggtcctgctcccttctaccctctagaggacggcaccgcaggcgagcaattgcacaaagccatgaagcggtacgcgttagtgccggggaccatcgccttcaccgacgctcacatcgaggtcgatatcacctatgccgagtacttcgagatgagcgtgcggctggccgaggccatgaagaggtacggcctgaacaccaaccaccggatcgtcgtgtgctctgaaaacagcttacagttcttcatgcccgtcctgggagcgctgttcatcggggtggccgtagctcctgccaacgacatctacaacgagcgggagctgctgaacagcatgggcatctctcagccaaccgtggtgttcgtgtcgaagaagggactacagaagattctgaacgtgcagaagaaactgcccattatccagaagatcataatcatggattccaaaacagactaccaaggctttcagagcatgtataccttcgtcaccagccacctgcccccagggttcaacgagtacgactttgtgccagagagctttgatagagacaagaccatcgctctgatcatgaactccagcgggagcaccggcctgccaaagggcgttgccctgcctcatagaaccgcctgcgtgcgcttcagccacgcccgtgatcctatcttcggcaaccagatcatccctgatacagctatcctgagcgtggtccccttccaccatggattcggaatgtttaccaccctcggctacctgatttgcgggttccgagtagtcctcatgtatagattcgaggaggagctgtttctcaggagcctccaggactacaagatccagtccgctcttctcgtgcctacccttttcagcttcttcgctaagagtacgttaatcgacaagtatgacttgagtaacctgcacgaaattgcgagcggaggcgccccgctgtccaaagaagtgggcgaggcagtggctaagagattccacttacccggaatccgtcagggctacggcctgacagaaactaccagcgccattctcattactccggagggcgacgacaagcccggcgccgtggggaaggtggtgcctttcttcgaggccaaggtggtggacttagacaccggcaaaaccctaggcgtcaaccagagaggagagctgtgcgtgcggggccctatgataatgtctggctacgttaacaacccagaggctaccaacgctctgatcgacaaggacggctggctgcacagcggcgacatcgcctattgggatgaggacgaacacttcttcatcgtggaccgtcttaagtctctgataaagtacaaaggctaccaggtcgcccctgctgagctggagtctatccttttgcagcaccctaatatttttgacgccggcgtggctggattacccgacgatgacgcgggcgagctgcctgccgccgtggtagttctcgaacacggtaagaccatgaccgaaaaggagatcgtggattacgtggcaagccaagtgaccaccgccaagaagctgcggggaggcgtagtatttgtcgacgaggtaccgaagggcctgactggtaagctggacgcccgcaaaatacgggagattctgatcaaggctaagaagggcggcaagatcgccgtgtccggcggtggatccggcggcggatccatggtgtccaaaggagaggagctgtttacaggcgtggtcccgatcctggtggagctggatggagatgtgaatggccacaaattttccgtgtctggggagggcgagggggacgcgacctatggcaagctgacactgaaatttatctgcactacaggcaagctacctgtgccgtggcctacgctggtgaccactctgacctacggcgtgcagtgttttagccgataccccgaccacatgaagcagcatgacttttttaaatcagccatgccagaaggctatgtgcaggagcgtacaatattcttcaaagacgacggcaactacaagaccagagccgaagtcaagttcgagggcgacaccctggtgaacagaatcgagctgaagggtatcgatttcaaagaggatggcaacattctgggccacaaattggagtacaactacaacagtcacaacgtatacatcatggcggacaagcagaagaatggtatcaaggtgaattttaagatcagacacaacattgaggatgggagcgttcagctggctgaccattaccagcagaacacccctatcggagatggacctgtgctcctgccagataaccactacctgtccacccagtccgctctgagcaaggaccccaatgagaagcgagatcacatggtgttgctggagttcgtgaccgccgccggcatcaccctgggcatggacgagctgtacaagtgaccggtgctggagcctcggtggccatgcttcttgccccttgggcctccccccagcccctcctccccttcctgcacccgtacccccgtggtctttgaataaagtctgacgggttcactatacacggcaaaaaacaaaaaacaaaagagacgctacggactt

SEQ ID NO: 203 - DNA, 1452 bp, synthetic construct
  source 1..1452: mol_type = other DNA
ttaaaacagcctgtgggttgatcccacccacaggcccattgggcgctagcactctggtatcacggtacctttgtgcgcctgttttataccccctcccccaactgtaacttagaagtaacacacaccgatcaacagtcagcgtggcacaccagccacgttttgatcaagcacttctgttaccccggactgagtatcaatagactgctcacgcggttgaaggagaaagcgttcgttatccggccaactacttcgaaaaacctagtaacaccgtggaagttgcagagtgtttcgctcagcactaccccagtgtagatcaggtcgatgagtcaccgcattccccacgggcgaccgtggcggtggctgcgttggcggcctgcccatggggaaacccatgggacgctctaatacagacatggtgcgaagagtctattgagctagttggtagtcctccggcccctgaatgcggctaatcctaactgcggagcacacaccctcaagccagagggcagtgtgtcgtaacgggcaactctgcagcggaaccgactactttgggtgtccgtgtttcattttattcctatactggctgcttatggtgacaattgagagatcgttaccatatagctattggattggccatccggtgactaatagagctattatatatccctttgttgggtttataccacttagcttgaaagaggttaaaacattacaattcattgttaagttgaatacagcaaaatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaa

SEQ ID NO: 204 - DNA, 336 bp, synthetic construct
  source 1..336: mol_type = other DNA
atggttcctcgcatgattgtcaccccccagagcaatcggcctgtcatgggtattgtgcaggacacactcacagcagcgcgcaaattcaccaagagagacgtcttcctggagcgggtggaacggcacatgtgtgatggggacattgttatcttcaaccggcagccaactctgcacaaaatgtccatgatggggcatcgggtccgcattctcccatggtctacctttcgcttgaatcttagtgtgacaactccgtacaatgcagactttgacggggatgagatgaacttgcacctgccacagtctctggagacgcgagcagagatccaggagctggcc